(19) United States
(12) Patent Application Publication
(10) Pub. No.: US 2024/0390364 A1
(43) Pub. Date: Nov. 28, 2024

(54) Novel Pyridine Compositions and their use in methods for preventing or treating diseases, disorders and conditions

(71) Applicant: Christopher Brian Reid, Los Angeles, CA (US)

(72) Inventors: Christopher Brian Reid, Los Angeles, CA (US); Que T. Collins, Santa Monica, CA (US)

(21) Appl. No.: 18/795,229

(22) Filed: Aug. 6, 2024

Related U.S. Application Data
(63) Continuation-in-part of application No. 16/134,723, filed on Sep. 18, 2018.
Application No. 16/134,723 is a continuation of application No. PCT/US2017/033234, filed on May 18, 2017.
Application No. 16/134,723 is a continuation of application No. 16/039,134, filed on Jul. 18, 2018, now Pat. No. 10,561,650.
Application No. 16/039,134 is a division of application No. 15/241,308, filed on Aug. 19, 2016, now abandoned.
Application No. 15/241,308 is a division of application No. 13/815,664, filed on Mar. 14, 2013, now abandoned.
(60) Provisional application No. 63/531,020, filed on Aug. 7, 2023.
(60) Provisional application No. 62/390,081, filed on Mar. 18, 2016.
(60) Provisional application No. 62/390,438, filed on Mar. 29, 2016.

(51) Int. Cl. A61K 31/496; A61K 31/12; A61K 31/122; A61K 31/194; A61K 31/327; A61K 31/355; A61K 31/375; A61K 31/4164; A61K 36/00; A61K 38/06; A61K 38/18; A61K 45/06

(52) U.S. Cl. CPC A61K 31/496; A61K 31/12; A61K 31/122; A61K 31/194; A61K 31/327; A61K 31/355; A61K 31/375; A61K 31/4164; A61K 36/00; A61K 38/063; A61K 38/1816; A61K 45/06; Y02A 50/30

(57) ABSTRACT

The claimed invention is directed towards a composition for promoting NR4A2 (nurr1) activity, CREB1 activity, NGF-independent TRKA activation, terpenoid backbone biosynthesis, or any combination thereof, in a cell, tissue, organ, subject, patient and/or organism, wherein the composition comprises an aminopyridine, its derivatives, its metabolites, its analogs, and/or its isomers. The invention also provides a method for promoting NR4A2 (nurr1) activity, CREB1 activity, NGF-independent TRKA activation, terpenoid backbone biosynthesis, or any combination thereof, in a cell, tissue, organ, subject, patient and/or organism, wherein said method comprises treatment of said cell, tissue, organ, organism, individual, and/or patient with the composition.

### CROSS REFERENCE TO RELATED APPLICATIONS

The application is a continuation-in-part of co-pending application Ser. No. 16/134,723 filed on Sep. 18, 2018, and claims the benefit of provisional application No. 62/390,081 filed on Mar. 18, 2016, and provisional application No. 62/390,438, filed Mar. 29, 2016, and provisional application 63/531,020 filed on Aug. 7, 2023.

### FIELD OF THE INVENTION

The invention relates to novel uses and compositions comprising known compounds, especially approved compounds and FDA approved drugs.

### BACKGROUND OF THE INVENTION

In industrialized countries, cancer, neurological disease, cardiovascular disease, chronic disease, inflammatory disease and degenerative disease represent major causes of morbidity and mortality, yet drug development is slow and expensive. As a consequence, effective drugs are frequently still lacking. Thus, there is a strong rationale for adding novel formulations, compositions and uses of previously approved drugs so as to benefit the largest number of people and animals possible. The present invention teaches such novel formulations, compositions and uses in conjunction with disclosing previously unknown activities of pyridines, especially aminopyridines.

### SUMMARY OF THE INVENTION

Any person, animal, and indeed some plants, people could eventually benefit from novel, efficacious drug compositions such as those describe herein. The present invention provides drug compositions for maintaining health, and/or preventing and/or treating various conditions, diseases, and maladies. The present invention also provides corresponding methods for producing such compositions and methods of maintaining health, and/or preventing a condition, disorder or disease and/or treating a condition, disorder or disease in a human, animal or plant, wherein the compositions, manufacture, or products of the present invention are provided to said plant, animal or human.

The Summary above is provided to introduce a selection of concepts in a simplified form that are further described below in the Detailed Description. This Summary is not intended to identify key features or essential features of the claimed subject matter, nor is it intended to be used to limit the scope of the claimed subject matter.

### DETAILED DESCRIPTION OF THE INVENTION

The development of new drugs is costly and protracted. Repurposing existing drugs leverages experimental, in Silico, and computational approaches to identify novel uses for these compounds.

The present invention provides compositions, manufacture, products, processes, methods, and/or methods of use, for prevention, treatment and health maintenance by increasing the recognized uses and benefits of individual drugs and compounds, including uses of aminopyridines and pyridines.

### NR4A2 (Nurr1), CREB, and Terpenoid Biosynthesis

NR4A2 is a transcription factor that is essential for embryonic development and maintenance of dopaminergic neurons. NR4A2 is a downstream target of CREB. In microglia and astrocytes, NR4A2 inhibits the expression of proinflammatory mediators. NR4A2 is implicated in AD progression; its activation may improve cognitive function; and it is downregulated in peripheral blood mononuclear cells of MS patients; (Jakaria et al., 2019).

Flavonoids are polyphenolic compounds found primarily in plants while terpenoids are present in all classes or organisms. Flavonoids display “a wide range of pharmacological activities such as antioxidant, anti-inflammatory, metal ions chelating, vasoprotective, anti-infective, hepatoprotective and anticancer. Flavonoids are also neurologically active as they have shown protection against neuroinflammation and neurotoxins. They have also been reported to improve cognitive functions” (Sharma et al. 2019).

Terpenoids, also known as isoprenoids, are a large class of natural products consisting of isoprene (C5) units. Terpenoid synthesis in animals occurs via the mevalonate pathway. The action of prenyltransferases then generates higher-order building blocks: geranyl diphosphate (GPP), farsenyl diphosphate (FPP), and geranylgeranyl diphosphate (GGPP), which are the precursors of monoterpenoids (C10), sesquiterpenoids (C15), and diterpenoids (C20), respectively. Condensation of these building blocks gives rise to the precursors of sterols (C30) and carotenoids (C40).
“The mevalonate acid pathway is an important primary, cellular, metabolic pathway, which is present in all higher eukaryotes and many bacteria. It serves, as the basis for the biosynthesis of DMAPP and IPP, not only for the production of useful for the defense of the organism secondary metabolites, but also for protein prenylation, cell membrane maintenance, protein anchoring, etc.” (Zografos and Anagnostaki, 2016).

Acetyl-CoA is a precursor molecule to terpenoids and fatty acid derived molecules (Yuan and Ching, 2016). Meanwhile, NR4A2 binds fatty acid metabolites and fatty acid mimetic (FAM) drugs.

According to Sharma et al. (2019),
“ . . . bioactive molecules that alter the activity of CREB in a way to have specialized effects in different brain regions and neural circuits involved could potentially be utilized for therapeutic purposes. Flavonoids are the polyphenolic compounds which lead to phosphorylation of CREB in the hippocampus . . . ”

In the present invention, we find that 4-aminopyridine surprisingly and dramatically regulates gene expression associated with NR4A2 and CREB1 transcription factors and the human, terpenoid backbone biosynthesis pathway. This regulation has significant implications across the spectrum of disorders, diseases, conditions, as well as for health maintenance, generally.

Embodiments, agents, compounds or drugs of the present invention may replace an equal or larger number of approved drugs in treating a patient.

In one aspect, the present invention relates to nitrogen heterocyclic compounds.

Nitrogen heterocyclic compounds, especially pyridine derivatives, represent important constituents of biologically active natural products and are frequently incorporated into synthetic compounds. The interest of Medicine and Pharmaceutical Science in pyridine containing molecules is persistent and increasing.

4-Aminopyridine (4-AP) is a nitrogen heterocycle known to be a non-selective blocker of voltage-dependent K+-channels and is believed to exert its effects and produce its benefits via that mechanism.

As used herein, 4 aminopyridine and 4-aminopyridine(s) refer to useful pyridine, aminopyridine, 4 aminopyridine, a member or members of the class of aminopyridines, any of their analogs, any of their derivatives, any pro-drugs or active metabolite(s)—whether naturally occurring or synthetically derived. To the extent that they have the same or similar benefits described herein, they are covered by this invention.

Other isomeric amines of pyridine, to the extent that they have the same or similar benefits as 4-aminopyridine, are covered by this invention.

As used herein, the term “benefit” represents, at various moments, maintenance of a healthy state. improvement, mitigation, amelioration, palliation, reduced severity, restoration of homeostasis, prevention, regeneration, rejuvenation, physical relief, mental relief, pain relief, life extension, slowed degeneration, slowed aging, or other desirable outcome.

As used herein, the term “4-aminopyridine transcriptome” refers to the global pattern of gene expression induced by 4-aminopyridine in animal cells, especially mammalian cells, and especially human cells, especially as defined and detailed in the research data herein.

As used herein, “modulates” is defined as regulates or refers to beneficially increasing or decreasing, e.g. modulating gene expression.

As used herein, “recovery” refers to a return to a prior state, prior healthy state, or state of health; or restoration of a prior state of health, e.g. a prior state of gene transcription.

As used herein “repair” refers to a return to a prior appropriate state, prior healthy state, or state of health and may relate to repair of a molecule, genetic sequence, protein, cell, tissue, organ, organism, or individual. The present invention teaches that the 4-aminopyridine transcriptome specifically commends 4-aminopyridine to a wide variety of uses detailed, described, listed and/or imputed herein.

In some embodiments, the compositions, manufacture, products, processes, methods, and/or methods of use, prevention and treatment of the present invention comprise an analog of a compound, agent, or drug named herein.

In some embodiments, the compositions or formulations described herein comprise nationally approved agents, compounds or drugs and/or otherwise available agents, compounds or drugs belonging to the drug classes, for example drugs of the class represented by dalfampridine (aka Ampyra), an example of a 4-aminopyridine. See Pruunsild et al (2016).

The present invention further discloses several, bioinformatics analyses of bicuculline and 4-aminopyridine activated neurons-simultaneously uncovering previously unknown biological activities and supporting new uses for compounds of the present inventionin a wide variety of health states, diseases, disorders, conditions, and contexts.

In some preferred embodiments, 4-aminopyridine, or a compound in the class of 4-aminopyridine, influences, treats and/or provides benefit(s) to a cell, tissue, organ, subject, patient, animal and/or organism by modulating its gene expression as described herein.

In some embodiments, 4-aminopyridine, or a compound in the class of 4-aminopyridine, modulates the expression of RNA and/or protein.

In some embodiments, 4-aminopyridine, or a compound in the class of 4-aminopyridine, or a compound in the class of 4-aminopyridine, modulates the expression of at least one genetic sequence, RNA, mRNA, coding RNA, non-coding RNA, long non-coding RNA, short RNA, miRNA, other RNA, and/or combinations thereof.

In some embodiments, 4-aminopyridine, or a compound in the class of 4-aminopyridine, beneficially modulates the expression of at least one or more genetic sequence(s) and/or protein(s) selected from structural proteins, signal transduction proteins, regulatory proteins, transcription factors, oncogenes, tumor suppressors, non-coding RNA, miRNAs, oncomirs, inflammamirs, pro-inflammatory mediators, cytokines, etc. and/or combinations thereof.

In some embodiments, 4-aminopyridine, or a compound in the class of 4-aminopyridine, influences, treats and/or provides benefit(s) to a cell, tissue, organ, subject, patient, animal and/or organism, at least in part, by modulating the expression of at least one or more protein and/or genetic sequence selected from those taught herein, and/or combinations thereof.

In various embodiments, 4-aminopyridine, or a compound in the class of 4-aminopyridine, influences, treats and/or provides benefit(s) to a cell, tissue, organ, subject, patient, animal and/or organism, nd the benefit is not treatment of multiple sclerosis.

In various embodiments, 4-aminopyridine, or a compound in the class of 4-aminopyridine, influences, treats and/or provides benefit(s) to a cell, tissue, organ, subject, patient, animal and/or organism, nd the benefit is not treatment or reduction of the signs or symptoms multiple sclerosis.

In various embodiments, 4-aminopyridine, or a compound in the class of 4-aminopyridine, influences, treats and/or provides benefit(s) to a cell, tissue, organ, subject, patient, animal and/or organism, nd the benefit is not treatment of an ataxia disorder.

In various embodiments, 4-aminopyridine, or a compound in the class of 4-aminopyridine, influences, treats and/or provides benefit(s) to a cell, tissue, organ, subject, patient, animal and/or organism, nd the benefit is not treatment or reduction of the signs or symptoms of an ataxia disorder.

In some embodiments, 4-aminopyridine, or a compound in the class of 4-aminopyridine, influences, treats and/or provides benefit(s) to a cell, tissue, organ, subject, patient, animal and/or organism, at least in part, by modulating the expression of one or more components identified in Gene Ontologyc Reactome, Wikipathways, and/or other curated biological pathways.

In some embodiments, 4-aminopyridine, or a compound in the class of 4-aminopyridine, influences, treats and/or provides benefit(s) to a cell, tissue, organ, subject, patient, animal and/or organism, wherein it modulates the expression of components identified in Gene Ontology, KEGG, Reactome, Wikipathways, and/or other curated biological pathways; and thereby influences actual biological pathway(s).

In some embodiments, said biological pathways are highly relevant to medicine, botany, agriculture and/or veterinary practice.

Multiple novel and detailed bioinformatics analyses described herein demonstrate the activity and or activities of 4-aminopyridine that indicate its usefulness, efficacy and benefit in numerous conditions and disorders wherein it has either not been previously considered or where others have discounted 4-aminopyridine or taught away from its use.

In some embodiments, 4-aminopyridine is a health maintaining, preventative, prophylactic, curative or treatment for one or more conditions, disorders or diseases.

In some embodiments, 4-aminopyridine may be considered a disease modifying drug.

In some embodiments, 4-aminopyridine may be considered not to be disease modifying.

In some embodiments, 4-aminopyridine provides unexpected benefit(s) addressing long held and unmet or poorly met needs related to one or more chronic conditions.

In addition to being used as a monotherapy, the pyridines and aminopyridines of the present invention will also find use in combination therapies.

An important feature of the present invention is the combination of synthetic compounds with other synthetic compounds or with naturally occurring compounds as such combinations may generally produce reduced side-effects as compared to combinations involving multiple pharmaceutical drugs (polypharmacy).

In some embodiments, 4-aminopyridine provides unexpected benefit(s) addressing long held and unmet or poorly met needs related to one or more acute or chronic condition(s), injury, traumatic injury, burn, or to the deleterious effects of other drug(s), agent(s), compounds, substances or treatment modalities.

In some embodiments, 4-aminopyridine may act as an adjuvant, pre-treatment, sensitizer, de-sensitizer, or be used in conjunction with other drugs, agents and/or treatment modalities.

The current experimental analyses demonstrate 4-amino pyridine is able to address long held and unmet needs and to provide surprising and/or novel benefits in a wide variety of chronic as well as acute conditions, including, but not limited to those exemplary diseases, disorders, conditions and maladies disclosed herein in corresponding manually curated, institutionally curated, and automatically curated lists.

It follows that the present invention teaches methods as well as compositions for maintaining, influencing, treating, and/or providing benefit(s) to a cell, tissue, organ, subject, patient and/or organism using 4-aminopyridine(s).

Modulation of gene expression in a cell, tissue, organ, subject, patient or organism using a 4-Aminopyridine provides a method of preventing, ameliorating, treating, mitigating and/or enhancing treatments of cancer, various chronic diseases and all manner of diseases, disorders or conditions featuring or characterized by unfavorable and occasionally contrary gene expression patterns.

Said method may be accomplished by administering to the cell, tissue, organ, subject, patient, and or organism an appropriate amount of a composition comprising 4-aminopyridine and/or another pyridine either in vitro, in vivo, or ex-vivo.

The present invention teaches an efficient method and process for screening and determining appropriate uses, as well as caveats (e.g. potential toxicities) related to the use of 4-aminopyridine including the analyses of gene expression modulation described herein.

In some embodiments, the present invention comprise the use of toxicity predictive platforms such as the Cellular Dynamics iCell and MyCell predictive toxicity testing platform(s), the BioMAP® Predictive Tox Panel, or similar platform for assessing potential toxicity, separately or in addition to the methodologies applied herein.

In some embodiments, the present invention comprises use of cell-based assay systems such as are available from Affymetrix, Illumina, Qiagen, Genecopoeia, Thermofisher BioMap Systems, BioMap Diversity Plus, BioMAP Oncology Systems, and BioMap EC50 ELECT, as well as the Cellular Dynamics iCell and MyCell efficacy and predictive toxicity testing platforms, separately or in addition to the methodologies applied herein.

In some preferred embodiments, less than 60 mg of a 4-aminopyridine is administered per day.

In some embodiments, doses comprise less than 30 mg of a 4-aminopyridine.

In some embodiments, doses comprise 0.1 to 10 mg/kg of a 4-aminopyridine.

In some embodiments, doses comprise 0.1 to 1 mg/kg of a 4-aminopyridine.

In some embodiments, the dosing range of the composition of the present invention is between a range between 0.1 mg/kg to 10 mg/kg. 4-aminopyridine.

In some embodiments, the dosing range of the composition of the present invention is between a range between 10 mg/kg to 100 mg/kg 4-aminopyridine when the route is topical, dermal, or transdermal.

In some embodiments, the dosing range of the composition of the present invention is between a range between 1 mg/kg to 50 mg/kg 4-aminopyridine when the route is topical, dermal, or transdermal.

In some embodiments, the dosing range of the composition of the present invention is between a range between 1 mg to 100 mg 4-aminopyridine when the route is topical, dermal, or transdermal.

In some embodiments, 4-aminopyridine is present at a concentration between 5 μm and 500 μm.

In some preferred embodiments, the compositions, manufacture, products, processes, methods, and/or methods of use, prevention and treatment of the present invention combine two or more drugs.

In some preferred embodiments, the compositions, manufacture, products, processes, methods, and/or methods of use, prevention and treatment of the present invention comprise two or more aminopyridines or pyridines.

In some preferred embodiments, the compositions, manufacture, products, processes, methods, and/or methods of use, prevention and treatment of the present invention combine two or more agents, compounds or drugs in ratios of about 1:1 to about 1:100, 1:1 to about 1:1000, 1:1 to about 1:10,000, and/or 1:1 to about 1:100,000.

In some embodiments, the composition comprises l-arginine.

In some embodiments, the composition comprises a second agent, compound or drug increasing BDNF expression in a cell, tissue, organ, organism, individual, and/or patient.

In some preferred embodiments, the compositions, manufacture, products, processes, methods, and/or methods of use, prevention and treatment of the present invention comprise drugs and/or aminopyridines or pyridines diluted at about 1:10 to about 1:1,000, or 1:100 to about 1:10,000.

In some preferred embodiments, the compositions, manufacture, products, processes, methods, and/or methods of use, prevention and treatment of the present invention comprise drugs diluted at about 1:100 to about 1:10,000, or 1:1,000 to about 1:100,000.

### Other Diseases Disorders and Conditions

ODDC comprise all conditions and diseases known to those skilled in the medical art, as the compounds and compositions described herein relate to a common pathway of cellular injury, cellular dysfunction, cellular derangement, and inflammation. For example, the present invention also provides compositions for preventing and treating parasitic diseases.

The present invention relates to various agents, compounds and drugs named herein. The agents, compounds and drugs of the present invention comprise i FDA approved drugs, ii. non-FDA approved drugs, and other agents.

In some embodiments, the present invention teaches the use of at least one drug selected from the group comprising pyridines and aminopyridines, said at least one drug of the present alone or in combination with others.

In some embodiments, the 4-aminopyridine is present alone or in combination with agents, compounds or other drugs, maintain health, or to treat, ameliorate, mitigate, improve, cure and/or prevent a large variety of disorders and conditions for which the use of 4-aminopyridine or said combinations has not been previously described or has been dismissed by prior teachings.

In some embodiments, the composition of the present invention is provided with dietary ketones and/or in the context of a ketone diet.

Similarly, the present invention teaches compositions, manufacture, products, processes, methods, and/or methods of use for delivering agents, compounds or drugs (including 4-aminopyridine) that have not been previously described or that have been dismissed by prior teachings.

### Additional Definitions

4-aminopyridine is defined herein as 4-aminopyridine or other aminopyridine, pyridine, niacin, niacin derivative, or their analogs or derivatives, whether naturally-occurring or synthetically-derived.

By “effective amount” is meant the amount of a required to ameliorate the symptoms of a disease relative to an untreated patient. The effective amount of an agent, compound or drug of the invention used to practice the present invention for therapeutic treatment of a disease may vary depending upon the manner of administration, the age, body weight, and general health of the subject. Ultimately, the attending physician or veterinarian will decide the appropriate amount and dosage regimen. Such amount is referred to as an “effective” amount.

By “ameliorate” is meant decrease, suppress, attenuate, diminish, arrest, or stabilize the development or progression of a disease. As used herein, the meaning of “ameliorate” includes lessening an effect, or reducing damage, or minimizing the effect or impact of an action, activity, or function, and includes, for example lessening the deleterious effects of a disease or condition. By “agent” is meant any small molecule chemical compound, antibody, nucleic acid molecule, or polypeptide, or fragments thereof.

By “modulation” is meant regulation, a change (increase or decrease) or alteration in the expression or activity levels or activity of a gene or polypeptide as detected by standard art known methods such as those described herein. As used herein, an alteration includes a 15% change in expression or activity levels, preferably a 25% change, more preferably a 40% change, and most preferably a 50% or greater change in expression or activity levels.” By “reduces” is meant a negative modulation of at least 5%, 25%, 50%, 75%, or 99%.

By “analog” is meant a molecule that is not identical, but has analogous functional or structural features. For example, a polypeptide analog retains the biological activity of a corresponding naturally-occurring polypeptide, while potentially having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

Likewise, analog herein refers to those compounds structurally related to the compound, agent or drug in question and which retains characteristic biological properties of the compound, agent or drug.

As used herein, the term “injury” refers to “organismal injury”, to damage, or to a lesion, wound, ulceration, scar, etc., without respect to the means, mode or cause of the injury. Causes, means and modes of injures may be ischemic, chemical, mechanical, traumatic, genetic, biochemical, physiological, environmental, exposure related, radiation related, sun related, light related, energy related, exogenous, endogenous, self-induced, etc.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

CDCP refers to the large number of serious chronic diseases such as cardiovascular disease, diabetes, obesity, hyperlipidemia, PCOS and hypertension that have been observed to cluster in patients, and includes disorders comprising metabolic syndrome or syndrome X. Such disorders are common in industrialized countries.

ODDC comprise all conditions and diseases known to those skilled in the medical art other than chronic diseases that cluster in patients (CDCP). In non-industrialized countries, infectious and parasitic diseases similarly threaten not only the lives of individuals, but the economic viability of families, communities, and societies as a whole. For example, protozoal illnesses continue to account for significant morbidity and mortality, especially in the tropical world. Malaria, which is caused by the protozoa, Plasmodium falciparum, Plasmodium vivax, Plasmodium ovale, is endemic in to 90 countries in Africa, Asia, Oceania, South America, and the Caribbean, infects approximately 300-500 million people and kills 2.5 million people every year.

As named and used herein, an agent, compound or drug of the present invention refers to the agent, compound or drug its analogs, and its derivatives including those derivatives described herein (e.g. glutathione conjugates, n-acetylcysteine conjugates, biotinylated derivatives, fluorinated derivatives, and derivatives having an NO donor moiety).

It is to be understood that this invention is not limited to the particular embodiments, techniques, active agents, and the like as such may vary. It is also to be understood that the terminology used herein is for describing particular embodiments only and is not intended to be limiting.

The compositions, methods, and/or methods of use, manufacture, products, processes, prevention and treatment of the present invention can be mixed with suitable pharmaceutical carriers (vehicles) or excipients known to the art (e.g. Kumar et al., 1996; Akers et al., 2002; Strickley et al., 2004; Jacob et al., 2010; Siddiqui et al., 2010; Pilcer et al., 2010). Examples include water-soluble organic solvents, non-ionic surfactants, water-insoluble lipids, organic liquids/semi-solids, cyclodextrins and phospholipids. They may also include gelatin, lactic acid, stearic acid or salts or complexes thereof, starch, milk, sugar, certain types of clay, including magnesium or calcium stearate, talc, oils, gums, vegetable fats, lipids, or and glycols. Said references are incorporated herein by reference.

Examples of suitable pharmaceutical vehicles are also described in Remington's Pharmaceutical Sciences, Alfonso R. Gennaro ed., Mack Publishing Co. Easton, Pa., 19th ed., 1995, pp. 1447 to 1676, incorporated herein by reference.

In some embodiments, natural compounds useful in the present invention may be administered in a dose of from about 0.01 mg/kg of the individual's body weight to about 500 mg/kg of the individual's body weight.

In some embodiments, the compositions, manufacture, products, processes, methods, and/or methods of use, prevention and treatment of the present invention comprise a compound, agent or drug extracted from cloves, black pepper, red chili, cinnamon, and ginger.

In some preferred embodiments, the compositions, manufacture, products, processes, methods, and/or methods of use, prevention and treatment of the present invention combine said extracts which act synergistically.

In one preferred embodiment of the present invention the subject is a mammal.

In one preferred embodiment of the present invention, the individual is a plant or animal.

In one embodiment of the present invention the subject is a non-mammal animal.

In various embodiments, the composition is utilized in a method of the present invention and said method exclusively involves: treatment of a cell only, treatment of a tissue only, treatment of an organ only, treatment of an organ system only, treatment of an organism only, treatment of an individual only, or treatment of a patient only.

Such compositions, manufacture, products, processes, methods, and/or methods of use, prevention and treatment can be administered to a subject animal such as mammals (rat, mouse, domestic animals or human) via various routes.

In preferred embodiments, the composition of the present invention is effective for preventing or treating cancers, diseases, disorders, and maladies and effective for improving health and well-being wherein said conditions, diseases and maladies include cancer and chronic diseases that cluster in patients, acute diseases, ischemic diseases, inflammatory diseases, metabolic disorders, injuries, degenerative disorders, neurodegenerative disorders, and other diseases, disorders and conditions.

Accordingly, said composition is preferably also suitable for reducing polypharmacy.

In some embodiments, said composition comprises one or more agents, compounds or drugs belonging to the class represented by 4-aminopyridine, its analogs or derivatives.

In some embodiments, said composition comprises one or more agents, compounds or drugs belonging to the class represented by pyridine its analogs or derivatives.

In some embodiments, said composition comprises one or more agents, compounds or drugs belonging to the class represented by an aminopyridine, its analogs or derivatives.

In some embodiments, said composition comprises one or more agents, compounds or drugs belonging to the class represented by 3,4-diaminopyridine, its analogs or derivatives.

In some embodiments, said composition comprises one or more agents, compounds or drugs belonging to the class represented by 4-aminopyridine, its analogs or derivatives.

In some embodiments, said composition comprises one or more agents, compounds or drugs belonging to the class represented by 3-aminopyridine, its analogs or derivatives.

In some embodiments, said composition comprises one or more agents, compounds or drugs belonging to the class represented by 2-aminopyridine, its analogs or derivatives.

In some embodiments, said composition comprises one or more agents, compounds or drugs belonging to the class represented by nicotinamide, its analogs or derivatives.

In some embodiments, said composition comprises one or more agents, compounds or drugs belonging to the class represented by NADPH, its analogs or derivatives.

In some embodiments, said composition comprises one or more agents, compounds or drugs belonging to the class represented by Phenazopyridine, its analogs or derivatives.

In some embodiments, said composition comprises one or more agents, compounds or drugs belonging to the class represented by sulfapyridine, its analogs or derivatives.

In some embodiments, said composition comprises one or more agents, compounds or drugs belonging to the class represented by pyridostigmine, its analogs or derivatives.

In some embodiments, said composition comprises one or more agents, compounds or drugs belonging to the class represented by neostigmine, its analogs or derivatives.

In some embodiments, said composition comprises one or more agents, compounds or drugs belonging to the class represented by rivastigmine, its analogs or derivatives.

In some embodiments, said composition comprises one or more agents, compounds or drugs belonging to the class represented by torsemide, its analogs or derivatives.

In some embodiments, said composition comprises one or more agents, compounds or drugs belonging to the class represented by nalidixic acid, its analogs or derivatives.

In some embodiments, said composition comprises one or more agents, compounds or drugs belonging to the class represented by alendroic acid, its analogs or derivatives.

In some embodiments, said composition comprises one or more agents, compounds or drugs belonging to the classes represented by ethionamide, prothionamide, vismodegib, their analogs or derivatives.

In some embodiments, the composition is a disease modifying composition or a non-disease modifying composition.

In some embodiments, the composition is effective for preventing or treating multiple diseases, and conditions, whereby the composition may reduce polypharmacy.

In some embodiments, the composition comprises an agent, compound or drug selected from the classes represented by pyridine, pyridostigmine, neostigmine, torsemide, alendroic acid, vismodegib, prothionamide, nalidixic acid, ethionamide, pyridine nucleotides, nicotinamide, NADP, NAADP, 4-aminopyridine, 4-aminopyridine-methanol, 3,4-diaminopyridine, 3-aminopyridine, Phenazopyridine, sulfapyridine, their analogs and derivatives.

In some embodiments, the composition modulates Terpenoid/Isoprenoid biosynthesis and/or metabolism.

In some embodiments, the composition modulates Terpenoid biosynthesis.

In some embodiments, the composition promoted Terpenoid/Isoprenoid biosynthesis and/or metabolism.

In some embodiments, the composition promotes Terpenoid biosynthesis.

In some embodiments, the composition modulates prenylation.

In some embodiments, the composition treats and/or prevents conditions, diseases and maladies selected from cancer and chronic diseases that cluster in patients, acute diseases, ischemic diseases, cardiovascular disease, cerebrovascular disease, bone disease, inflammatory diseases, metabolic disorders, injuries, diseases of aging, degenerative disorders, neurodegenerative and other diseases, disorders and conditions.

In some embodiments, the composition of the present invention modulates expression of one or more RNA described herein.

In some embodiments, the composition of the present invention modulates expression of one or more miRNA described herein.

In some embodiments, the composition of the present invention modulates expression of one or more curated biological pathway described herein.

In some embodiments, the composition promotes terpenoid backbone biosynthesis and sesquiterpene activity, promotes Linoleic acid (LA) metabolism, modulates neurite outgrowth, promotes neurotransmission, promotes transmission across electrical synapses, promotes cardiac conduction, promotes Ephrin signaling, and/or promotes transmission across gap junctions in a cell, tissue, organ, subject, patient and/or organism.

In some embodiments, the composition promotes POU5F1 (OCT4), SOX2, KLF4 and/or NANOG activation of genes related to proliferation and pluripotency, promotes skeletal muscle hypertrophy, promotes miRs in muscle differentiation, modulates osteoclast function, modulates estrogen biosynthesis, reduces chemotherapeutic resistance, reduces Imatinib Resistance, reduces oncogene expression, promotes transmission across chemical synapses, promotes recovery from cardiac infarction, reduces type II diabetes, promotes fatty acid metabolism, and/or reduces FTO obesity mechanism in a cell, tissue, organ, subject, patient and/or organism.

In some embodiments, the composition promotes NGF independent trkA activation, promotes neurotrophin signaling, promotes NGF signaling via trkA, promotes BDNF signaling, promotes IFN stimulated Antiviral mechanisms, reduces TNFR1-induced NFkappaB signaling pathway, promotes Leptin and Adiponectin signaling, reduces prion disease, and/or promotes ISG15 antiviral mechanisms in a cell, tissue, organ, subject, patient and/or organism.

In some embodiments, the composition promotes glucose transport, reduces galactose catabolismpromotes hyaluronan synthesis, promotes chondroitin sulfate biosynthesis, promotes glycosaminoglycan biosynthesis, and/or promotes transcriptional activation of mitochondrial biogenesis in a cell, tissue, organ, subject, patient and/or organism.

In some embodiments, the composition modulates ATF4, NPTX1, NPAS4, BDNF, mir-547, and/or mir-21 activity in a cell, tissue, organ, subject, patient and/or organism.

In some embodiments, the composition promotes serotonin neurotransmission, promotes Serotonin Receptor 4/6/7 and NR3C Signaling, promotes dopaminergic neurotransmitter release, promotes acetylcholine neurotransmitter release, promotes acetylcholine signaling, reduces choline catabolismpromotes circadian gene expression, promotes Platelet homeostasis, and/or promotes RSK activation (cognition learning) in a cell, tissue, organ, subject, patient and/or organism.

In some embodiments, the composition promotes neurotransmitter release, promotes GABA synthesis, release, uptake and degradation, promotes GABA A receptor activation, promotes glutamate neurotransmitter release, promotes activation of kainate receptors upon glutamate binding, promotes presynaptic function of kainate receptors, promotes ADP signaling through P2Y purinoreceptor, promotes norepinephrine neurotransmitter release, reduces nicotinic activity on dopaminergic neurons, reduces activation of postsynaptic nicotinic receptors, reduces amyotrophic lateral sclerosis, promotes heme biosynthesis, promotes MAPK activity, reduces prion disease and/or promotes metal SLC transporters in a cell, tissue, organ, subject, patient and/or organism.

In some embodiments, the composition promotes metal SLC transporters (https://www.sciencedirect.com/science/article/pii/S2211383519309360), promotes heme biosynthesis, and/or promotes regulation of ERK/MAPK.

In some embodiments, the composition promotes circadian gene expression (hypertension), promotes cardiac cell calcium regulation, promotes tandem pores domain K channels, reduces cardiac hypertrophy, reduces MicroRNAs in Cardiomyocyte Hypertrophy, regulates lipoxin synthesis, and/or promotes CREB phosphorylation (downstream of NGF) in a cell, tissue, organ, subject, patient and/or organism.

In some embodiments, the composition promotes nuclear receptors (esp for steroids and hormones which directly bind DNA and act as transcription factors), promotes PDGF pathway (postinfarction repair), regulates EGFR1 signaling (myocardial repair), regulates HSF1 mediated heat shock response, promotes G protein signaling, reduces clot formation, reduces blood clotting cascade, downregulates EGFR1 signaling, and/or promotes nuclear receptors in a cell, tissue, organ, subject, patient and/or organism.

In some embodiments, the composition reduces the integrated cancer pathway, inhibits basal cell carcinoma, promotes PUMA activation and translocation to the mitochondria, promotes fas pathway, reduces EGR1, reduces EGR2, modulates EGFR expression, regulates activation of NIMA receptors, and/or downregulates the extrinsic clot formation pathway in a cell, tissue, organ, subject, patient and/or organism.

In some embodiments, the composition downregulates the extrinsic clot formation pathway, promotes TP53 regulation of transcription of death receptors and ligands, reduces oncostatin M signaling, reduces WNT signaling, downregulates Beta-catenin phosphorylation cascade, reduces Glioblastoma signaling pathways, downregulates Matrix Metalloproteinases, downregulates MFAP5-mediated ovarian cancer cell motility and invasiveness, reduces DNA replication, promotes sensing of double strand breaks, and/or regulates the cd28 dependent vav1 pathway in a cell, tissue, organ, subject, patient and/or organism.

In some embodiments, the composition reduces Notch1 signaling, reduces Endometrial cancer pathways, reduces Melanogenesis, downregulates Gastric Cancer Network pathways, downregulates viral carcinogenesis, downregulates thyroid cancer pathways, promotes selenium and selenoprotein metabolism, and/or reduces cysteine and homocysteine degradation in a cell, tissue, organ, subject, patient and/or organism.

In some embodiments, the composition increases Notch2 domain regulation of transcription, promotes MAPK, reduces androgen receptor signaling, regulates hair follicle development, promotes selenium and selenoprotein metabolism, reduces cysteine and homocysteine degradation, regulates the ACE Inhibitor pathway, and/or promotes neurotransmitter receptor binding and downstream transmission in postsynaptic cell in a cell, tissue, organ, subject, patient and/or organism.

In some embodiments, the composition regulates gene expression in endocrine committed progenitors, reduces type 2 diabetes, promotes insulin secretion, signaling and processing, promotes glucagon signaling, promotes glucagon like peptide regulating insulin secretion, promotes IRS activation in insulin signaling, promotes glucagon type ligand receptors, promotes orexin, neuropeptide FF and QRFP binding to their respective receptors, promotes glucose transport, regulates the Longevity regulating pathway, reduces fructose metabolism in a cell, tissue, organ, subject, patient and/or organism.

In some embodiments, the composition modulates glycosphingolipid biosynthesis, promotes glycosaminoglycan biosynthesis, promotes cholesterol biosynthesis, promotes biosynthesis of unsaturated fats, promotes muscle hypertrophy, promotes SREBP signaling, promotes SREBF activation, and/or promotes prostacyclin signaling in a cell, tissue, organ, subject, patient and/or organism.

In some embodiments, the composition regulates toll like receptor activation, regulates myD88 cascade, regulates induction of NFKB and MAP kinases, promotes mitochondrial gene expression, regulates the SIDS susceptibility pathway, promotes IL1, promotes IL2, promotes Signal amplification, and/or promotes biological oxidation enzymes associated with metabolic disorders in a cell, tissue, organ, subject, patient and/or organism.

In some embodiments, the composition promotes adipogenesis, promotes Leptin and Adiponectin signaling, promotes white and brown fat differentiation, promotes phospholipid metabolism, promotes Synthesis of very long-chain fatty acyl-CoAs, promotes TGF-beta Signaling, promotes Gastrin-CREB signaling pathway, promotes Serotonin Receptor 2 signaling, promotes ELK-SRF/GATA4 signaling, reduces IL4 signaling, promotes TOR signaling, promotes organic ion transporters, promotes vitamin C metabolism, promotes Ion transport by P-type ATPases, promotes G protein signaling, and/or regulates copper homeostasis.

In some embodiments, the composition of the present invention promotes satiety, weight loss and/or gene expression regulating Leptin and adiponectin.

In some embodiments, the composition promotes increased gene expression in conjunction with one or more of NGF-stimulated transcription; NPAS4 regulation of target genes; Nuclear Events (kinase and transcription factor activation); Tandem pore domain potassium channels; Transcriptional Regulation by NPAS4; Phase 4-resting membrane potential; Signaling by NTRKs; Signaling by NTRK1 (TRKA); Tandem of pore domain in a weak inwardly rectifying K+ channels (TWIK); TNF receptor superfamily (TNFSF) members mediating non-canonical NF-kB pathway; RUNX2 regulates genes involved in differentiation of myeloid cells; Regulation of NPAS4 gene expression; Regulation of thyroid hormone activity; RUNX1 regulates transcription of genes involved in WNT signaling; NEIL3-mediated resolution of ICLs; Defective Base Excision Repair Associated with NEIL3; Defective pro-SFTPB causes SMDPI and RDS; G alpha (q) signalling events; Regulation of NPAS4 mRNA translation; Calcitonin-like ligand receptors; RUNX1 regulates transcription of genes involved in differentiation of myeloid cells; Regulation of NPAS4 gene transcription; Calcineurin activates NFAT; MECP2 regulates transcription of neuronal ligands; Tandem pore domain halothane-inhibited K+ channel (THIK); and/or TGFBR2 MSI Frameshift Mutants in Cancer.

In some embodiments, the composition promotes increased gene expression in conjunction with one or more of: Deposition of new CENPA-containing nucleosomes at the centromere; Nucleosome assembly; G0 and Early G1; G1/S-Specific Transcription; Cell Cycle, Mitotic; Transcriptional regulation of granulopoiesis; Mitotic G1 phase and G1/S transition; Transcriptional regulation by small RNAs Transport of the SLBP independent Mature mRNA; G2 Phase; Transport of the SLBP; Dependant Mature mRNA; Cell Cycle Checkpoints; SUMOylation of DNA replication proteins; G1/S Transition; PRC2 methylates histones and DNA; Assembly of the ORC complex at the origin of replication; Amplification of signal from the kinetochores; Amplification of signal from unattached kinetochores via a MAD2 inhibitory signal; Activation of gene expression by SREBF (SREBP); Collagen chain trimerization; Senescence-Associated Secretory Phenotype (SASP); Mitotic Prophase; Transport of Mature mRNA derived from an Intron-Containing Transcript; Mitotic Spindle Checkpoint; Regulation of cholesterol biosynthesis by SREBP (SREBF); Transport of Mature mRNA Derived from an Intronless Transcript; NS1 Mediated Effects on Host Pathways; Packaging Of Telomere Ends; RNA Polymerase I Promoter Opening; RNA Polymerase I Promoter Escape; Defective binding of RB1 mutants to E2F1, (E2F2, E2F3); TNF receptor superfamily (TNFSF) members mediating non-canonical NF-kB pathway; Aberrant regulation of mitotic G1/S transition in cancer due to RB1 defects; SUMOylation of RNA binding proteins; Transcription of E2F targets under negative control by DREAM complex Recognition and association of DNA glycosylase with site containing an affected purine; Transport of Mature mRNAs Derived from Intronless Transcripts; Transport of Mature Transcript to Cytoplasm; Chromosome Maintenance; SUMOylation of SUMOylation proteins Activation of ATR in response to replication stress; Cell Cycle; DNA methylation; SIRT1 negatively regulates rRNA expression; Potassium Channels; Condensation of Prophase Chromosomes; Defective TPR may confer susceptibility towards thyroid papillary carcinoma (TPC); Aberrant regulation of mitotic cell cycle due to RB1 defects; Cyclin A/B1/B2 associated events during G2/M transition; Nuclear import of Rev protein; Recognition and association of DNA glycosylase with site containing an affected pyrimidine; Nuclear Pore Complex (NPC) Disassembly; B—WICH complex positively regulates rRNA expression Transcription of E2F targets under negative control by p107 (RBL1) and p130 (RBL2) in complex with HDAC1; Nuclear Receptor transcription pathway; Tachykinin receptors bind tachykinins; Phosphorylation of proteins involved in the G2/M transition by Cyclin A: Cdc2 complexes; G1 Phase; Cyclin D associated events in G1; Diseases of mitotic cell cycle; SUMOylation of chromatin organization proteins; snRNP Assembly; Metabolism of non-coding RNA; SUMOylation of ubiquitinylation proteins; G beta: gamma signalling through CDC42; and/or POU5F1 (OCT4), SOX2, NANOG activate genes related to proliferation and pluripotency.

In some embodiments, the composition promotes increased gene expression in conjunction with one or more of: Nucleosome assembly; Deposition of new CENPA-containing nucleosomes at the centromere; G0 and Early G1; G1/S-Specific Transcription; Cell Cycle, Mitotic; Transcriptional regulation of granulopoiesis; Mitotic G1 phase and G1/S transition; Transcriptional regulation by small RNAs; Transport of the SLBP independent Mature mRNA; G2 Phase; Transport of the SLBP Dependant Mature mRNA; Cell Cycle Checkpoints; SUMOylation of DNA replication proteins; G1/S Transition; PRC2 methylates histones and DNA; Assembly of the ORC complex at the origin of replication; Amplification of signal from the kinetochores; Amplification of signal from unattached kinetochores via a MAD2 inhibitory signal; Activation of gene expression by SREBF (SREBP); Collagen chain trimerization; Senescence-Associated Secretory Phenotype (SASP); Mitotic Prophase; Transport of Mature mRNA derived from an Intron-Containing Transcript; Mitotic Spindle Checkpoint; Regulation of cholesterol biosynthesis by SREBP (SREBF); Transport of Mature mRNA Derived from an Intronless Transcript; NS1 Mediated Effects on Host Pathways; Packaging Of Telomere Ends; RNA Polymerase I Promoter Opening; RNA Polymerase I Promoter Escape; Defective binding of RB1 mutants to E2F1, (E2F2, E2F3); TNF receptor superfamily (TNFSF) members mediating non-canonical NF-kB pathway; Aberrant regulation of mitotic G1/S transition in cancer due to RB1 defects; SUMOylation of RNA binding proteins; Transcription of E2F targets under negative control by DREAM complex; Recognition and association of DNA glycosylase with site containing an affected purine; Transport of Mature mRNAs Derived from Intronless Transcripts; Transport of Mature Transcript to Cytoplasm; Chromosome Maintenance; SUMOylation of SUMOylation proteins; Activation of ATR in response to replication stress; Cell Cycle; DNA methylation; SIRT1 negatively regulates rRNA expression Potassium Channels; Condensation of Prophase Chromosomes; Defective TPR may confer susceptibility towards thyroid papillary carcinoma (TPC); Aberrant regulation of mitotic cell cycle due to RB1 defects; Cyclin A/B1/B2 associated events during G2/M transition; Nuclear import of Rev protein; Recognition and association of DNA glycosylase with site containing an affected pyrimidine; Nuclear Pore Complex (NPC) Disassembly; B—WICH complex positively regulates rRNA expression; Transcription of E2F targets under negative control by p107 (RBL1) and p130 (RBL2) in complex with HDAC1; Nuclear Receptor transcription pathway; Tachykinin receptors bind tachykinins; Phosphorylation of proteins involved in the G2/M transition by Cyclin A: Cdc2 complexes; Cyclin D associated events in G1 and/or G1 Phase.

In some embodiments the composition of the present invention reduces gene expression in conjunction with Attenuation phase; NGF-stimulated transcription; HSF1 activation; HSF1-dependent transactivation; Regulation of HSF1-mediated heat shock response; Nuclear Events (kinase and transcription factor activation); Cellular response to heat stress; Activation of C3 and C5; NFE2L2 regulating inflammation associated genes; Signaling by MAPK mutants; Activation of PUMA and translocation to mitochondria; RUNX3 Regulates Immune Response and Cell Migration; RAF-independent MAPK1/3 activation; Dermatan sulfate biosynthesis; Formation of intermediate mesoderm; HDMs demethylate histones, and/or Prostanoid ligand receptors.

In some embodiments the composition of the present invention reduces gene expression in conjunction with Butyrophilin (BTN) family interactions; Highly calcium permeable nicotinic acetylcholine receptors; Signaling by MAPK mutants; IRAK4 deficiency (TLR5); Caspase-mediated cleavage of cytoskeletal proteins; Attenuation phase; HSF1 activation; Defective CHST14 causes EDS, musculocontractural type; Regulation of gene expression in endocrine-committed (NEUROG3+) progenitor cells; Dermatan sulfate biosynthesis; Highly calcium permeable postsynaptic nicotinic acetylcholine receptors; RUNX2 regulates genes involved in differentiation of myeloid cells; HSF1-dependent transactivation; Activation of PUMA and translocation to mitochondria; MyD88 deficiency (TLR5); Formation of the posterior neural plate; Formation of the anterior neural plate; Presynaptic nicotinic acetylcholine receptors; Activation of C3 and C5; ECM proteoglycans; Regulation of TP53 Expression; RUNX3 regulates RUNX1-mediated transcription; HHAT G278V doesn't palmitoylate Hh-Np Rhesus blood group biosynthesis; NFE2L2 regulating MDR associated enzymes; TFAP2 (AP-2) family regulates transcription of growth factors and their receptors; Loss of function of TP53 in cancer due to loss of tetramerization ability; Loss of Function of TP53 in Cancer; RUNX1 regulates expression of components of tight junctions; VEGF ligand-receptor interactions; VEGF binds to VEGFR leading to receptor dimerization; Acetylcholine binding and downstream events Postsynaptic nicotinic acetylcholine receptors; Formation of Senescence-Associated Heterochromatin Foci (SAHF); Elevation of cytosolic Ca2+ levels; Formation of intermediate mesoderm; Apoptosis induced DNA fragmentation; Molecules associated with elastic fibres; YAP1- and WWTR1 (TAZ)-stimulated gene expression; RUNX1 regulates transcription of genes involved in WNT signaling; RUNX3 regulates YAP1-mediated transcription; Defective CHST3 causes SEDCJD; Transport of gamma-carboxylated protein precursors from the endoplasmic reticulum to the Golgi apparatus; TFAP2 (AP-2) family regulates transcription of other transcription factors; Regulation of gene expression in early pancreatic precursor cells Transcriptional regulation of granulopoiesis; Defective B3GALTL causes PpS; EGR2 and SOX10-mediated initiation of Schwann cell myelination; Elastic fibre formation TRIF-mediated programmed cell death; Defective CHSY1 causes TPBS; Removal of aminoterminal propeptides from gamma-carboxylated proteins; Interleukin-6 family signaling Regulation of beta-cell development; TP53 Regulates Transcription of Caspase Activators and Caspases; Defective GALNT12 causes CRCS1; O-glycosylation of TSR domain-containing proteins; Regulation of HMOX1 expression and activity; TLR3-mediated TICAM1-dependent programmed cell death; Extrinsic Pathway of Fibrin Clot Formation; Activation of NOXA and translocation to mitochondria; Zinc efflux and compartmentalization by the SLC30 family Transcriptional regulation by the AP-2 (TFAP2) family of transcription factors; NOTCH4 Intracellular Domain Regulates Transcription; TP53 Regulates Transcription of Genes Involved in G2 Cell Cycle Arrest; RUNX1 regulates transcription of genes involved in differentiation of myeloid cells; and/or Axonal growth inhibition (RHOA activation).

In some embodiments, the composition modulates ATF4 activity, VEGF activity, EGFR activity, and/or telomerase activity; promotes absorption, bioavailability, hair growth, and/or intracellular glutathione; extends the lifespan of a cell, tissue, organ or organism; reduces the amount of an approved drug required to achieve clinical benefit; reduces or prevents hypertension, obesity, hyperglycemia, hyperlipidemia, atherosclerosis, cancer or chemotherapeutic resistance; comprises one or more second agent, compound, drug, or approved drug; and/or reduces or prevents one or more inflammatory disease, oncological disease, genetic disease, ischemic disease, infectious disease, neurological disease, hematological disease, kidney disease, vascular disease, dermatological disease, opthalmological disease, rheumatoid disease, orthopedic disease, gynecological disease, obstetric disease, pediatric disease, sepsis, contrast-induced nephropathy, chronic kidney disease, pulmonary fibrosis, hypoxic condition, chemical-induced lung injury, respiratory distress disorder, anon gap acidosis, nephritis, lupus, interstitial lung disease, graft dysfunction, hepatitis, acute kidney injury, noise-induced hearing injury, poisoning, retinopathy, neurotoxicity, cancer-induced injury, ototoxicity, respiratory infection, autism, condition involving vasospasm, traumatic injury, traumatic brain injury, and/or traumatic blast injury.

Examples of disorders prevented or ameliorated by administration of the compositions of this invention include but are not limited to inflammatory diseases that may be, oncological, genetic, ischemic, infectious, neurological, hematological, ophthalmological, rheumatoid, orthopedic, neurological, hematological, kidney, vascular, dermatological, gynecological, obstetric, otherwise physical, psychological, or psychiatric.

In some embodiments, the compositions, manufacture, products, processes, methods, and/or methods of use, prevention and treatment of the present invention are useful for preventing or treating a Neurodegenerative disease or condition. Examples of such neurodegenerative diseases include Parkinson disease, Alzheimer's disease, Multiple Sclerosis, Myasthenia gravis, myasthenic syndrome, Schizophrenia, Dementia, and Huntington's disease.

In some embodiments, the compositions, manufacture, products, processes, methods, and/or methods of use, prevention and treatment of the present invention are useful for preventing or treating diseases or conditions related to injury, traumatic injury, chemical injury, ischemic injury, crush injury, inflammation, autoimmunity, and/or aging.

In some embodiments, the composition comprises a lipid, an omega-3 fatty acid, a liposome, a nanoparticle, a nanotube, a nanovault, a nanofiber, Arabic gum, an excipient, a diluent, a soluent, a solvent, a stabilizer, surfactant, buffering agent, second agent, a second drug, or any combination thereof.

In some embodiments, the composition takes the form of solution, liquid, stable liquid, gel, suspension, emulsion, lotion, tablet, pill, pellet, capsule, powder, sustained-release formulation, suppository, emulsion, aerosol, spray, drop, buccal or sublingual form, a transdermal patch, cream, lotion, ointment, shampoo, gum, troche, gummy, beverage, sublingual composition, functional food, condiment, nutritional drink, polyethylene glycol-coated preparation, suspension, syrup, soft gel, topical formulation, nanoemulsion, nanoparticle preparation, powder mixture, topical gel, sunscreen, lozenge, cream, aqueous formulation, injectable formulation, or any combination thereof.

Aminopyridines are associated with serious side effects that have limited their use.

In some embodiments of the present invention, a transdermal patch provides for a very consistent delivery of a drug at a relatively consistent concentration over time, thereby avoiding pharmacokinetic peaks and valleys associated with existing formulations, while reducing the risk of seizures and other side effects.

Similarly, in some embodiments, targeted, localized treatments with novel formulations described herein (in contrast to existing, systemic formulations) will likewise dramatically reduce or eliminate the risk of serious side effects.

In some embodiments, a nasal spray provides limited doses of the drug for shorter periods of time, while reducing or eliminating the risk of serious side effects.

In some embodiments, a troche provides limited doses of the drug for shorter periods of time, while reducing or eliminating the risk of serious side effects.

In some embodiments, the composition enables one or more methods for reducing oncogene expression, or preventing, treating or ameliorating one or more disease, disorder, or condition wherein one or more disease disorder, or condition is inflammatory, oncological, genetic, ischemic, infectious, neurological, hematological, urological, nephrological, cardiovascular, dermatological, disorder, ophthamological, rheumatological, orthopedic, gynecological, obstetric, pulmonary, dermatological, pediatric, degenerative, neurodegenerative, age related, traumatic, injurious, or other manner of disease disorder, or condition

In some embodiments, the composition reduces gene expression in a cell, tissue, organ, subject, patient or organism that promotes a malady, disease, disorder or undesirable condition.

In some embodiments, the composition increases gene expression in a cell, tissue, organ, subject, patient or organism that is deficient in a malady, disease, disorder or undesirable condition.

In one preferred embodiment of the present invention the disease is selected from the group comprising cancer and psoriasis.

Examples of cancer related diseases and conditions include prostate cancer, breast cancer, lung cancer, colorectal cancer, bladder cancer, uterine cancer, ovarian cancer, lymphoma, skin cancer, stomach cancer, liver cancer, wasting diseases, and other cancers.

In some embodiments, the compositions of the present invention are useful for preventing or treating diseases and conditions related to the Prostate such as prostate enlargement and prostate cancer.

In some embodiments, the present invention provides means or adjunctive means of treating cancer (e.g. multiple myeloma, colorectal cancer, leukemic cells, Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain tumor, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, childhood, Carcinoid tumor, gastrointestinal, Carcinoma of unknown primary, Central nervous system lymphoma, primary, Cerebellar astrocytoma, childhood, Cerebral astrocytoma/Malignant glioma, childhood, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) Cancer, Gastric (Stomach) Cancer, Childhood, Gastrointestinal Carcinoid Tumor, Gastrointestinal Stromal Tumor (GIST), Germ Cell Tumor, Extracranial, Childhood, Germ Cell Tumor, Extragonadal, Germ Cell Tumor, Ovarian, Gestational Trophoblastic Tumor, Glioma, Adult, Glioma, Childhood Brain Stem, Glioma, Childhood Cerebral Astrocytoma, Glioma, Childhood Visual Pathway and Hypothalamic, Gastric Carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, childhood, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Macroglobulinemia, Waldenstrom, Malignant Fibrous Histiocytoma of Bone/Osteosarcoma, Medulloblastoma, Childhood, Melanoma, Melanoma, Intraocular (Eye), Merkel Cell Carcinoma, Mesothelioma, Adult Malignant, Mesothelioma, Childhood, Metastatic Squamous Neck Cancer with Occult Primary, Mouth Cancer, Multiple Endocrine Neoplasia Syndrome, Childhood, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Chronic, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, pancreatic cancer, islet cell cancer, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, Salivary gland cancer, Sarcoma, Ewing family of tumors, Sarcoma, Kaposi, Sarcoma, soft tissue, Sarcoma, uterine, Sezary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma-see Skin cancer (nonmelanoma), Squamous neck cancer with occult primary, metastatic, Stomach cancer, Supratentorial primitive neuroectodermal tumor, childhood, T-Cell lymphoma, cutaneous-see Mycosis Fungoides and Sezary syndrome, Testicular cancer, Throat cancer, Thymoma, childhood, Thymoma and Thymic carcinoma, Thyroid cancer, Thyroid cancer, childhood, Transitional cell cancer of the renal pelvis and ureter, Trophoblastic tumor, Ureter and renal pelvis, transitional cell cancer, Urethral cancer, Uterine cancer, endometrial, Uterine sarcoma, Vaginal cancer, Visual pathway and hypothalamic glioma, childhood, Vulvar cancer, Waldenstrom macroglobulinemia, Wilms tumor (kidney cancer), childhood, etc.) and other cancers, in vitro or in vivo, comprising the steps of: contacting said cells with an amount of agent(s), compound(s) or drug(s) of the present invention delivered by a composition effective to inhibit the proliferation of the cancer cells.

In some embodiments, the present invention provides the means of inducing apoptosis in cancer cells in vitro or in vivo, comprising the steps of: contacting said cells with an amount of at least one agent, compound or drug of the present invention delivered by a composition effective to induce apoptosis in the cancer cells.

In some embodiments, the present invention provides the means of increasing the cytotoxic effects of at least one chemotherapeutic agents against the cancer cells, comprising the steps of: contacting said cells with said at least one chemotherapeutic agent, compound or drug of the present invention delivered by a composition wherein said composition of the present invention increases the cytotoxic effects of said one or more chemotherapeutic agent against the cancer cells.

In some embodiments, at least one chemotherapeutic agent is selected from the group consisting of vincristine, BCNU, melphalan, cyclophosphamide, Adriamycin, prednisone, velcade, thalidomide, and dexamethasone.

In some embodiments, said cancer cells are CD 138+ plasma cells.

In some embodiments, the present invention provides the means of treating multiple myeloma or other cancer in an individual, comprising the step of administering a therapeutically effective amount of a composition of the present invention to said individual.

In one preferred embodiment the composition according to the present invention is produced by C0₂ extraction, DMSO extraction, combination of C0₂ extraction and DMSO extraction, cold-press extraction and steam distillation extraction.

Methods for producing an extract (e.g. natural oils, absolutes, and concretes, etc.) are well known to the art (e.g. Harborne, 1998. Phytochemical Methods A Guide to Modern Techniques of Plant Analysis; I. Walinga, J. J. van der Lee, V. J. G. Houba, W. van Vark, I. Novozamsky, 1995, Plant Analysis Manual; Elizabeth M. Williamson, David T. Okpako, Fred J. Evans, 1996, Selection, Preparation and Pharmacological Evaluation of Plant Material; U.S. Pat. No. 6,241,975 B1; Schnaubelt, K. (2002). Biology of Essential Oils. San Rafael, CA: Terra Linda Scent; Guenther, E. (1982). The Essential Oils. Melbourne, Fl: Krieger Publishing; Food and Agriculture Organization of the United Nations (1995). Basic Principles of Steam Distillation. Retrieved Aug. 18, 2005, from http://www.fao.org/docrep/V5350e/V5350el3.htm; Catty, S. (2001). Hydrosols: The Next Aromatherapy. Rochester, VT: Healing Arts Press; Burnett, C. (2014) Safety Assessment of Citrus-Derived Peel Oils as Used in Cosmetics, Cosmetic Ingredient Review, Personal Care Products Council; NTP. (2000), Lemon Oil, Lime Oil, National Toxicology Program, U.S. Department of Health & Human Services; Guba, R. (2002); The Modern Alchemy of Carbon Dioxide Extraction. International Journal of Aromatherapy 12 (3), 120-126; http://www.edenbotanicals.com/extraction-methods, CN102391911: Supercritical extraction method for orange peel essential oil; Parameters optimization of supercritical fluid-C0₂ extracts of frankincense using response surface methodology and its pharmacodynamics effects.”/Jing Zhou, Xing-miao Ma, Bi-Han Qiu, Jun-xia Chen, Lin Bian, Lin-mei Pan//Journal of Separation Science, Volume 36, Issue 2, January 2013, Pages 383-390) incorporated herein in their entireties.

### Compositions and Formulations

In some preferred embodiments, the composition comprises at least one diluent, and/or at least one stabilizer, and/or at least one surfactant, and/or at least one salt or buffering agent.

In some embodiment, the surfactant is a nonionic surfactant (e.g. polysorbate or Tween 80).

In some embodiments, Tween 80, a polyethylene glycol or a polyoxyethylene polyoxypropylene glycol is included at approximately 0.001% (w/v) to about 10% (w/v).

In embodiments involving a stabilizer, the stabilizer may be any suitable stabilizer known to the art (e.g. Stella and Rajewski, 1997; Merisko-Liversidge and Liversidge, 2003; U.S. Pat. No. 5,376,359). The stabilizer, may for example, be an amino acid, such as for instance, glycine; or an oligosaccharide, such as for example, sucrose, tetralose, lactose or a dextran. The stabilizer may also be a sugar alcohol, such as mannitol or a combination the stabilizer types described above.

In some embodiments, a stabilizer or stabilizers constitute approximately 0.1% to about 10% weight for weight of the compound.

Examples of acceptable salts useful in the invention include, but are not limited salts formed with inorganic acids (e.g. those selected from the group consisting of hydrochloric, hydrobromic, sulfuric, phosphoric, nitric or equivalent), or salts formed with acids or organic acids (e.g. acetic, oxalic, tartaric, succinic, malic, fumaric, aleic, ascorbic, benzoic acid, tannic, alginic, polyglutamic, naphthalene sulfonic acid, naphthalene disulfonic acid and polygalacturonic).

Treatment, exposure, combining or complexing of the drug(s), compounds, extracts, oils, agents, and/or drugs, etc. of the present invention “with acids” will frequently result in beneficial changes to bioavailability, pharmacokinetics, metabolism, toxicity and/or excretion of the products of said acid treatments, and/or their metabolites, as compared to the untreated, unexposed, uncombined, and uncomplexed, compounds, extracts, oils, agents, and/or drugs, etc.

The same may often be true with respect to treatment with various chemical bases. Accordingly, such products of acid or base treatment, exposure, combining or complexing are covered by the various embodiments described herein. In some embodiments, the compositions, manufacture, products, processes, methods, and/or methods of use, prevention and treatment useful in the methods of the present invention contain one or more conventional additives. Additives include a solubilizer (e.g. US20070021325; U.S. Pat. No. 6,669,964; WO2009126950; WO2009101263). Additives may comprise glycerol or an antioxidant such as for example, benzalkonium chloride, benzyl alcohol, chloretone or chlorobutanol. Additives may also include an anesthetic.

To reduce oxidation and spoilage, the pharmaceutical compositions, manufacture, products, processes, methods, and/or methods of use, prevention and treatment may be stored under nitrogen gas or argon gas in sealed vials.

In one preferred embodiment a buffering agent is selected from the group comprising sodium biphosphate, potassium biphosphate, sodium bicarbonate, potassium bicarbonate, carboxylic acids and their salts.

The buffering agents of the present invention may be any salt or buffering agent. Examples include sodium chloride, potassium chloride, or sodium phosphate or potassium phosphate.

In some embodiments, the salt and/or buffering agent is useful in maintaining osmolality in a suitable range for administration of the composition to a human or an animal. The salt or buffering agent may preferably be present at isotonic concentration of about 150 mM to about 300 mM.

Examples buffers include sodium biphosphate, potassium biphosphate, sodium bicarbonate, potassium bicarbonate, carboxylic acids and their salts, such as, ascetic acid/sodium acetate and citric acid/potassium citrate.

The buffering agent will in some embodiments, maintain the pH of the composition in the range of about 5.5 to about 7.5.

In one preferred embodiment the composition further comprises at least one second active agent having therapeutic benefit.

In some embodiments, the composition and method further comprises an additional agent, drug or compound such as an FDA approved at least one agent, compound or drug or non-FDA approved at least one agent, compound or drug.

In some embodiments the invention also specifically covers the use of compounds, agents, and drugs specified or named herein (and their analogs), in conjunction with other anti-hypertensive agents, cardioprotectant agents, anti-obesity agents, fertility agents, glycemic control agents, anti-hyperlipidemic agents, anti-atherosclerotic agents, anti-cancer agents, anti-chemotherapeutic resistance agents, and other approved agents and drugs as part of combination therapies and medicinal compositions.

In some embodiments, the invention relates to novel compositions and delivery methods that increase the availability of various compounds, agents and drugs to the body, especially via the oral route, particularly when such drugs and compounds otherwise lack significant oral bioavailability.

In some embodiments, the invention relates to a composition comprising a natural oil or extract.

Further, in some embodiments, the ratio of at least one agent, compound, or drug of the present invention to other active compounds or agents in the compositions, manufacture, products, processes, methods, and/or methods of use, prevention and treatment ranges from 1:10 to 10:1.

In some embodiments, the ratio of at least one agent, compound, or drug of the present invention to glutathione is 1:1.

In some embodiments, the ratio of at least one agent, compound, or drug of the present invention to glutathione is 1:4 to 1:10.

In some embodiments, the ratio of at least one other agent, compound, or drug of the present invention to other active compounds or agents in the compositions, manufacture, products, processes, methods, and/or methods of use, prevention and treatment ranges from 1:50 to 50:1 based upon dry weight.

In some embodiments, at least one agents, compounds, products, derivatives, structural variants, and/or drugs described herein are combined with an agent, agents, a compound, compounds, a drug, drugs, and/or their structural variants.

In some embodiments, the compositions, manufacture, products, processes, methods, and/or methods of use, prevention and treatment of the present invention comprise at least one selected from a methoxyflavone (especially a dimethoxyflavone), n-acetylcysteine, glutathione, a glutathione precursor or a glutathione enhancing agent or a known intracellular glutathione promoting agent, folinic acid, folic acid, trimethylglycine, vitamin D, and medicinal iron.

In some embodiments, the composition comprises glutathione, especially reduced glutathione. In some embodiments, this invention relates to compositions, manufacture, products, processes, methods, and/or methods of use, prevention and treatment for achieving clinical benefit related to an increase in intracellular glutathione.

In some embodiments, the compositions, manufacture, products, processes, methods, and/or methods of use, prevention and treatment comprise at least one agent, compound, or drug of the present invention and an approved drug.

In some embodiments, at least one other named agent, compound, or drug of the present invention and/or reduced glutathione is incorporated into the approved drug's composition along with the approved drug without otherwise altering the approved drug's composition.

In some embodiments, at least one other agent, compound, or drug of the present invention and/or reduced glutathione is incorporated into the approved drug's composition along with the approved drug while adjusting the concentration of at least one of the compositions active drug, stabilizer, buffer, vehicle, excipient, etc.

In some embodiments, reduced glutathione (in doses ranging from about 200 mg to 2000 mg) is added to any of the compositions, manufacture, products, processes, methods, and/or methods of use, prevention and treatment described herein.

In some embodiments, the compositions, manufacture, products, processes, methods, and/or methods of use, prevention and treatment of the present invention comprise an analog of a compound, agent, or drug named herein.

In some embodiments, reduced glutathione is added to any of the compositions, manufacture, products, processes, methods, and/or methods of use, prevention and treatment described herein.

In some embodiments, a compound or a drug of the classes exemplified herein are combined in a nanoemulsion, nanoparticles (e.g. WO/2010/013224), nanovault, nanofiber, nanotube or other nanostructure.

In some embodiments, the analogs/derivatives of the present inventions are produced through addition of a mono-phenyl ring, addition of a heterocycle, addition of a substituted amide, addition of an unsubstituted amide, addition of a carbonyl imidazole, addition of a CN functional group, addition of a CO H2 functional group, addition of a CO HNH2 functional group, addition of a CO-D-Glu (OAc) 4 functional group, and/or addition of a ketone to one of the following: an approved drug (e.g. one named or described herein), an OTC drug, an ATF4 modulator, an FST1 modulator, an FST1 modulator, an NRF2 modulator, a KEAP1 modulator, a vitamin.

The present invention covers compositions, manufacture, products, processes, methods, and/or methods of use, prevention and treatment comprising agents, compounds and drugs of the present invention, their derivatives, analogs, and isomers. These derivatives, analogs, and isomers include, but are not limited to acetyl, acetate, phenylacetate, hydro, dihydro, formate, methyl ether, dimethylether, caprylate, valeriate, isovaleriate, alcohol, aldehyde, ketone, epoxide, lactone and cyclases derivatives.

In some embodiments, the compositions, manufacture, products, processes, methods, and/or methods of use, prevention and treatment of the present invention comprise agents, compounds, or drugs.

In some embodiments, the composition and method of the present invention comprises agents, compounds or drugs of the various classes named herein.

In some embodiments, the composition and method of the present invention comprises an anti-EGR1 agent, an anti-EGR2 agent, an anti-EGFR agent and/or another approved drug to prevent or treat cancer metastasis.

In some embodiments, the present invention provides the means of increasing the cytotoxic effects of at least one chemotherapeutic agents against multiple myeloma or other cancer cells in an individual, comprising the steps of: administering to said individual said at least one chemotherapeutic agents and an agent, compound or drug of the present invention, wherein said composition of the present invention increases the cytotoxic effects of said one or more chemotherapeutic agents against multiple myeloma cells in said individual.

In some embodiments, at least one chemotherapeutic agents is selected from the group consisting of vincristine, BCNU, melphalan, cyclophosphamide, Adriamycin, prednisone velcade, thalidomide, and dexamethasone or other approved chemotherapeutic listed herein.

In some embodiments, the composition of may comprise at least one synthetic compound, and/or at least one approved drug.

In some embodiments, the FDA approved drug is selected from one or more of the following drug classes: Analgesics, Antacids, Antianxiety Drugs, Antiarrhythmics, Antibacterials, Antibiotics, Anticoagulants and Thrombolytics, Anticonvulsants, Antidepressants, Antidiarrheals, Antiemetics, Antifungals, Antihistamines, Antihypertensives, Anti-Inflammatories, Antineoplastics, Antipsychotics, Antipyretics, Antivirals, Barbiturates, Beta-Blockers, Bronchodilators, Cold Cures, Corticosteroids, Cough Suppressants, Cytotoxics, Decongestants, Diuretics, Expectorant, Hormones, Hypoglycemics (Oral), Immunosuppressives, Laxatives, Muscle Relaxants, Sedatives, Sex Hormones (Female), Sex Hormones (Male), Sleeping Drugs, Tranquilizer, and Vitamins.

In some embodiments, the composition comprises at least two agents, compounds, or drugs of the present invention or their analogs, derivatives, and isomers.

In some embodiments, the composition of the present invention comprises a VEGF inhibitor.

In some embodiments, a D vitamin or a B vitamin is added to any of the compositions, manufacture, products, processes, methods, and/or methods of use, prevention and treatment described herein. In some embodiments, an ATF4 modulator (see Roybal et al. 2005 and WO/2009/020601) and/or FST1 modulator is added to any of the compositions, manufacture, products, processes, methods, and/or methods of use, prevention and treatment described herein.

In some embodiments, an NRF2 and/or KEAP1 modulator is added to any of the compositions, manufacture, products, processes, methods, and/or methods of use, prevention and treatment described herein.

In some embodiments, the compositions, manufacture, products, processes, methods, and/or methods of use, prevention and treatment comprise an ATF4 modulator.

In some embodiments, the compositions, manufacture, products, processes, methods, and/or methods of use, prevention and treatment comprise an NRF2 and/or KEAP1 modulator.

In some embodiments, the agents, compounds and drugs of the present invention are biotinylated (e.g. U.S. Pat. Nos. 4,794,082; 5,521,319).

Additional chemical definitions, chemical structures, and formulae for compounds and compound classes useful in the present invention, as well as exemplary compositions, are further described in patent applications WO/2005/065667 as well other the other patent applications and articles referenced herein. They are all incorporated herein in their entirety. In some embodiments, the non-approved agent(s), compound(s) or drug(s) modulate enzymes in the NRF2 pathway.

In addition to being used as a monotherapy, the therapeutic methods of the present invention will also find use in combination therapies.

In some embodiments, the composition described herein comprises nationally approved agents, compounds or drugs listed herein.

In some embodiments, the composition described herein comprises FDA approved agents, compounds or drugs in 2023.

In some embodiments, the composition described herein comprises approved agents, compounds or drugs belonging to the drug classes represented in the list of FDA approved drugs, especially the list of FDA approved drugs in 2024.

These approved drugs include, but are not limited to drugs in the classes represented by Abacavir, Abametapir, Abarelix, Abatacept, Abciximab, Abilify, Abreva, Abrocitinib, Acalabrutinib, Acamprosate, Acarbose, Accolate, Accretropin, Acebutolol, Acellular, Acetaminophen, Acetate, Acetazolamide, Acetic, Acetohexamide, Acetohydroxamic, Acetonide, Acetrizoate, Aciphex, Aclidinium, Acrisorcin, Acrivastine, Actemra, Actiq, Activella, Actonel, Actoplus, Actos, Acular, Acuvail, Acyclovir, Adagrasib, Adalimumab, Adalimumab-aaty, Adalimumab-adbm, Adalimumab-afzb, Adalimumab-aqvh, Adalimumab-bwwd, Adalimumab-fkjp, Adapalene, Adcirca, Adderall, Adefovir, Aducanumab-avwa, Advicor, Advil, Aerolizer, Aerosol, Af, Afatinib, Afinitor, Aflibercept, Against, Agalsidase, Agenerase, Aggregated, Aggrenox, Agrylin, Ak-con-a, Akten, Alafenamide, Alamast, Albendazole, Albenza, Albumin, Albuterol, Alcohol, Aldara, Aldesleukin, Aldurazyme, Alefacept, Alendronate, Alesse, Alfacon-1, Alfa-tycv, Alfuzosin, Alirocumab, Alglucosidase, Alimta, Alinia, Aliskiren, Aliskiren+amlodipine, Alitretinoin, Allegra, Allegra-d, Allopurinol, Almotriptan, Alogliptin, Alora, Alosetron, Aloxi, Alphagan, Alphanine, Alprostadil, Alrex, Alseroxylon, Altabax, Altocor, Altretamine, Aluminum, Alvesco, Alvimopan, Amaryl, Ambrisentan, Amerge, Amevive, Amide, Amifampridine, Amifostine, Amiloride, Amino, Aminocaproic, Aminoglutethimide, Aminohippurate, Aminosalicylate, Aminosalicylic, Amisulpride, Amitiza, Amlexanox, Amlodipine, Amlodipine/atorvastatin, Ammonia, Amodiaquine, Amoxicillin, Amoxicillin/clavulanate, Amoxil, Amphetamine, Ampicillin/ampicillin, Amprenavir, Ampyra, Amrix, Anacaulase-bcdb, Anagrelide, Anastrozole, Androderm, Androgel, Aneuvysion, Anexsia, Angiomax, Angiotensin, Anidulafungin, Anifrolumab-fnia, Anisindione, Ansuvimab-zykl, Antazoline, Antihemophilic, Antithrombin, Antizol, Anzemet, Apalutamide, Aphthasol, Apixaban, Aplenzin, Apokyn, Apomorphine, Apraclonidine, Apremilast, Aprepitant, Apthasol, Aptivus, Arava, Arbutamine, Ardeparin, Aredia, Arestin, Arformoterol, Argatroban, Arginine, Aricept, Arimidex, Aripiprazole, Arixtra, Armodafinil, Aromasin, Arranon, Arsenic, Artemether, Artemether/lumefantrine, Arthrotec, Arzerra, Asacol, Asciminib, Ascorbate, Asenapine, Asfotase, Aspart, Aspirin, Astelin, Astepro, Atacand, Atazanavir, Atenolol, Atogepant, Atoltivimab, Atomoxetine, Atorvastatin, Atovaquone, Atracurium, Atridox, Atropine, Atrovent, Atryn, Augmentin, Auranofin, Avacopan, Avanafil, Avandamet, Avandia, Avastin, Avatrombopag, Avelox, Avinza, Avita, Avobenzone, Avonex, Axert, Axetil, Axid, Axona, Azacitidine, Azasite, Azatadine, Azathioprine, Azelaic, Azelastine, Azithromycin, Azmacort, Azor, Azt, Aztreonam, Azulfidine, Bacitracin, Baclofen, Bactroban, Baloxavir, Balsalazide, Banzel, Baraclude, Barium, Baycol, Bayer, Becaplermin, Beclomethasone, Belantamab, Belatacept, Bempedoic, Benazepril, Bendamustine, Benefix, Benicar, Benralizumab, Bentiromide, Benzamycin, Benzhydrocodone, Benzoate, Benzoyl, Benzyl, Bepotastine, Bepreve, Bepridil, Beractant, Berinert, Berotralstat, Besifloxacin, Besilate, Besivance, Besylate, Betamethasone, Betapace, Betaxolol, Betaxon, Bethanechol, Bevacizumab, Bevacizumab-awwb, Bevacizumab-bvzr, Bexarotene, Bextra, Bexxar, Biaxin, Bicarbonate, Bicisate, Bictegravir, Bicuculline, Bidil, Bimatoprost, Binimetinib, Biotin, Biperiden, Bisacodyl, Bismuth, Bisoprolol, Bisulfate, Bitartrate, Bivalent, Bivalirudin, Bleomycin, Boniva, Bortezomib, Bosentan, Botox, Botulinum, Bravelle, Bretylium, Brexpiprazole, Brigatinib, Brimonidine, Brincidofovir, Brinzolamide, Bromfenac, Bromide, Bromocriptine, Bromodiphenhydramine, Brovana, Buccal, Budesonide, Buffered, Bupivacaine, Buprenorphine, Buprenorphine/naloxone, Bupropion, Busulfan, Busulflex, Butabarbital, Butalbital, Butenafine, Butoconazole, Butorphanol, Butoxide, Butyrate, Byetta, C-13, Cabazitaxel, Cabergoline, Cabotegravir, Cabozantinib, Caduet, Cafcit, Caffeine, Calaspargase, Calcipotriene, Calcitonin, Calcitonin-salmon, Calcium, Calfactant, Cambia, Campath, Campostar, Campral, Camptosar, Camsylate, Canagliflozin, Canakinumab, Canasa, Cancidas, Candesartan, Candicidin, Capecitabine, Capreomycin, Caproate, Capromab, Caprylidene, Capsaicin, Capsule, Capsules, Captopril, Carbaglu, Carbamazepine, Carbatrol, Carbenicillin, Carbidopa, Carbon, Carbonate, Carboplatin, Cardizemâ®, Carglumic, Cariprazine, Carisoprodol, Carmustine, Carvedilol, Casimersen, Caverject, Cayston, Cea-scan, Cedax, Cedazuridine, Cefaclor, Cefamandole, Cefazolin, Cefdinir, Cefditoren, Cefiderocol, Cefotaxime, Cefoxitin, Cefpiramide, Cefprozil, Ceftaroline, Ceftazidime, Ceftibuten, Ceftin, Ceftolozane, Ceftriaxone, Cefuroxime, Celexa, Cellcept, Cellulose, Cemiplimab-rwlc, Cenestin, Cenobamate, Cephapirin, Cerivastatin, Cerliponase, Cernevit, Certolizumab, Cervarix, Cetirizine, Cetrotide, Cetuximab, Cevimeline, Chantix, Chenodiol, Chlophedianol, Chlorambucil, Chloramphenicol, Chloraprep, Chlordiazepoxide, Chlorhexidine, Chloroquine, Chlorothiazide, Chlorotrianisene, Chlorpheniramine, Chlorprothixene, Chlorthalidone, Cholestyramine, Choriogonadotropin, Chorionic, Chromate, Chromic, Chymopapain, Cialis, Ciclesonide, Ciclopirox, Cidofovir, Cilexetil, Cilostazol, Cimetadine, Cimetidine, Cimzia, Cinacalcet, Cinoxacin, Cinryze, Ciprofloxacin, Cisatracurium, Cisplatin, Citrate, Citric, Cladribine, Clarinex, Clarithromycin, Claritin, Claritin-d, Clascoterone, Clavulanate, Clemastine, Cleocin, Clevidipine, Cleviprex, Climara, Clindamycin, Clioquinol, Clobazam, Clobetasol, Clofarabine, Clolar, Clomiphene, Clomipramine, Clonazepam, Clonidine, Clostridium, Clopidogrel, Clotrimazole, Clotrimazole/betamethasone, Clozapine, Co-57, Co-58, Coagulation, Coartem, Cobicistat, Cocaine, Codeine, Colazal, Colchicine, Colcrys, Cold, Colesevelam, Colfosceril, Collagenase, Colloid, Combination, Combivir, Complete, Complex, Comtan, Concent, Concerta, Condylox, Confide, Conivaptan, Contraceptive, Copaxone, Corlopam, Corticorelin, Corticotropin-zinc, Cortisone, Corvert, Cosopt, Cosyntropin, Covera-hs, Cq, Cr, Cr-51, Crestor, Crinone, Crixivan, Crizanlizumab-tmca, Cromolyn, Cryptenamine, Curosurf, Cuvposa, Cyanocobalamin, Cyclobenzaprine, Cyclophosphamide, Cycloset, Cyclosporine, Cyclothiazide, Cycrimine, Cylert, Cymbalta, Cypionate, Cysteamine, Cytovene, Dabigatran, Dabrafenib, Daclatasvir, Daclizumab, Dacogen, Dactinomycin, Dalbavancin, Dalfampridine, Danazol, Dantrolene, Dapagliflozin, Dapiprazole, Dapsone, Daptacel, Daptomycin, Daratumumab, Darbepoetin, Darolutamide, Darunavir, Dasabuvir, Dasatinib, Daunorubicin, Ddavp, Decanoate, Decitabine, Defibrotide, Deflazacort, Degarelix, Degludec, Delavirdine, Demecarium, Denosumab, Dentipatch, Depakote, Depot, Depreotide, Derisomaltose, Dermagel, Dermagraft-tc, Deruxtecan-nxki, Deserpidine, Deslanoside, Desloratadine, Desmopressin, Desogestrel, Desonate, Desonide, Desoximetasone, Detrol, Deucravacitinib, Dexamethasone, Dexbrompheniramine, Dexchlorpheniramine, Dexfenfluramine, Dexmethylphenidate, Dextromethorphan, Dextrose, Diclofenac, Didanosine, Differin, Difluprednate, Dihydrate, Dihydrochloride, Diltiazem, Dimesylate, Dinitrate/hydralazine, Diovan, Dipivoxil, Dipropionate, Disodium, Disoproxil, Ditropan, Divalproex, Docetaxel, Docosanol, Donepezil, Doribax, Doripenem, Dornase, Dostinex, Double, Doxepin, Doxercalciferol, Doxil, Doxorubicin, Doxycycline, Dronedarone, Drospirenone/ethinyl, Drotrecogin, Droxia, Dulera, Duloxetine, Duoneb, Durezol, Dutasteride, Dutasteride+tamsulosin, Dynabac, Dynacirc, Ec, Ecallantide, Eculizumab, Edex, Edluar, Efferdose, Effexor, Efient, Egrifta, Elaprase, Electrolyte/dextrose, Elestrin, Eletriptan, Elidel, Eligard, Elitek, Ella, Ellence, Elmiron, Eloxatin, Eltrombopag, Embeda, Emend, Enalapril, Enbrel, Enfuvirtide, Enoxaparin, en-tabs, Entecavir, Entereg, Entocort, Epivir, Eplerenone, Epogen, Eprosartan, Eraxis, Erbitux, Eribulin, Erlotinib, Erythromycin, Escitalopram, Esclim, Esomeprazole, Esterase, Estradiol, Estradiol/norethindrone, Estratab, Estrogel, Estrogens, Estrogens/medroxyprogesterone, Estrostep, Eszopiclone, Etanercept, Etexilate, Ethinyl, Ethinylestradiol+levonorgestrel, Ethyol, Etodolac, Etravirine, Eulexin, Evamist, Everolimus, Evista, Evolocumab, Evoxac, Evra, Exalgo, Excedrin, Exelon, Exenatide, Extavia, Extina, Ezetimibe, Fabrazyme, Factor/coagulation, Famciclovir, Famvir, Fanapt, Faslodex, Febuxostat, Felodipine, Femara, Femhrt, Fempatch, Femstat, Fenofibrate, Fentanyl, Feraheme, Feridex, Ferrlecit, Fertinex, Ferumoxytol, Fesoterodine, Fexofenadine, Finacea, Finevin, Fingolimod, Flagyl, Flomax, Flonase, Flovent, Floxin, Flumist, Fluorouracil/leucovorin, Fluoxetine, Flutamide, Fluticasone, Fluvastatin, Fluvoxamine, Fluzone, Foam, Focalin, Follistima, Follitropin, Folotyn, Foradil, Formoterol, Forteo, Fortovase, Fosamax, Fosamil, Fosamprenavir, Fospropofol, Fosrenol, Fragmin, Frova, Frovatnptan, Fsh, Fulvestrant, Fumarate, Furoate, Furoate+formoterol, Fusilev, Fuzeon, Gabapentin, Galantamine, Galsulfase, Galzin, Ganciclovir, Gardasil, Gastrocrom, Gastromark, Gatifloxacin, Gefitinib, Gelnique, Gemcitabine, Gemtuzumab, Gemzar, Genotropin, Geodon, Geref, Gilenya, Glargine, Gleevec, Gliadel, Glimepiride, Glipizide, Glipizide/metformin, Globulin, Glucagon, Gluconate, Glyburide, Glycol-epoetin, Glycopyrrolate, Glyset, Golimumab, Gonadotropin, Gonal-f, Goserelin, Granisetron, Grepafloxacin, Guanfacine, Halaven, Handihaler, Havrix, Hectorol, Hepsera, Herceptin, Hfa, Hiberix, Histolyticum, Histrelin, Humalog, Humatrope, Humira, Hycamtin, Hyclate, Hydrochlorothiazide, Hydrochlorotiazide, Hydrocodone, Hydromorphone, Hylan, Ibandronate, Ibritumomab, Ibuprofen, Ibutilide, Idursulfase, Ilaris, Iloperidone, Imagent, Imatinib, Imiquimod, Imitrex, Immune, Implant, Inactivated, Incobotulinumtoxina, Increlex, Indinavir, Infanrix, Infasurf, Infergen, Infliximab, Inform, Innohep, Inspra, Insulin, Integrilin, Intelence, Interferon, Interstim, Intuniv, Invanz, Invega, Invirase, Iodixanol, Iontocaine, Ipratropium, Iressa, Iron, Irrigating, Isentress, Isopropyl, Isosorbide, Istodax, Itraconazole, Ivermectin, Ivyblock, Ixabepilone, Ixempra, Ixiaro, Jalyn, Januvia, Jevtana, Kadian, Kalbitor, Kaletra, Kapvay, Keppra, Ketek, Ketoconazole, Ketoprofen, Ketorolac, Kineret, Kit, Klaron, Kogenate, Krystexxa, Kunecatechins, Kuvan, Kytril, Lacosamide, Lamictal, Lamin, Lamisil, Lamivudine, Lamotrigine, Lanreotide, Lansopraxole, Lanthanum, Lantus, Lapatinib, Laronidase, Latuda, Lenalidomide, Lescol, Letairis, Letrozole, Leukine, Leuprolide, Levaquin, Levitra, Levocetirizine, Levofloxacin, Levoleucovorin, Levonorgestrel/20, Levonorgestrel-releasing, Levo-t, Levothyroxine, Levoxyl, Lexapro, Lexidronam, Lexiva, Lexxel, Lidocaine, Lidoderm, Liraglutide, Lisdexamfetamine, Lisinopril, Lispro, Lithium, Lithobid, Livalo, Lodine, Loratadine, Lotemax, Lotion, Lotrisone, Lotronex, Lovastatin, Lovenox, Lubiprostone, Lucentis, Lumigan, Lunesta, Lupron, Lurasidone, Lusedra, Lustra, Luvox, Luxiq, Lyrica, Lysteda, Macugen, Malarone, Maleate/diltiazem, Maraviroc, Marplan, Mavik, Maxalt, Mecasermin, Medoxomil, Medoxomil+amlodipine+hydrochlorothiazide, Meloxicam, Memantine, Menotropins, Mentax, Menveo, Meridia, Meropenem, Merrem, Mesalamine, Mesnex, Mesylate, Metadate, Metaglip, Metaprotereol, Metformin, Methylphenidate, Metoclopramide, Metoprolol, Metozolv, Metrolotion, Mevacor, Mg, Miacalcin, Micardis, Miconazole, Microspheres, Microzide, Midazolam, Midodrine, Miglitol, Migranal, Milnacipran, Minocycline, Minoxidil, Miraluma, Mirapex, Mircera, Mircette, Mirena, Mirtazapine, Mitoxantrone, Mobic, Mometasone, Monistat, Monurol, Monviala®, Morphine, Motrin, Mousse, Moxatag, Moxifloxacin, Mozobil, Multaq, Mupirocin, Muse, Mylotarg, Myobloc, Myozyme, Naglazyme, Naltrexone, Namenda, Naphazoline, Naprelan, Naproxen, Naproxen+esomeprazole, Nasacort, Nasal, Nasalcrom, Nascobal, Nasonex, Natalizumab, Natazia, Natrecor, Nedocromil, Nelarabine, Nelfinavir, Nesiritide, Neulasta, Neumega, Neupogen, Neupro, Neurontin, Nevirapine, Nexavar, Nexium, Niacin/lovastatin, Niaspan, Nicoderm, Nicorette, Nicotine, Nicotrol, Nilotinib, Nitazoxanide, Nitisinone, Nitrate, Nitroglycerin, Nitrostat, Nizatidine, Nolvadex, Norco, Norditropin, Norethindrone, Norgestimate/ethinyl, Noritate, Normiflo, Norvir, Novantrone, Novolog, Novothyrox, Noxafil, Nplate, Nuedexta, Nutropin, Nuvaring, Nuvigil, Ocuflox, Ocuhist, Odt, Ofatumumab, Ofloxacin, Oleptro, Olmesartan, Olopatadine, Omalizumab, Omeprazole, Omnicef, Onabotulinumtoxina, Ondansetron, Onglyza, Onsolis, Ophthalmic, Opthalmic, Oravig, Orencia, Orfadin, prefest, Osi, Osmocyte, Otic, Ovidrel, Oxaliplatin/5-, Oxcarbazepine, Oxybate, Oxybutynin, Oxycodone, Oxycontin, Oxytrol, Ozogamicin, Ozurdex, Paliperidone, Palonosetron, Pamidronate, Pamoate, Pancreaze, Pancrelipase, Panitumumab, Panretin, Pantoprazole, Paroxetine, Patanase, Patch, Paxil, Pazopanib, PCSK9 inhibitor, Pediarix, Pegaptanib, Pegasys, Peginterferon, Pegloticase, Pegol, Pegvisomant, Pemetrexed, Pennsaid, Pentavalent, Pentosan, Pentoxifylline, Pepcid, Perflexane, Periostat, Phoslo, Phosphate, Photofrin, Pindolol, Pioglitazone, Piperacillin, Pitavastatin, Plavix, Plenaxis, Plerixafor, Pneumococcal, Pokofilox, Polacrilex, Polifeprosan, Polyethylene, Polysulfate, Posaconazole, Posicor, Pradaxa, Pralatrexate, Praluent, Pramipexole, Pramlintide, Prandin, Prasugrel, Pravachol, Pravastatin, Precose, Pregabalin, Premarin, Premphase, Prempro, Prevacid, Preven, Prevnar, Prevpac, Prezista, Priftin, Prilosec, Prinivil, Proamatine, Procainamide, Procanbid, Prochloroperazine, Prochlorperazine, Progabide, Progesterone, Prograf, Proguanil, Proleukin, Prolia, Promacta, Prometrium, Propecia, Proscar, Protonix, Protopic, Provenge, Proventil, Prozac, Pseudoephedrine, Pulmozyme, Quadramet, Quadrivalent, Quetiapine, Quinidine, Quixin, Qutenza, Qvar, Rabeprazole, Raloxifene, Raltegravir, Ramelteon, Ranexa, Ranibizumab, Ranitidine, Ranolazine, Rapamune, Raplon, Rasburicase, Raxar, Rdna, Rebetol, Rebetronâ,¢, Rebif, Reclast, Recombinant, Reconstitution, Reditabs, Reduction, Redux, Refludan, Regranex, Release, Relenza, Relpax, Remeron, Remicade, Reminyl, Remodulin, Renagel, Renagelrenagel, Renova, Renvela, Reopro, Repatha, Repro, Requip, Rescriptor, Rescula, Respigam, Respiratory, Restasis, Retapamulin, Retavase, Reteplase, Retin-a, Revlimid, Reyataz, Rhinocort, Ribavirin, Rid, Rifaximin, Right, Rilutek, Riluzole, Risperdal, Ritalin, Ritonavir, Rituxan, Rivastigmine, Rizaben, Rocephin, Rofecoxib, Romidepsin, Romiplostim, Ropinirole, Rosiglitazone, Rosuvastatin, Rotadisk, Rotarix, Rotateq, Rotavirus, Rotigotine, Rozerem, Rufinamide, Rx, Rythmol, Sabril, Saizen, Salagen, Salts, Samarium, Samsca, Sanctura, Sancuso, Saphris, Sapropterin, Saquinavir, Sargramostim, Savella, Saxagliptin, Sclerosol, Sd, Seasonale, Seasonique, Secreflo, Secretin, Selegiline, Self-examination, Selzentry, Sensipar, Seprafilm, Serevent, Sermorelin, Seroquelâ®, Serpacwa, Sertraline, Sevelamer, Silenor, Simponi, Simulect, Single-entity, Singulair, Sipuleucel-t, Sirolimus, Sitagliptin, Skelid, Sodium, Sodium/tazobactam, Solifenacin, Soliris, Soltab, Soluble, Solution, Somatropin, Somatropin-rdna, Somatuline, Somavert, Sonata, Sorafenib, Sparfloxacin, Spectracef, Spiriva, Sporanox, Spray, Sprix, Sprycel, Stavudine, Stavzor, Stelara, Sterile, Strattera, Strength, Stromectol, Subutex/suboxone, Succinate, Sucrose, Sulfacet, Sulfamylon, Sulfasalazine, Sulfate, Sumatriptan, Sunitinib, Supartz, Supprelin, Suspension, Sustained-release, Sustiva, Sutent, Symlin, Synagis, Syncitial, Synercid, Synthroid, Synvisc, Synvisc-one, T4, Tacrolimus, Tadalafil, Tamiflu, Tarceva, Tartrate, Tasigna, Tasmar, Tavist, Taxol, Taxotere, Tazorac, Teczem, Teflaro, Tegaserod, Tegretol, Tekamlo, Tekturna, Telavancin, Telbivudine, Telithromycin, Telmisartan, Temodar, Temsirolimus, Tenofovir, Tequin, Terbinafine, Teriparatide, Tesamorelin, Test, Testim, Testoderm, Testosterone, Tetanus, Tetrabenazine, Teveten, Thalomid, Therapy, Tiazac, Tigecycline, Tikosyn, Tilade, Tiludronate, Timentin, Tindamax, Tinidazole, Tinzaparin, Tiotropium, Tipranavir, Tiuxetan, Tizanidine, Tobi, Tocilizumab, Tolmetin, Tolterodine, Tolvaptan, Topamax, Topical, Topiramate, Topotecan, Toprol-xl, Torisel, Toviaz, Toxin, Toxoid, Toxoids, Tracleer, Tramadol, Trandolapril, Tranexamic, Tranilast, Transdermal, Transoral, Trastuzumab, Travatan, Travoprost, Trazadone, Trazodone, Treanda, Trelstar, Treprostinil, Tretinoin, Triamcinolone, Tribenzor, Tricor, Tri-cyclen, Trileptal, Trilipix, Tri-nasal, Trioxide, Tripedia, Triptorelin, Trisenox, Trivagizole, Trivora-21, Trivora-28, Trizivir, Tromethamine, Trospium, Trovan, Tts, Twinrix, Tygacil, Tykerb, Type, Types, Tysabri, Tyvaso, Tyzeka, Ulipristal, Uloric, Ultracet, Ultraject, Unoprostone, Urofollitropin, Uroxatral, Urso, Usp, Ustekinumab, Uvadex, Vaccine, Vaginal, Valacyclovir, Valcyte, Valerate, Valerate+dienogest, Valganciclovir, Valproic, Valsartan, Valstar, Valtrex, Vancenase, Vanceril, Vaprisol, Vardenafil, Varenicline, Vectibix, Velaglucerase, Velcade, Veltin, Venlafaxin, Venofer, Ventolin, Veramyst, Verapamil, Verdeso, Veregen, Versed, Verteporfin, Vesicare, Vfend, Viadur, Viagra, Vibativ, Victoza, Vidaza, Videx, Vigabatrin, Viii, Vimovo, Vimpat, Vioxx, Viracept, Viramune, Viread, Viroptic, Virus, Visicol, Visipaque, Vistide, Visudyne, Vitrasert, Vitravene, Vivelle, Vivelle-dot, Vivitrol, Von, Voriconazole, Votrient, Vpriv, Vyvanse, Wafer, Warfare, Warfarin, Weekly, Welchol, Western, Wilate, Willebrand, With, Women, Wound, Xeloda, Xenazine, Xenical/orlistat, Xeomin, Xgeva, Xiaflex, Xifaxan, Xigris, Xolair, Xopenex, Xr, Xyrem, Xyzal, Yasmin, Zaditor, Zagam, Zanaflex, Zantac, Zelnorm, Zemaira, Zemplar, Zenapax, Zenpep, Zerit, Zestril, Zevalin, Zidovudine, Zileuton, Zinc, Ziprasidone, Zipsor, Zirgan, Zithromax, Zocor, Zofran, Zoladex, Zoledronic, Zolmitriptan, Zoloft, Zolpidem, Zometa, Zomig, Zonegran, Zonisamide, Zortress, Zosyn, Zuplenz, Zyban, Zyclara, Zyflo, Zymaxid, Zyprexa, and Zyrtec.

In such instances, the approved drug(s) may typically be provided at or about the same dosage as usually prescribed for a particular indication, although lower or higher dosages may be desirable depending upon the clinical picture.

In some embodiments, the compositions or drug combinations of the present invention comprise one or more erythropoietin-like agent selected from erythropoietin, Darbepoetin (Aranesp), Epocept (Lupin pharma), Epogen, Epogin, Eprex, Procrit, NeoRecormon, Recormon, Methoxy polyethylene glycol-epoetin beta (Mircera), Dynepo, Epomax, Silapo (Stada), Retacrit, Epocept, EPOTrust, Erypro Safe, Repoitin, Vintor, Epofit, Erykine, Wepox, Espogen, ReliPoietin, Shanpoietin, Zyrop, or EPIAO (rHuEPO).

In some embodiments, one or more agents, compounds and drugs of the present invention are selected from the list comprising oltipraz, CDDO, a neurite outgrowth promoting prostaglandin, vitamin D, a B vitamin, and andrographolide.

In some embodiments, one or more agent, compound or drug of the present invention is an agent, compound, extract or drug derived from oils or extracts in the classes represented by the following exemplars: Agarwood; Agarwood; Almond, Aloe Vera; Bitter; Amber Oil, Avocado, Fossilized; Amber Oil, Fossilized; Ambrette Seed Fine; Ambrette Seed; Amyris; Angelica Root; Angelica Seed; arborvitae; Armoise (Mugwort); Balsam of Peru Oil; Balsam of Peru Resin; Basil, Sweet ct Linalool; Basil, Sweet ct Linalool; Basil, Sweet ct Methyl Chavicol; Beeswax Absolute; Bergamot; Bergamot k; Bergamot; Bergamot; Black Cumin; Black Currant; Caraway; Cardamom; Carnation Absolute; Carnation Extract; Carrot Seed; Cassie Absolute; Cedarwood, Atlas; Cedarwood, Himalayan; Cedarwood, Texas; Cedarwood, Virginia; Celery Seed; Chamomile, Blue; Chamomile, Roman; Champaca; Cilantro; Cinnamon; Cinnamon Bark; Cistus Traditional; Citronella; Citronella Wild; Clary Sage Absolute; Clary Sage, Bulgaria; Clary Sage, Russia; Clary Sage, USA; Clove Bud; Clove; Cocao Absolute; Coconut; Coffee Bean; Coffee Bean Oil; Cognac, Green; Coriander Seed; Coriander Seed; Cucumber Hydrosol; Cumin Seed; Cypress Leaf; Cypress, Blue; Davana; Eucalyptus, Blue Gum; Eucalyptus, Blue Mallee; Eucalyptus, Lemon; Eucalyptus, Narrow Leaf; Eucalyptus, Narrow Leaf, Fennel, Sweet; Fennel, Sweet; Fenugreek; Fir Needle; Fir, Balsam; Fir, Balsam Absolute; Fir, Balsam Absolute; Fir, Douglas; Fir, Silver; Frankincense; Frankincense, Somalia; Frankincense Frereana; Frankincense, Oman; Frankincense, Oman Rare; Frankincense, Somalia; Galbanum; Geranium Absolute; Geranium, Egypt; Geranium, Rose; Geranium, South Africa; Ginger; Ginger; Ginger; Ginger Lily; Ginger, Fresh; Goji; Grapefruit, Pink; Grapefruit, Ruby Red; Grapefruit, White; Hay Absolute; Helichrysum, Albania; Helichrysum, Croatia; Hemp; Hyssop Decumbens; Immortelle Absolute; Jasmine Absolute, Egypt; Jasmine Absolute, Egypt; Jasmine Absolute; Jasmine Absolute; Jasmine; Jasmine Concrete; Jasmine Extract; Jasmine Sambac Absolute; Jasmine Sambac Absolute; Juniper Berry; Juniper Berry; Juniper Leaf/Berry, Nepal; Juniper Leaf/Branch; Kava Kava; Kava Kava; Labdanum Absolute, Clear; Laurel Leaf; Lavandin, Grosso; Lavender High Elevation; Lavender Wild; Lavender Absolute; Lavender Hydrosol; Lavender, Bulgaria; Lavender, France; Lavender, Maillette; Lemon; Lemon Tea Tree; Lemongrass; Lemongrass Wild; Lime Distilled; Lime Expressed; Lime Essence Oil; Lime, Distilled; Liquidambar (Styrax; Lotus Absolute, Pink; Lotus Absolute, White; Availability; Mandarin, Green; Mandarin, Red; Mandarin, Yellow; Mango' Marjoram; Melaleuca; Melissa; Myrrh; Myrrh, Somalia; Myrrh, Somalia; Myrtle, Green; Nagarmotha (Cypriol; Neroli Extra; Neroli Extra; Neroli, Egypt; Neroli, Egypt; Neroli, France; Neroli, France; Neroli, Morocco; Niaouli; Oakmoss Absolute; Orange Wild; Orange Blossom Absolute; Orange Blossom Absolute Fine; Orange Blossom Extract; Orange Essence Oil; Orange, Bitter Green; Orange, Bitter Red; Orange, Blood; Orange, Wild; Orange, Sweet; Oregano, Turkey; Orris Butter (15 irones; Osmanthus Absolute; Palmarosa, Nepal Wild; Palmarosa, Sri Lanka; Palo Santo; Patchouli Double Distilled; Patchouli; Patchouli, Dark; Patchouli, Light; Patchouli, Sri Lanka; Pepper, Black; Peppercorn, Pink; Peppermint, Chocolate; Peppermint, France; Peppermint; Peppermint, USA; Petitgrain Absolute; Petitgrain Bigarade; Petitgrain sur Fleurs; Petitgrain, Mandarin; Petitgrain, Mandarin; Piper, Aduncum; Piper, malacophyllum; Pomegranate Seed; Ravensara Wild; Rhododendron Leaf; Rosalina; Rose Absolute, Bulgaria; Rose Absolute, Bulgaria; Rose Absolute, Egypt; Rose Absolute, Egypt; Rose Absolute, Morocco; Rose Absolute, Morocco; Rose de Mai Absolute; Rose de Mai Concrete; Rose de Mai Extract; Rose Hip Seed; Rose Otto, Bulgaria; Rose Otto, Turkey; Rose Otto, White; Rosemary Antioxidant; Rosemary ct Cineole; Rosemary ct Cineole; Rosemary ct Verbenone; Sage; Sandalwood Rare; Sandalwood Absolute, New Caledonia; Sandalwood, Australian Premium; Sandalwood, New Caledonia; Sandalwood, New Caledonia Extra; Sandalwood, Royal Hawaiian; Sea Buckthorn; Seaweed Absolute; Spearmint; Spearmint, USA; Spikenard; Spikenard, Green Wild; Spruce, Black; St. John's Wort; Tagetes; Tamanu (Foraha Oil; Tangerine Murcott; Tansy, Blue; Tea Tree; Thyme ct Linalool; Tobacco Absolute; Tonka Bean Absolute; Tonka Bean Absolute 20; Tuberose Absolute; Tuberose Extract; Turmeric; Turmeric; Vanilla Absolute; Vanilla Bourbon; Vanilla Bourbon; Vanilla Bourbon; Verbena; Vetiver Double Distilled; Vetiver, El Salvador; Vernonia, polyanthes; Vetiver, Haiti; Vetiver, Sri Lanka; Violet Leaf Absolute; Violet Leaf Absolute; Virola, surinamensis; Vitamin E Oil; White Sage; Wintergreen Wild; Yarrow, Blue; Ylang Ylang Absolute; Ylang Ylang Complete, Comoros; Ylang Ylang Extra; Ylang Ylang I; Ylang Ylang II; Ylang Ylang III; Ylang Ylang, Fine; and Yuzu. etc. Such oil agents, compounds, drugs, oils and/or extracts may be used alone or in combination, and/or in combination with approved drugs and/or other agents and compounds of the present invention.

In some embodiments, combination therapies will involve one or more approved drugs of the drug classes exemplified herein or listed above. In such instances, an accompanying approved agent(s), compound(s) or drug(s) may typically be provided at or about the same dosage as usually prescribed for a particular indication, although lower or higher dosages may be desirable depending upon the clinical picture.

Typically, the resulting novel combination will still be useful in preventing or treating a condition, disease or disorder for which the approved drug is considered to be approved for use. The agents, compounds, and drugs of the present invention (including their analogs and derivatives) may be administered before or after the other agent in intervals ranging from seconds to weeks.

In embodiments where the other approved therapy and the agents, compounds, and drugs of the present invention are applied separately to the cell, tissue, organ, subject, patient, or organism, one would generally ensure that a sufficient amount of time does not pass such that the agent and the agents, compounds, and drugs of the present invention would still be able to exert an advantageous, combined effect.

Approved therapies include drug therapies, immunotherapy, gene therapy, radiotherapy, chemotherapy, surgery, etc.

With respect to the approved drugs provided within compositions, manufacture, products, processes, methods, and/or methods of use, prevention and treatment of the present invention, the amount that will be effective in the treatment of a particular disorder or condition disclosed herein will depend on the nature of the disorder or condition, and can be determined by clinical techniques and in vitro or in vivo assays.

The precise dose of a drug to be employed will also depend on the route of administration, and should be decided according to the judgment of the practitioner and each patient's circumstances. However, suitable dosage ranges for oral administration are generally about 0.001 mg to about 1000 mg of an approved drug of the invention, or a pharmaceutically acceptable salt or complex thereof, per kg body weight/day. More preferably, the dose will be between 1 mg and 500 mg, between 5 mg and 250 mg, or between 10 mg and 100 mg. A bioequivalent amount of the approved drug will typically be provided by routes other than the oral route, if such a route of delivery is selected.

In one preferred embodiment, the composition takes the form of solution, liquid, gel, suspension, emulsion, lotion, tablet, pill, pellet, capsule, powder, sustained-release formulation, suppository, emulsion, aerosol, spray, drop, nanoemulsion, buccal or sublingual form, a transdermal patch or other form suitable for use, such as cosmetic cream, body lotion, body milk, ointment or shampoo.

The present compositions, manufacture, products, processes, methods, and/or methods of use, prevention and treatment therefore, can take the form of solutions, liquids (e.g. WO2010106191), gels (e.g. WO2007126915), suspensions, emulsion, tablets, pills, pellets, capsules, capsules containing liquids (e.g. WO2010106191), powders (US20040162273), sustained-release suppositories, emulsions, aerosols, sprays (e.g. WO/2010/109482), drops, suspensions, nanoemulsions (e.g. WO2010070675), sublingual compositions (e.g. WO2009067536), a transdermal patch (e.g. U.S. Pat. Nos. 5,004,610; 5,342,623; 5,344,656; 5,364,630; 5,462,745; and 5,508,038; 5,077,104; 5,268,209; 4,908,027; 5,633,008; 4,839,174; 4,943,435; and 5,167,242) or any other form suitable for use.

Pharmaceutical compositions containing the at least one agent, compound, or drug of the present invention may be prepared in any form, such as oral dosage form (powder, tablet, capsule, soft capsule, aqueous medicine (e.g. U.S. Pat. No. 6,068,850), syrup, elixirs pill, powder, sachet, granule), or topical preparation (cream, ointment, lotion, gel, emulgel (e.g. WO2007129162), balm, patch, paste, spray solution, aerosol and the like), or injectable preparation (solution, suspension, emulsion).

The compositions, manufacture, products, processes, methods, and/or methods of use, prevention and treatment of the invention relate to preventing the effects of aging and cell death induced by UV radiation. The invention also relates to the use of these compositions as tan extenders.

The compositions of the invention can be in the form of cosmetic creams, gels, lotions, milks, emulsions and solutions, ointments, sprays, oils, body lotions, shampoos, lotions after-shave, deodorants, soaps, lip sticks protectors, sticks and pencils for makeup.

The compositions of the present invention may comprise flavorings (e.g. extract of ginger, mint, strawberry, vanilla, etc).

In some embodiments, the compositions, manufacture, products, processes, methods, and/or methods of use, prevention and treatment of the present invention are the product of mixing the compounds and drugs in their wet or liquid forms.

In some embodiments, the compositions, manufacture, products, processes, methods, and/or methods of use, prevention and treatment of the present invention are the product of mixing the compounds and drugs in their wet or liquid forms, and subsequently preparing solutions, suspensions, emulsion, tablets, pills, pellets, capsules capsules containing liquids (e.g. WO2010106191), powders, sustained-release suppositories, emulsions, aerosols, sprays.

In some embodiments, the compositions, manufacture, products, processes, methods, and/or methods of use, prevention and treatment of the present invention are the product of mixing the compounds and drugs in their dry or solid forms.

In the form of a gel, the compositions, manufacture, products, processes, methods, and/or methods of use, prevention and treatment include suitable excipients such as cellulose esters or other gelling agents such as carbopol, guar gum, etc.

The compositions in pharmaceutical dosage forms may be used in the form of their pharmaceutically acceptable salts and complexes, and also may be used alone or in appropriate association, as well as in combination with other pharmaceutically active compounds.

According to the present invention a buccal or sublingual dosage form may be also be a “T”- or “L”-shaped solid dosage form. Perforated and fenestrated versions, as well as non-perforated and non fenestrated versions of this dosage form are also possible.

In one preferred embodiment the composition takes the form of nanoparticles, nanovaults, nanotubes, nanofibers, etc. and/or liposomes, micelles, other lipid based carriers, etc.

In one preferred embodiment the composition is a gum, stable liquid, troche, tablet, capsule, gummy, sports drink, sublingual composition, condiment, nutritional drink, polyethylene glycol-coated preparation, suspension, syrup, soft gel, topical formulation, nanoemulsion, nanoparticle preparation, food mixture, powder mixture, topical gel, sunscreen, lozenge, cream, aqueous formulation, injectable formulation.

The present invention includes any compositions known to the art that is suitable for administration of the agents, drugs, and compositions useful in the methods of the present invention. Examples include tablets (U.S. Pat. No. 4,209,513), capsules (e.g. US 2010/0021535; U.S. Pat. No. 7,011,846), such as gelatin capsules (e.g. U.S. Pat. No. 5,698,155), pills, troches (e.g. U.S. Pat. No. 3,312,594), elixirs, suspensions, syrups (e.g. U.S. Pat. No. 6,790,837), wafers (e.g. Wen and Park, 2010), chewing gum (e.g. Chaudhary and Shahiwala, 2010; Semwal et al. 2010); U.S. Pat. No. 6,531,114; Surana et al, 2010), etc.

In specific some embodiments, the oral dose of an approved drug is about 0.01 mg to about 100 mg/kg body weight/day, and more preferably about 0.1 mg to about 75 mg/kg body weight/day, and more preferably about 0.5 mg to 5 mg/kg body weight/day, or 0.1 mg to 0.5 mg/kg body weight/day.

In some embodiments, the compositions, manufacture, products, processes, methods, and/or methods of use, prevention and treatment of the present invention utilize a soft gel capsule (U.S. Pat. Nos. 2,780,355, 4,497,157, 4,777,048, 4,780,316, 5,037,698 and 5,376,381).

In some embodiments, the compositions, manufacture, products, processes, methods, and/or methods of use, prevention and treatment of the present invention are the product of mixing compounds, agents and drugs in their dry or solid forms and subsequently encapsulating those compounds, agents and drugs in a capsule for oral administration.

In some embodiments, the compositions, manufacture, products, processes, methods, and/or methods of use, prevention and treatment of the present invention are the product of mixing compounds, agents and drugs in their dry or solid forms and subsequently suspending those compounds, agents and drugs in a suspension.

In some embodiments, the compositions, manufacture, products, processes, methods, and/or methods of use, prevention and treatment of the present invention are the product of mixing compounds, agents and drugs in their dry or solid forms and subsequently preparing solutions, suspensions, emulsion, tablets, pills, pellets, capsules, capsules containing liquids, powders, sustained-release suppositories, emulsions, aerosols, sprays.

In some embodiments, the compositions, manufacture, products, processes, methods, and/or methods of use, prevention and treatment of the present invention are the product of mixing compounds, agents and drugs in their dry or solid forms, preparing tablets, pills, pellets, capsules, capsules, etc. and subsequently coating those compounds, agents and drugs with an enteric coating.

In some embodiments, at least one agent, compound, or drug of the present invention is provided in 5 mg, 10 mg, 20 mg, 50 mg, 100 mg, 50 mg, 500 mg, or 1000 mg doses at a frequency suitable to maintain a desirable plasma concentration.

In some embodiments, one agent, compound, and/or drug of the present invention at least one agent, compound, or drug of the present invention is provided at a dosage suitable to achieve a plasma concentration of between 1 and 1000 uM or 0.01 and 50 uM.

In some embodiments, at least one agent, compound, or drug of the present invention is provided at a dosage suitable to achieve a plasma concentration of between 15 and 100 uM.

In one preferred embodiment the composition is a functional food.

The compositions, manufacture, products, processes, methods, and/or methods of use, prevention and treatment described herein also allow for the production of a “functional health food” comprising the agent(s), compound(s), or drug(s) of the present invention for the prevention and improvement of a condition, disease, or disorder in a subject.

The term “a functional health food” defined herein is the functional food providing enhanced physical; psychological, physiological, or other functionality by adding the compositions, agent(s), compound(s), drug(s), analogs, derivatives, or compositions of the present invention to conventional food for the benefit of a human or mammal.

In one preferred embodiment the composition further comprises at least one of omega-3 fatty acids, olive oil, or other source of lipid.

In some embodiments, at least one agent, compound, or drug of the present invention further comprises at least one omega 3 fatty acids, olive oil, or other source of lipid.

In some embodiments, one or more agents, compounds, or drugs of the present invention is conveyed to the body in conjunction with omega-3 fatty acids.

In some embodiments, one or more agents, compounds, or drugs of the present invention is conveyed to the body in conjunction with omega-3 fatty acids together in a capsule.

It will be apparent to those skilled in the art that various modifications and variations can be made in the compositions, use and preparations of the present invention without departing from the spirit or scope of the invention.

In some embodiments, the agents, compounds, or drugs of the present invention are modified chemically using novel means as well as any means known to the art (e.g. Brandi et al., 2003; Kassouf et al., 2006; Chao et al., 2007; Cho et al., 2007; Weng et al., 2007; Lin et al., 2008; U.S. Pat. No. 6,974,801).

In one preferred embodiment the method comprises topical or systemic administration of said composition for the treatment of a disease associated with mir-21 and/or other oncogene expression in a subject in need thereof.

Such compositions, manufacture, products, processes, methods, and/or methods of use, prevention and treatment can be administered to a subject animal such as mammals (rat, mouse, domestic animals or human) via various routes. All modes of administration are contemplated (e.g. orally, rectally or by intravenous, intramuscular, subcutaneous, intra-cutaneous, intrathecal, epidural or intra-cerebroventricular injection).

In some embodiments, said method comprises orally, parenterally, sublingually or topically, or by various routes simultaneously administration of said composition or combination of the present invention.

With respect to the present invention, an agent, compound, or drug may, for example, be administered orally, parenterally (e.g. intravenously, intramuscularly, subcutaneously), intranasally, topically (e.g. WO/2009/153373; WO/2010/070675; WO2007126915), or transdermally (e.g. Cevc and Blume, 2001).

Topical compositions include, for example, emulsions, gels, and sunscreens (e.g. WO2010129213; WO2007001484; WO2006099687). The CTFA Cosmetic Ingredient Handbook, Seventh Edition, 1997 and the Eighth Edition, 2000 (both incorporated by reference herein in their entirety) describe a wide variety of cosmetic and pharmaceutical ingredients suitable for use in the compositions of the present invention. Examples of these functional classes disclosed in this reference include: absorbents, skin protectants, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, SPF boosters, reducing agents, skin bleaching agents, skin-conditioning agents (emollient, humectants, miscellaneous, and occlusive), solvents, foam boosters, hydrotropes, solubilizing agents, suspending agents (nonsurfactant), sunscreen agents, ultraviolet light absorbers, waterproofing agents, and viscosity increasing agents (WO2010129213).

Other routes of administration include rectal administration, intrathecal administration, administration involving mucosal absorption, and administration in aerosolized form (e.g. U.S. Pat. Nos. 5,126,123; 5,544,646).

The present invention covers the administration of compositions, manufacture, products, processes, methods, and/or methods of use, prevention and treatment useful in the methods of the invention to an animal by sustained release. Such administration is selected when it is considered beneficial to achieve a certain level of the drug in a body compartment over a longer period of time (e.g. serum or plasma concentration).

In some embodiments, the compositions, manufacture, products, processes, methods, and/or methods of use, prevention and treatment of the present invention are suitable for oral administration including extended release compositions (e.g. Pouton, 2000; Prasad et al., 2003; WO/2010/137027; WO/2020/129337; WO/2010/127100; WO/2010/127191; WO/2010/119300; WO/2010/114801; WO/2010/103544), and controlled release compositions (WO 02/083106; U.S. Pat. Nos. 5,567,439; 6,838,094; 6,863,902; 6,905,708).

The present invention calls for the administration of an agent, compound, or drug to a human in an amount effective for achieving its benefit. Typical daily doses of compounds comprising the composition vary approximately in the range of 0.5 mg to 5000 mg.

The effective amount of the compound to be administered can be readily determined by those skilled in the art, for example, through pre-clinical trials, clinical trials, and by methods known to scientists, physicians and clinicians.

The present invention covers in vivo methods for the administration of a compound, agent or drug to an animal. These methods may vary and are not limited to those described herein.

Within the pre-clinical and clinical period, any method known to the art may be employed for contacting a cell, organ or tissue with an agent, compound, or drug. Suitable methods include in vitro, ex vivo, or in vivo methods.

In vitro methods include cultured samples. For example, a cell can be placed in medium and incubated with a compound, agent or drug under conditions suitable for assaying its activity. Appropriate incubation conditions may be readily determined by those skilled in the art.

An effective amount of a compound, agent or drug useful in the methods of the present invention, may be administered to an animal by known methods. The compound may be administered systemically or topically.

In one preferred embodiment the method comprises administration of said composition in the form of nanoparticles, nanovaults, or liposomes.

In some embodiments, the compounds, drugs or agents of the present invention may be administered to a cell in vitro, ex vivo, or in vivo utilizing nanoparticles (Martins et al., 2009; WO/2010/013224), Such delivery allows for improved absorption and/or pharmacokinetics of the compounds, drugs or medicinal compositions.

In some embodiments, the compounds, drugs or agents of the present invention may be administered to a cell utilizing liposomes, nanoparticles, nanocapsules, nanovaults, etc. (see Goldberg et al., 2007; Li et al., 2007; Martins et al., 2009; Hu et al., 2010; Huang et al., 2010).

In some embodiments, the agents, compounds, drugs of the present invention may be administered to a cell in vitro, ex vivo, or in vivo utilizing nanoparticles, liposomes (WO/2010/009186; WO/2009/141450; WO/2009/065065; WO/2004/069224; WO/1999/013865), nanocapsules, nanovaults.

In some embodiments, the compounds, drugs or medicinal compositions of the present invention may be administered to a cell utilizing liposomes, nanocapsules, nanovaults, nanosuspensions, etc. (see Sholer et al., 2001; Goldberg et al., 2007; Li et al., 2007; Hu et al.; 2010; Huang et al., 2010).

In some embodiments, the compounds, drugs or medicinal compositions of the present invention may be administered using nanovaults engineered to allow cell type specific targeting (Kickhoefer et al. ACS Nano 3, 27-36 (2009)).

In some embodiments, the compounds, drugs or medicinal compositions of the present invention may be administered using recombinant nanovaults.

In some embodiments, the compounds, agents, or drugs of the present invention are incorporated into nanoparticles allowing absorption of orally administered compositions, manufacture, products, processes, methods, and/or methods of use, prevention and treatment increasing bioavailability (especially oral bioavailability).

In some embodiments, a compound, agent or drug of the present invention is incorporated into nanoparticles, liposomes, and/or nanovaults allowing increased bioavailability of the compound, agent or drug.

In some embodiments, the compounds, agents, or drugs of the present invention are loaded into solid lipid nanoparticles by ultrasonic and high-pressure homogenization. In some embodiments, compounds, agents and drugs of the present invention are loaded into solid lipid nanoparticles by ultrasonic and high-pressure homogenization along with Sodium Carboxymethyl Cellulose.

In some embodiments, the drugs and compounds of the present invention are 44 incorporated into engineered nanomaterials, nanoliposomes, nanoemulsions (e.g. WO2010070675), nanoparticles and nanofibers (Weiss et al., 006; 2007) for further incorporation into all manner of medicinal compositions and food items of all types, including, for example, milkshakes, muffins, hamburgers, fruit cocktails, granola, trail mix, vitamin drinks, sports drinks (U.S. Pat. Nos. 5,780,094; 4,981,687), nutritional supplements and energy drinks (U.S. Pat. No. 5,744,187; etc. (see Handbook of Functional Lipids; and “Food Nanotechnology, an overview” by Sekhon (2010), as well as Milk and Milk Products: Technology, Chemistry and Microbiology by Varnam and Sutherland (2001) for reviews).

In some embodiments, the compositions, manufacture, products, processes, methods, and/or methods of use, prevention and treatment of the present invention comprise a combination of compositions, manufacture, products, processes, methods, and/or methods of use, prevention and treatment named herein.

In some embodiments, the compounds, agents, or drugs of the present invention are loaded into solid lipid nanoparticles by ultrasonic and high-pressure homogenization.

In some embodiments, the compounds, agents, or drugs of the present invention are loaded into solid lipid nanoparticles (e.g. KR1020080014379; WO/2006/102768; WO/2000/006120).

In some embodiments, the compounds, agents, or drugs of the present invention are loaded into solid lipid nanoparticles by ultrasonic and high-pressure homogenization along with Sodium Carboxymethyl Cellulose (Hu et al., 2010).

In some embodiments, the compounds, agents, or drugs of the present invention are encapsulated into solid lipid nanoparticles (SLN) utilizing a double emulsion solvent evaporation (w/o/w) method (Li et al., 2010).

In some embodiments, the compound, agent or drug of the present invention may be delivered in the form of an aqueous solution (e.g. WO/2000/025,765), a lipid, or in a lyophilized form.

In some embodiments, the compositions, manufacture, products, processes, methods, and/or methods of use, prevention and treatment of the present invention are the product of mixing compounds, agents and drugs in their dry or solid forms and subsequently loading those compounds, agents and drugs into lipid.

In some embodiments, the compositions, manufacture, products, processes, methods, and/or methods of use, prevention and treatment of the present invention are the product of mixing compounds, agents and drugs in their dry or solid forms and subsequently loading those compounds, agents and drugs into liposomes (e.g. see Langer, 1990. Science 249:1527-1533; Treat et al, in Liposomes in the Therapy of Infectious Disease and Cancer, Lopez-Berestein and Fidler (eds.), Liss, N.Y., pp. 353-365 (1989); Lopez-Berestein, ibid., pp. 317-327).

In some embodiments, the compositions, manufacture, products, processes, methods, and/or methods of use, prevention and treatment of the present invention are the product of mixing compounds, agents and drugs in their dry or solid forms and subsequently loading those compounds, agents and drugs into solid lipid nanoparticles.

In some embodiments, the compositions, manufacture, products, processes, methods, and/or methods of use, prevention and treatment of the present invention are the product of mixing compounds, agents and drugs in their dry or solid forms followed by loading those compounds, agents and drugs into solid lipid nanoparticles and/or liposomes followed by drying or lyophilizing the mixture.

In some embodiments, the dried or lyophilized liposomes and/or solid lipid nanoparticles are encapsulated for oral administration.

In some embodiments, compounds, agents, or drugs of the present invention are PEGylated by Chemical conjugation with PEG.

In some embodiments, the compounds, agents, or drugs of the present invention are complexed with crystalline ascorbic acid in solid lipid nanoparticles.

In some embodiments, the agents, compounds, or drugs of the present invention are conjugated, coupled, linked or complexed with a nitric oxide (NO)-donor moiety.

In one embodiment, the agents, compounds, or drugs of the present invention are conjugated, coupled, linked or complexed with a nitric oxide (NO)-donor moiety.

Relevant example methods for coupling or conjugating a nitric oxide moiety to an agent, compound, or drug named herein have previously been described (e.g. WO92/01668, WO 95/30641, WO 97/16405; U.S. Pat. No. 5,859,053; WO/2002/011706; WO2010118968).

In some embodiments, the agents, compounds, or drugs of the present invention covalently linked through an aromatic spacer to an NO-releasing moiety (e.g. —ON02) (Del Soldato et al., 1999; Bratasz et al., 2006).

In some embodiments, a drug of the present invention is conjugated, coupled, linked or complexed with a nitric oxide-donor moiety.

In some embodiments, a drug of the present invention is covalently linked through an aromatic spacer to an NO-releasing moiety (e.g. —ON02) (Del Soldato et al., 1999; Bratasz et al., 2006).

In some embodiments, drugs of the present invention are biotinylated, fluorinated, or difluorinated.

In some embodiments, the present invention relates to compounds, drugs, and agents, or compositions, manufacture, products, processes, methods, and/or methods of use, prevention and treatment that improve drug action, e.g. reduce chemotherapeutic resistance including, but not limited to, cancer chemotherapeutic resistance, chemotherapeutic resistance to anti-hypertensive agents, cardioprotectant agents, chemotherapeutic resistance to anti-obesity agents, fertility agents, chemotherapeutic resistance to glycemic control agents, chemotherapeutic resistance to anti-hyperlipidemic agents, chemotherapeutic resistance to an anti-atherosclerotic agent, etc.

For example, a composition that reduces hyperlipidemia may be one that appropriately modulates the amount or activity of a hyperlipidemia related gene (e.g. SREBP-lc or other hyperlipidemia related gene listed herein).

In some embodiments the novel compositions and delivery methods likewise provide for increased palatability, especially via the oral route, particularly when such drugs and compounds otherwise are unpalatable.

The invention also specifically covers the use of compounds, agents, and drugs specified or named herein (and their analogs).

In some embodiments, the present invention relates to a method of identifying agents, compounds or drugs useful in preventing or treating CDCP related diseases and conditions as well as other disorders diseases and conditions treatable or preventable.

In part, the invention relates to a method for preventing or treating a CDCP or ODDC disease or condition. The method comprises administering a composition comprising an agent, compound, or drug of the present invention.

Non-limiting examples of other disorders (herein termed “other disorders”, or ODDC) preventable or ameliorated by administration of the compositions, manufacture, products, processes, methods, and/or methods of use, prevention and treatment described herein include, but are not limited to inflammatory diseases, oncological diseases, genetic diseases, ischemic diseases, infectious diseases, neurological diseases, hematological diseases, kidney diseases, vascular diseases, dermatological diseases, opthamological diseases, rheumatoid diseases, orthopedic diseases, gynecological diseases, obstetric diseases, pediatric diseases, etc. Additional non-limiting examples include sepsis, contrast-induced nephropathy, chronic kidney disease, pulmonary fibrosis, hypoxic conditions, chemical-induced lung injury, respiratory distress disorder, anon gap acidosis, nephritis, lupus, interstitial lung disease, graft dysfunction, hepatitis, acute kidney injury, noise-induced hearing injuries, poison ingestion, retinopathy, neurotoxicity, cancer-induced injury such as ototoxicity, respiratory infections, autism, Parkinson's or Parkinson's related disorders, Alzheimer's disease or other amyloid related disorders, movement disorders or other neurodegenerative disorders, conditions involving vasospasm, and conditions considered treatable by provision of n-acetylcysteine, injectable reduced glutathione, or a known intracellular glutathione enhancing agent.

In some embodiments, the agents, compounds or drugs of the present invention act as a fade cream for reducing dark spots or evening skin color or skin tone.

In some embodiments, the agents, compounds or drugs of the present invention are incorporated into compositions, manufacture, products, processes, methods, and/or methods of use, prevention and treatment for treating or preventing signs and symptoms of CDCP and ODDC.

In some embodiments, the compositions, manufacture, products, processes, methods, and/or methods of use, prevention and treatment of the present invention are useful for preventing or treating a Neurodegenerative disease or condition. Examples of such neurodegenerative diseases include Parkinson disease, Alzheimer disease, Multiple Sclerosis, Schizophrenia, Dementia, and Huntington's disease.

In some embodiments, the compositions, manufacture, products, processes, methods, and/or methods of use, prevention and treatment of the present invention are useful for preventing or treating a mental illness.

In some embodiments, the compositions, manufacture, products, processes, methods, and/or methods of use, prevention and treatment of the present invention are useful for preventing or treating diseases or conditions related to Aging. Examples of aging related diseases include Arthritis, Diabetes, Osteoarthritis, Cataracts, Macular Degeneration and Prostate enlargement.

In some embodiments, the compositions, manufacture, products, processes, methods, and/or methods of use, prevention and treatment of the present invention are useful for preventing or treating diseases or conditions related to Liver Dysfunction. Examples of such conditions and diseases include Toxic Hepatitis, Viral Hepatitis (A, B, and C), Chronic Hepatitis, Acute alcoholic Hepatitis, Alcoholic Hepatic fibrosis, Hepatic toxin exposure, and Cirrhosis.

In some embodiments, the compositions, manufacture, products, processes, methods, and/or methods of use, prevention and treatment of the present invention are useful for preventing or treating diseases or conditions related to Lung dysfunction. Examples of such diseases and conditions include Asthma, Emphysema, Pneumonia, Bronchitis (chronic and acute), Cystic fibrosis, Pulmonary fibrosis, Chronic obstructive pulmonary disease (COPD), Adult respiratory distress syndrome (ARDS).

In some embodiments, the compositions, manufacture, products, processes, methods, and/or methods of use, prevention and treatment of the present invention are useful for preventing or treating diseases or conditions related to the Cardiovascular System. Examples of such diseases and conditions include Ischemia, Atherosclerosis & its consequences, Heart failure, Heart Attack, Reperfusion injury, Kidney failure, High blood pressure, Stroke, Impaired circulation, vasculitis, and various viral and non-viral carditis.

In some embodiments, the compositions, manufacture, products, processes, methods, and/or methods of use, prevention and treatment of the present invention are useful for preventing or treating diseases or conditions related to the Digestive System.

Examples of conditions and diseases related to the Digestive System include inflammatory bowel disease, Ulcerative colitis, Crohn's disease, Gastritis, Stomach cancer, Pancreatitis, Peptic ulcer disease.

In some embodiments, the compositions, manufacture, products, processes, methods, and/or methods of use, prevention and treatment of the present invention are useful for preventing or treating diseases or conditions related to Kidney Failure & Dialysis, Examples of such diseases and conditions include Kidney failure, Renal toxicity, and Injury related to dialysis.

In some embodiments, the compositions, manufacture, products, processes, methods, and/or methods of use, prevention and treatment of the present invention are useful for treating a condition involving the skin, especially allergic conditions and conditions related to immune dysfunction.

In some embodiments, the compositions, manufacture, products, processes, methods, and/or methods of use, prevention and treatment of the present invention are useful for treating Infectious diseases.

In some embodiments, the compositions, manufacture, products, processes, methods, and/or methods of use, prevention and treatment of the present invention are utilized as anti-infectives (e.g. antibiotics, anti-microbials, anti-fungals, and antivirals, anti-helminthics, etc.)

In some embodiments, the compositions, manufacture, products, processes, methods, and/or methods of use, prevention and treatment of the present invention are useful for preventing or treating Immune System related diseases and conditions. Such diseases and conditions include viral infection, HIV and AIDS, Toxic Hepatitis & cirrhosis, Viral hepatitis (type A, B, & C), Herpes virus infection, Common Cold, various Bacterial infections, Chronic fatigue syndrome, and autoimmune dysfunction.

In some embodiments, the compositions, manufacture, products, processes, methods, and/or methods of use, prevention and treatment of the present invention are useful for preventing or treating Skin Disorders. Examples of such diseases and conditions include Pruritus, Psoriases, Eczema, SLE (lupus), Vasculitis, Polymyositis, Mycosis fungoides, Scleroderma Pemphigoid, Atopic dermatitis, Contact dermatitis, Sebborrheic dermatitis, Dermatitis herpetiformis, Acne conglobata, Acne vulgaris, Vitiligo, Alopecia areata, and UV radiation skin damage.

The invention also provides compositions, manufacture, products, processes, methods, and/or methods of use, prevention and treatment (including but not limited to oral and topical compositions) for promoting hair growth.

In some embodiments, the compositions, manufacture, products, processes, methods, and/or methods of use, prevention and treatment of the present invention are useful for preventing or treating diseases and conditions related to the Eye, Ear, Nose, Throat & Teeth. Such conditions and diseases include Cataract, Glaucoma, Macular degeneration, Hearing loss, Ear infection, Sinusitis, Periodontal (gum) disease, and upper respiratory tract disease.

In some embodiments, the compositions, manufacture, products, processes, methods, and/or methods of use, prevention and treatment of the present invention are useful for preventing or treating diseases and conditions related to the Pregnancy, Lactation & Childbirth. Examples of such disorders include Pre-eclampsia, Eclampsia Hypertension, and Diabetes.

In some embodiments, the compositions, manufacture, products, processes, methods, and/or methods of use, prevention and treatment of the present invention are useful for treating neurological disorders such as schizophrenia, multiple sclerosis, epilepsy, seizures, depression and bipolar disorder.

In some embodiments, the compositions, manufacture, products, processes, methods, and/or methods of use, prevention and treatment of the present invention are useful for treating fragile X syndrome.

In some embodiments, the compositions, manufacture, products, processes, methods, and/or methods of use, prevention and treatment of the present invention are useful for preventing or treating injuries and conditions related to Exercise & Athletic Performance. Such conditions and diseases may, for example, occur in the context of over training (e.g. Over-Training Syndrome) & the related cellular stress.

In some embodiments, the compositions, manufacture, products, processes, methods, and/or methods of use, prevention and treatment of the present invention are useful for treating a newborn.

In some embodiments, the compositions, manufacture, products, processes, methods, and/or methods of use, prevention and treatment of the present invention are useful for treating a child.

In some embodiments, the compositions, manufacture, products, processes, methods, and/or methods of use, prevention and treatment of the present invention are useful for treating an adult human.

In some embodiments, the compositions, manufacture, products, processes, methods, and/or methods of use, prevention and treatment of the present invention are useful for preventing or treating diseases and conditions related to hormonal influences such as loss of hair and fertility. In some embodiments, the compositions, manufacture, products, processes, methods, and/or methods of use, prevention and treatment of the present invention are useful for preventing or treating diseases and conditions related to toxic exposures.

In some embodiments, the compositions, manufacture, products, processes, methods, and/or methods of use, prevention and treatment of the present invention are useful for increasing telomerase activity in a cell when such an increase is desirable or preventing or treating diseases and conditions related to reduced or insufficient telomerase activity.

In some embodiments, the compositions, manufacture, products, processes, methods, and/or methods of use, prevention and treatment of the present invention are useful for the alleviation of pain, inhibition of platelet aggregation, lowering of fever and for prevention of cardiovascular disorders with reduced toxicity and/or reduced polypharmacy.

In some embodiments, the compositions, manufacture, products, processes, methods, and/or methods of use, prevention and treatment of the present invention are useful for vasorelaxant, antianginal, anti-inflammatory, analgesic and anti-thrombotic activity with lower gastrointestinal toxicity as compared to aspirin.

In one embodiment, chronic use of the compositions, manufacture, products, processes, methods, and/or methods of use, prevention and treatment of the present invention extend the lifespan of a cell, a tissue, an organ or an organism.

In one embodiment, chronic use of the compositions, manufacture, products, processes, methods, and/or methods of use, prevention and treatment of the present invention extend the lifespan of a human.

In one embodiment, the compositions, manufacture, products, processes, methods, and/or methods of use, prevention and treatment of the present invention are utilized as anti-infectives (e.g. antibiotics, anti-microbials, anti-fungals, and antivirals, anti-protozoals, anti-helminthics, etc.).

Furthermore, the compositions, manufacture, products, processes, methods, and/or methods of use, prevention and treatment of the present invention may, in some embodiments, also be beneficial in critical surgical patients, patients in intensive care settings, patients receiving hemodialysis.

In another part, the invention relates to compositions, manufacture, products, processes, methods, and/or methods of use, prevention and treatment for reducing an animal's body fat, increasing energy expenditure, and increasing oxygen consumption. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for increasing lipolysis, increasing expression of uncoupling protein 2 (UCP2) and beta-oxidation genes, decreasing expression of lipogenic genes in white adipose tissue, thereby increasing utilization and decreasing synthesis of fatty acids. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for increasing UCP1, 2 and 3 expression in brown adipose tissue (BAT), thereby increasing thermogenesis. Such activity represents organismal responses that may be assayed as a means of identifying compounds, drugs, and medicinal compositions suitable for preventing or treating CDCP, diseases related to CDCP, and for chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for improving ovulatory function (and thus fertility) in a female in need of such improvement, regularizing her menstrual cycle, and reducing hirsutism. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for lowering levels of circulating carbohydrate, preventing or treating age-related obesity, preventing or treating diet-related obesity, and preventing or treating steatosis. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for preventing or treating chronic hyperglycemia, and preventing or treating diet-induced diabetes. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In a further part, the invention relates to compositions, manufacture, products, processes, methods, and/or methods of use, prevention and treatment for preventing or treating chemotherapeutic resistance in a cell, tumor, or cancer cell. Such activity represents organismal responses that may be assayed as a means of identifying compounds, drugs, and medicinal compositions suitable for preventing or treating CDCP, diseases related to CDCP, and/or chemotherapeutic resistance. Likewise, these organismal responses may be assayed to measure the efficacy of such compounds, drugs, and medicinal compositions.

In one aspect, the invention relates to compositions, manufacture, products, processes, methods, and/or methods of use, prevention and treatment with anti-hypertensive agent, cardioprotectant agent, anti-obesity agent, glycemic control agent, anti-hyperlipidemic agent, an anti-atherosclerotic agent, and/or an agent preventing or treating chemotherapeutic resistance.

In some embodiments, the compositions, manufacture, products, processes, methods, and/or methods of use, prevention and treatment of the present invention are utilized to counteract a high fat diet.

In some embodiments, the compositions, manufacture, products, processes, methods, and/or methods of use, prevention and treatment of the present invention are utilized to counteract a diet of excessive calories.

In some embodiments, at least one agent, compound, or drug of the present invention is provided to reduce resistance to an approved drug, including, but not limited to anti-cancer drugs, glycemic control drugs, anthypertensie drugs, lipid reducing drugs, etc.

In some embodiments, at least one agent, compound, or drug of the present invention is provided to reduce resistance to an FDA over-the-counter (OTC) drug.

Vitamin C may be provided whenever reduced glutathione is selected for inclusion in the compositions, manufacture, products, processes, methods, and/or methods of use, prevention and treatment of the present invention in an amount to 0.5% w/v as needed.

Disease related activity (including signs and symptoms of disease) is considered reduced according to the invention if it is reduced at least about 10%, preferably, at least about 20%, more preferably at least about 30%, even more preferably at least about 40%, and most preferably at least about 50% or more than in the absence of the compositions, manufacture, products, processes, methods, and/or methods of use, prevention and treatment. Optimally, at least about 70%, more optimally at least about 85%, and most optimally 100% of the symptoms or signs of CDCP, or a disease related to CDCP, are reduced in vitro, ex vivo, or in vivo.

Likewise, the compositions, manufacture, products, processes, methods, and/or methods of use, prevention and treatment of the invention may modulate one or more of the following: tissue inflammation or swelling; pro-atherogenic cytokine production by endothelial cells, endothelial dysfunction, an invasion of blood vessel walls by monocytes, conversion of monocytes/macrophages to foam cells, smooth muscle proliferation, smooth muscle migration from tunica media to intima, plaque initiation, plaque progression, and plaque rupture; production of adipokines (e.g. TNF-alpha, IL-6, leptin, plasminogen activator inhibitor-1 (PAI-1), angiotensinogen, resistin, and C-reactive protein (CRP) by fat cells; an increase in plasma cholesterol, an increase in plasma LDL, an increase in plasma triacylglycerols, a decrease in plasma FIDL; an increase in blood glucose, an increase in fasting blood glucose, glucose intolerance, hyperinsulinemia, insulin resistance, HbAl, a dependence upon exogenous insulin; systolic and/or diastolic blood pressure, an angiotensin II, microalbuminuria; or cellular resistance to a chemotherapeutic agent.

In various embodiments, the composition for preventing or treating diseases related to CDCP, such as cardiovascular disease, diabetes, obesity, PCOS, steatosis, hyperlipidemia, and hypertension, as well as chemotherapeutic resistance, comprises one or more compounds and drugs selected from those named herein.

Anti-atherosclerotic activity refers to a composition's ability to induce a beneficial effect on blood vessels in vitro, ex vivo, or in vivo administration of the composition. Such beneficial effects include, but are not limited to preventing or reducing the likelihood of at least one of the following events: pro-atherogenic cytokine production by endothelial cells, endothelial dysfunction, an invasion of blood vessel walls by monocytes, conversion of monocytes/macrophages to foam cells, lipid oxidation, smooth muscle proliferation, smooth muscle migration from tunica media to intima, plaque initiation, plaque progression, and plaque rupture.

Anti-obesity activity refers to a composition's ability to induce a beneficial effect regarding excess weight gain upon in vitro, ex vivo, or in vivo administration of the composition. Such beneficial effects include, but are not limited to preventing or reducing the likelihood of one or more of the following events: production of adipokines (e.g. TNF-alpha, IL-6, leptin, plasminogen activator inhibitor-1 (PAI-1), angiotensinogen, resistin, and -reactive protein (CRP)) by fat cells.

Anti-hyperlipidemic activity refers to a composition's ability to induce a beneficial effect on lipid levels upon in vitro, ex vivo, or in vivo administration of the composition. Such beneficial effects include, but are not limited to preventing or reducing the likelihood of one or more of the following events: an increase in plasma cholesterol, an increase in plasma LDL, an increase in plasma triacylglycerols, and a decrease in plasma HDL.

For example, a composition that reduces hyperlipidemia and/or atherosclerosis may be one that appropriately modulates the amount or activity of a hypertension related gene (AZ Fernandez, 2010; Masuyama and Hiramatsu, 2012; Khalil, 2014; ERS Cheyou, 2014).

Glycemic control refers to a composition's ability to induce a beneficial effect on glucose levels, insulin levels, glucose tolerance, and/or insulin tolerance upon in vitro, ex vivo, or in vivo administration of the composition. Such beneficial effects include, but are not limited to preventing or reducing the likelihood of one or more of the following events: an increase in random blood glucose, an increase in fasting blood glucose, glucose intolerance, hyperinsulinemia, insulin resistance, an increase in HbAl, an increased, an increased dependence upon exogenous insulin.

For example, a composition that provides from improved glycemic control may be one that appropriately modulates the amount or activity of a glycemia related gene (e.g. V. Lyssenko, 2008; El-Osta, 2008; McCarthy, 2010; A L Siebel, 2010; Keating and El-Osta, 2013; HZ Huri, 2014; Rajasekar, 2015).

Anti-hypertensive activity refers to a composition's ability to induce a beneficial effect upon in vitro, ex vivo, or in vivo administration of the composition. Such beneficial effects include but are not limited to preventing or reducing the likelihood of one or more of the following events: an increase in systolic and/or diastolic blood pressure, an increase in angiotensin II, and an increase in microalbuminuria.

For example, a composition that reduces hypertension may be one that appropriately modulates the amount or activity of a hypertension related gene (R M Millis, 2011; G H Kim, 2011; S. Friso 2015; Y P C Chang, 2007; H J Dai, 2013, E. Larsson, 2013; Marlene, 2012).

Anti-chemotherapeutic resistance activity refers to a composition's ability to induce a beneficial effect upon in vitro, ex vivo, or in vivo administration of the composition. Such beneficial effects include, but are not limited to preventing or reducing the likelihood of at least one of the following events: cellular resistance to a chemotherapeutic agent as demonstrated by increased or persistent dysfunction in spite of the application of an otherwise effective chemotherapeutic agent.

Anti-CDCP activity refers activity refers to a composition's ability to induce a beneficial effect upon in vitro, ex vivo, or in vivo administration of the composition. Such beneficial effects include, but are not limited to preventing or reducing the likelihood of at least one of the following events: pro-atherogenic cytokine production by endothelial cells, endothelial dysfunction, an invasion of blood vessel walls by monocytes, conversion of monocytes/macrophages to foam cells, smooth muscle proliferation, smooth muscle migration from tunica media to intima, plaque initiation, plaque progression, and plaque rupture; production of adipokines (e.g. T F-alpha, IL-6, leptin, plasminogen activator inhibitor-1 (PAI-1), angiotensinogen, resistin, and C-reactive protein (CRP)) by fat cells; an increase in plasma cholesterol, an increase in plasma LDL, an increase in plasma triacylglycerols, a decrease in plasma HDL; an increase in blood glucose, an increase in fasting blood glucose, glucose intolerance, hyperinsulinemia, insulin resistance, an increase in HbAl, an increased dependence upon exogenous insulin; an increase in systolic and/or diastolic blood pressure, an increase in angiotensin II, an increase in microalbuminuria; or cellular resistance to a chemotherapeutic agent.

In some embodiments, the invention relates to a method for preventing or treating a variety of diseases and conditions including chronic diseases related to CDCP, such as cardiovascular disease, diabetes, obesity, PCOS, steatosis, hyperlipidemia, and hypertension and other disorders and conditions.

Contrary to the teachings of others, 4-aminopyridine appears to be associated with reduced appetite, increased satiety and weight loss in some patients.

Dalfampridine/Ampyra are reputed not to cause weight loss or hair loss. However, anecdotal patient self reports indicate that weight loss may be a feature of 4-aminopyridine treatment.

Enhanced BDNF expression in response to 4-aminopyridine is consistent with reduced appetite, increased satiety, and improvements in glucose metabolism and energy expenditure.

### Self Reports

Patient 1: ˜20 lbs lost over one month.

Patient 2: Reduced appetite/

Increased satiety.

Patient 3: weight loss/change to healthy diet

Patient 4: 65 lbs lost over 18 months. Increased strength/mow able to walk 1.5 miles daily.

### BRIEF DESCRIPTION OF THE DRAWINGS

FIG. 1 depicts a PCA Plot of 4-Aminopyridine treated cells (perturbation) and controls.

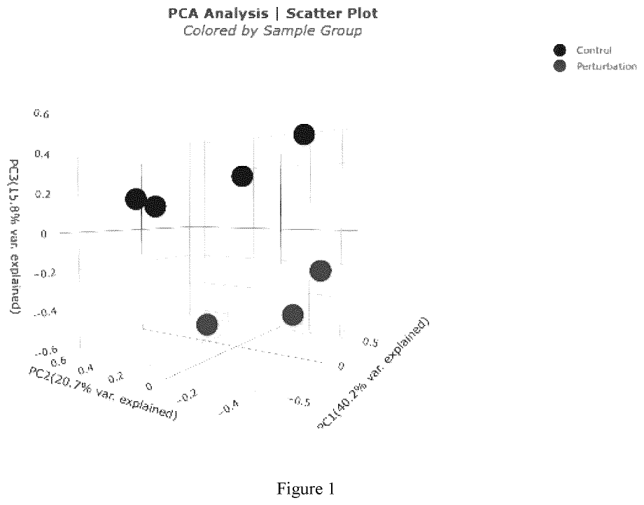

FIG. 2 depicts a Volcano Plot derived from differential gene expression in 4-Aminopyridine treated cells (perturbation) and controls.

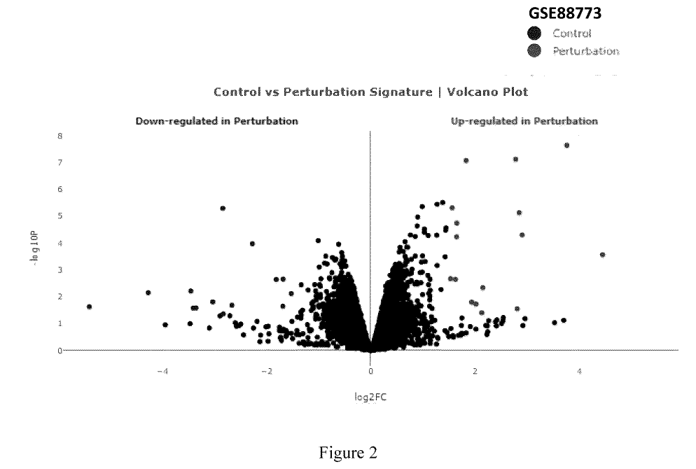

FIG. 3 depicts an MA Plot derived from differential gene expression in 4-Aminopyridine treated cells (perturbation) and controls.

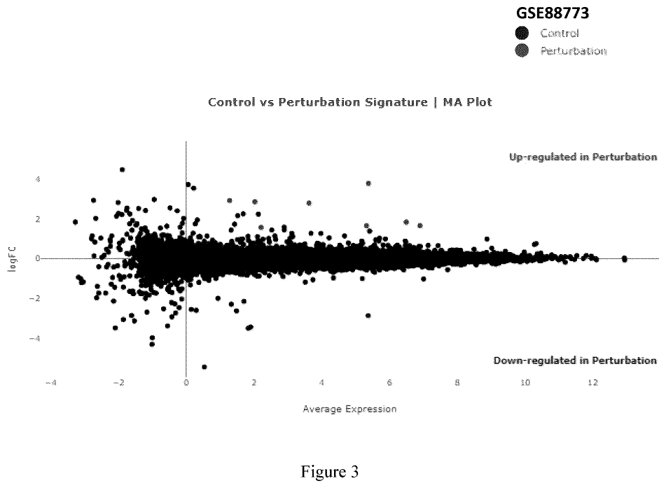

### EXAMPLES

The invention is now considered with respect to specific examples, though not limited thereby. As used within the following examples, the term at least one agent, compound, or drug of the present invention” refers to all conjugates and derivatives of at least agent, compound, or drug of the present invention. Thus, examples directed toward the inclusion of at least one agent, compound, and/or drug of the present invention one agent, compound, and drug described herein, as well as their analogs, isomers and/or derivatives at appropriate dosages. Accordingly, the amounts of the other components of the compositions, manufacture, products, processes, methods, and/or methods of use, prevention and treatment may likewise be appropriately scaled in relation to the mass of actually included agents, compounds or drugs. Thus, it should be understood that the example compositions and formations below may be altered by inclusion of OTC, and/or approved drug(s) in accordance with some embodiments of the invention. As used within the following examples, the term “glutathione” refers to all analogs, conjugates and derivatives of glutathione (e.g, glutathione monoethylester).

### Example 1 Preparation of Solid Lipid Nanoparticles

In one embodiment, the method chosen for the preparation of nanoparticles is an adaptation of the w/o/w double emulsion technique (Garcia-Fuentes et al 2003; Zhang et al 2006; Sarmento et. al., 2007). Approximately 200 mg of acetyl palmitate is dissolved in about 4 mL of dichloromethane. 7 mg of at least one agent, compound, and/or drug of the present invention and/or an equivalent effective amount of other agent(s), compound(s), or drug(s) of the present invention and glutathione) are dissolved in 0.5 mL of HCL 0.1 M. The drug solution is added to the lipid solution and then homogenized for 30 seconds in an ultra-turrax T25 (IKA-Labortechnik, Germany) or a similar apparatus. The primary emulsion is then poured into 25 mL of 2% poloxamer 407 solution and homogenized for another 30 seconds. The solvent is subsequently discarded and the emulsion is concentrated in a rotavapor until ˜10 mL. Optionally, particle size can be analyzed using photon correlation spectroscopy (PCS); and electrophoretic mobility can be measured with Laser Doppler Anemometry (LDA) using a Malvern Zetasizer 5000 (Malvern Instruments, UK) or similar apparatus. Samples can be diluted with Milli-Q-water having a conductivity adjusted to 50 μ8/αη by addition of a 0.9% NaCl solution.

The amount of the agent(s), compound(s) or drug(s) incorporated into SLN may be calculated by the difference between the total amount used to prepare the systems and the amount of compound or drug remaining in the aqueous phase after SLN isolation. After preparation, aqueous SLN dispersions may be centrifuged (by ultracentrifuge, rotor type 80Ti, Beckman Instruments, German or analogous instrument or similar apparatus) for about 2 hours at 45000 rpm (corresponding to approx. 190000×g). Compound, agent or drug concentration in the supernatant may be determined by HPLC (Sarmento et al 2006).

### Example 2 Preparation of a Liposomal Composition

A liposomal composition comprising at least one agent, compound, and drug of the present invention may be prepared according to Good Manufacturing Practices by the method of (Paul et al. (1997), previously described by Fessi et al. (1988). Briefly, an organic phase containing phospholipids, or at least one agent, compound or drug of the present invention is introduced under magnetic stirring in an aqueous phase. The organic solvent is evaporated, and the liposomes obtained are filtered and lyophilized. Prior to administration, 50 mg of lyophilized liposomes are resuspended in sterile distilled water (20 ml), shaken for 3 min, and then diluted in 5% dextrose.

### Example 3 Preparation of a Tablet Composition

Compressed tablets of the invention may be prepared by uniformly mixing at least one active ingredient with a solid carrier to provide a mixture. The mixture is then compacted to the shape and size desired. Molded tablets maybe made in a suitable machine. To prepare a tablet composition containing agents, compounds, or drugs of the present invention, the selected active components (e.g., at least one agent, compound, or drug of the present invention (80 g) and reduced glutathione (400 g)) may be mixed in the dry state for 10 minutes in a Z-blade mixer. Likewise, a solution is prepared containing gelatin (16 g), dioctyl sodium sulphosuccinate (1 g), alcohol (57 g) and purified water (80 g). The solution is then wet-mixed with the powders for 10 minutes using a slow speed. The wet mass is passed through a 1000μπι screen. Subsequently, the granules are dried in a fluidized bed at 60° C. for 30 minutes. The dried granules can then be sifted through a 1000μπι screen. Likewise, magnesium stearate (4.8 g) is sifted to 125μπι, and can be blended with the granules. Finally, the resulting mixture compressed on a Manesty D3 Rotary machine to provide tablets (U.S. Pat. No. 4,209,513).

### Example 4 Preparation of a Stable Liquid Composition

On order to prepare a stable liquid composition comprising the agents, compounds, or drugs of the present invention, the following are combined: 1 Excipient Amount/20 mL % of composition, the active components (e.g. at least one r agent, compound, or drug of the present invention 2.5 mg), 0.25 mg/mL water or pH 8 15.1 mL 75.5% v/v phosphate buffer glycerin 4 mL 20% v/v HPMC-K4 400 μl, of 0.1% solution 0.4 mg 0.002% w/v TWEEN® 80 100 μl, 0.5% v/v ethanol 200 μl, 1% v/v saccharin 400 μl, of 0.1% solution 0.4 mg 0.002% w/v (see U.S. Pat. No. 7,259,185).

### Example 5 Preparation of a Syrup Composition

To prepare a syrup composition comprising at least one other agent, compound, or drug of the present invention, 35% of the final batch volume of the purified water (USP/EP qs 1 L) is charged and heated to or at 60-80° C. The sugar (Sucrose Extra Fine Granulated USP 300.0 g/L), sodium benzoate (NF/EP 1.0 g/L), sodium citrate (Dihydrate USP/EP 5.27 g/L) and citric acid (Anhydrous USP/EP 2.15 g/L) are added and mixed until they dissolve. The solution is then cooled to 25-30° C. The sorbitol solution (USP/EP 142.0 g/L) and glycerin (Glycerol Anhydrous USP 150.0 g/L) are added, followed by a solution that contains propylene glycol (USP/EP 100.0 g/L) and a flavorant (1.0 g/L) mixed together. Finally, the at least one agent, compound, or drug of the present invention (40 g/L) is added and dissolved. The batch is finally brought to final volume by weight, and subsequently passed through a 1.2 micron filter. The concentration of at least one agent, compound, or drug of the present invention in this syrup composition may be reduced to accommodate the addition of other agents, compounds, or drugs named herein to produce desirable compositions.

### Example 6 Preparation of a Soft Gel Composition

To prepare a soft gel composition comprising at least one agent, compound, and drug of the present invention (e.g. at least one agent, compound, or drug of the present invention): polyoxyethanyl-a-tocopheryl-sebacate (PTS) (150 mg) and/or at least one other agent, compound, or drug of the present invention (100 mg) are melted and mixed them together at 60° C. To the cooled compositions are added oil (either rice bran oil, or omega-fatty acid enriched fish oil (ONC Oil 18/12) (30 mg) and beewax (50 mg). The composition is then incubated at 60° C. until the beeswax melts. The composition is finally mixed again and sealed under argon gas. The concentration of at least one agent, compound, or drug of the present invention or other agents, compounds, and drugs of the present invention in this soft gel composition may be reduced to accommodate the addition of other agents, compounds, or drugs named herein to produce desirable compositions.

### Example 7 Preparation of a Chewing Gum Composition

To prepare a chewing gum composition comprising the agents, compounds, and drugs of the present invention, the active medicaments are preferably added early-on into the mix. The smaller the amount of active ingredient used, the more important it is to preblend that particular ingredient to assume uniform distribution. Whether a pre-blend is used or not, in one embodiment, the agent or medicament should be added within the first five minutes of mixing. If the selected agents, compounds, and drugs are water soluble in the chewing gum, it preferably will include a base/emulsifier system which leads to the desired concentration of the medicament in the saliva (more hydrophilic balance). If the selected agents, compounds, and drugs are water insoluble, the chewing gum preferably includes a base/emulsifier system which leads to the desired concentration of the medicament in the saliva (more lipophilic balance). In manufacturing the gum ingredients may include the following Sugar (54.77%), Gum Base (21.80%), Corn Syrup (11.20%), Fructose (5.60%) Glycerine (3.40%) Active drug(s) (1.70%), Flavors (1.00%), Artificial Sweetener (0.26%), Soluble Saccharin (0.21%) and Insoluble Saccharin (0.06%). The precise percentages and many of the ingredients may vary (U.S. Pat. No. 7,078,052).

### Example 8 Preparation of an Overcoated Chewing Gum Composition

To prepare an overcoated chewing gum composition comprising the agents, compounds, and drugs of the present invention, the Gum Center is made as follows: Gum Base 33%, Calcium Carbonate 13%, Sorbitol 44.23%, Glycerin 4%, Flavors 2.32%, and/or at least one other agent, compound, or drug of the present invention 2%, Lecithin 0.6%, Sweeteners 0.9%. The center is sprayed with dried maltodextrin/and/or at least one other agent, compound, or drug of the present invention at 50% at least one active agent, compound, or drug of the present invention. The Gum Coating is composed of Coating Syrup 3, Coating Syrup 4, Xylitol 64.14%), Water 11.14%, 40% Gum Tahla Solution 20.87%, Titanium Dioxide Whitener 0.40% Peppermint Flavor 3 1.40%, Sweeteners 0.27%, Talc Polishing Agents 1.78%. The Flavor is added in 3 additions after 3 separate syrup additions within the coating syrup (1.4%). Finally, after completion of coating, the overcoated gum is polished. Following this protocol, the initial center piece achieves a weight of about 0.995 grams. The Gum is then coated to a finished piece weight of 1.52 grams to give a 34.5% coating. Coating syrup 3 is used to coat the first 60% of the coating to a piece weight of 1.30 grams. Coating syrup 4 is used to coat to the final piece weight (U.S. Pat. No. 6,290,985). The amount of at least one agent, compound, or drug of the present invention and the other ingredients in this gum may be adjusted to accommodate the addition of other agents, compounds, or drugs named herein to produce desirable compositions.

### Example 9 Preparation of a Troche Comprising at Least One Agent, Compound or Drug of the Present Invention

Long-lasting troches gradually release an active ingredient thereby prolonging absorption and duration of drug action. Troches also allow for sublingual absorption of agents that may have poor intestinal bioavailability (U.S. Pat. No. 3,312,594). To prepare a troche comprising at least one agent, compound, or drug of the present invention, and/or at least one the other agent, compound, or drug of the present invention, equal amounts of carboxymethylcellulose, pectin, and gelatin (e.g. 330 g each), are thoroughly admixed with magnesium stearate (e.g. 10 g) and with the active compounds, agents, or drugs (in appropriate concentrations). Afterwards, the mixed powder is compressed in a Stokes machine (or similar apparatus) to form troches of 500 mg each. The amount of at least one agent, compound, or drug of the present invention and the other ingredients may be adjusted to accommodate the addition of other agents, compounds, or drugs named herein to produce desirable compositions.

### Example 10 Preparation of a Sports Drink

To prepare a sports drink, desired and workable amounts of at least one compound, agent, or drug of the present invention may be added to sugar(s) selected from Galactose, Fructose, and Glucose (e.g. 2.5 g/100 ml), Sodium Chloride (e.g. 0.2 g/100 ml), Potassium (0.04 g/100 ml), Dihydrogen orthophosphate Magnesium (e.g. 0.01 g/100 ml). Citric acid or citrate may be used in an amount of 0.1 to 0.5% w/v as needed. When sodium citrate is used, the quantity of sodium chloride may be reduced in exact molar proportion to the sodium ions added as sodium citrate (up to 34 mmoH-l). Furthermore, caffeine and flavorings may be incorporated as desired. Preservatives, for example sodium benzoate or sorbic acid may likewise be employed. Vitamin C may be used as an antioxidant in an amount to 0.5% w/v as needed. The proportions set out above may be varied, but typically by 25% or less.

Alternatively, a composition for providing the health benefits listed herein while also providing a rapid source of energy, electrolyte balance, blood volume, and performance enhancement, may be produced by combining desired and workable amounts of each compound, agent, or drug of the present invention (e.g at least one agent, compound, or drug of the present invention 0.5 to 30% (preferably 5%), glutathione 0.5-10% (preferably 3%)), with electrolytes selected from e.g. sodium, potassium, chloride, phosphate, bicarbonate, sulfate, magnesium and calcium (e.g. about 1 meq/l to 6 meq/l potassium, 12 meq/l to 33 meq/l sodium, about 2 meq/l to about 8 meq/l phosphate), 0.5% to 5% glycerol (e.g. 1%), and about 2% to 8% sugar compound (e.g. 5% fructose, sucrose, glucose or other sugar). Specifically, the composition may have a glucose concentration of from about 2% to about 8%).

Preferably, the sugar concentration may be about 4%. The drink may be carbonated. In addition, caffeine may also be added (e.g. about 120-180 mg/I), as may other compounds such as vitamins, minerals, citric acid, citrate, preservatives, flavorings, sweeteners, and others. The proportions, set out above may be varied, but typically by 25% or less.

### Example 11 Preparation of a Stable Aqueous Composition Comprising Peptide Compounds in Water

To prepare a stable aqueous composition suitable for the provision of at least one r agent, compound or drug of the present invention, in combination with peptides including oligopeptides (e.g. reduced glutathione), the at least one agent, compound, and/or drug of the present invention is weighed (to achieve desired concentrations—e.g. total=40%) and then added to a weighed amount of vehicle (sterile distilled water, ethanol/water or water with non-ionic surfactant) at the appropriate concentration (w/w), then gently stirred to dissolve.

### Example 12 Preparation of a Powder Pharmaceutical Preparation Dissolvable in a Liquid to Form a Solution Prior to Ingestion

The powder pharmaceutical composition comprises safe and effective amount of the active agents. To prepare a Powder Pharmaceutical composition suitable for the provision of at least one agent, compound, or drug of the present invention, one may mix Ascorbic Acid (1.20%), Citric Acid (10.50%), Honey Buds Flavor (3%), Honey Powder Flavor (4%), Natural Lemon Flavor (5%), Natural Lime Flavor (6%)), Sweet-Ung (7%), Sodium Saccharin (0.30%), and Sugar Extra Fine Granulated (69.4985%).

### Example 13 Preparation of Encapsulated Nanoparticles

To prepare encapsulated nanoparticles of at least one agent, compound, or drug of the present invention, one may employ a single emulsion technique (Shaikh et al., 2009; 20090312402), a double emulsion technique, or a multi-emulsion technique.

### Example 14 Complexation of Polyphenols (e.g. Flavones and Flavonoids) of the Present Invention to Provide for Increased Absorption

Phosphatidylcholine (PC) may be used to increase the bioavailability of polyphenol compounds. Upon oral ingestion, the amphipathic PC molecules facilitate movement of the polyphenol through the intestinal epithelium to the bloodstream (Kidd, 2009).

### Example 15 the Preparation of a Spray or Drops

To prepare a spray (nasal or oral)/drops (e.g. ocular drops) composition comprising at least one agent, compound, or drug of the present invention, a borate buffer may be prepared by dissolving 3.81 g of sodium tetraborate in 100 ml mixture of water and or at least agent, compound or drug of the present invention dissolving 6.8 g of boric acid in 100 ml of water; and adjusting the pH of the sodium tetraborate solution to a pH of 7.1-7.3 by the addition of boric acid to provide a buffer. Subsequently, 60 mg agent(s), compound(s), or drug(s) of the present invention, 1 g of Tween80 and 1 g PEG may be combined and stirred well using a glass rod prior to sonication for 30 min or until the at least one agent, compound, or drug of the present invention is completely solubulized.

To prepare an ophthalmic composition, HPMC is added to 100 ml water and stirred until the HPMC is fully dissolved. Subsequently, the at least one agent, compound, or drug of the present invention/tween80 solution is added drop by drop and stirred for 15 minutes. NaCl, BAC, and EDTA are added and stirred until all the contents dissolve completely before adjusting the pH to 6.5 with borate buffer.

### Example 16 Preparation of a Nanoemulsified Topical Composition

To prepare a nanoemulsified topical composition comprising at least one agent, compound, or drug of the present invention, 5.28 g of glyceryl monosterate, 2.64 g of polyethylene glycol (PEG400), (+/−1 ml DMSO) and 2.64 g cetyl alcohol are transferred to a clean 50 ml beaker, followed by adding 2 ml light liquid paraffin and 100 mg Isopropyl myristate into the emulsifiers; then adding 1 ml Phenyl-2-Ethanol to the above mixture; followed by soaking 13.2 g of collagen in 10 ml of demineralized water till the solution becomes clear (˜25 min); followed by adding 100 mg niacinamide. Afterwards, 250 mg of at least one agent, compound, or drug of the present invention may be transferred into a clean container and solubilized into a nanoemulsion by mixing and sonicating with Tween 80 and PEG400. At about the same time, the solid emulsifiers, glyceryl monosterate, polyethylene glycol (PEG 400) and Cetyl alcohol are melted at 70 C, and the demineralized water (65 ml) simultaneously heated to 70 C. Then about half of the solubilized and/or other agent(s), compound(s), or drug(s) of the present invention is added into the hot emulsifiers to be mixed thoroughly. At this point, the melted emulsifiers may be added into the boiled demineralized water and mixed vigorously at the room temperature; until a creamy consistency is achieved. To this cream, at least one agent, compound, and/or drug of the present invention, collagen and niacin may be added to form a smooth cream before adding another half the amount of solubilized at least one agent, compound, or drug of the present invention and mixing. Then 100 μl Bronidox may be dissolved in 1 ml of PEG 400 is then added to the mixture as can be 100 μl of lavender oil to the above cream for fragrance.

### Example 17 Preparation of a Soluble, Liquid Composition Comprising the Agents, Compounds, or Drugs of the Present Invention

Agent or compound are provided as powder with fine granulometry (having the preferred and advantageous granulometry comprised between about 100 and about 200μπι) may be mixed with citric acid crystals (e.g. granulometry below 150μπι) and the resulting mixture stirred into at least one agent, compound, and/or drug of the present invention and Polysorbate 80. After heating to 300 C for adequate homogenization, this completed mixture may be mixed for 45 min. then milled with a three-roll-mill (e.g. a Coball mill) and closing aerated with nitrogen to remove present air. The preparation may then be encapsulated in gelatin capsules, preferably about 700 mg per capsule. Both non-coated capsules and enteric coated capsules with addition of E 904 (SHELLAC) may be used. Preferably, the concentration of one pure agent, compound, or drug of the present invention is preferably 6%, with 0.5% citric acid completed to 100% with at least one other agent, compound, and/or drug of the present invention and Polysorbate 80.

### Example 18 Preparation of a Hard Shell Capsule or Tablet Composition

The preparation is made as in example 17, though using a high viscosity emulsifier such as Polysorbate 60. SiO₂ may be added until a homogenous and fluid powder is achieved and to produce a percentage of 5% to 50% (preferably 30% to 35%). The resultant powder may then be used to fill hard shell capsules (preferably about 500 mg per capsule) or compressed into a tablet. Preferably, the concentration of at least one agent, compound, or drug of the present invention, in the final composition is 4%, with 0.35% citric acid and preferably a final concentration of SiO₂, of 30%). All percentages are on a weight by weight (w:w) basis.

### Example 19 Preparation of Agents, Compounds, and Drugs of the Present Invention Bound to Chitosan Nanoparticles

To prepare Chitosan Nanoparticles, a solution of 0.2% Chitosan (w/v) in 1% acetic acid may be prepared by heating the mixture to 75° C. The mixture may then be rapidly cooled to 4° C. and this process repeated several times until a solution of chitosan is obtained. This solution is then heated to 75 C again and sprayed under pressure into water kept stirring very rapidly at 4 C to produce uniformly dispersed chitosan nanoparticles. Such nanoparticles may be concentrated by centrifugation. Subsequently, 1 g at least one r agent, compound, or drug of the present invention in 1000 ml of absolute ethanol is added under pressure to vigorously stirred aqueous suspension of chitosan nanoparticles in 1% acetic acid and the resulting suspension may then be stirred overnight at 200-1400 rpm at room temperature to load and at least one agent, compound, or drug of the present invention on the chitosan nanoparticles.

### Example 20 Preparation of Nanoparticles

1 g of at least one agent, compound, or drug of the present invention, and/or other at least one agent, compound or drug of the present invention, may be dissolved in absolute ethanol. The solution may then be kept at 40° C. and then sprayed under nitrogen atmosphere and high pressure into 0.1% aqueous acetic acid solution. The solution is to be kept stirring at 200-1400 rpm at room temperature. The particle size can be controlled by varying the pressure at which the solution is sprayed into 0.1% aqueous acetic acid kept at different temperatures (25° C.-40° C.).

### Example 21

A Preparation of at least one agent, compound, or drug of the present invention combined under heat with methanol, while a lysine or arginine base is dissolved in water. Subsequently, the Lysine/Arginine Solution is Stirred into the at Least One Agent, Compound, or Drug of the Present Invention/Methanol Solution

### Example 22 Process for Reducing the Crystalline Nature of at Least One Agent, Compound or Drug of the Present Invention to Increase Solubility and Enhance Activity

To prepare at least one agent, compound, or drug of the present invention with diminished crystalline state, a process may be undertaken comprising: 1. preparing a mixed solution containing at least one agent, compound, or drug of the present invention and water-soluble or insoluble polymer in organic solvent or purified water; and 2. solid-dispersing at least one agent, compound, or drug of the present invention in the mixed solution in a polymer solution by using a spray dryer or fluidized bed granulator. In this context, the water-soluble polymer may be alginic acid, alginate or its derivatives, a-cyclodextrin or its derivatives, β-cyclodextrin or its derivatives, polyvinylpyrrolidone or its derivatives: polyvinylpyrrolidone-vinylacetate copolymer, y-cyclodextrin or its derivatives, polyoxyethylene-polyoxypropylene copolymer, polyethyleneglycol or its derivatives, polyvinylalcohol, xanthan gum, or arabic gum, or a combination of polymers.

### Example 23 Preparation of Cyclodextrine-Containing Derivatives for Increased Solubility

In order to prepare a more aqueous soluble, at least one agent, compound, or drug of the present invention suitable for administration, at least one is dissolved under heat in methanol, while lysine or arginine base is dissolved in water (see example 21 above). Subsequently, the lysine/arginine solution is stirred into at least one agent, compound, or drug of the present invention/methanol solution. The combined solution is then subjected to shaking and evaporation under vacuum, dissolving the non-dissolved residue in ethanol and bringing the mixture to the boiling point. Subsequently, non-dissolved residue is filtered out and the ethanol-based solution is maintained at about-200 C for approximately one hour. Once at least one agent, compound, or drug of the present invention lysinate and/or argininate is cooled and collected, it can be added to an aqueous cyclodextrin solution such as HP-beta-CD or HP-gamma-CD at once while agitating well. This new solution is then filtered.

### Example 24 At Least One Agent, Compound, or Drug of the Present Invention Dissolved in DMSO

To increase its solubility, at least one agent, compound, or drug of the present invention may be dissolved in 3% DMSO in sterile phosphate buffered saline (PBS). Subsequently, a 667 μM solution of at least one agent, compound, or drug of the present invention can be prepared for injection into an animal.

### Example 25 A Carbopol Dispersion Comprising the Agents, Compounds and Drugs of the Present Invention

In order to prepare a gel comprising at least one agent, compound, or drug of the present invention, one may first dissolve disodium edetate (0.05% by weight) in about 90% of the needed water (100% by weight). The at least one agent, compound, or drug of the present invention (1-5% by weight) may then be dissolved in solution by mixing until the at least one drug are dissolved to form a drug solution. After dissolving methylparaben (0.17%) and propylparaben (0.03% by weight) in propylene glycol (10% by weight) using heat as needed up to about 80 C and propeller mixing, one may add this solution slowly while mixing to the drug solution. Then 85% sodium docusate (1% by weight) may be dissolved in the drug solution with propeller mixing. Afterwards, Carbopol (0.6% by weight) is mixed into the drug solution to form a uniform dispersion. After dissolving oxybenzone (1% by weight) in octyl methoxycinnamate (7.5%) by weight), one may slowly pour this sunscreen solution into the Carbopol dispersion while mixing with a propeller mixer until uniform. Then one may make a 1% sodium hydroxide solution, with continuous mixing add it slowly and stepwise to the Carbopol® dispersion until the desired pH is attained. Add the remaining water and mix into the gel uniformly.

### Example 26 A Water-In-Oil Emulsion Suitable for Topical Administration

In preparing a water-in-oil emulsion (wherein preferably the base composition is included in the water phase and the water phase has a pH of about 5.8 to about 8, and an osmolarity between about 175 to about 330), the composition may include about 2.5 wt. % to about 3 wt. % base composition (e.g. electrolyte, buffer, mild preservative, lubricant) and about 20 wt. % to about 35 wt. % at least one agent, compound, or drug of the present invention. If the emulsion is intended for use in a sunscreen, then at least one sunscreen agent may be selected (e.g. from the group consisting of: octyl methoxycinnamate, octyl salicylate, homosalate, titanium dioxide, or a combination of such sunscreen agents).

### Example 27 A Water-Proof Sunscreen

In preparing another sunscreen composition comprising agents, compounds, or drugs of the present invention, the sunscreen composition may include (in the OIL phase) a solvent (10% w/w), a film former (8% w/w), a fatty acid (5% w/w), an emulsifier (2% w/w), a waterproofer (3%) w/w), a UV filter (10% w/w), agents, compounds, or drugs of the present invention (33% w/w), Wax (4%) w/w), and a preservative (0.7%); may include in the (WATER Phase) water (5% water w/w), humectant (10% w/w), thickener (3% w/w), Neutraliser (0.7% w/w), emulsifier (3% w/w), sequestering agent (0.5%), preservatives (1% w/w), and fragrance (1% w/w).

### Example 28 Preparation of a Glutathione-Conjugated or n-Acetylcysteine-Conjugated at Least One Agent, Compound or Drug of the Present Invention

In order to enhance activity, at least one agent, compound, and drug of the present invention may be modified by conjugation with glutathione and or n-acetylcysteine by any means known to the art. When said agents, compounds, or drugs of the present invention contain a carbonyl group suitable for reaction with nucleophilic glutathione (GSH) or n-acetylcysteine. However, non-carbonyl agents, compounds, or drugs of the present invention are likewise capable of conjugation, coupling, linkage, or complexing with glutathione. The reaction mixture may, for example, comprise between 5 and 25 uM carbonyl-containing substrate in 10 mM potassium phosphate, pH 7.0, and 1 mM GSH. The addition of an effective amount of GSTPI-1 will accelerate the initial rate of GSH-mediated consumption of carbonyl-containing substrate. The mixture is stirred (up to 3 days) at room temperature until a clear solution is obtained.

### Example 29 Preparation of a Glutathione-Conjugated at Least One Agent, Compound or Drug of the Present Invention

At least one agent, compound, or drug of the present invention (4 mmol) and Glutathione (20 mmol, 6.15 g) may be dissolved in H20 (20 ml) and CH2C12 (2 ml) by stirring at room temperature until a clear solution is obtained. The clear, colorless solution may then be concentrated to about 10 ml, followed by a slow addition of small amount of MeOH. The mixture is then to be kept in the refrigerator overnight as a white solid precipitates out. The at least one agent, compound, or drug of the present invention-GSH complex may then be filtered and dried. Thereafter, the newly GSH conjugate may be incorporated into tablets, troches, gels, capsules, etc. as described herein.

### Example 30 Addition of at Least One Agent, Compound, and/or Drug of the Present Invention to a Composition of the Present Invention

In one embodiment, the bioavailability of at least one agent, compound, or drug of the present invention is enhanced by addition of natural oil. Thereafter, the selected agent, compound or drug and the oil may be incorporated into tablets, troches, gels, capsules, etc., as described herein.

### Example 31 Addition of an Agent, Compound or Drug Containing an No Donor Moiety to the Compositions, Manufacture, Products, Processes, Methods, and/or Methods of Use, Prevention and Treatment of the Present Invention

In some embodiments, the selected agent, compound or drug (e.g. glutathione or at least one agent, compound, or drug of the present invention) is first treated in accordance with the methods of WO 92/01668, WO 95/30641, WO 97/16405, U.S. Pat. No. 5,859,053, WO/2002/011706, WO2010118968, Del Soldato et al., (1999), or Bratasz et al., (2006) to obtain a NO-donor derivative. Thereafter, the newly derived NO donor derivative is incorporated into tablets, troches, gels, capsules, etc., as described herein.

### Example 32 A Tablet for Administering at Least One Agent, Compound, or Drug of the Present Invention

At least one agent, compound, or drug of the present invention (25 mg), Glutathione (200 mg), Lactose (50 mg), Starch (10 mg) and Magnesium stearate (in appropriate amounts) may be mixed by propeller mixing and a tablet prepared according to methods known to the art for tablet preparation. Alternatively, at least one agent, compound, or drug of the present invention (250 mg), Glutathione (250 mg), Lactose (50 mg), Starch (10 mg) and Magnesium stearate (in appropriate amounts) may be mixed by propeller mixing and a tablet prepared according to methods known to the art for tablet preparation.

### Example 33 A Capsule for Administering at Least One Agent, Compound, or Drug of the Present Invention

At least one agent, compound, or drug of the present invention conjugate (250 mg), Lactose (30 mg), Starch (28 mg), Talc (2 mg) and Magnesium stearate (in appropriate amounts) may be mixed by propeller mixing and a gelatin hard capsule prepared according to methods known to the art for gelatin hard capsule preparation.

Alternatively, at least one agent, compound, or drug of the present invention 125 mg, Glutathione (125 mg), Lactose (30 mg), Starch (28 mg), Talc (2 mg) and Magnesium stearate (in appropriate amounts) may be mixed by propeller mixing and a gelatin hard capsule prepared according to methods known to the art for gelatin hard capsule preparation.

### Example 34 A Suspension for Administering at Least One Agent, Compound, or Drug of the Present Invention

At least one agent, compound, or drug of the present invention (250 mg), Isomerized sugar (10 g), Sugar (30 mg), Sodium CMC (100 mg), Lemon Flavor (in appropriate amounts), and distilled water (sufficient to produce a total volume of 100 ml) may be combined to prepare a suspension in accordance with methods known to the art for the preparation of suspensions. A 100 ml darkly colored bottle bottle may then be filled with the suspension and sterilized.

Alternatively, at least one agent, compound, or drug of the present invention (200 mg), Isomerized sugar (20 g), Sugar (20 mg), Sodium arginate (100 mg), Orange Flavor (in appropriate amounts) and distilled water added to achieve a total volume of 100 ml may be combined to form a suspension in accordance with methods known to the art for the preparation of suspensions. A 100 ml darkly colored bottle may then be filled with the suspension and sterilized.

### Example 35 A Polyethylene Coated Preparation for Administering at Least One Agent, Compound, or Drug of the Present Invention

At least one agent, compound, or drug of the present invention (250 mg), Glutathione (200 mg), Lactose (30 mg), Starch (20 mg) and Magnesium stearate (in appropriate amounts) may be combined to fill a polyethylene coated envelope and sealed to prepare a powder.

### Example 36 A Soft Capsule for Administering at Least One Agent, Compound, or Drug of the Present Invention

Polyethylene glycol (400 mg) may be mixed with concentrated glycerin (55 mg) before adding distilled water (35 mg). The mixture may then be maintained at 60° C. Afterwards, at least one agent, compound, or drug of the present invention (200 mg) and Glutathione (200 mg), may be added. The mixture may then be stirred to uniformity at approximately 1,500 rpm, and then cooled to room temperature under slow stirring. When air bubbles are removed with a vacuum pump, the remaining mixture is appropriate for inclusion in a soft capsule. The soft capsule membrane may have been manufactured according to methods known to the art using a widely known soft gelatin-plasticizer formula containing gelatin (132 mg), concentrated glycerin (52 mg), 70% disorbitol solution (6 mg per capsule), an appropriate amount of ethyl vanillin flavoring agent, and carnauba wax as the coating agent.

### Example 37 A Composition for Administering at Least One Agent, Compound, or Drug of the Present Invention

A composition containing at least one agent, compound, or drug of the present invention, vitamin C and vitamin E and having a synergistic effect. At least one agent, compound, or drug of the present invention, vitamin C, and vitamin E may be combined in a weight ratio of 1-50:0.01 to 50:0.01 to 50 along with at least one pharmaceutically acceptable carrier. The composition is formulated into a tablet, hard gelatin capsule, soft gelatin capsule, liquid or suspension, or an injected solution. For example, 50 mg at least one agent, compound, or drug of the present invention, 200 mg vitamin C, 200 mg vitamin E and a suitable amount of an excipient are combined for administration to a human or animal.

### Example 38 A Liquid, Nutritional Supplement Comprising at Least One Agent, Compound, or Drug of the Present Invention

A liquid nutritional supplement may be prepared by combining agents, compounds, or drugs of the present invention (e.g. at least one agent, compound, and/or drug of the present invention (5 g), glutathione (4 g), with electrolytes: sodium (at about 170 mg), potassium (at about 600 mg), calcium (at about 400 mg), chloride (about 500 mg), phosphate (at about 400 mg), magnesium at about 100 mg; vitamins and minerals: iron (about 5 mg), Folic acid (at about 200 meg), Pantothenic acid (at about 2.5 mg), Biotin (about 10 meg), selenium (at about 30 meg), manganese (about 1 mg), molybdenum (about 25 meg), chromium (about 35 meg), vitamin A (about 1000 IU), vitamin B 1 (at about 1 mg), Niacin (about 10 mg), vitamin B2 (at about 1 mg), vitamin B6 (at about 1 mg), vitamin B12 (at about 10 mg), vitamin C (about 60 mg), vitamin D (about 200 IU), vitamin E (about 30 IU), iodine (about 60 meg), and (optionally) vitamin K (about 30 meg). Vitamin K is excluded in compositions for individuals taking certain anticoagulation medicines. The liquid composition further contains additional sources of amino acids/protein (about 11 g (from glutathione, milk protein concentrate, calcium caseinate and sodium caseinate) or about 16%, Carbohydrate (about 45 g (inclusive of about 25% sugar compounds or at about 50%), Fat (about 14 g at about 34% (preferably with the majority being unsaturated fat and including omega 3 fatty acids and about 10 mg cholesterol)), Water (at about 180 mL or about 770/1000 ml), and appropriate or desirable amounts of Flavorings (e.g. chocolate sugar, French vanilla, cherry, pecan, mint, cherry, rocky road, ginger, chocolate chip, oreo, strawberry, etc.) and preservatives. Preferably the method of composition conforms to Kosher and Halal standards.

### Example 39 A Powder for Preparing a Nutritional Drink Comprising the Ingredients of Example 38 in Dried Form with Appropriate Preservatives

### Example 40 A Food Mixture for Baking Comprising the Ingredients of Example 39 to which an Appropriate Amount of Flour, Eggs, Baking Powder or Other Rising Agent is Added

### Example 41 A Seasoning or Condiment Comprising Agents, Compounds, or Drugs of the Present Invention for Addition to Foods

To prepare a seasoning comprising agents, compounds, or drugs of the present invention, about 1-2 g of at least one agent, compound, or drug of the present invention is mixed with varying amounts of seasonings to a total amount of about 5 g. Examples of seasonings useful in the invention include saline seasonings (e.g. salt, spiced salt, saltpeter), acid seasonings (e.g. vinegar (sodium diacetate), or vinegar aromatized with tarragon; verjuice, lemon and orange juices), hot seasonings (e.g. peppercorns, ground or coarsely chopped pepper, or mignonette pepper; paprika, curry, cayenne, and mixed pepper spices), saccharine seasonings (e.g. sugar and honey).

Likewise, a condiment comprising agents, compounds, or drugs of the present invention, may be prepared by mixing about 1-2 g of at least one agent, compound, or drug of the present invention with varying amounts of condiments to a total amount of about 5 g. Examples of condiments to be mixed with at least one agent, compound or drug include pungents (e.g. onions, shallots, garlic, chives, and horseradish), hot condiments (e.g. mustard, gherkins, capers, English sauces, such as Worcestershire, Baron Green Seasoning, Harvey, ketchup, etc. and American sauces such as chili, Tabasco, A-1 Steak Sauce, etc.), wines used in reductions and braisings, finishing elements of sauces and soups, and fatty substances (e.g. animal fat, butter, edible oils and margarine. If the condiments or seasonings are cooked ones, the agent, compound or drug of the present invention will typically be added to the other ingredients after they have been cooked and cooled.

### Example 42 Production of a “Gummy” Containing at Least One Agent, Compound or Drug of the Present Invention

To prepare 100 g of gummy, about 10-200 mg of the at least one agent, compound or drug of the present invention are mixed with about 6.1 g protein, about 75 g carbohydrate (of which 56.2 g is sugar), about 0.2 g fat (of which 0.2 g is saturated fat), 0.03 g sodium, and 0.08 g equivalents as salt—these amounts being derived from glucose syrup, sugar, modified corn starch, concentrated vegetable extracts (e.g. black carrot, spinach, stinging nettle, turmeric, flavorings, glazing agent canuba wax, paprika extract, lutein). The ingredients may be combined by any methods known to the art for producing a gummy.

### Example 43 Preparation of a Carbonyl-Containing at Least One Agent, Compound or Drug

A 6-liter glass reactor that is equipped with a stirrer, a dropping-funnel, and a reflux condenser, may be charged with 6 moles of at least one agent, compound, and/or drug of the present invention containing hydrocarbons having one olefinic linkage as well as 975 g ethyl formate (13.2 moles). The contents may then be heated to about 57° C. At this point, 975 g. hydrogen peroxide (concentration 30% by wt, 8.6 moles) may be added at such rate that no excessive foaming occurs (1-2 hours) after which refluxing is continued for another 6 hours. During the reaction the temperature will gradually increase to about 73° C. The reaction mass is then cooled to about 25° C. and the aqueous bottom layer drained off and discarded. The top layer is then to be washed in succession with 900 ml saturated sodium bicarbonate solution and 900 ml water and then dried over anhydrous magnesium sulphate. The resulting at least one agent, compound, and/or drug of the present invention may then be incorporated into a composition of the present invention as described in the accompanying examples described herein.

### Example 44 Compositions Comprising a NO-Containing Derivative from at Least One Agent, Compound, and Drug of the Present Invention

Agents, compounds, and drugs of the present invention may be modified to bear a nitric oxide (NO) donating moiety by any means known to the art (e.g. WO92/01668, WO 95/30641, WO 97/16405; U.S. Pat. No. 5,859,053; WO/2002/011706; WO2010118968) and subsequently incorporated into the compositions described herein.

### Example 45 Compositions Comprising a Biotinylated Derivative from at Least One Agent, Compound, and Drug of the Present Invention

At Least One agent, compound, and drug of the present invention may be modified by biotinylation (e.g. U.S. Pat. Nos. 4,794,082; 5,521,319), and subsequently incorporated into the compositions described herein.

### Example 46 Preparation of a Tripepetide Composed of Allicin, 1-Glutamate, and Glycine

The tripepetide wherein allicin is substituted for cysteine is synthesized according to any method known to the art (e.g. U.S. Pat. Nos. 4,332,892; 5,968,767; Spirin and Swartz, 2008).

### Example 47 Sublingual/Buccal Composition

To prepare a sublingual composition at least one agent, compound, and/or drug of the present invention of the present invention may be combined with a rapidly dissolving base comprising a polyethylene glycol such as PEG (3350, 1450 or 4500) mannitol, sodium bicarbonate, citric acid, and sucrose, acesulfame potassium, and a flavoring such as raspberry flavor concentrate.

The sublingual composition may comprise an the agent, compound, or drug of the present invention (0.5-5.0 g), PEG 60 g, silica gel 0.56 g, polysorbate 80 3.75 ml, an artificial sweetener such as nutrasweet 0.56 g and sodium saccharine.

Other suitable methods and best practices for preparing a sublingual/buccal preparation are described by Ashraf, 2014 and are incorporated herein.

### Example 48 A Transdermal Patch

To prepare a transdermal patch suitable for administration of the agent, compound or drug, the patch will comprise from about 7 mg to about 21 mg of the agent, compound or drug dosage. The transdermal patches comprise about 7, about 14 or about 21 mg dosage for use in preferred methods of the invention. Such patches may further comprise ethylene vinyl-acetate-copolymer, polyisobutylene and high density polyethylene between pigmented and clear polyester backings. Transdermal patches will in general be applied to dry, clean and hairless skin; worn for about 24 hours and a new one put on after rising the next day); and removing the old patch, cleaning the skin, and replacing the new or used patch at approximately the same time every day as directed by a clinician.

### Example 49 Additional Synthesis Involving Agents, Compounds, and Drugs of the Present Invention a) Preparation of Mono-Phenyl Analogs with Improved Activity

Boric anhydride (0.7 eq) may be added to a solution of 2,4-pentanedione or 4-acetyl-5-oxo-hexanoate in EtOAc (3 eq). The solution is stirred at 70° C. for 0.5 h. To the solution, the agent, compound, or drug of the present invention (1 eq) and tributyl borate (1 eq) are then added. The mixture is stirred for another 30 min. At 85° C., butylamine (1 eq) dissolved in EtOAc is added dropwise over 15 min. The stirring continued for 1 h at 100° C. The mixture is then hydrolyzed by adding 1 N HCl at 50° C. and stirring for 0.5 h at 50° C. The organic layer is separated, and the aqueous layer may be extracted with EtOAc. The combined organic layers are washed until neutral and dried over anhydrous sodium sulfate. After removing the solvent in vacuo, the crude products were purified by flash column chromatography eluting with a hexane-EtOAc gradient. b) Preparation of heterocycle-containing analogs with improved activity

An agent, compound, or drug of the present invention along with boric anhydride (0.7 equiv.) may be dissolved in EtOAc and stirred at 70° C. for 30 min. An appropriate benzaldehyde (1 equiv.) and tributylborate (2 equiv.) may be added, and the mixture stirred for a further 30 min. Piperidine, having been dissolved in EtOAc, may be added dropwise. After increasing the temperature to 100° C., stirring is continued for 1 h. The mixture is then hydrolyzed by adding IN HCl, and stirring at 60° C. for 0.5 h. The organic layer is separated, and the aqueous layer is extracted with EtOAc three times. The combined organic layers were washed with water until neutral. The solvent is removed in vacuo. The crude products may be purified by flash column chromatography, eluting with hexane-EtOAc. c) 1.5 ml of a 25% w/w aqueous solution of cetyltrimethylammonium bromide is added to a solution composed of an agent, compound, or drug of the present invention (10 mmol) in 50 ml of a 0.25 M solution of aqueous NaOH with acetone (0.36 ml, 5 mmol). The mixture is allowed to stir vigorously at room temperature for 20 h, diluted with brine and extracted with EtOAc. The EtOAc solution is concentrated and then subjected to column chromatography to obtain the target product. d) To a solution of acetaldehyde (0.84 ml, 15 mmol) in EtOH (10 ml), 3 M NaOH (5 ml, 15 mmol) is added at 0° C.

The solution is stirred for an additional 20 min. Afterwards, an agent, compound, or drug of the present invention (15 mmol) in EtOH (5 ml) is added to the stirring solution dropwise, the reaction is brought to room temperature and stirred for 2 h. Then the mixture is poured into water and adjusted to pH 7 by adding IN HCl. After extraction with EtOAc, the organic layer is washed with water three times and dried over anhydrous sodium sulfate. After removal of the solvent under vacuum, the crude product is purified with flash column chromatography. e) To a stirring solution of lithium diisopropylamine (0.29 ml, 0.58 mmol) in THF (3 ml), a THF (3 ml) solution of 3,4-dimethoxycinnamone (100 mg, 0.48 mmol) may be added at −78° C. After 15 min, an agent, compound, or drug of the present invention (0.5 mmol) in THF (3 ml) is added and stirred for an additional 20 min at −78° C. Then, the mixture is quenched with saturated NH4C1 solution. The solution is allowed to warm to ambient temperature and extracted with EtOAc. The organic layer is washed with water and saturated NaCl solution and dried over anhydrous sodium sulfate. The crude product is purified by flash column chromatography.

### Example 50

An agent, compound or drug of the present invention (3 mmol) is dissolved in 15 mL of dry methylene chloride. Thionyl chloride (0.3 mL, 3.6 mmol) is added at 0° C. The solution is stirred under reflux for 5 h. The solvent is removed under vacuum to give a solid. In the same flask, 10 mL of anhydrous THF is added, and the mixture heated to reflux. HMDA (0.3 mL) is added very slowly to the refluxing solution, followed by the addition of triethylamine (0.4 mL). The solution is stirred under reflux overnight. The solvent is then removed in vacuo. The solid is extracted with CH2C12×3. The combined CH2C12 solution is washed with water three times and brine once, and then dried over anhydrous sodium sulfate. The crude product is obtained after flash column chromatography.

### Example 51

To prepare a bovine serum albumin (BSA) conjugated agent, compound, or drug of the present invention, nitrous acid may be generated by the addition of a solution of 0.85 niEq of sodium nitrite to an excess of HCl. This reaction can be maintained at a temperature of 5° C. A solution of 0.85 mEq of 4-aniinobenzoic acid in EST HCl chilled to 50° C. may be prepared with continuous stirring in ice bath for 20 minutes, not exceeding the pH of 1.0. Diazotized 4-minobenzoic acid may then be added dropwise to an equivalent concentration, (0.85 mEq) of the agent, compound, or drug of the present invention (compound I) dissolved in ethanol at pH 11.0 with continuous stirring at 50° C.

The solution is then to be acidified to pH 2.0 at which time the derivative (compound II) is precipitated. The precipitate may be centrifuged and redissolved in ethanol at pH 11.0 again. After repeating the acid and base cycle twice, the crude derivative (II) can be chromatographed on a column of silica gel. Reduced pressure evaporation of the elution solvent will give a derivative of about 98% purity as checked by TLC. The bovine serum albumin conjugate (III), of this invention may then be synthesized in a medium of 1% NaCl/dioxane/NaOH solution of pH 8-10, at 50 C, by adding 0.1M solution of I-cyclohexyl-3-(2-morpholinoethyl) carbodiimide metho-p-toluene sulfonate to the purified crystalline derivative (compound II) in the same medium with continuous stirring. Bovine serum albumin is then to be added to the foregoing mixture at 50 C, pH 8-10 with continuous stirring for 1 hr until the intermediate azopseudourea has conjugated to bovine serum albumin, after which the mixture is to be centrifuged off, acidified to pH 4.2, salted out, recentrifuged, redissolved then dialyzed for 24 hr at 50 C against 0.5M sodium carbonate, pH 8.2 until the reaction is complete (or about 2 hours). A final dialysis is performed against bi-distilled water for 24 hours at 5° C., after which the protein conjugate (III) may be lyophilized.

### Example 52 Synthesis of Additional Analogs and Derivatives

In one embodiment, the agents, compounds or drugs of the present invention are modified by chlorination, addition of an imidazole, a methyl amide, the formation of additional amide derivatives, such as the ethyl amides, and/or fluorination of imidazole and amide derivatives. The current invention encompasses derivatives with varying substituent groups (e.g., substituted and unsubstituted carbonyl imidazoles, cyano, esters, glycosides, and amides). Accordingly, reactions relating to the preparation additional analogs and derivatives may be accomplished according to the methods of U.S. Pat. Nos. 4,550,176; 5,389,634; Johnson and Shelberg, 1945; Clinton et al., 1961; Dean, 1965; and Sharpless et al, 1973. For example, derivatives may be produced according to schemes comprising the following steps: 1. Formylation in the presence of sodium methoxidein benzene (Clinton et al., 1961). 2. Introducing a double bond with phenylselenenyl chloride with sequential addition of 30% hydrogen peroxide (Sharpless et al, 1973) followed by halogenolysis (Dean, 1965). 3. Formylation in sodium methoxide (Clinton et al., 1961). 4. Introducing a double bond with phenyl selenenyl with sequential addition of 30% hydrogen peroxide (Sharpless et al, 1973). 5. Cleavage with sodium methoxide (Johnson and Shelberg, 1945).

### Example 53 A Topical Composition Suitable for Treating Acne or Disorder of the Skin

### Example 54 A Lotion Comprising an Agent Compound or Drug of the Present Invention

To prepare a lotion comprising an agent compound or drug of the present invention (e.g at least one agent, compound, or drug of the present invention) in combination with benzoyl peroxide 2.5%, and inert ingredients selected from water, allantoin, aloe barbadensis leaf juice, aluminum silicate, benzophenone-4, carbomer, cetearyl alcohol, cetyl esters, ceteareth-20, color agents, cyclomethicone, diazolidinyl urea, dimethicone, dimethyl isosorbide, disodium dimethicone copolyol sulfosuccinate, ethoxydiglycol, flower extract, fruit extract, fragrance agents, glycerinhydroxyethylcellulose, glycolic acid, glyceryl stearate, Hamamelis virginiana (witch hazel) extract, imidazolidinyl urea, imidazolidinyl urea, magnesium methylparaben, neopentyl glycol dicaprylate, neopentyl glycol dicaprate, panthenol, PEG-100 stearate, polyethylene, polysorbate-20, propylene glycol, propylparaben, sodium hyaluronate, sodium hydroxide, sodium PCA, sorbitol, stearate, tridecyl trimellitate, tetrasodium EDTA, triethanolamine, tridecyl stearate, and xanthan gum.

### Example 55 A Topical Composition Promoting Absorption

To prepare a topical composition comprising water (66%), propylene glycol (5%), Sepigel 305 (2%), Mygliol 812 (4%), and Cremophor RH40 (4%), and active ingredients (at least one agent, compound, or drug of the present invention) (19%), may be added in small portions to a mixture of cremophor and mygliol, at temperatures below that at which the active ingredients are degraded. An aqueous phase may be prepared by adding Sepigel 305 in small amounts with continuous slow mixing to the solution of water and propylene glycol. The final composition may be achieved by adding the oily phase to the aqueous one prior to storage in refrigerator.

### Example 56 A “Vanishing Cream” Composition with Ointment and Cream Properties

To prepare a vanishing cream composition, at least one r agent, compound, or drug of the present invention (1-10% w/w), may be added to preservative, methyl paraben i. p. (0.08% w/w), propyl paraben i.p. (0.04% w/w) and excipients.

### Example 57

Cultured L. donovani promastigotes were exposed to the typical serum concentrations of metronidazole, itraconazole, and ciprofloxacin in clinical settings (e.g. 5 μg/ml) either alone or in two-drug combinations. While ciprofloxacin had limited effects on promastigote motility and growth in DMEM culture medium, the combination of metronidazole and itraconazole led to a 95%) reduction of promastigotes after 144 hours in culture compared to control. Metronidazole/itraconazole also completely inhibited cell motility in the surviving promastigotes. Metronidazole or itraconazole alone caused a significant, but more modest reduction (˜50%) in cultured promastigotes at 144 hours indicating the drugs displayed synergistic effects with respect to promastigote killing. In one method, at least one agent, compound, and/or drug of the present invention may be provided in conjunction with an anti-infective drug to a patient in need of prevention or treatment for a protozoal infection.

Accordingly, agents, compounds, or drugs belonging to the classes represented by metronidazole and itraconazole may be included in the compositions described in the previous examples to provide for a therapy effective in preventing or treating a parasitic disease, especially a protozoal disease.

### INDUSTRIAL APPLICABILITY

The pharmaceutical composition comprising agents, compounds and drugs of the present invention (e.g. at least one agent, compound, or drug of the present invention) are clinically useful in preventing or treating various human diseases (as well as animal diseases?).

“Comprises/comprising” when used in this specification is taken to specify the presence of stated features, integers, steps or components but does not preclude the presence or addition of one or more other features, integers, steps or components or groups thereof.

miRNAs whose target genes were Upregulated in GSE88773 (the miRNAs are listed in order of and in accordance with the degree of their upregulation)

miR547; miR1263; miR7116-3p; miR6951-3p; miR3069-5p; miR7013-3p; miR221; miR1928; miR222; miR3086-5p; miR433-3p; miR208a; miR208b; miR669j; miR669i; miR137; miR654-3p; miR183; miR697; miR193a-3p; miR5134-5p; miR3115; miR365; miR655-5p; miR681; miR155; miR448-3p; miR499b-5p; miR219a-5p; miR500-3p; miR34b-3p; miR467h; miR467e; miR606; miR669k; miR669h-3p; miR468; miR1298; miR4734; miR501-3p; miR3061-5p; miR130b-3p; miR880; miR4678; miR490-3p; miR448-5p; miR4696; miR466q; miR4703-5p; miR3177-5p; miR3087; miR98-5p; miR2139; miR219-1-3p; miR21; miR590-5p; miR1284; miR1539; miR3060; miR4760-3p; miR2116-3p; miR4495; miR19b-3p; miR202-5p; miR4637; miR505-3p; miR3171; miR3661; miR532-5p; miR193b-3p; miR3083-5p; miR878-3p; miR5126; miR669g; miR3144-3p; miR208b; miR208a-3p; miR181c-5p; miR363-3p; miR92b; miR367; miR32; miR92a; miR25; miR4718; miR490-5p; miR1194; miR4661-5p; miR1278; miR1279; miR6852-3p; miR1288-3p; miR1304-5p; miR4484; miR3102-3p.2; miR1196; miR4445; miR682; miR631; miR2183; miR548e-5p; miR467b; miR218-5p; miR3684; miR4304; miR465c-3p; miR465b-3p; miR465a-3p; miR4704-5p; miR151-3p; miR704; miR708-3p; miR1897-5p; miR601; miR1191; miR15a-5p; miR3093-3p; miR3942-5p; miR4715-5p; miR4782-3p; miR219-5p; miR376b; miR2116-5p; miR3086-5p; miR3964; miR1941-5p; miR5117; miR1195; miR878-5p; miR3909; miR2277-5p; miR3146; miR466f-5p; miR4305; miR698; miR6083; miR878-3p; miR361; miR1839-3p; miR302c-5p; miR15b-5p; miR503; miR19a-3p; miR496; miR181a-5p; miR6868-5p; miR3680; miR3967; miR154; miR770-5p; miR669a-3-3p; miR6690-3p; miR669a-3p; miR875-5p; miR1198-3p; miR3686; miR875-5p; miR654-3p; miR5128; miR548ad-3p; miR92a-3p; miR202-3p; miR130a; miR301b; miR721; miR130b; miR301a; miR3471; miR4759; miR664-3p; miR499; miR4503; miR1929; miR300; miR30e-5p; miR1941-3p; miR195-5p; miR4280; miR3061-3p; miR3968; miR327; miR3967; miR192-5p; miR5119; miR491-3p; miR187; miR26a-5p; miR675-3p; miR26a-5p; miR6792-5p; miR669c; miR411; miR10a; miR10b; miR181d-5p; miR496; miR1911; miR548ao-5p; miR93-5p; miR3073-3p; miR344b; miR3072; miR101c; miR153; miR181b-5p; miR1949; miR301b-3p; miR3096-3p; miR3079-5p; miR1244; miR678; miR607; miR33; miR646; miR4742-3p; miR216a; miR6807-3p; miR1263; miR1297; miR297b-5p; miR297a; miR297c; miR548ax; miR653; miR30a-5p; miR190b; miR190; miR4714-3p; miR6508-5p; miR767; miR6846-3p; miR20b-5p; miR21; miR590-5p; miR4677-5p; miR523-5p; miR526a; miR518e-5p; miR518f-5p; miR520c-5p; miR518d-5p; miR519b-5p; miR519c-5p; miR519a-5p; miR4778-5p; miR8067; miR1295; miR3076-3p; miR3195; miR5121; miR2964a-5p; miR27a-3p; miR297a-5p; miR15a-5p; miR1843-3p; miR645; miR4520-2-3p; miR522-5p; miR517b; miR376b-3p; miR3093-5p; miR126a-3p; miR5121; miR3057-5p; miR331-5p; miR5046; miR3143; miR4276; miR1946a; miR519d-3p; miR302d-5p; miR382; miR4774-5p; miR17-5p; miR5585-5p; miR451; miR3939; miR6793-5p; miR376a-2-5p; miR409-5p; miR4652-3p; miR4786-3p; miR101-3p; miR707; miR3081; miR3074-1-3p; miR5108; miR4740-5p; miR6690-5p; miR500; miR501-3p; miR1897-3p; miR4796-3p; miR921; miR4687-5p; miR6790-5p; miR701; miR191; miR139-3p; miR21-5p; miR4796-5p; miR719; miR6868-3p; miR467c; miR467d; miR302b-5p; let-7c-5p; miR216b; miR19b-3p; miR696; let-7f; let-7b; let-7a; let-7g; miR98; miR1961; let-7c; let-7e; let-7i; let-7d; miR582-5p; miR103b; miR106b-5p; miR8485; miR669f-5p; miR669l; miR669p; miR669a-5p; miR494-3p; miR3651; miR155-5p; miR4535; miR505; miR34b-5p; miR376c; miR584; miR215; miR192; miR3140-5p; miR6840-3p; miR6765-5p; miR1901; miR293; miR324-3p; miR467a; miR290-3p; miR292-3p; miR669h-5p; miR4465; miR103a-3p; miR190a-3p; miR667; miR5097; miR142a-3p; miR5120; miR339-3p; miR3941; miR191-3p; miR543-3p; miR598-3p; miR 720; miR466h-3p; miR142-3p; miR6838-5p; miR695; miR3102-5p.2; miR215-5p; miR181a-5p; miR4791; miR4288; miR10a-3p; miR4313; miR101-5p; miR582-3p; miR4255; miR466j; miR669m-5p; miR466m-5p; miR222-5p; miR6854-5p; miR466h-5p; miR654-5p; miR5118; miR1932; miR1258; miR18a; miR18b; miR375; miR4529-3p; miR497-5p; miR379-3p;

miR3182; miR687; miR9-5p; miR7853-5p; miR548u; miR3066; miR193b; miR193; miR4671-3p; miR4713-5p; miR 194-5p; miR23b-3p; miR4777-3p; let-7a-5p; miR3689f; miR3689e; miR3689a-5p; miR3689b; miR151-3p; let-7d-5p; miR627-5p; miR301a-3p; miR454-3p; miR508-3p; miR548v; miR7161-5p; miR1298-5p; miR1-5p; miR183-5p; miR424-5p; miR3101; miR136-5p; miR3086-3p; miR210-3p; miR25-3p; miR696; miR150-3p; miR3201; miR686; miR383; miR4790-5p; miR3201; miR4791; miR3682-3p; miR509-3p; miR5094; miR181b-5p; miR4777-5p; miR3140-3p; miR511-5p; miR3103; miR378b; miR3200-3p; miR409-5p; miR4423-3p; miR871-3p; miR28-3p; miR4423-3p; miR3659; miR672; miR 1287-3p; miR1251-5p; miR7a-5p; miR3945; miR632; miR188-5p; miR4520b-3p; miR501-3p; miR502-3p; miR1225-5p; miR6759-5p; miR5190; miR4259; miR3093-5p; miR541-5p; miR4759; miR4517; miR105-5p; miR210; miR603; miR5116; miR3077; miR323-5p; miR452-3p; miR648; miR411-3p; miR344c; miR344; miR3666; miR804; let-7g-5p; miR5103; miR548ah-5p; miR4295; miR1247; miR5101; miR4753-5p; miR2052; miR4742-5p; miR1306-5p; miR3609; miR519b-3p; miR1197-3p; miR3084-3p; miR3473b; miR196b-3p; miR4479; miR683; miR144-3p; miR1973; miR3678-3p; miR6823-5p; miR342-3p; miR222-3p; miR487b; miR453; miR30e-5p; miR3160-5p; miR323a-3p; miR1951; miR3099; miR552-5p; miR3676-3p; miR680; miR18b-5p; miR3089-3p; miR1973; miR4440; miR190b-5p; miR5099; miR548ad; miR451b; miR3935; miR718; miR463; miR586; miR4803; miR519a-3p; miR1898; miR18b-3p; miR20a-5p; miR125b-1-3p; miR130a-3p; miR1910-5p; miR519c-3p; miR521; miR770-3p; miR340-5p; miR4633-5p; miR1982.1; miR142-3p; miR3096b-3p; miR375; miR2277-3p; miR324-5p; miR3085-5p; miR3152-3p; miR6722-5p; miR556-5p; miR466f; miR3622a-5p; miR374c-5p; miR503; miR99b; miR99a; miR487b; miR100; miR520a-3p; miR520b; let-7b-5p; miR602; miR4735-3p; miR4444; miR4315; miR204-5p; miR210; miR362-3p; miR3147; miR4458; miR4500; miR4661-3p; miR302b-3p; miR302d-3p; miR449b-3p; miR329-3p; miR7156-5p; miR133a; miR133b; miR1982.2; miR16-5p; miR4726-3p; miR3155b; miR3155a; miR7b-5p; miR4724-5p; miR182-5p; miR486-5p; miR3162-3p; miR1273c; miR340-5p; miR302a-3p; miR217; miR3618; miR4681; miR1277-5p; miR5480-3p; miR4798-3p; miR7157-5p; miR4310; miR5011-5p; miR3134; miR4658; miR331-5p; miR5135; miR4474-5p; miR136-5p; miR4444; miR3529; miR379; miR602; miR4306; miR636; miR3115; miR154; miR517-5p; miR7158-5p; miR6874-3p; miR1323; miR1469; miR4712-5p; miR770-5p; miR6503-3p; miR1952; miR1892; miR501-5p; miR27b-3p; miR569; miR591; miR503-5p; miR302e; miR655-3p; miR128-3p; miR192; miR215; miR434-3p; miR424-3p; miR24-2-5p; miR520d-3p; miR493; miR342-3p; miR107; miR513a-5p; miR2355-5p; miR4694-5p; miR3104-3p; miR574-3p; miR4733-5p; miR301b-3p; miR4450; miR3622a-5p; miR3659; miR3908; miR1227-5p; miR125b-5p; miR373-3p; miR29c-3p; miR520e; miR3102; miR3613-3p; miR3094; miR610; miR132-3p; miR302c-3p; miR329-5p; miR545-3p; miR520c-3p; miR1271-5p; miR6074; miR1237-3p; miRlb-3p; miR660; miR3614-3p; miR4655-5p; miR124-3p; miR219-3p; miR99a; miR100; miR99b; miR32-3p; miR338-5p; miR1911-5p; miR572; miR878-5p; miR7a-1-3p; miR3094-5p; miR669h-5p; miR7a-2-3p; miR146a-3p; miR3088-3p; miR6761-3p; miR4523; miR6731-5p; miR8085; miR4524a-5p; miR3133; miR4524b-5p; miR4734; miR3970; miR615-3p; miR3621; miR188-5p; miR291a-5p; miR4445-3p; miR291b-5p; miR372-3p; miR619-3p; miR129-1-3p; miR148a-3p; miR6499-3p; miR4506; miR548n; miR185-5p; miR598; let-7i-5p; miR 192-3p; miR562; miR4781-5p; miR5111; miR3141; miR4633-3p; miR6500-5p; miR152-5p; miR19b-2-5p; miR4727-3p; miR411-5p; miR190; miR190b; miR6862-5p; miR2861; miR106a-5p; miR22-5p; miR3960; miR362-5p; miR4317; miR466n-5p; miR574-5p; miR3117-3p; miR3605-3p; miR4684-3p; miR30b-5p; miR877-3p; miR744; miR29b-3p; miR148b-5p; miR4661-5p;

miR523; miR7111-3p; miR3128; miR4253; miR3472; miR122; miR1305; miR1948; miR339-3p; miR676; miR3104-5p; miR17-3p; miR3681; miR6871-5p; miR19a-5p; miR4473; miR125a-5p; miR5585-3p; miR340-3p; miR1269a; miR4661-5p; miR1247-5p; miR585-3p; miR526b-3p; miR4661-5p; miR466d-5p; miR466k; miR937; miR7151-5p; miR548ba; miR671-3p; miR4427; miR4777-3p; miR4672; miR100-3p; miR4757-3p; miR219-5p; miR7515; miR4719; miR323b-5p; miR325; miR1306; miR1282; miR3124-5p; miR224-5p; miR548ai; miR5590-5p; miR4645-3p; miR634; miR3968; miR4660-5p; miR466b-5p; miR466c-5p; miR664a-3p; miR3195; miR1267; miR296-5p; miR4681; miR24-1-5p; miR6502-5p; miR677-5p; miR4529-3p; miR1269b; miR1301-5p; miR651-3p; miR483; miR4442; miR3687; miR570-5p; miR882; miR1249; miR3180-3p; miR3196; miR3180; miR934; miR1187; miR466a-5p; miR466e-5p; miR466p-5p; miR434-3p; miR4447; miR4472; miR574-3p; miR4644; miR19b-1-5p; miR4327; miR423-3p; miR3177-3p; miR3960; miR8072; miR1965; miR540-5p; miR337-5p; miR3473a; miR548ag; miR3965; miR6830-3p; miR471-5p; miR4789-5p; miR873-3p; miR197-3p; miR380-5p; miR423-3p; miR7150; miR1933-5p; miR933; miR30d-5p; miR520g-5p; miR668-3p; miR211-5p; miR344g-3p; miR4670-5p; miR4757-3p; miR490-5p; miR135b-5p; miR6825-3p; miR3101-5p; miR1967; miR5128; miR1964-3p; miR3545-5p; miR3161; miR4518; miR212-3p; miR140; miR876-3p; miR3680-5p; miR147; miR3529-3p; miR8073; miR6073; miR1266-5p; miR151-5p; miR1905; miR185-5p; miR489-3p; miR3177-5p; miR374a-3p; miR4720-3p; miR142-3p; miR4635; miR4750; miR376c-3p; miR4766-3p; miR30b-5p; miR4750-3p; miR4286; miR5105; miR367-5p; miR6818-5p; miR5582-5p; miR3935; miR3200-5p; miR4475; miR3141; miR492; miR6831-5p; miR688; miR601; miR6880-3p; miR421; miR380-3p; miR8064; miR4477a; miR675-5p; miR5099; miR5696; miR22-3p; miR3098-5p; miR5589-3p; miR548f-5p; miR652; miR561-3p; miR548x-5p; miR548aj-5p; miR548g-5p; miR2182; miR615-5p; miR3616-5p; miR126-5p; miR29b-2-5p; miR4738-5p; miR362-5p; miR30d-5p; miR4524a-3p; miR885-3p; miR4260; miR380-3p; miR4761-5p; miR3121-3p; miR141-3p; miR6718-5p; miR203b-5p; miR625-3p; miR3655; miR567; miR6780a-3p; miR1277; miR3656; miR3068; miR3651; miR3962; let-7f-5p; miR872; miR6885-3p; miR943; miR3653-5p; miR4764-3p; miR3923; miR1296-3p; miR2114-5p; miR455-5p; miR6864-5p; miR669c-3p; miR3619-3p; miR3080-5p; miR6075; miR5110; miR1940; miR144-3p; miR691; miR101b-3p; miR1251; miR3121-5p; miR944; miR5195-5p; miR4515; miR3572-3p; miR941; miR16-1-3p; miR1946b; miR3129-5p; miR3681-3p; miR216a-3p; miR6087; miR649; miR1179; miR26b-5p; miR573; miR30a-3p; miR4670-5p; miR3618; miR1205; miR595; miR4418; miR4273; miR532-3p; miR1193-5p; miR379; miR6768-5p; miR126-3p; miR656-3p; miR4736; miR2277-5p; miR4722-3p; miR614; miR4431; miR590-5p; miR28b; miR28c; miR653-5p; miR468-3p; miR199a-3p; miR199b; miR744; miR 145-3p; miR302b-3p; let-7a-2-3p; miR3158-5p; miR383; miR525-3p; miR524-3p; miR140-5p; miR6834-3p; miR509-3p; miR3102-5p; miR526b-5p; miR5697; miR6727-3p; miR122-5p; miR149-5p; miR1186b; miR34a-3p; miR153-5p; miR4772-5p; let-7e-5p; miR 150-5p; miR509-5p; miR137; miR5123; miR1954; miR760-5p; miR3621; miR521; miR384-3p; miR324-5p; miR662; miR3123; miR3963; miR339-3p; miR294-3p; miR295-3p; miR302d-3p; miR 140-3p; miR1967; miR578; miR3658; miR4776-5p; miR199b-3p; miR6739-3p; miR936; miR4499; miR509-3-5p; miR1976; miR714; miR142-5p; miR4481; miR3171; miR4772-3p; miR3150a-5p; miR3150b-5p; miR96-5p; miR3646; miR3684; miR544a; miR6747-3p; miR335-3p; miR296-3p; miR4258; miR3664-3p; miR483-3p; miR6814-5p; miR942-5p; miR9-3p; miR139-5p; miR291a-3p; miR629-5p; miR4277; miR4451; miR4745-5p; miR95-5p; miR4328; miR541; miR187-5p; miR4749-5p; miR3070b-3p; miR3070a; miR331-5p; miR1900; miR4468; miR2113; miR1228-3p; miR6856-5p; miR143-5p; miR1893; miR590-3p;

miR504-3p; miR150-5p; miR6810-5p; let-7g-3p; miR425-5p; miR299; miR5126; miR1183; miR32-5p; miR3907; miR6764-5p; miR374a-5p; miR883a-3p; miR1955-3p; miR881-3p; miR883b-3p; miR6078; miR4760-5p; miR302a-3p; miR6776-3p; miR758-5p; miR6853-3p; miR341; miR8061; miR6866-5p; miR4536; miR3653-3p; miR3613-5p; miR5109; miR1895; miR563; miR1957; miR570-3p; miR543; miR924; miR621; miR223-5p; miR3676; miR7159-5p; miR4802-3p; miR488-3p; miR6507-5p; miR3713; miR218-5p; miR221-3p; miR6873-5p; miR6758-3p; miR1181; miR1915-3p; miR4725-3p; miR6780b-5p; miR1251-3p; miR4705; miR1252-3p; miR6758-5p; miR186-5p; miR466f-3p; miR4781-3p; miR770-5p; miR4714-3p; miR3976; miR6895-3p; miR6811-3p; miR147b; miR3091-5p; miR6844; miR3178; miR4433a-3p; miR29b-3p; miR4669; miR718; miR6071; miR4640-3p; miR3970; miR582-3p; miR3063-3p; miR127-3p; miR1224-5p; miR135a-5p; miR486b-5p; miR743b-5p; miR6785-3p; miR344c-3p; miR4798-5p; miR25-5p; miR3108-5p; miR883a-5p; miR659-5p; miR3074-5p; miR18a-5p; miR4783-5p; miR613; miR4707-3p; miR4271; miR548 am-3p; miR548ah-3p; miR3927-3p; miR6845-3p; miR1231; miR3197; miR30c-5p; miR548j-3p; miR4703-3p; miR423-3p; miR564; miR5590-3p; miR4652-5p; miR4704-3p; miR4275; miR4789-3p; miR548ae-3p; miR548x-3p; miR548aj-3p; miR548aq-3p; miR361-5p; miR467f; miR200b-3p; miR4282; miR548aw; miR141-5p; miR3092; miR216a-5p; miR8079; miR4738-3p; miR376a-3p; miR580-5p; miR4706; miR767-5p; miR3071-5p; miR3920; miR3656; miR584-5p; miR1306-3p; miR6715b-5p; miR450a; miR1248; miR377-3p; miR4425; miR494-3p; miR4262; miR4269; miR669e; miR5680; miR4713-3p; miR4749-5p; miR4706; miR4307; miR1245a; miR129-5p; miR7974; miR4467; miR921; miR4764-5p; miR3591-3p; miR337-5p; miR223-3p; miR3613-5p; miR5684; miR1899; miR5571-5p; miR3148; miR769-3p; miR548f-3p; miR1260b; miR4783-3p; miR105; miR6882-5p; miR4744; miR5004-5p; miR219a-1-3p; miR520f-5p; miR501-3p; miR432-3p; miR548e-3p; miR548az-3p; miR548ar-3p; miR506-5p; miR4330; miR431; miR18b-5p; miR554; miR367-3p; miR6080; miR20b-5p; miR574-5p; miR539-5p; miR3940-3p; miR136-3p; miR1268b; miR4512; miR4632; let-7b-5p; miR5582-3p; miR6877-3p; miR5587-5p; miR598-5p; miR344-3p; miR5116; miR551a; miR937-3p; miR883b-5p; miR369-5p; miR324-5p; miR3939; miR517b-3p; miR6502-3p; miR1266-3p; miR7852-3p; miR3144-5p; miR31-3p; miR1839-5p; miR1-3p; miR466; miR658; miR4740-3p; miR628-3p; miR4788; miR1204; miR548a-3p; miR551b; miR5189-3p; miR4290; miR203a-3p; miR6086; miR4787-3p; miR517c; miR517a; miR299-3p; miR4433a-5p; miR129-2-3p; miR6512-5p; miR30a-5p; miR1178-5p; miR597-5p; miR221-5p; miR663a; miR551b-5p; miR425-5p; miR935; miR6819-3p; miR8063; miR516a-5p; miR877; miR4460; miR324-3p; miR6782-5p; miR20b-3p; miR548m; miR548g-3p; miR4494; miR6746-3p; miR23a-3p; miR7-2-3p; miR6881-3p; miR3944-3p; miR496; miR7843-3p; miR4453; miR3191-3p; miR4538; miR876-5p; miR203b-3p; miR204-5p; miR4520a-5p; miR4520b-5p; miR33a-3p; miR344d-3p; miR6514-5p; miR410-3p; miR344e-3p; miR344f-3p; miR6716-5p; miR548av-3p; miR892c-5p; miR4781-5p; miR5187-3p; miR6775-3p; miR549a; miR4799-3p; miR92a-3p; miR6760-5p; miR191; miR3157-5p; miR4497; miR363-3p; miR3679-3p; miR486; miR3107; miR4539; miR374b-5p; miR3167; miR6748-3p; miR6884-3p; miR5692c; miR5692b; miR3186-5p; miR553; miR374b-3p; miR4655-3p; miR1947; miR9500; miR296-3p; miR132-3p; miR106b-5p; miR669k-3p; miR669h-3p; miR5687; miR4783-5p; miR3146; miR5588-5p; miR4720-5p; miR186-5p; miR6890-3p; miR4795-3p; miR8066; miR7845-5p; miR223-3p; miR369-5p; miR502-3p; miR6508-3p; miR4659a-5p; miR4659b-5p; miR4800-5p; miR1224-3p; miR184; miR3619-5p; miR673-3p; miR509-3p; miR518a-3p; miR518b; miR518d-3p; miR518c; miR518f; miR615-3p; miR550b; miR518a-3p; miR18a-5p; miR1306-3p; miR3193; miR6749-5p; miR452-5p; miR1839-5p; miR3058-5p; miR124-5p; miR518d-3p; miR1936; miR551b-3p; miR1894-3p; miR4731-3p; miR3607-3p; miR434-5p; miR4747-3p; miR1290; miR1911-3p; miR4424; miR1268a; miR4684-3p; miR429; miR6514-3p; miR4740-3p; miR6890-5p; miR138-5p; miR466m-3p; miR4660-3p; miR214-3p; miR9-5p; miR4795-5p; miR761; miR8056; miR 106a-5p; miR451a; miR10b-5p; miR4540; miR669f-3p; miR1250-3p; miR106b-3p; miR3977; miR3131; miR611; miR377-5p; miR377-3p; miR3080-3p; miR497-3p; miR3617-5p; miR616-3p; miR3682-5p; miR638; miR6787-5p; miR371-3p; miR5130; miR4785; miR1468-3p; miR658; miR4493; miR610; miR4438; miR5008-5p; miR657; miR6857-5p; miR6756-3p; miR6859-5p; miR6769b-3p; miR3065-5p; miR3671; miR891a-3p; miR3650; miR3183; miR4723-3p; miR3125; miR5136; miR4467; miR5004-3p; miR6806-5p; miR592; miR4466; miR617; miR5098; miR4674; miR371b-3p; miR181c-3p; miR6881-5p; miR548az-5p; miR6832-5p; miR548t-5p; miR4766-5p; miR4762-5p; miR641; miR583; miR3145-5p; miR3917; miR4454; miR7-1-3p; miR513c-3p; miR450a; miR758-3p; miR527; miR2137; miR1913; miR3074-5p; miR1249; miR4528; miR2053; miR3688-5p; miR1245b-3p; miR4707-5p; miR1291; miR3972; miR3471; miR6740-5p; miR501-5p; miR204-3p; miR513a-3p; let-7d-5p; miR6070; miR1969; miR3605-3p; miR6082; miR3098-3p; miR4536-3p; miR486a-5p; miR504-5p; miR335-5p; miR5708; miR517c-3p; miR92b-3p; miR517a-3p; miR3135; miR6788-5p; miR6794-3p; miR3916; miR3606-3p; miR139-3p; miR5009-5p; miR5092; miR8058; miR4535; miR6755-3p; miR4769-5p; miR6757-3p; miR155-5p; miR593-5p; miR564; miR6867-5p; miR214-5p; miR579-3p; miR30d-3p; miR518a-5p; miR5588-3p; miR3145-3p; miR7155-3p; miR6499-5p; miR4490; miR3663-3p; miR3136-3p; miR6833-3p; miR4768-5p; miR99a-5p; miR4646-5p; and miR585.

miRNAs whose target genes were Downregulated in GSE88773 (the miRNAs are listed in order of and in accordance with the degree of their downregulation)

miR1929-5p; miR138-5p; miR1956; miR669g; miR125b-2-3p; miR452-3p; miR344c-3p; miR216b-5p; miR590-5p; miR710; miR344-3p; miR1934-5p; miR3103-3p; miR409-3p; miR1942; miR1306-3p; miR4727-3p; miR125b-1-3p; miR4460; miR6861-3p; miR547-3p; miR504-5p; miR548q; miR3124-5p; miR4281; miR153-3p; miR3096b-5p; miR4791; miR3960; miR3108-5p; miR6882-5p; miR3156-3p; miR344f-5p; miR146b-5p; miR3201; miR7978; miR587; miR523; miR1288-5p; miR532-5p; miR4790-5p; miR3084-3p; miR1249; miR3649; miR892a; miR33-5p; miR3076-5p; miR873a-5p; miR1946b; miR450a-5p; miR492; miR662; miR1249; miR3186-5p; miR3152-5p; miR4754; miR606; miR4707-5p; miR4299; miR6817-3p; miR3078-5p; miR4690-5p; miR1958; miR6861-5p; miR3938; miR6735-3p; miR125b-5p; miR878-5p; miR465c-3p; miR465b-3p; miR465a-3p; miR6690-5p; miR192-5p; miR669h-5p; miR5188; miR5196-3p; miR182-5p; miR8074; miR4315; miR711; miR147; miR8071; miR6873-3p; miR1178-3p; miR4787-5p; miR1269a; miR542-5p; miR693-3p; miR3648; miR1964-5p; miR212-5p; miR3940-3p; miR4482-5p; miR3130-5p; miR3162-3p; miR7110-3p; miR634; miR7704; miR1982.1; miR188-3p; miR1269b; miR5192; miR3077; miR4738-5p; miR3137; miR1306-5p; miR933; miR4285; miR 1946b; miR743b-3p; miR211-5p; miR4485-3p; miR490-5p; miR4750-5p; miR215-5p; miR4757-3p; miR5124a; miR7157-5p; miR4310; miR743a-3p; miR216b-5p; miR6870-3p; miR3680-5p; miR4456; miR6772-3p; miR296-5p; miR4750; miR3102-3p; miR4305; miR6836-3p; miR325-3p; miR554; miR4259; miR4254; miR4661-3p; miR4640-3p; miR3591-5p; miR34a-5p; miR4509; miR6856-3p; miR6731-3p; miR7845-5p; miR5685; miR6742-5p; miR4264; miR377-3p; miR9-5p; miR4690-3p; miR4448; miR122-5p; miR4429; miR4714-3p; miR5707; miR337-3p; miR3195; miR182-3p; miR7975; miR150-3p; miR320b; miR320d; miR4268; miR10b-5p; miR1247; miR330-3p; miR10a-5p; miR695; miR3069-3p; miR5112; miR1905; miR6872-5p; miR639; miR5003-5p; miR5008-5p; miR223-3p; miR129-5p; miR3068; miR658; miR4767; miR759; miR133a-3p; miR324-5p; miR492; miR6820-5p; miR3104-5p; miR4692; miR9-3p; miR937-5p; miR6729-3p; miR1900; miR877; miR1973; miR376c-3p; miR4446-3p; miR3166; miR4514; miR6089; miR4798-3p; miR3085-3p; miR3064-5p; miR666-5p; miR320c; miR669b-5p; miR10b; miR10a; miR205-5p; miR4301; miR3921; miR1929; miR4660-3p; miR466m-3p; miR718; miR548ad; miR5136; miR1931; miR669c-5p; miR183-5p; miR4636; miR3186-3p; miR582-3p; miR553; miR878-3p; miR542-5p; miR323b-5p; miR690; miR613; miR4653-5p; miR1914-5p; miR4781-5p; miR4787-5p; miR3147; miR3675-5p; miR204-5p; miR5100; miR383-5p; miR4507; miR5009-3p; miR3080-3p; miR4764-3p; miR525-3p; miR524-3p; miR759; miR4529-3p; miR1247; miR4474-5p; miR804; miR6745; miR4796-5p; miR410-5p; miR448-3p; miR6509-3p; miR8073; miR5130; miR4431; miR193b; miR193; miR224-3p; miR3940-5p; miR4467; miR3682-3p; miR6510-5p; miR522-3p; miR363-5p; miR627-5p; miR4535; miR494-5p; miR3178; miR711; miR4327; miR4286; miR3922-3p; miR883b-5p; miR698-3p; miR455-3p; miR5117-5p; miR382-5p; miR1941-5p; miR682; miR1204; miR876-3p; miR615-5p; miR7151-5p; miR4727-3p; miR139-5p; miR1204; miR1268b; miR1268; miR4436b-5p; miR4433a-5p; miR26a-1-3p; miR597-5p; miR4693-5p; miR221-5p; miR126-5p; miR3967; miR6782-5p; miR18b-3p; miR23a-3p; miR4540; miR127-3p; miR4467; miR598; miR3099; miR292a-5p; miR290a-5p; miR503-3p; miR152-5p; miR3141; miR532-5p; miR3157-5p; miR548as-3p; miR4726-3p; miR199a-3p; miR199b; miR6748-3p; miR8070; miR206; miR6764-5p; miR517b; miR3093-5p; miR3092; miR1285-3p; miR4783-5p; miR8086; miR628-3p; miR6789-5p; miR132-3p; miR596; miR345-5p; miR6832-3p; miR3073-3p; miR4258; miR6128; miR502-5p; miR660-3p; miR139-3p; miR3912; miR1915-3p; miR1936; miR296-3p; miR 124-5p; miR3058-5p; miR327; miR471-3p; miR3109-3p; miR1296-3p; miR196a-3p; miR489-3p; miR2114-3p; miR889-5p; miR4259; miR200a-3p; miR99a; miR99b; miR4783-5p; miR100; miR182-5p; miR1965; miR3177-5p; miR3181; miR3940-3p; miR378j; miR6839-5p; miR1273f; miR362-5p; miR1537; miR4328; miR503; miR5121; miR3195; miR6838-3p; miR3677-5p; miR3157-3p; miR592; miR3960; miR8072; miR3471; miR621; miR323-5p; miR3165; miR6777-3p; miR150-5p; miR151a-5p; miR185-5p; miR3094; miR210; miR4661-5p; miR671-5p; miR2137; miR552-5p; miR1982.2; miR339-5p; miR4669; miR3184-5p; miR4715-5p; miR635; miR409-5p; miR1843-3p; miR4497; miR546; miR3687; miR3683; miR5121; miR877-5p; miR702-3p; miR5708; miR92b-3p; miR3080-5p; miR3141; miR1908; miR663; miR6751-5p; miR210; miR324-5p; miR466i-5p; miR466d-5p; miR466k; miR7702; miR4535; miR338-5p; miR8058; miR5090; miR146a-5p; miR5009-5p; miR203-3p; miR3968; miR4655-3p; miR1292; miR4787-3p; miR6715a-3p; miR694; miR1277; miR4790-5p; miR3605-3p; miR5001-3p; miR3176; miR5588-3p; miR6824-3p; miR5581-3p; miR4473; miR3663-3p; miR340-3p; miR3925-3p; and miR4777-3p.

RNA Modulated by 4-Aminopyridines in GSE88773 (listed in order of and in accordance with the degree of their upregulation or downregulation)

### Top Upregulated RNA

AC073333.1; FOSL2; BLOC1S5-TXNDC5; AS3MT; PNMA6B; CH507-9B2.4; FOSB; NPAS4; RP11-98J23.1; GABRQ; GPR75-ASB3; RP11-426L16.10; RP4-777O23.3; TLR9; LSM12P1; LRRC37A; RP5-864K19.6; AC104534.3; RP11-20123.1; RP11-466H18.1; PLGLB2; CDRT4; RP11-305M3.2; UTS2; C1DP1; TMED7-TICAM2; SEMA3G; PWP2; PCSK1; ARMCX7P; CH507-39619.7; RP11-561B11.2; RP11-613M10.9; RP1-37E16.12; RP1-130H16.18; NPTX1; BDNF; AGAP10P; MINOS1-NBL1; IKBKGP1; RELB; HSD17B7P2; NFATC2; CTD-2510F5.6; CH17-140K24.8; CEMIP; HTR2A; ARL2-SNX15; RPLPOP6; DIO3; SH2D4B; RP11-618P17.4; AC006116.27; GPR50; CITED1; FAM46A; CTD-2207023.3; EIF4EBP3; SPDEF; RP11-146B14.1; RPL14P1; BNIPL; CTC-454I21.3; TNFRSF11A; CD302; CSNK2A3; RTL1; TECRP1; LINC00643; CTD-2369P2.12; POSTN; WNT11; RP11-144L1.8; AL136097.1; GPAT3; CSRNP1; GEM; TTC29; CALCB; YBXIP10; RP11-196G11.1; ZIC2; NEURL2; ZNF878; KCNK9; RP11-104G3.7; PRKXP1; SV2C; ADRA1B; NONOP2; FAAHP1; ROR2; PCDHGA3; SULTIA4; RP4-734P14.4; RPL7P1; KIAA1211L; CH507-42P11.8; DUSP4; TNFRSF9; VSIG2; KCNK1; NPFFR2; URGCP-MRPS24; ZRSR1; PER1; RP11-195E2.2; RP11-1319K7.1; NPM1P40; RP11-903H12.5; ADAMTS17; COL5A3; TMEM71; C4ORF19; HES2; AC234582.1; AL135745.1; AC008810.1; HAPLN1; NPIPB9; AC108448.2; HNRNPA1P7; NAB1; GPR39; OR7E38P; GCNT1; CLK1; PTCHD1; RP11-111F5.2; EPHA1; AKNAD1; FAM83G; MS4A10; TGIF2-C20ORF24; TMEM155; TGFBR2; NEK2; NPIPA2; IL7; CBARP; MSANTD1; CYP27B1; AC003002.6; GABRA4; CARTPT; ATP10A; MT1X; FAM21FP; AC026348.1; CALML4; PDK4; RSPO3; CCDC150P1; CTD-2021K4.1; RP11-113C12.4; AC024937.6; HMGBIP5; EME1; TMEM249; RP11-570L14.1; ST6GALNAC2; NR4A3; C2ORF66; NTS; COLIA1; DUSP5; DMRTC1; PRDM6; HDACIP2; SCN1B; NFKBIZ; DND1; RP11-216M21.1; STC1; FAM167A; ZNF248; DEPDC1; AC009060.2; TMEM100; SPTBN5; VGF; KCTD8; FBXO4; HVCN1; PRH1; RP11-392A14.9; KLF5; GCH1; LSMEM1; KISS1R; MBNL3; CLK4; ANKRD37; TMEM35B; TM4SF19; FAM163A; TLR10; RP5-994D16.11; GTF2IP7; SYCP3; GCNT4; GGT6; SCG2; CTB-134H23.3; ISG15; AK3P3; RP11-240B13.2; SHISA3; HOMER1; VNIR83P; ZBED6; HS3ST2; UBBP4; NFATC1; LRRD1; HS3ST1; UBE2SP2; RGS4; AC006486.9; LGI2; SERPINA10; ZC3H12C; CLEC18A; SIK1; HLA-V; KRT19; RAB4B-EGLN2; SLC6A17; SLC39A12; BMS1P9; STAC2; PMS2P5; RP11-536C10.3; OLFML2A; FGFR1OP; TMC8; ZIC4; LRRC4; HSPEIP3; IDI1; CORIN; SIK2; ZNF449; BUD13P1; TUNAR; FAM46C; SLC7A9; LILRA6; PROX2; MB21D1; DEPDC1B; CCL16; LTBR; TBCID27; DGKK; LGALS3; TC2N; SAP25; CCDC13; RAD21L1; PDE3A; RP11-974F13.5; KCNK13; C11ORF70; KRTCAP3; RRAD; OSBPL3; RSPH10B2; TMPRSS3; SGCZ; DAO; RP11-1021N1.1; RPSAP70; PTGER4; ZFP36L2; PMEPA1; ERAS; NPIPA5; CPNE9; ADCYAP1; RP11-745010.4; ABCB1; FGF23; ZFP36L1; GNB4; PHLDA2; RP11-169L17.3; OXTR; RGS2; BMP6; TTC16; NEIL3; TNFRSF13C; RP11-727A23.1; VSX2; TFRC; DTL; AC006509.7; NAB2; KDR; AC019097.7; FBXL21; SFTPB; AICDA; ADAM3A; UBE2F-SCLY; SOX7; LRP2BP; SYNDIG1; NKD2; CTCFL; RASAL1; PCDH11Y; PRELID1P1; CD34; STAC3; RP11-507M3.1; BMP2K; RP11-311P8.2; NID2; RELT; MALL; LRRC19; RP11-849H4.2; FAM72B; RASSF8; SLC18A2; BMF; NT5C1A; ZNF571; SFMBT2; MAGEL2; AC007040.11; SLC9B1; SLC34A3; LINC00933; MUC4; CCDC173; AC022182.2; RFX4; CKS2; TSPAN12; SORD2P; CLEC3B; FUT5; RP11-465B22.3; INHBA; SIK3; UPB1; ODCP; CEP83; CICP16; RSPO2; RGCC; F2RL1; ADAMTS6; CDC6; RP11-481A20.10; TCTE3; YBXIP2; ABC13-48559800H2.1; INSIG1; AC109486.1; RP11-381O7.3; TP53INP2; ELOVL5; TRPV3; AC016712.1; RP11-77K12.7; NEUROD4; ZGRF1; IQSEC3; LRRC74B; SDC4; HIST1H2BK; HCFC2; LRR1; KITLG; TRIM54; EYA1; GAD2; ESYT2; RARA; ADAMTS9; GABRE; ZNF285; SNRNP48; AC005281.1; MSANTD3-TMEFF1; PI4K2B; COL13A1; ISG20; B3GALNT2; IRX4; RP11-61N20.3; ADARB1; STK17B; RGPD6; MTCYBP18; SNURF; CH507-9B2.9; KIAA1210; RP11-339B21.8; SKA1; ATPIB1; COL24A1; TROAP; RP11-680G24.4; NR4A1; WISP2; AURKB; ERFE; C3ORF52; TMEM213; ULK4P1; CTNNAL1; RP11-29H23.5; BAGE2; TMEM56-RWDD3; ENO4; ANKRD34C; CD2AP; HSD3B1; RP11-153M3.1; EGR4; SAMD9L; SHCBP1; CFAP157; ARHGAP29; KIAA0226L; ELL2; 12-Sep; GINS4; ZNF600; TMPRSS6; EFCAB10; SLC43A3; CYP2E1; NUTM2F; NDUFV2P1; XXBAC-B562F10.11; ATL2; RHOQ; KCNJ14; KIF14; AC009014.3; EGFEM1P; ASPM; AURKC; RP11-134F2.8; RGPD5; MELK; QRFP; LMNA; ACE2; TAF5; CRIP3; TMEM2; KRT222; ITGA8; TMEM243; SLC5A7; YBXIP1; RPS6KA3; AMD1; SSH3; SMCO4; MEF2C; NR6A1; GLA; RP4-701O16.5; KPNA2; LFNG; MFAP3L; EXOSC9; TMEM132C; KCNA4; BAIAP2; GPR101; GOLGA8K; EIF4HP2; PTP4A1; TAF1D; TMC5; RP11-371E8.4; PARPBP; CPOX; UBE2Q2L; ZGLP1; SOX1; BNIP3P37; SMGIP2; CLDN9; ELOVL7; RASGRP3; HIC1; RP11-302B13.5; CTRL; RNF128; NOS1; PRKAA1; AC073065.3; ADRA1D; CNIH4; MOSPD2; C1ORF162; SEC14L4; SLC12A2; USP53; MPZ; SPP2; RGS16; TAC1; KCTD20; MSTN; AC007969.5; NPHP4; FLVCR2; ZFP28; HERC2P8; ADM; CREBZF; MIS18BP1; GLIPR1L2; CCL28; AGFG2; OR7E14P; LRRN4CL; PLG; GLUD1P3; FAM150B; NPTX2; HRH1; C4ORF33; CREM; SLC6A20; IFITM10; TRPV6; GPR26; FAM78A; RTN4RL1; CCDC58; RP11-673E1.3; MMRN2; CNOT6LP1; AC004057.1; JARID2; DSEL; RRM2; ETV5; LRRC2; SLC14A1; C3ORF58; CHST8; POU5F1B; HTR7; HERC2P5; AC068134.10; AP000769.1; NOXRED1; ESCO2; RAB7B; LRRC6; ESRRB; FNDC1; VENTX; VMO1; NHSL2; GALR1; SVILP1; RP11-382D8.5; SQRDL; CD46; A2ML1; SLC2A14; NAPRT; CTD-3088G3.8; ATP2B1; CSF3R; IMPG1; SMTNL1; HSBPIL1; ZNF10; NTRK1; NCMAP; C1ORF167; SPATA2L; TCEAL5; IL12RB1; CD58; SPDYE15P; CCNA2; ATP8B1; ERLECIP1; MLANA; ADGRE2; RP5-966M1.7; MLKL; SLC4A1; PDE7A; LRRC8C; RP11-726G1.1; MT-ATP8; GSC2; CCL25; SELK; PABPN1L; TMEM200A; RP11-613M10.8; TGFB2; RPL23AP21; F2RL2; LRRC37A5P; FNDC9; HES5; SS18L2; CDC45; SPDYE12P; FLVCR1; FAM106A; KCNMB1; SMIM22; CHD1; PDE5A; INTS6L; NDEL1; PTGFR; ABTB2; MORF4; CYP3A43; PGM2; PRLHR; MFSD2B; CASP7; GRIN2A; CTC-471F3.5; SSUH2; ST3GAL1; L3HYPDH; GPR153; RP13-104F24.1; CPLX1; CAMTA1; TPBG; SYAP1; ORC1; GPR158; GNG11; LRRC39; HS3ST4; IAH1; ARG2; BRCA2; C2ORF15; RP11-15J10.8; GIPC2; RHBDL3; ARFGEF3; SHC4; IL6R; SPDYE9P; GNL1; SSX2IP; JUND; HIVEP2; CEP152; SGMS2; KCNA1; RP11-1264N5.1; FOXD3; KDM7A; SLCO1C1; TBXAS1; RP11-58E21.1; CYP51A1; TP53AIP1; PABPC1L; MMP10; FCRL5; SUV39H2; SLC25A33; UBASH3B; MPEG1; SLC20A1; ETF1; BGLAP; AQP1; HELLS; CDK1; SCML1; PARD6A; MAPK15; GDPD3; FDX1; CRX; IGFBP3; ETV3L; IGFBP7; FAM24B; AJAP1; B4GALT6; STK17A; STXBP2; OLA1P3; GTF2IRD2P1; CSF2RA; A2M; TMEM196; BACH2; RASL11A; SLC6A5; C8ORF88; WNT8A; LRRC1; PHF20L1; GSDMA; ALOX12; ESRRA; POLD3; RP11-419C5.2; MVD; UAP1; SPSB1; MTND4P12; HIST2H2BE; REM2; RP11-85G20.2; STX16-NPEPL1; ZHX2; FRMD7; ACAT2; GAPDHP33; RP11-344E13.1; MTCP1; PCDHGA2; MTHFD2L; SLC45A4; STAG3L1; SNAPC1; TNFRSF12A; IQCJ; SOCS1; GPR35; STXBP5; C22ORF42; ATG16L1; FNDC3A; MAK16; RP11-823P9.3; SLC22A16; KLC3; ADAMTS3; MIDN; RIPPLY2; AUNIP; CRY1; AGGF1P1; PPP1R14BP3; ZNF724P; RP11-648015.1; SYNJ2; RSRP1; DDX3P1; ADCY4; SERTAD1; GNL2; EHD4; PUS7; DBF4; NID1; CLN8; TRMT13; CPM; LYAR; ZNF154; XYLT1; RBM44; RBM47; CH17-258A22.4; NNAT; SLC22A7; AC090498.1; SUCLA2P1; CHST9; CTD-2207023.12; RP11-756A22.7; NUF2; PDCD5; RPF2; TYMP; TTK; SNX25; HIST2H2AA4; PLK4; TRAPPC3L; TAC3; HMGB1P20; TSEN54; BMS1P16; CLK3; BICDL1; AP1G2; ZIC1; GP6; COL1A2; IFRD1; GP1BB; PAPD7; ASTE1; MOBP; NUFIP1; NRARP; ASB12; C8ORF48; MUC20; ERF; TOP2A; ZC3H12B; SIGLEC15; FAM196A; ENPP6; RPSAP36; DDX21; TESC; CENP1; TMEM265; BTNL9; TEX30; AIFM3; USP15; HTATIP2; HHIP; SLC30A5; SYNE4; RP11-51F16.8; COL2A1; AL356053.1; CD37; LRRC10B; PLAG1; GPR89B; SNTG2; NOP2; KCNH6; RP11-353N4.5; ZNF750; KCNAB3; NHLH2; C4BPB; LONRF1; BCL2L11; PAPD5; SLC6A6; CASP10; SMIM4; H2BFM; EVA1C; FOXD4L1; MUC1; PAM; DPH3; PTPN7; PNN; TEN1; NUP54; DNAI1; PPWD1; MTHFS; CDC20; FAM60DP; RP11-835E18.4; QPCT; PCP4; SEHIL; CTD-2583A14.9; ZNF330; EPT1; HIST1H2AC; MTERF3; FSD2; FAM103A2P; RP11-1074O12.1; NEDD8-MDP1; HIST1H2BG; POPDC3; GALNT3; RP11-551L14.4; PHF10; MAFB; CBWD5; GABRA6; NCR3LG1; FAM76B; DYSF; FBXO22; RGS20; STK40; GDF6; PITPNC1; CIITA; RNF222; CALU; RP13-104F24.3; CCNH; ELF1; LA16C-431H6.6; TASP1; HIST1H2AE; IMPG2; PDZK1; CYSLTR2; BTAF1; DDX10; HGFAC; NDUFAF4; CKM; MYLK4; LYPD6B; OPN3; AOC3; CCDC114; ZNRF2P2; DDIAS; RPAP3; MPV17L; PPPICC; GS1-393G12.13; MAPKAPK2; NIPAL1; C10ORF11; GGT5; RP11-468O2.1; FAM98A; PAICSP1; AMMECR1; TMEM136; ZNF511; FYTTD1; NEMP2; KIF20A; IGLV5-52; ERRFI1; ADM5; APOL1; CASC5; TLE2; RP4-669P10.19; ORC2; CHORDC1; ZMYND19; NAA25; CDC14B; NOC3L; ANGPTL1; VDR; REL; DTX2P1; TBK1; PECAM1; LYSMD3; NCAPD3; VPS26A; APEX2; RP11-15G8.1; ITIH5; ALG1L13P; SLC18A3; HMGCS1; PDEIC; SH3RF3; PPIG; ENO1P1; EFHD2; SLC39A14; GLRX2; HCN1; C6ORF163; GREM1; FAM133B; RP4-756H11.5; RGAG4; ETS1; NAALADL2; GFI1B; HIST1H2BN; PRPF4B; PDZD8; POFUT2; TRIM73; CYCS; RAB21; LY6G5C; ZIC3; TMEM198; RP11-22B23.1; PLS1; CENPA; RHOJ; CTD-3105H18.14; DOK5; KRT42P; USP12; MPHOSPH6; ARL2BP; MST1R; RP4-583P15.15; BIRC5; RABGEF1; PDE10A; PTGER3; SRSF2; PTGS2; SACM1L; SERAC1; RFPL3S; C1ORF52; ANKRD19P; PPTC7; NCALD; ZFAND5; IVNS1 ABP; PRPF38B; MKNK2; SLC31A1; ANKRD65; MED23; MME; PINLYP; CENPN; RASGRF1; LPAR6; NDUFAF5; PRPF4; DDX27; CHAF1A; ST8SIA5; KCNQ3; SLC19A2; POLR2J2; SPIB; AKIRIN1; SLC47A1; RRP1; CSGALNACT2; KIAA0895; HMGCR; FAM222A; RSRC2; DEPDC7; TLE3; DOK3; REXO1L1P; BRIP1; C1QL3; MIA; DOK7; TMEM189; NFIL3; RP11-515E23.1; UQCRHL; ZFC3H1; ADCY8; SPTSSA; NUDT15; ZNF559-ZNF177; TMEM52B; TRIB1; THOC6; SLC28A2; PPP1R13B; APCDD1; PHLDA1; ITGAI1; NAA16; ATAD3B; SEC24D; TLL2; RBM25; MMP25; MTNDIP23; SLC7A6; TMEM170B; CLDND1; CMC2; RPRML; KCTD14; ONECUT3; SPDYE2; PPP2R5A; TRPM8; PCED1B; CHSY1; OTULIN; BRINP1; PWWP2B; USP44; SULT4A1; VTN; ATP11A; FAM83F; GSDMB; CDCA3; ICMT; KIF24; TACR2; CEACAM8; PIGH; MMP23B; CCDC86; HIPK3; C2ORF69; RAB38; IMMP1L; PELI1; IL4R; NUP50; CXCR4; WDR26; ADGRG2; MKRN2OS; C3ORF14; TAF13; SLC35G2; FGF22; HIST1H2BD; LUC7L2; GABRR1; UBA7; C14ORF142; JSRP1; GPR82; PPP4R2; CYP2C8; ICOSLG; FBXO34; AHI1; RNF19A; PRIM2; FGF9; SAMD3; GMPR; WNK4; IQCK; TRMT11; CHUK; FAM7F1; BLZF1; SPN; REEP3; ADPRHL1; CCZ1B; PDE7B; RP11-302B13.1; AC026248.1; FUT11; EML4; ENTPD7; SNX16; MS4A4E; VDACIP8; NAA20; SPDL1; PDYN; LATS2; POMP; MMP24-AS1; ELK4; GPR137C; GNG10; ZNF285B; FSBP; ACOXL; STAM2; 11-Mar; ASFIB; TMPO; ING2; CTD-2192J16.15; NEFM; MMRN1; AC255361.1; EFNA1; PAXBP1; CCDC84; STX16; SOCS4; MAP3K1; NAMPT; NETO2; NEDD1; HEG1; MED30; ST20; GNB5; SSTR2; NSDHL; LINC01125; PFDN4; RP3-382110.7; LIPG; COX6C; FN1; RPL9P9; LRRC69; KCNE1; CORT; AKR7A3; UCHL3; CYP4A11; USP31; PPP1R13L; UBE2F; FGL2; C1ORF174; LGR5; MAMLD1; NUP35; RP1-274L7.4; HMMR; CCDC39; GSG1L; TAF2; IRX6; HDAC5; DNMBP; YWHAQP5; CFAP74; SRSF1; DAW1; DNAJC25; CYSTM1; KIF18B; IPMK; WBP4; MSH5-SAPCD1; PYURF; BBS12; AK6; ITIH3; CRHBP; FBXW7; COL9A1; MESDC1; DACH2; ABHD17C; TOMM5; NAPG; MRPL13; TNFAIP8L3; PSME4; SLC35A3; C6ORF106; ROPN1L; BRAF; ARHGAP19-SLIT1; PUS1; MAPK6; C9ORF40; RCOR1; EFNA4; ING3; SNRPF; FAM19A2; HECA; CRISPLD2; DUSP8P3; FAM131A; EEF1A1P14; CCL27; C7ORF25; AC026369.1; ENY2; CISD1; UPK2; AC090774.2; LBH; NR3C1; CDK6; ACY1; HIST1H4H; PEG10; DPY19L2P4; MAP3K2; PILRA; PCTP; CREB3L2; OPRK1; NOP58; GBAS; TRABD2A; SLC2A4; POLR2K; DDHD1; AC241377.1; NBPF2P; ZNF354B; EFR3A; JRKL; PCGF5; SLC16A8; PDE8A; SLC35B3; TCEAL7; ATP6V0A2; XBP1; UPP2; NOP16; VKORC1L1; DLK1; TGM1; PFNIP2; MRPL33; CLK2; KCNMB3; FAM174A; PRKAR1A; FOXD4; SMN2; KLRD1; TMTC3; SLC22A4; CDR2L; NPPC; UBXN4; GS1-124K5.2; CGRRF1; SLC7A1; ZNF300; LYPD1; MAP2K4; U2AF1; USP14; TEAD4; ZNF615; KRAS; RYBP; PDE4A; NUP58; POU2F3; NUDT4P2; NUDT4P1; CCSAP; EFNB2; OSBPL11; ATAD3C; PIM3; TFDP1; RP11-76217.5; NUP98; PRKG1; HMCES; MSMO1; THOC1; LPGAT1; HMGB1P8; GSG2; DIRC3; LYN; CCDC184; DNAH10; CARNMT1; CCDC181; RP11-526A4.1; PNPLA8; MTMR11; CCND1; SRPK1; ARHGAP36; SPON1; ZNF141; PABPN1; GXYLT1; PCDHB1; SNPH; USP36; SYT4; PRSS53; NAA50; TMEM239; PIGA; CLIC2; KIAA0101; CTD-2021A8.2; STOX1; AC136289.1; FOXO1; CACNB2; PRDM8; PSMD12; PLEKHH2; POP1; ANKAR; RP11-34P13.15; PRKC1; GPR83; TOPBP1; C17ORF53; SPRED1; NPIPB5; MTCYBP21; CYP26B1; GJC1; XAF1; SERPINB8; THSD7B; CENPU; AC011330.5; PINX1; MFSD14A; RGL4; PRKY; MFHAS1; RAP2A; AC016582.2; SMC4; UGCG; TPM4; CA2; CBWD7; CCNE2; TWF1; ACSM2A; TOP1; PHF19; GDF11; DNAJC2; TMEM74; DYRK3; JADE3; CBFB; PTS; KCNG4; MED4; HSPH1; INPP1; TEAD1; CDK2; SLC26A10; SMIM17; COL25A1; ZPR1; HUS1; AC005752.10; SP110; DPP10; MPPED1; ARGLU1; TNFSF14; SNRK; ATOH7; CEACAM22P; DLGAP5; RABIF; NBPF25P; MMD; UBR7; RP11-111K18.1; KIAA0513; MT-ND3; SLC7A2; NIPA2; SLIRP; CEP85L; CALB1; RFC3; ZNF540; WWTR1; KDELR2; CYB5R4; ABHD18; APOC1; PRKRA; MAN2A1; GORASP1; C6ORF141; NET1; SNRPD1; YAF2; RCAN2; GOLGA8B; TRIB2; JDP2; IER3IP1; AP1S2; TMX1; MOB1B; RBM18; IGF2BP3; NPFF; PPARGC1A; LHFPL2; ITCH; NOM1; SC5D; LARP1B; SLC10A5; HS3ST3A1; GMNN; RWDD1; EFCAB12; PRR5L; PXMP2; MSANTD2; SQLE; RP11-795F19.5; C4ORF50; CTA-313A17.3; ASTL; CCDC150; CKS1B; BZW1; PPP1R32; AGFG1; FANCB; ZNF564; KCTD6; SATB2; EP400NL; SCD; FAM126B; EXOC6; MRPS36; ERICH2; LZTS1; AC016739.2; MISP; ATP6V1G1; UTP18; MYRIP; DPP8; ITGB1; NOS2P3; STBD1; PLEKHF2; CD84; CARD19; CELP; RP11-228M15.1; RP11-261C10.7; OPA3; C17ORF51; BEX5; C17ORF105; CCNA1; CNDP1; AHR; SYT2; PLEKHA8; GMFB; PER2; COL4A1; CAPG; PTPRC; AC006538.4; CCDC117; DYNLT3; PPRC1; SUZ12; DHCR24; ERCC4; GFRA3; HMGA2; DCAF10; NOLI1; EFCAB5; SUB1; TCEAL9; E2F3; MAF; CACYBP; DENND5B; COL28A1; C16ORF87; SPAG4; RPA3; DYNLL1; MPZL3; TMEM110-MUSTN1; UHMK1; DRAM2; RNF138; FOXA1; MYB; RCN3; HAMP; SRSF12; GDF10; NME7; SLC25A25; SLC2A3; RPL17P50; C12ORF29; MRPL47; LLNLF-176F2.1; YTHDC2; DESI2; HNRNPA3; SARIA; HNRNPA2B1; SP100; RPIA; WFDC3; PDXDC2P; ZNF711; GPATCH2; KLK10; RASIP1; PROSC; HACD1; ZNF331; HIST1H2AG; CACNB1; USP32P2; TXNDC17; PLAGL1; CHTOP; PPP6C; ODF2; HTR5A; SGPP1; KLK14; ADAT3; APOO; NPAS2; CDC26; ETV2; ZNF266; ISCA1; RAB22A; SLC38A6; SUZ12P1; MINOS1; YWHAZ; CSNKIG2; ZNF726; SLC25A3; CTA-963H5.5; G3BP1; SLC25A37; TMPRSS9; KCNC3; COX16; ANKRD10; EXOG; TIAF1; IDE; TTC39C; TIA1; EMILIN2; POLR1C; MRPS18C; ZNF630; PPP2R1B; TMC7; DDX20; HCCS; RP11-113D6.10; UBE2G1; SCIMP; CNPY1; BEGAIN; UXS1; C15ORF61; PGRMC2; TMEM97; RAB12; COL22A1; ABO; RIMS2; DCAF4L1; NDFIP2; GTF2E2; IFT57; RPRDIA; RP11-309L24.4; ATP2A1; PBK; CDC25A; DPY30; CTC-448F2.6; DUOX1; ARPP21; POLQ; TFAP2E; CLDNI1; NEUROG2; TXNDC15; RNF11; IGSF22; RIOK2; CDHR3; ABCA9; NIFK; LAMA4; PSRC1; TMEM45B; OTUD6B; RPL23AP49; TWSG1; RBM22P2; WDR93; ASIC2; GATSL3; PTAR1; WDFY1; HECTD2; RP11-45M22.4; C10ORF82; FBXW2; GRB10; ZPLD1; MTCO2P12; FAM118B; LUZP2; KCMF1; PI4KAP1; ACP7; E2F1; FERMT1; PLEKHB2; HPS5; TUBE1; GOLT1B; EEF1E1; FKBP11; NASP; RP11-505K9.4; TBCID3B; HFE; PPAT; AP000347.2; CMTM6; MITD1; WARS2; ATG4D; SELT; EIF4E; SEPT7P2; OAT; LARP4B; TAL2; AC004985.12; PAX3; WTIP; PDCD2; TNFRSF11B; TBCE; TMC3; DGKI; CRK; COL14A1; RHBDF2; PPP1R2; SLC17A5; HMGN1; IGIP; CPA4; RP11-5P18.10; HS3ST3B1; LLNLF-173C4.1; WEE1; C1ORF186; TCEAL8; CENPH; GNG5; CTXN3; KCP; NDUFA5; KCTD9; SRP19; ITGBIP1; CLIP4; EIF4A3; KIF9; THBS2; SLTM; SHISA6; MSH6; SIGIRR; SNAP25; PARM1; RP11-643G16.4; FAM91A1; ARL5B; EFHD1; EN1; ZNF251; HABP4; ABHD1; CUL3; RP3-449O17.1; SAXO2; RPL39P5; AVEN; ACBD3; DDX18; COQ10A; HMGB3; GDF1; TSPAN9; SKP2; SLC39A4; RECK; SLC15A1; TYW5; C6ORF132; FRS2; AC010547.9; NPEPL1; CCNC; HMGXB3; TSPYL2; CDC123; FAM19A5; GSPT1; EIF3J; STEAP4; CDADC1; STK32B; RAD54L2; RFK; MAP2K1; ERICH3; RPL5P30; SYNJ2BP—COX16; ZBTB16; NTRK2; FAM122B; APOD; AMER2; ZNF322; LUC7L3; RHEB; GLMN; ANOS1; PACSIN3; ATPIB3; ZBTB26; CCDC144A; PRPS2; NECTIN2; PKD1L2; SLCO4A1; PRKAR2A; ARMCX3; CDKN2C; UBE2N; ETFDH; TCHH; ZCCHC12; DCUN1D4; MAP3K8; SLC6A16; CDV3; SRSF11; MPC1; RPSAP14; UBL3; SOCS2; EIF2B4; SLC25A32; NUSAP1; KIF2C; DCLK1; ESAM; KLHL1; BDP1; SIX4; CENPK; CENPQ; RUNDC1; GPR180; GCC2; RBKS; PLEKHH3; TMEM126A; LPL; UBE2B; SLF2; MVK; CXCL14; WASH5P; SLX1A; ATG14; SNTB2; SAMD4A; LRRC40; AP3M1; PSMB8; GOLGA8A; TRAC; BET1; PLD5; KIF3A; ACTR2; MOB4; ADCY10; ATAD2B; FBXO43; USPL1; DUSP3; GTF3C6; G3BP2; RP11-124L9.1; MTND5P11; CEBPZ; XRCC2; TMEM267; DCAF1; FAM84A; DNASEIL2; RUBCN; MURC; ARF4; CTU2; MTATP6P1; ARTN; KCNV1; NAMPTP1; SLCIA3; TMEM126B; SMC5; PRODH; XKR9; CHCHD7; NUTM2A; SKOR2; HSD17B12; ANK1; PIWIL4; ZBTB11; GATC; USMG5; CAPZA1; LARP4; RC3H2; TXNL4A; ZDHHC3; HSPE1-MOB4; EDRF1; ELOVL4; PREX2; CCDC169; PANK3; STRN3; SLC25A44; MROH6; SLC30A7; MAFF; UBE2W; ARIH2OS; FBXO45; RINT1; TRMT6; SCML2; CXADR; CRY2; C18ORF25; ARMT1; SPOCK3; UBE20; AGAP9; TBCA; EIF4A1; DDX17; FAM120A; DHX29; AGAP6; RP11-113I24.1; SUCLA2; USP37; MTRR; BORA; MAPILC3A; RAB39B; NDUFB6; VPS29; KLRAIP; MRPL48; CCBE1; HIST1H4J; SBSPON; CNTN5; EXOSC3; SPDYE11; SSNA1; COMMD8; WDR25; B3GNT2; C1ORF159; ZUFSP; HNRNPH1; R3HDM4; EGFL8; KIF18A; STK24; NAA15; MRPL36; HSPA4; STARD4; PSMA6; DBNDD2; PI4K2A; HDX; CHKA; GTF2A2; CYB561; CCDC7; PRKAG2; FH; CH17-5A7.1; NDUFA12; YMEIL1; ADAMTS12; CCR6; TMED2; ADCY10P1; FDPS; 1-Sep; MKLN1; FKBP9P1; HSPA14; HNRNPK; OSGIN2; PNPLA3; SAMD4B; GNG4; DENR; KRTAP5-2; FAM220A; FMR1; CCNT2; TSPYL4; GAR1; SRPX; URB2; SOCS7; HERC2P2; PHLDB2; ICA1L; BDH1; STK38L; MCM10; BTG3; FBXO33; WASL; PIGG; CNBD2; GCLM; WNT5A; RP11-213G2.3; FGFR1; MRPL22; RALGAPA1P1; GRIK2; ZNF131; MIER3; TUBB6; AL021546.6; SPOPL; BTBD3; C3ORF70; PGAP1; RNF169; RP2; TYW1B; AKAP2; NANP; CCNL2; UTP4; MT-ND6; TMTC1; RPS6KA6; ADGRG3; RHOT1; CCDC3; CTPS1; YWHAH; HEATR3; RAB14; GABRP; TMEM191C; UBE2QL1; RNF2; PIP4K2C; KCNJ8; ADAMTS7P4; CAMK2N1; UBE2J1; ECD; UBP1; GLRA1; DCK; CLEC18C; SSC5D; ARHGAP15; OXSR1; TMUB1; TMCC3; FGD2; CCNI2; NDUFC2-KCTD14; C5ORF63; CHCHD4; ZNF780A; GJC2; CD164; RAB11A; NTAN1P2; EIF5A2; RP11-44D5.1; SMNDC1; PGAM5; MOV10L1; CSTF3; PAK3; TCEAL6; NDUFB3; CCDC125; TXNDC9; FAM101B; OSGEP; BUD13; LARP6; ATP6V1C1; EHBPIL1; AKAP8L; CMTR2; TMOD1; FAM177A1; PTBP2; SMURF1; ARPP19; AC112497.1; LRRC34; PQLC1; DDX50; PTGES3L; NFAM1; CNGB1; DHFR; WIPF2; TVP23B; C18ORF32; PTP4A2; TMA7; CCAR1; NPM3; TPP2; SEC31B; RBM27; DNAJBI1; USP7; PITPNM2; DNAJC27; PPP1CB; PDHX; KIAA1644; CXORF58; IGHVII-30-43; NACC1; BST1; TPCN2; CEP128; NXF1; ZNF26; TMEM60; SIRT7; GOLGA6L4; TMEM117; NGLY1; ING1; NFKB1; CTSS; NKIRAS1; RARS; LBHD1; RP11-229P13.2; HAUS6; ZNF826P; PANK1; PTPN2; IFI44L; ETNK1; CDC27; FUT7; C5ORF15; DNAH9; SAT1; ADO; LCORL; ATP6VIB2; MYEF2; SPTSSB; PPP1R12C; RP5-1009N12.1; PDCD10; PITPNA; PPAN; B3GNT5; NCBP1; GTF2IRD2; DYNLL2; IGSF6; TMEM132B; FHOD1; ACADL; FAM57A; HMGB1; ACSL3; ANKS1A; TMEM67; MBIP; SET; SLC15A2; TCF7; ZNF410; TAFIA; WSCD1; TEX10; TMEM151B; EIF1AX; ADHFE1; ERBB3; TBCEL; GALNT7; ELFN1; RIMKLA; ADAMTS16; C10ORF88; RP5-1000K24.2; BIRC2; RNF146; LEFTY1; SUMO1; RAB31; USP6; ZNF576; PITPNB; TTC14; SGK494; PHF13; SOCS6; ALPK2; LY6G5B; SMARCD2; ARL4C; FRMD6; ISPD; DENND4A; GTF3A; IL1RAPL1; CDK7; MRAP2; RP11-435I10.4; ZNF355P; MC1R; CDH13; FZD10; QPCTL; LIPT1; SEC23IP; CAPRIN2; RDH5; JOSD2; DNAJB14; SS18; ZDHHC20; HLA-L; PIK3C2A; MRPL9; TPO; ATP5E; NPIPB4; SLC25A51; COL6A3; EEFIAIP8; RIIAD1; STAP2; KCNN1; GLMP; PCDHA13; SEC61G; CYBA; RANBP2; CDC42; IFNGR1; FGFBP3; RAN; DSTN; PHTF2; C16ORF52; SRPRA; TM2D3; SEC62; ACSL4; TMED5; and C20ORF24.

### Top Downregulated

KIAA1107; AC009133.23; RP5-1022E24.6; COX6A1P2; FOXD4L3; RPL41P2; CFAP126; RP11-651P23.4; AC002310.11; XXBAC-BPG246D15.9; EGR1; ANKRD20A2; RP11-544O24.2; RP11-321N4.5; TMEM189-UBE2V1; SPDYE10P; RP11-1012A1.4; RP13-582O9.6; AC096949.1; EGR2; PRR5-ARHGAP8; RP11-468E2.2; H3F3AP4; SENP3-EIF4A1; EIF1AXP1; SLC2A3P1; HIST1H2AK; BNIP3P1; SPOCD1; BP-21264C1.1; TRIM74; AL109923.1; RP11-1280N14.3; SRP9P1; RP11-729L2.2; RP11-468E2.1; AL031664.1; CNTNAP3P2; ANKRD20A1; AC006011.4; PPT2-EGFL8; PRH1-PRR4; RP11-123K3.4; AL023875.1; RP11-574E24.3; CTD-2616J11.11; RP11-234B24.6; NPIPA3; SLC24A5; FAM35DP; ANKRD20A3; Z83313.1; RPL7P9; ANKRD18A; VIPR1; CRIP1; KLHDC7B; POTEE; RGPD1; AC002398.9; RP5-972B16.2; RP5-850E9.3; CH17-264B6.3; TRIM61; AL513122.2; AGAP12P; TLR5; KBTBD11-OT1; RP11-848G14.5; SRGAP2D; ISY1-RAB43; AC079780.3; SRL; AP003419.11; SSR4P1; TMEM144; TGM2; GUSBP5; RP11-248C1.3; RNF103-CHMP3; BMP3; TERB1; TCF15; TIGIT; RP11-371A22.1; PCDHA7; EIF3CL; TMX2-CTNND1; SOWAHD; USHIG; CHRNB3; CXCR5; ANG; RHEBP1; JUN; LTC4S; EDARADD; RNF2P1; RP4-559A3.7; SLC6A10P; BBC3; RP11-392E22.9; RTP1; RP11-463D19.2; ZC2HC1C; RPL13AP5; VWA3B; ARC; CTD-3214H19.4; FAM90A24P; RP11-1305N21.3; RDM1; AC113404.1; GS1-184P14.2; UBE2FP3; RP11-848G14.2; AC090945.1; DCT; NUTM2E; SLC35E1P1; PARP12; MTNRIA; RP11-927P21.5; CFAP99; FRAT1; RP11-977G19.10; RP11-47I22.4; LHFPL1; BCL2L14; AC129492.1; ACTG1P10; SPDYE6; SLC29A3; GTF2H2B; AC255093.1; GATA2; RIBC1; MST1P2; PSPC1P2; PRKCH; RP4-706A16.3; TBCID26; GPR68; RP11-287D1.3; PAQR5; NPAS1; DCST1; GPR146; RP13-512J5.1; MMP11; AC213203.1; ARHGAP25; HMGB1P10; ZHX1-C8ORF76; RPS3AP5; RAB17; MADCAM1; SFRP4; CYR61; RP11-61L23.2; TUBB4AP1; CNTD1; FTHIP7; SMCO3; F10; RGS9BP; CENPVP3; C14ORF93; TPTE2P6; AC098614.2; KCNA5; CDK3; ANKRD31; SPAG11B; HRC; IFI30; LIN28A; IQUB; RPL4P4; C16ORF89; SLC25A52; SEC1P; ABC13-47488600E17.1; GFAP; GRID2IP; HIST1H4C; SMGIP4; ARHGEF37; RAB37; FAM86B2; LAG3; SLC45A3; FRAT2; FEMIAP2; GPX7; CCNB3; ANKRD35; AL022397.1; TMEM156; PNPLA1; HSPA1A; RP1-2705.3; HAPLN4; KCNIP3; ARHGAP30; AC234582.2; VASN; HIST2H2BC; CTD-2192J16.17; AMPD3; TNNI1; ATP5FIP5; AP000687.1; RPL13AP7; RAG1; DHRS3; RPS2P7; PYGM; GRAP; CATSPERB; ORIF1; DUSP1; ATOH8; SDADIP1; LKAAEAR1; EPX; FGD3; EGR3; HIST2H4A; CCL5; KDM4D; FAM185BP; ZSCAN23; TMEM79; CYP4F62P; CSPG4; DDX28; SYCE1; BORCS7-ASMT; CFLIP1; C2ORF71; PTGER2; SOX21; DLEC1; SERPINA5; CF1; RP11-958N24.2; RP11-2711.4; ZNF296; RP11-312J18.5; AC009022.1; PTPRB; IL16; MOB3C; RHEBL1; ZBTB32; RP11-1023L17.2; RPL23AP53; HSPA6; RP11-553P9.1; EEFIAIP4; RP4-740C4.5; FCRLB; C1ORF158; NMUR1; MYO15B; RD3; FSCNIP1; ARSE; RAB41; DRC1; FABP5P7; HMGN2P5; HMGN2P3; AC243587.1; CCT8P1; CH17-264B6.4; CTD-2561J22.2; ARL17A; LCN12; ANXA2R; RP11-98J23.2; RP11-467H10.1; NLRC5; RP11-542C16.2; PLEKHF1; NUDT18; FAM86JP; TFAP2D; GPR149; E2F3P2; GPRC5C; ITGA4; LLOXNC01-16G2.1; CD4; SNX18P3; RP11-514P8.6; DNAH12; SCML4; PLCXD2; FAM201B; KIF19; TMCO4; PAX8; RP11-815J4.6; DDX11L2; ZNF438; MXRA5; CH17-296N19.1; DUSP10; SLC25A35; PLAUR; ENPP3; GXYLT2; HACD4; CCL2; SLC2A5; PCK2; GAS2; CMTM8; NRIP2; DCST2; H1F0; SLC16A13; THEMIS2; RP11-219A15.1; YPEL2; PGM5; TSSK6; TXNL4B; PIRT; RBM14-RBM4; FAM180B; CD99P1; PDE11A; PTPRR; SH2D3C; C4B; TPI1P2; FOXD4L4; GABRR3; GVINP1; GPER1; UCN; BDKRB2; CIQTNF2; CCDC144NL; FXYD1; RP11-174O3.1; MEI1; TBX2; HMGA1P1; FAM47E-STBD1; OR7E94P; ZNF114; HIST3H2BB; LL0XNC01-116E7.4; FBXO17; DNDIP1; POM121L9P; IGSF5; DSTNP1; CPLX3; CCDC103; TVP23C-CDRT4; SFRP5; RGS13; LINC00854; ARID5B; RAE1; JAK3; CHDH; DYRK1B; EFHB; MUC20P1; NMNAT3; SPATA4; C1QTNF3-AMACR; DUSP16; BAIAP2L2; XKR8; FAR2P3; ZNF474; MAP1LC3C; MAP2K6; DSE; IGSF10; AL354828.1; FBXO24; NHEJ1; RP11-1016B18.1; BTN3A1; GALK1; TEX40; AC007256.5; ADAM21; RP11-14713.1; CHURC1-FNTB; RNF152; PFKFB1; RAPGEF3; NUDT8; HMCN1; KLF11; AFAP1L2; HTR7P1; AQP6; MRM1; PKDREJ; SOWAHA; KRBA2; FUT4; RGS5; P2RX6; FAR2P4; TST; INPP5D; RARRES2; NPAP1; CAV2; UGT8; MYCL; UBALD2; RP11-778D9.4; RP11-7D5.2; LMOD3; CLDN5; CHRNA6; ZNF670-ZNF695; C9ORF129; MFNG; CLCF1; GPR21; CYB561D2; AC010642.1; ADAMTSL4; IER2; GPR6; NFE2L3P1; RP11-762H8.2; SCRN2; ZC3H4; CHI3L1; PROCA1; MATN3; PRRX1; TUBBP5; SULT1A3; TMEM187; B3GNT9; RP11-646I6.3; HEBP1; ID1; ATP6VOD2; RP1-16IN10.1; PROKR1; MIXL1; PLEKHA8P1; SPATA18; UNC5CL; HIST2H2BD; EIF1AY; CLEC18B; SLC47A2; ZNF488; DEPTOR; TCAF1P1; DACT2; ADCY7; HNF4G; EEF1A1P5; LOXL1; ARSF; RIMBP3; IFI16; LAYN; RPS2P46; TRADD; TFDP2; ZNF135; NYNRIN; SHANK3; ZNF57; HMGCL; ZBTB45P2; DLEU7; EXOC3L1; SLC30A2; FAM187A; B3GALT4; GPNMB; XYLB; RIN2; FOXO3B; RP11-109L13.1; RPS28P7; AC107983.4; AC018738.2; GJD3; RANBP3L; NTN3; SLC29A1; C1ORF64; GCNT2; RP11-480I12.9; NR2E3; PNRC1; LRRTM2; PAFAH2; HKDC1; LILRB5; C20ORF144; KCNJ10; ZNRF2P1; IKZF2; AC007919.18; CD3G; EVX2; C1ORF115; GADL1; CTD-307407.11; NGFR; CA6; RP11-347C12.3; RP11-514P8.7; EFS; ANXA3; NUTM2G; UNC93B1; ALOX12B; UCP2; PIGV; CXADRP3; PLCH1; HPGD; RP11-364B14.3; RSPH10B; CRLF2; GPR37L1; BTBD18; C10ORF54; ROR1; WFIKKN2; MEIS3P2; CNTN3; PRIM1; STMND1; GYG2P1; ZNF563; SLC12A1; PSMC3IP; CTGF; GLRA3; DBX1; CPEB3; ZNF887P; PLIN4; ANO7; AMDHD1; RBM43; IRF5; TXLNB; ANKRD55; BHLHE23; C17ORF96; RP11-82H13.2; PADI2; TRERF1; BRS3; TTC28; PLA2G7; MSL3P1; CREBRF; ZNF215; KCNRG; PDZRN3; DHX58; UPF3B; TCEA3; APLN; FBXL13; ACADS; MARVELD1; MACROD1; DBP; RIMBP3C; IER5L; ITGB3; ALG1L6P; LAMA3; C4B_2; RFX2; RP11-56215.1; CORO2A; ZNF658B; TBX6; CABYR; HSD17B14; RP11-17M15.2; LHX9; GPR150; TIGD3; CD248; DDX12P; C1QTNF5; GRIN3B; TCP11L2; GAL3ST1; MAP10; CDPF1; RCL1; RUNX1; AMACR; ABHD15; RP11-848P1.9; MKS1; XDH; AC090094.1; STK32A; LLOXNC01-116E7.1; RP11-495P10.10; FAM218A; SYTL2; BBX; CCDC151; ZBBX; CEP97; C19ORF81; DNASE2B; ATP2A3; ARMC12; SERPINF1; RNASEL; IGFBP6; TNFAIP2; SOCS3; NCCRP1; SLC25A20; SLC7A4; NCOA5; PIK3CD; KCNH4; C21ORF62; NPIPA7; ONECUT1; ALDHIL2; FAM90A12P; SLC25A21; MYH7; TPSG1; CTB-63M22.1; ENOIP4; PITHD1; NFATC4; PKDIL1; 2-Mar; ZNF185; PARP3; OPRL1; BTN2A3P; LAPTM5; TSTD3; ADAMTS1; SPEF1; RHCE; CMYA5; KLF7P1; RP11-274B21.1; GLTSCR1L; TMIE; CTD-2026D20.3; BTF3L4P2; ZNF628; DSC2; IMPA2; FANCE; SLCIA5; HK2; ZFHX2; LIMK2; DENNDIC; AKNA; SLC16A3; PLSCR4; ITGAX; HNMT; RNASE4; INO80B-WBP1; CHCHD10; SYCEIL; SLC27A3; FAM117B; ALOX5AP; AC003006.7; GLUD2; PPP1R42; FBP2; ABC14-864958H18.2; CLK3P2; VGLL3; KIF16B; NMRAL1; MUC3A; STARD8; KIAA1683; C8ORF37; C6ORF118; BTG2; CTAGE3P; TGFBR3L; TATDN2P2; RP11-104G3.2; SPDYE5; BCKDHA; POU3F1; SPINT1; TLE6; DYDC2; FBF1; KCTD19; ITPRIPL2; CTD-2054N24.1; PCDHA5; HCG27; IL17RC; CHRNB4; VIPAS39; RND1; SLC39A8; PTENP1; AC004381.6; FAM72D; ALDHIL1; SLC52A1; AC009967.3; RP5-1021I20.4; ZNF99; METTL4; CITED4; NKPD1; GRM1; STEAP3; AC125232.1; TMEM184A; RP11-262H14.5; FAM20A; TAS2R15P; STAR; C16ORF54; ZCWPW2; RASA4DP; MGST2; RP3-337H4.6; CCDC163P; ACOT1; PCDHA11; IGF1R; SMARCAL1; DCDC2; SOCS5P4; MTND6P21; COX19; PDCD4; HAS1; IER5; MUC12; EFEMP1; ZNF503; REPS2; JMY; SLC7A3; ASNSP1; ODF3B; GPR156; EIF4BP7; CAD; RGR; KCNJ12; RP11-694115.7; TOX2; ITFG2; ABCD2; IPCEF1; PLOD1; ADAM29; DIXDC1; CASZ1; IRS4; TRIM14; APOBEC3A; SLC24A1; NOX4; PTAFR; MEFV; TSSK5P; FZD8; PGF; PLD6; PRLR; ICAM5; CRYBB2; AC007318.5; TP53; BOLA1; ACTA2; THBS4; FAN1; BORCS5; AMOTL2; ST6GALNAC4; OXCT2; TRIM68; MCEE; GPRC5A; CCDC170; SEC14L5; CDHR5; TCEANC2; CAND2; CERKL; PEX11G; ERI2; C1ORF56; RUNX2; CHRNA2; PGBD2; DUSP15; GPANK1; ATF7IP; HP; DOCK6; TEX21P; RPL7P11; TMED10P1; FILIP1L; TMEM141; PTCHD4; EMP3; SYNE1; ORAI1; LIAS; NPR3; DDN; DMKN; MORN3; TMEM42; ATF7IP2; KCNJ11; KCTD16; KCNK2; FAM26E; F8A1; NEK8; C3ORF33; PIHID2; FTCD; ABCA12; LIMD1; EPPK1; CFHR1; PPMIN; PDGFRB; MAP3K6; SYTL1; C2ORF16; RP11-452D2.2; PALM2-AKAP2; UNC5B; KIAA1024L; FILIP1; SYNE3; EEF2KMT; CH17-64J14.4; PRDM16; GCSHP3; C14ORF169; EEFIDP2; PRRG3; RHOH; SLC10A4; ATF5; HMGN2P15; JAKMIP3; JMJD4; SULF1; FAM110A; RPS27P25; CHTF8; POLL; WDR81; TRAPPC12; HSPG2; CDC25C; KCNA3; ZNF385C; RP11-34P1.2; WDR31; PCDHB3; C19ORF44; RP11-216L13.17; LIF; FAM181A; PCDHB6; FAM90A11P; MUM1; ACBD4; PCDHAC1; NEU4; EFCAB13; EXTL1; INTS9; SPAG8; SORBS2; STOML1; TASIR3; ASS1; OLIG2; TYSND1; RP11-168A11.4; ST6GAL1; RBM20; PRPSIP2; ACER2; CASP6; RP11-96L14.8; RP11-411B6.6; METTL23; TNFAIP8L1; FOXJ1; ADAMTS5; LAIR1; KRBOX4; TMBIM1; RP11-844P9.2; CHEK2; SULTIC2; SVEP1; HYLS1; DUSP19; HHAT; MGMT; PLCD4; FHAD1; ARHGEF39; CDC42EP2; APOBEC3G; SUOX; ASB18; PLCD1; ZDHHC1; AL022067.1; CIB1; TSPAN10; ELMSAN1; DCHS2; CD8A; SH3D21; RP11-252A24.2; C16ORF86; UTS2B; AB019441.29; FAM63A; SLX4IP; GALNT12; SLC26A1; CIART; TEX22; SCRT2; GATM; ATXN7L1; CEP44; B3GALT5; A4GALT; KATNBL1; TMEM159; RP11-75L1.2; MANBA; FANCC; RP11-44F14.1; RPL17-C18ORF32; PIGZ; LINC00959; AFMID; SHANK2; NKAPP1; HSPB8; MGARP; MYD88; KCNJ3; PCDH8; SEC14L6; FZD1; IL11; C17ORF100; TNFSF13; CLDN4; PRR18; PQLC3; PLEKHG4B; ZFP36; PDZRN4; ZSWIM3; VRK2; LRRC75B; TMEM101; ROM1; RSC1A1; AKAP3; SLC36A2; IFT140; CTD-2021A8.3; PDLIM2; ING4; MED11; AMT; EDA2R; NR3C2; EXOSC5; TRIM56; BBS4; SOGA3; NATD1; SLC2A10; ZNF460; SNCAIP; FAHD2B; IRAK4; COL15A1; CISH; CISD3; JUP; Z97634.3; MATN1; DPYD; GOT2P3; FAM110C; MOCS1; ZNF823; SERTAD4; AMIGO1; ZNF805; AL034550.1; KHDC1; RADIL; RP11-949J7.8; ADCK3; AF011889.5; VTCN1; FAM92A1P2; RARG; STARD5; ALG12; TSPAN15; SMG1P1; GABRA1; PIPOX; UGT2A1; C8ORF58; ZNF860; TSPY26P; RP11-403113.4; KCTD11; LRP5L; MAP2K5; MXD4; EIF4E1B; CYP39A1; RCOR2; WIF1; TFB2M; TP53TG5; ZBTB7B; HAUS8; ABHD14B; PIP5KL1; IGSF9; SUGT1P3; LEF1; GEMIN8; SYNE2; CATSPER2P1; MIEF2; SHROOM1; IL34; RP11-480I12.5; KANK2; ADIPOQ; CDC42EP1; GRM2; FUT1; ZC3H6; SIPA1; PHLDB3; LENG9; C20ORF194; ZNF663P; RNASEK-C17ORF49; ANKRD18B; TRIM34; OVCA2; PMS2P3; TMEM54; CHST14; C19ORF45; DLL4; THAP8; TRPC6; LRRC23; FYCO1; XPC; C1ORF54; AF131216.1; CCDC15; SLC7A10; CDT1; MFSD3; ARMC5; F7; RAD54B; FAM107A; NPR2; RP11-452G18.2; ZNF846; AC004076.9; CACNB4; LAMC2; BMSIP4; STRADA; CMPK2; FAM173B; SIX2; LRTM2; TMEM266; C19ORF73; IFI27L2; NR1D2; DRP2; ACAP1; JADE2; ENGASE; DHRS7B; LRRC56; ARHGAP18; HTR6; CHRNA3; HHIPL1; GTF2H2C; KIZ; TSPAN32; GDPGP1; GPR1; APOM; TRPM4; HEXIM2; KCNC2; THEM6; C11ORF63; SIRPA; AGPAT2; TMEM150C; INSM1; STC2; RECQL5; LYRM9; POTE1; FAM134C; NKX2-2; ST8SIA4; FAM184B; TNFSF12; MYO18A; ACAA2; ZNF610; DNAJC5B; PCDHB5; SZT2; PFKFB3; TAF4B; KLHL32; LETMIP2; SUN2; RP11-474D14.2; CNNM2; PRIMA1; BTN3A3; CTD-2583A14.10; TEK; GLDC; PRELP; BRINP3; DUSP6; OR51E2; BMPER; ARMC6; PLCB2; TRAPPC6A; KCNB2; CIQTNF1; NAGA; SMIM20; COL4A3; CCDC71L; TNRC18P1; COL9A3; ALS2CL; GPR88; TBCC; RRAS; AJUBA; UFSP1; NIPAL3; ATRAID; TRIM65; KLHL3; GLUDIP7; C2ORF83; U47924.6; HES1; NEK11; SLITRK1; HSD17B8; URAHP; BAHCC1; C21ORF33; FRY; KIAA0355; TRIM27; GSTZ1; CCDC8; MRVI1; GPRIN2; HOGA1; RP11-446H18.1; FAM181B; MTA3; PCLO; FOS; TP53I13; ZNF668; C2; CDCP1; SGK3; BEST1; ANKS4B; MSRA; FUZ; TPTE2P2; LDHD; ARHGEF19; ABHD16B; ZNF682; BUBIB-PAK6; SP140L; SLC9A9; CROCC; PLCL1; TLR4; OMG; MDK; PCDHA9; ITGA9; PEX6; RASA4B; FAM13A; MPRIPP1; ADRA2A; FBLN7; PAX2; ASB14; RYR1; BLM; FRMD3; PCYT1B; CYP17A1; DNPH1; CBX4; PPP1R10; PCDHB11; LGALS1; TNIP1; GCKR; BEND3; GTF2A1; DLL1; PPP1R17; XRCC4; C21ORF58; GPR75; MAST4; ICK; ESYT3; HEMK1; RP11-449H3.3; SCARF2; SMIM3; MRPS24; DGAT2; CCDC180; ZFAT; PIK3AP1; UBALD1; CRB2; MAB21L1; RP11-677M14.5; ADAM20P1; ZNF396; FAM69C; TMEM129; SPHKAP; NBPF14; TBC1D4; FBXO6; ARMC4; DNAJC30; ISLR2; AARS2; PLAGL2; TRAF5; SLC41A3; SLC22A18; CCDC142; TREX1; CASP8; KIAA1217; RNASEH1; ADSSL1; MRO; CXXC4; APOL2; PDK1; RP11-697E2.6; IGSF3; SLC38A3; TRIM7; TRIL; UPF2; CEP126; SERINC2; ANKDDIA; PIK3IP1; SWSAP1; ACSM3; KIAA0922; PLIN3; WNT7A; TAB1; NEK9; PHF11; C19ORF54; TEKT2; CNTNAP3; TAGLN; FAT4; TKFC; STYK1; SFR1; VCAN; PDF; KIF25; RP11-15H20.8; DCLREIA; AGER; SLC25A24; FOXO3; GLTPD2; AKRIB15; FBXO32; DDTL; SCN2B; SHISA2; NAGLU; FAM90A1; PRTN3; RNF212; MEX3B; OIP5; PPM1M; CLDND2; TRIM5; MED12; TIGD4; RP5-859M6.1; ZNF165; P4HA2; PDE1A; PALMD; YIPF2; CENPBD1; SLC9A3R1; ZNF702P; ITPRIP; SHH; PAIP2B; LINC01529; KHK; AP5B1; KDM8; LRRFIPIP1; NEK7; HSD17B13; LHX5; ESR1; ABCC6; ZNF837; RHOV; GBA; DNASE2; TMEM63A; CFC1B; OR7E154P; STARD9; AC245100.1; BTN2A2; TRANK1; ELAC1; SLFN5; ABHD11; HDDC3; GLI4; SIDT1; CCDC71; GPR143; TATDN3; TARS2; TMEM241; EPOR; RSPH4A; FOXP3; DHRSX; RP11-568K15.1; FECH; CYSRT1; TIMELESS; PITX2; KCNE3; RPS24P8; ZNF137P; CDH23; CYS1; BTD; OTOF; PIDD1; MFAP4; SLC2A12; CTD-2010116.1; ZFHX4; KCNH1; ACAD11; FAM86C1; GRPR; POC5; FCRL2; SNX29P2; USH2A; HGD; PKD2L2; FZD5; FAM13B; HCRTR2; GATA3; SPHK2; TMEM191A; RP11-444E17.6; UNKL; RP11-176H8.1; PRRT3; CCDC77; ZNF404; RP11-274B21.2; HACL1; ISM2; LY6K; ARHGEF10; H2AFJ; ACAD10; CTD-2521M24.4; NR1D1; DDIT4; ARHGEF16; ITGA10; SYT9; NUDT12; RP11-15K19.2; MXD3; ZNF684; PYCR1; CYP27A1; GPR45; CPXM1; RNF32; CTC-487M23.8; ZNF781; HMCN2; PTPN3; DOCK9; ZSCAN18; CTF1; ZKSCAN7; AHNAK2; ADM2; ZNF416; CMBL; ANKRA2; EAPP; FNDC11; TMEM150A; C3ORF49; RPP25; RASL10A; RPLPOP2; CHRM5; KTI12; IL27RA; SPRN; TMC06; CASC1; MEGF6; C19ORF57; SORD; ABCC3; PRDM7; FAM110D; ZNF491; RP11-244H3.4; ENOSF1; NOXA1; ST8SIA6; GANC; TRIM22; SIL1; SNX7; IL21R; KLF2P4; DMGDH; RP11-266L9.4; GNL3LP1; WSCD2; PRKD1; CYB5D2; CDH7; RNF144B; NBR1; RP11-347C12.1; CBX3P2; SDHAP2; RPS10-NUDT3; RGS9; TIFA; DBF4B; MRPL10; SIGLEC10; PRMT3; WDR63; ZBTB42; ZNF763; TTC12; MTF2; BLOC1S3; TNRC18; FOXO4; LRRC27; ITPR3; FAHD2CP; XRRA1; BGN; WNT9A; C17ORF97; FAM101A; PLEKHM3; FZD2; ARAP1; H1FX; GATB; RP11-29813.5; AC156455.2; MYRF; KRT18P34; LPAR4; GPR89A; PPP1R3G; THSD1; GSN; WBP2NL; SPON2; SGK223; FKBPL; WDSUB1; MECR; MED26; SBF1P1; SPEF2; LRFN5; C22ORF46; C2ORF88; POLRIE; PCDHGA9; LRCH2; ADGRF3; SYNPO2; NBR2; RBMX2; ZNF280D; ADGRL2; PRX; CTD-2245F17.3; THNSL2; ADPRH; CCDC102A; MPHOSPH9; UNC79; C1ORF106; CDKN2AIP; GADD45G; CCM2L; RTN4RL2; AQP7; SMG9; HRG; NTN4; SCRG1; GBX2; ZNF75D; NUPR1; PRSS16; LMAN2L; PNMAL2; C19ORF47; CTTNBP2; PCDHB7; ALG3; OLIG1; RP11-791G16.2; AC083899.3; ZNF425; APH1B; TOP1MT; PPCDC; HSPA1B; LRRC32; KCNT2; GNL3L; HMG20A; SLXIB; LRP1; ZNF20; UPK3B; TOE1; MOCS3; DHRS4; ZSCAN5A; ABCA13; ZNF177; GABRB1; MUC5B; KLHL35; PEX5L; PPM1J; CSRNP3; PPOX; PRKACB; KIAA1671; FEZF2; PCDH1; LRRC37A4P; ADCK2; ZNF525; RGPD3; DSC3; RMI1; C1ORF53; CHKB-CPTIB; TOB1; IGFALS; C11ORF45; PCDHAC2; AC012314.3; AC012314.2; AC012314.1; AC012314.4; AC012314.6; AC012314.5; PML; NAIF1; PTPDC1; PLEKHG3; TBCID31; SLC35E2; POU5F1; TDRKH; DLEU1; CYYR1; DMRTA2; RAB30; PTPMT1; C9ORF91; CNGA4; ALDH3B1; PARP16; HHLA3; ZNF256; STPG1; SDHAP3; KRT18P59; TANGO6; IRF2BPL; IGFBP5; KSR2; HDAC11; PLA2G4C; SLC35C2; BCAM; CUL7; COLQ; HNRNPAIP10; TCAF2; N4BP2L1; APBB3; SLC43A2; TMEM185B; SEMA4B; OGFOD2; C1ORF204; EGFL6; VCAM1; ZNF44; EP400; ZNF587B; TRIM16L; C3ORF18; NR5A2; RHOB; C2ORF91; FAXDC2; RCAN3; TCFL5; IFIT3; C17ORF67; MTMR8; TSPEAR; PPP1R21; EDN1; NFYC; EX05; TMEM262; ZNF462; GRM6; ZNF543; SLC29A2; TRIM66; RP11-460N11.2; ACP6; EPHB3; ABHD12B; PATE4; CRYAB; CTD-2027119.2; SP8; ZMYND8; SYDE1; ZNF701; GCK; C10ORF2; DHX35; KLF7; EHD2; ATR; DDRGK1; NACA3P; ZNF740; ARRDC4; DDX11; TRPC4; DOCK10; GADD45B; EMILIN3; MST1; MKNK1; TOX; TEPSIN; TTC38; CTRC; RPS16P9; NCOA2; TAL1; CRYL1; PIBF1; TESK2; RP11-192H23.4; BTN3A2; C1S; MYO3A; ZNF581; CEBPA; ARHGAP28; RP11-548H18.2; TNRC6B; IQCA1; IGFL4; ADAMTS2; CRB1; LAMP3; CHCHD5; TK2; MTMR9LP; ZBTB44; PTCH2; DEGS2; MTHFSD; RAPGEF5; COX6B2; RPL32P29; FANCL; PLIN2; ZNRD1ASP; FDXR; CABIN1; RPP38; HSP90B2P; CNR2; TMEM200B; SLC39A11; ZNF611; TIPIN; SRBD1; TIRAP; TPPP; MPI; SLC12A6; ZNF497; FLT4; NPIPA8; ZNF775; ZC3H8; SSC4D; DUXAP9; OTX1; FAM86B1; YBEY; AIM1; TENM3; BORCS8-MEF2B; TRIB3; GEMIN4; ZNF433; NBPF19; KIAA0825; PAPLN; LRRN2; WDR24; CDC42BPG; SMYD4; TRIM16; STK11IP; HRH2; POLH; PRR12; PLA2G15; IRF7; RFXANK; ADA; HNRNPA1P6; AC008132.13; PTGIS; AC000095.9; TMEM63C; KCNN2; OXLD1; EID2B; MACC1; PTGES; ABCA17P; HS3ST5; PTK2B; RP11-73M18.2; STK16; FBN2; FAM132A; SASS6; R3HCC1L; CHRAC1; SUSD2; CBFA2T2; ZNF490; PLPP3; MTTP; TNN; ST3GAL6; C2CD2; UBXN10; ANKRD44; RP11-958N24.1; ARSG; LINGO3; RPSAP41; ENTPD2; ZNF221; RP11-730A19.5; UBE3B; ATG2A; FBXL20; ABCB6; RP11-274B21.3; RP11-72304.6; RNF215; KLF2P1; COLCA2; DAB2; RASGRP1; LRFN2; CCDC102B; PAPPA2; SHF; MDH1B; LRRC3; LAMA5; ANKRD20A19P; HBP1; ZNF608; FUK; RSG1; MYHI1; LHX4; ZCCHC18; RGS12; BARHL1; TSNARE1; STON1; NUSIP2; CTC-435M10.3; SNED1; CBX7; RP1-317E23.6; ATE1; VPS51; WDR49; MAST2; ZSCAN25; TMPPE; GRTP1; ALX3; ZBED9; DPH1; TPCN1; SLC46A1; AP3B1; SLC6A4; AZU1; NFKBIL1; ZBTB37; ACSL5; OCEL1; WFDC1; CSGALNACT1; RGS19; BIVM-ERCC5; ZNF573; RP11-201K10.3; KLHDC1; INHBB; SETD4; SMTNL2; TTC23; CAPN14; NUDT16; FBXL22; OBFC1; DSTNP2; USP35; SDHAF3; TMEM220; UNC13D; EPHX2; FGF11; PRPSAP1; NDOR1; PLGLB1; TMEM44; VWCE; DHRS11; SLA2; PDE4B; ZNF467; EPS8L1; ERBB2; ZNF577; ACSF2; NUDT6; HMGB2; RP11-313J2.1; SAV1; FIGN; BTBD17; SMARCA2; SHROOM3; S1PR2; MST1L; ADPRM; WNT10B; IKBKE; AC009060.1; METTL15; XK; ADAMTS8; TDRD6; PAK5; TEAD3; ZFP41; SLC16A4; LONRF3; 1-Mar; NUDT7; PARP4; ADRA1A; PHACTR2; RPSAP53; ENOX1; RTKN; FAM86DP; P2RY1; AC105052.1; RP11-989E6.13; TMEM185A; KLF10; MCF2; ZNF687; RGS3; STIM1; SETBP1; TIAM1; MUC6; PODN; FAM117A; RIPK4; AL365181.1; ABCC1; MYOF; HNRNPUL2-BSCL2; RAB42; CYP2S1; PAN2; ZFR2; ZFPM2; KLHDC9; CICP27; GYPC; GPR162; ZNF350; ZNF833P; FRMPD1; TBX19; GRIN2C; DCDC1; ENPP7P4; SPATA20; PLOD2; ZNF816; ITGA2B; GOLT1A; TOMM40L; C10ORF10; SEMA3F; RBM41; PCDHB13; SNAI3; GALE; KCTD1; TLCD1; SORBS1; KCNS2; ZBTB3; CTB-50E14.5; ZNF37A; RAMP2; RTEL1; CCDC122; FMO5; RP13-726E6.2; MPST; NXPH3; TNFAIP3; PLXND1; CCDC177; SAMD12; TCTE1; IVD; FAM26F; HIST1H1E; EREG; NLRP12; TIGD5; HRK; LRRTM3; SERPINB1; EXPH5; FLT3LG; QRICH2; PLPP4; CTD-2576F9.1; QPRT; F11R; LHFPL5; ARFGAP2; STKLD1; LTBP2; ALMS1P1; MAK; PEX16; HEXIM1; OTUD1; FAAP24; APC2; ABTB1; SMOC2; TGFB3; ZNF112; LRP4; ZMAT5; KLHL31; DIS3L; TMEM52; KIAA1614; RNF44; CTD-2192J16.20; MICA; CMTM7; ZNF324; SLC35G1; ZNF827; DRICH1; ZBTB25; FAM188B; RFT1; PCCB; TRPM3; PALB2; RAB4B; EXD3; LRRC26; RAB36; ATP6VIC2; IPP; SLC26A11; SLC51A; PEX10; POCIA; TMEM8B; CBS; CNTLN; CHRDL1; MOCOS; AHRR; ECT2; STON2; GSTM4; MFSD4A; BTBD11; PPIC; ETV3; NIP7; ABCD1; SHMT2; ALDOC; MED22; CHMP6; LYPD3; OBSCN; SPDYE1; ATPAF2; NR2F1; SEMA3B; WDR86; ANKRD45; ZBTB2; PTPN13; CCNE1; PRCD; MLLT3; ZRANB3; ATP7B; USP51; IL1RAP; ZNF69; DCAF12L2; PNPLA4; PRNP; NIMIK; ZNF80; CEP63; CFAP44; FAM162A; ALDHIA2; DOC2GP; MASP1; RNASEH2A; CACHD1; C6ORF25; CPEB4; TTLL9; C14ORF159; GNRHR; ZNF521; MESP1; ARVCF; LCNL1; CFAP61; HMGN2P46; CACNG5; SYNC; FLNC; TTF2; SWI5; EML2; LGALS3BP; CNTNAP3B; RP11-1415C14.3; RP11-1212A22.1; C9ORF85; VSTM4; C20ORF96; RPP25L; ZNF730; RHBDD1; CXORF38; POLA2; CHD6; NINL; ARSA; P3H4; AKRIC3; ZNF157; FNDC5; RP11-408P14.1; PAAF1; CIQTNF6; TMEM39B; LRSAM1; SUFU; C5ORF49; SLC27A2; FOXK1; HLTF; AMOT; C11ORF68; TAS2R5; TMEM143; ZNF839; SCIN; BANP; AZIN2; ELP3; KIF13A; SALL2; MID1IP1; FIBIN; RP11-5A19.5; STARD13; ZC3H7A; BAX; PLA2G6; CREG2; ENKD1; OR2L13; SLC6A9; NT5C; CUBN; FAM175A; C5ORF34; PCDHB16; CORO1B; ZNF517; FCGBP; MLLT6; MBTD1; SETDB2; PCOLCE2; OPHN1; ZNF34; MAP7; ZNF321P; RNASEHIP1; HTN1; DCAF4; NCKAP5; L3MBTL4; CEP164; VPS13B; PROC; G6PC3; RAD51AP2; RP11-234A1.1; RP11-108M9.5; DUS2; LYPD6; SLC37A1; XPNPEP3; LAMB1; KIAA0556; TMEM94; DNAJC4; BNC2; TRAF1; FAM86C2P; MCTP2; OMA1; BMP8B; FBLN2; ASXL3; HBQ1; ERAL1; P2RX3; ADGRE5; SLC16A7; CECR1; PCDHGC3; TMEM53; TNFSF9; HLA-A; ZSCAN20; TMEM115; PRIMPOL; PPL; BIK; NEDD9; SPACA9; KLLN; CRISPLD1; ADAMTSL2; LMLN; DKKL1; ZFYVE19; ERLIN2; ZEB1; PLCXD1; TMEM240; NTPCR; FRMPD4; PVRIG; KIAA1024; NR1H3; PHOSPHO2; ZNF626; BLVRB; VEGFA; CHMP1B; FIGNL2; KLHL22; RP11-641D5.1; CYP7B1; D2HGDH; PHYHIP; DPM3; ANKRD33B; GDF9; CNPY4; TRIM8; ZNF783; ZDHHC22; ADCK4; C7ORF31; TP53I3; CCDC85A; HMOX1; CYP2R1; CTD-2311M21.2; CTXN2; INPP5E; TPPP3; MITF; PSMB10; ERICH1; RAD51D; KNOP1; STOX2; SPATA13; SIX5; RPA1; LRRC20; ATP10D; PDPK2P; BCL9; FSCN2; GALK2; HPCA; MANSC1; CELSR1; DUSP9; ANXA11; CEBPB; ADAT1; HOMEZ; KANK3; DRGX; KCTD18; CCSER1; FAM71F2; MED15P9; EZH1; MRPL53; NARFL; RILP; SARDH; MCTS2P; PRSS12; MXD1; SEMA5B; HLA-DOA; UBE4B; RPS4XP16; RUNX1T1; GTF2H3; CTD-3138B18.4; FAM3A; NDST4; STAT1; SLC30A8; PRDM15; and PLCB3.

Modulated Reactome, KEGG and Wikipathways in GSE88773 (listed in order of and in accordance with the degree of their upregulation or downregulation)

(Pathway Name/Combined Score=log(p)×Zscore/Regulated)

Catabolism of glucuronate to xylulose-5-phosphate Homo sapiens R-HSA-5661270/133.82/Down; Sulfide oxidation to sulfate Homo sapiens R-HSA-1614517/133.82/Down; NGF-independent TRKA activation Homo sapiens R-HSA-187024/133.82/Up; Highly calcium permeable nicotinic acetylcholine receptors Homo sapiens R-HSA-629597/132.46/Down; Cholesterol Biosynthesis Homo sapiens WP197/110.99/Up; Cholesterol Biosynthesis Mus musculus WP103/110.99/Up; Terpenoid backbone biosynthesis Homo sapiens hsa00900/100.00/Up; Synthesis of Lipoxins (LX) Homo sapiens R-HSA-2142700/92.75/Down; Activation of TRKA receptors Homo sapiens R-HSA-187015/92.75/Up; Highly calcium permeable postsynaptic nicotinic acetylcholine receptors Homo sapiens R-HSA-629594/89.96/Down; Nuclear Events (kinase and transcription factor activation) Homo sapiens R-HSA-198725/84.93/Up; SREBF and miR33 in cholesterol and lipid homeostasis Homo sapiens WP2011/78.80/Up; Presynaptic nicotinic acetylcholine receptors Homo sapiens R-HSA-622323/76.44/Down; Highly sodium permeable acetylcholine nicotinic receptors Homo sapiens R-HSA-629587/69.19/Down; Phosphate bond hydrolysis by NUDT proteins Homo sapiens R-HSA-2393930/69.19/Down; Transport of nucleosides and free purine and pyrimidine bases across the plasma membrane Homo sapiens R-HSA-83936/69.19/Down; CREB phosphorylation Homo sapiens R-HSA-199920/69.19/Up; ERK/MAPK targets Homo sapiens R-HSA-198753/59.23/Up; Activation of Nicotinic Acetylcholine Receptors Homo sapiens R-HSA-629602/57.55/Down; Acetylcholine Binding And Downstream Events Homo sapiens R-HSA-181431/57.55/Down; Postsynaptic nicotinic acetylcholine receptors Homo sapiens R-HSA-622327/57.55/Down; MAPK targets/Nuclear events mediated by MAP kinases Homo sapiens R-HSA-450282/55.98/Up; Activation of PUMA and translocation to mitochondria Homo sapiens R-HSA-139915/54.12/Down; BH3-only proteins associate with and inactivate anti-apoptotic BCL-2 members Homo sapiens R-HSA-111453/54.12/Up; RAF-independent MAPK 1/3 activation Homo sapiens R-HSA-112409/50.08/Down; Cholesterol biosynthesis Homo sapiens R-HSA-191273/50.08/Up; BMAL1:CLOCK, NPAS2 activates circadian gene expression Homo sapiens R-HSA-1368108/48.95/Up; Regulation of cholesterol biosynthesis by SREBP (SREBF) Homo sapiens R-HSA-1655829/44.73/Up; Degradation of cysteine and homocysteine Homo sapiens R-HSA-1614558/43.76/Down; Fanconi Anemia Pathway Homo sapiens R-HSA-6783310/41.91/Down; Serotonin and anxiety Mus musculus WP2141/40.43/Down; Butyrate Response Factor 1 (BRF1) binds and destabilizes mRNA Homo sapiens R-HSA-450385/40.43/Up; FAS pathway and Stress induction of HSP regulation Mus musculus WP571/39.74/Up; SREBP signaling Homo sapiens WP1982/39.43/Up; Sulfur metabolism Homo sapiens hsa00920/36.26/Down; SREBF and miR33 in cholesterol and lipid homeostasis Mus musculus WP2084/36.26/Up; Adrenaline, noradrenaline inhibits insulin secretion Homo sapiens R-HSA-400042/34.79/Up; Circadian Clock Homo sapiens R-HSA-400253/34.23/Up; MAPK signaling pathway Homo sapiens hsa04010/34.23/Up; NS1 Mediated Effects on Host Pathways Homo sapiens R-HSA-168276/34.13/Up; Myometrial Relaxation and Contraction Pathways Mus musculus WP385/34.11/Up; MAPK Signaling Pathway Homo sapiens WP382/33.84/Up; Nuclear Receptor transcription pathway Homo sapiens R-HSA-383280/33.66/Up; Prostacyclin signaling through prostacyclin receptor Homo sapiens R-HSA-392851/33.04/Up; MAPK signaling pathway Mus musculus WP493/32.18/Up; Host Interactions with Influenza Factors Homo sapiens R-HSA-168253/31.02/Up; Gene regulatory network modelling somitogenesis Homo sapiens WP2854/30.63/Up; Activation of Kainate Receptors Up; on glutamate binding Homo sapiens R-HSA-451326/30.59/Up; Glucagon-type ligand receptors Homo sapiens R-HSA-420092/30.59/Up; Glycosaminoglycan biosynthesis-chondroitin sulfate/dermatan sulfate Homo sapiens hsa00532/30.10/Up; Mitochondrial Gene Expression Homo sapiens WP391/30.10/Up; Hypertrophy Model Mus musculus WP202/30.10/Up; Hypertrophy Model Homo sapiens WP516/30.10/Up; G beta: gamma signaling through PLC beta Homo sapiens R-HSA-418217/30.10/Up; FAS pathway and Stress induction of HSP regulation Homo sapiens WP314/29.61/Up; Glucagon-like Peptide-1 (GLP1) regulates insulin secretion Homo sapiens R-HSA-381676/29.61/Up; Activation of gene expression by SREBF (SREBP) Homo sapiens R-HSA-2426168/29.61/Up; Toll Like Receptor 10 (TLR10) Cascade Homo sapiens R-HSA-168142/29.41/Up; Toll Like Receptor 5 (TLR5) Cascade Homo sapiens R-HSA-168176/29.41/Up; MyD88 cascade initiated on plasma membrane Homo sapiens R-HSA-975871/29.41/Up; TRAF6 mediated induction of NFkB and MAP kinases Up; on TLR7/8 or 9 activation Homo sapiens R-HSA-975138/28.68/Up; Vasopressin regulates renal water homeostasis via Aquaporins Homo sapiens R-HSA-432040/28.30/Up; Circadian rythm related genes Homo sapiens WP3594/28.21/Up; CD209 (DC-SIGN) signaling Homo sapiens R-HSA-5621575/27.53/Up; Presynaptic function of Kainate receptors Homo sapiens R-HSA-500657/27.53/Up; Calcium Regulation in the Cardiac Cell Homo sapiens WP536/27.42/Up; MyD88 dependent cascade initiated on endosome Homo sapiens R-HSA-975155/27.30/Up; Toll Like Receptor 7/8 (TLR7/8) Cascade Homo sapiens R-HSA-168181/27.30/Up; Calcium Regulation in the Cardiac Cell Mus musculus WP553/27.01/Up; Tandem pore domain potassium channels Homo sapiens R-HSA-1296346/26.27/Up; ERKs are inactivated Homo sapiens R-HSA-202670/26.27/Up; NOTCH2 intracellular domain regulates transcription Homo sapiens R-HSA-2197563/26.27/Up; TP53 Regulates Transcription of Death Receptors and Ligands Homo sapiens R-HSA-6803211/26.27/Up; CD28 dependent Vav1 pathway Homo sapiens R-HSA-389359/26.27/Up; Glucagon signaling in metabolic regulation Homo sapiens R-HSA-163359/25.57/Up; Toll Like Receptor 9 (TLR9) Cascade Homo sapiens R-HSA-168138/25.38/Up; ADP signaling through P2Y purinoceptor 12 Homo sapiens R-HSA-392170/25.29/Up; MAP kinase activation in TLR cascade Homo sapiens R-HSA-450294/24.39/Up; Morphine addiction Homo sapiens hsa05032/23.64/Up; Thromboxane signaling through TP receptor Homo sapiens R-HSA-428930/23.31/Up; SIDS Susceptibility Pathways Homo sapiens WP706/23.31/Up; Toll Like Receptor 2 (TLR2) Cascade Homo sapiens R-HSA-181438/23.10/Up; MyD88:Mal cascade initiated on plasma membrane Homo sapiens R-HSA-166058/23.10/Up; Toll Like Receptor TLR1:TLR2 Cascade Homo sapiens R-HSA-168179/23.10/Up; Toll Like Receptor TLR6:TLR2 Cascade Homo sapiens R-HSA-168188/23.10/Up; Non-homologous end-joining Homo sapiens hsa03450/22.81/Down; Ganglio Sphingolipid Metabolism Homo sapiens WP1423/22.81/Down; GABA A receptor activation Homo sapiens R-HSA-977441/22.81/Up; EGF/EGFR Signaling Pathway Homo sapiens WP437/22.33/Up; Sema4D induced cell migration and growth-cone collapse Homo sapiens R-HSA-416572/21.56/Down; Glycosaminoglycan biosynthesis-heparan sulfate/heparin Homo sapiens hsa00534/21.56/Up; Regulation of HSF1-mediated heat shock response Homo sapiens R-HSA-3371453/21.00/Up; Synthesis and Degradation of Ketone Bodies Homo sapiens WP311/20.80/Down; Synthesis and Degradation of Ketone Bodies Mus musculus WP543/20.80/Down; Estrogen biosynthesis Homo sapiens R-HSA-193144/20.80/Down; Regulation of gene expression in endocrine-committed (NEUROG3+) progenitor cells Homo sapiens R-HSA-210746/20.80/Down; Extrinsic Pathway of Fibrin Clot Formation Homo sapiens R-HSA-140834/20.80/Down; Galactose catabolism Homo sapiens R-HSA-70370/20.80/Down; Toxicity of botulinum toxin type D (BoNT/D) Homo sapiens R-HSA-5250955/20.80/Up; Galactose catabolism Homo sapiens R-HSA-70370/20.80/Up; Activation of RAS in B cells Homo sapiens R-HSA-1169092/20.80/Up; PTK6 Expression Homo sapiens R-HSA-8849473/20.80/Up; FBXW7 Mutants and NOTCH1 in Cancer Homo sapiens R-HSA-2644605/20.80/Up; Loss of Function of FBXW7 in Cancer and NOTCH1 Signaling Homo sapiens R-HSA-2644607/20.80/Up; IRS activation Homo sapiens R-HSA-74713/20.80/Up; Transmission across Electrical Synapses Homo sapiens R-HSA-112307/20.80/Up; Electric Transmission Across Gap Junctions Homo sapiens R-HSA-112303/20.80/Up; Hyaluronan biosynthesis and export Homo sapiens R-HSA-2142850/20.80/Up; Sodium-coUp; led phosphate cotransporters Homo sapiens R-HSA-427652/20.80/Up; Toxicity of botulinum toxin type F (BoNT/F) Homo sapiens R-HSA-5250981/20.80/Up; Oncostatin M Signaling Pathway Homo sapiens WP2374/20.73/Down; Aquaporin-mediated transport Homo sapiens R-HSA-445717/20.25/Up; TSH signaling pathway Homo sapiens WP2032/20.09/Down; Glycosphingolipid biosynthesis-globo series Homo sapiens hsa00603/20.01/Down; Glycosphingolipid biosynthesis-globo series Homo sapiens hsa00603/20.01/Up; Activation of Rac Homo sapiens R-HSA-428540/20.01/Up; ADP signaling through P2Y purinoceptor 1 Homo sapiens R-HSA-418592/19.99/Up; Metal ion SLC transporters Homo sapiens R-HSA-425410/19.99/Up; EGFR1 Signaling Pathway Mus musculus WP572/19.95/Up; Nuclear Receptors Homo sapiens WP170/19.53/Up; miRs in Muscle Cell Differentiation Homo sapiens WP2012/19.53/Up; Wnt Signaling Pathway Homo sapiens WP428/19.48/Down; Signaling Pathways in Glioblastoma Homo sapiens WP2261/19.42/Down; Insulin Signaling Mus musculus WP65/19.36/Up; Transport of Mature mRNA derived from an Intron-Containing Transcript Homo sapiens R-HSA-159236/18.89/Up; Myometrial Relaxation and Contraction Pathways Homo sapiens WP289/18.78/Up; GPCRs, Class C Metabotropic glutamate, pheromone Mus musculus WP327/17.70/Down; GPCRs, Class C Metabotropic glutamate, pheromone Homo sapiens WP501/17.70/Down; Insulin Signaling Homo sapiens WP481/17.69/Up; Negative regulation of MAPK pathway Homo sapiens R-HSA-5675221/17.68/Down; Transcriptional activation of mitochondrial biogenesis Homo sapiens R-HSA-2151201/17.68/Up; IL-1 signaling pathway Homo sapiens WP195/17.40/Up; Sema4D in semaphorin signaling Homo sapiens R-HSA-400685/17.32/Down; BDNF signaling pathway Homo sapiens WP2380/16.77/Up; ISG15 antiviral mechanism Homo sapiens R-HSA-1169408/16.76/Up; Antiviral mechanism by IFN-stimulated genes Homo sapiens R-HSA-1169410/16.76/Up; TRAF6 Mediated Induction of proinflammatory cytokines Homo sapiens R-HSA-168180/16.76/Up; G-protein activation Homo sapiens R-HSA-202040/16.18/Up; IL-2 Signaling Pathway Homo sapiens WP49/16.07/Down; Glucose transport Homo sapiens R-HSA-70153/16.07/Up; Adipogenesis genes Mus musculus WP447/15.87/Up; Transport of Mature Transcript to Cytoplasm Homo sapiens R-HSA-72202/15.82/Up; Sema3A PAK dependent Axon repulsion Homo sapiens R-HSA-399954/15.78/Up; PKA activation Homo sapiens R-HSA-163615/15.78/Up; Wnt Signaling Pathway Mus musculus WP403/15.62/Down; Neuronal System Homo sapiens R-HSA-112316/15.33/Up; Choline catabolism Homo sapiens R-HSA-6798163/15.31/Down; Telomere C-strand synthesis initiation Homo sapiens R-HSA-174430/15.31/Down; DNA replication initiation Homo sapiens R-HSA-68952/15.31/Down; Pyrimidine biosynthesis Homo sapiens R-HSA-500753/15.31/Down; Synthesis of 5-eicosatetraenoic acids Homo sapiens R-HSA-2142688/15.31/Down; Fructose metabolism Homo sapiens R-HSA-5652084/15.31/Down; Regulation of Cardiac Hypertrophy by miR-208 Mus musculus WP1526/15.31/Down; mir-124 predicted interactions with cell cycle and differentiation Homo sapiens WP3595/15.31/Up; Sensing of DNA Double Strand Breaks Homo sapiens R-HSA-5693548/15.31/Up; Metabolic disorders of biological oxidation enzymes Homo sapiens R-HSA-5579029/15.31/Up; RSK activation Homo sapiens R-HSA-444257/15.31/Up; Vasopressin-like receptors Homo sapiens R-HSA-388479/15.31/Up; Synthesis of Dolichyl-phosphate Homo sapiens R-HSA-446199/15.31/Up; DNA Damage Response (only ATM dependent) Homo sapiens WP710/15.31/Down; G alpha (q) signaling events Homo sapiens R-HSA-416476/15.25/Up; Activation of BH3-only proteins Homo sapiens R-HSA-114452/15.15/Up; MAPK Cascade Homo sapiens WP422/15.15/Up; MAPK Cascade Mus musculus WP251/15.15/Up; SIDS Susceptibility Pathways Mus musculus WP1266/15.08/Up; Activated TLR4 signaling Homo sapiens R-HSA-166054/15.00/Up; GPCRs, Other Mus musculus WP41/14.95/Down; NEP/NS2 Interacts with the Cellular Export Machinery Homo sapiens R-HSA-168333/14.21/Up; Regulation of Glucokinase by Glucokinase Regulatory Protein Homo sapiens R-HSA-170822/14.21/Up; Transport of Ribonucleoproteins into the Host Nucleus Homo sapiens R-HSA-168271/14.21/Up; Circadian rhythm Homo sapiens hsa04710/14.21/Up; Cellular response to heat stress Homo sapiens R-HSA-3371556/14.16/Up; Beta-catenin phosphorylation cascade Homo sapiens R-HSA-196299/14.16/Down; Formation of Senescence-Associated Heterochromatin Foci (SAHF) Homo sapiens R-HSA-2559584/14.16/Down; RHO GTPases Activate ROCKs Homo sapiens R-HSA-5627117/14.16/Down; activated TAK1 mediates p38 MAPK activation Homo sapiens R-HSA-450302/14.16/Down; Tristetraprolin (TTP, ZFP36) binds and destabilizes mRNA Homo sapiens R-HSA-450513/14.16/Up; Zinc transporters Homo sapiens R-HSA-435354/14.16/Up; PKA-mediated phosphorylation of CREB Homo sapiens R-HSA-111931/14.16/Up; Acetylcholine Neurotransmitter Release Cycle Homo sapiens R-HSA-264642/14.16/Up; PKA activation in glucagon signaling Homo sapiens R-HSA-164378/14.16/Up; MAPK signaling pathway Mus musculus WP493/14.08/Down; MicroRNAs in Cardiomyocyte Hypertrophy Mus musculus WP1560/13.76/Down; G alpha (z) signaling events Homo sapiens R-HSA-418597/13.42/Up; Regulation of insulin secretion Homo sapiens R-HSA-422356/13.39/Up; Disassembly of the destruction complex and recruitment of AXIN to the membrane Homo sapiens R-HSA-4641262/13.35/Down; Export of Viral Ribonucleoproteins from Nucleus Homo sapiens R-HSA-168274/13.35/Up; Insulin signaling pathway Homo sapiens hsa04910/13.08/Up; NOTCH1 Intracellular Domain Regulates Transcription Homo sapiens R-HSA-2122947/12.85/Up; Structural Pathway of Interleukin 1 (IL-1) Homo sapiens WP2637/12.85/Up; TP53 Regulates Transcription of Genes Involved in G2 Cell Cycle Arrest Homo sapiens R-HSA-6804114/12.78/Down; Mitochondrial Gene Expression Mus musculus WP1263/12.78/Up; cGMP effects Homo sapiens R-HSA-418457/12.78/Up; Longevity regulating pathway-multiple species Homo sapiens hsa04213/12.73/Down; Vpr-mediated nuclear import of PICs Homo sapiens R-HSA-180910/12.56/Up; Thrombin signaling through proteinase activated receptors (PARs) Homo sapiens R-HSA-456926/12.56/Up; Signal amplification Homo sapiens R-HSA-392518/12.56/Up; Nuclear import of Rev protein Homo sapiens R-HSA-180746/12.56/Up; Monoamine GPCRs Mus musculus WP570/12.56/Up; p38 MAPK Signaling Pathway Mus musculus WP350/12.56/Up; Glycosaminoglycan metabolism Homo sapiens R-HSA-1630316/12.56/Up; Wnt Signaling Pathway and Pluripotency Homo sapiens WP399/12.36/Down; Hexose transport Homo sapiens R-HSA-189200/12.32/Up; Toll Like Receptor 4 (TLR4) Cascade Homo sapiens R-HSA-166016/12.32/Up; Class B/2 (Secretin family receptors) Homo sapiens R-HSA-373080/12.03/Up; Transport of the SLBP independent Mature mRNA Homo sapiens R-HSA-159227/11.84/Up; Monoamine GPCRs Homo sapiens WP58/11.84/Up; Rev-mediated nuclear export of HIV RNA Homo sapiens R-HSA-165054/11.84/Up; Activation of NIMA Kinases NEK9, NEK6, NEK7 Homo sapiens R-HSA-2980767/11.83/Down; Non-homologous end joining Homo sapiens WP438/11.83/Down; PTK6 promotes HIFIA stabilization Homo sapiens R-HSA-8857538/11.83/Down; Constitutive Signaling by NOTCH1 t (7;9) (NOTCH1:M1580 K2555) Translocation Mutant Homo sapiens R-HSA-2660825/11.83/Down; Synthesis of Ketone Bodies Homo sapiens R-HSA-77111/11.83/Down; Zinc efflux and compartmentalization by the SLC30 family Homo sapiens R-HSA-435368/11.83/Down, Signaling by NOTCH1 t(7;9)(NOTCH1:M1580 K2555) Translocation Mutant Homo sapiens R-HSA-2660825/11.83/Down; Zinc efflux and compartmentalization by the SLC30 family Homo sapiens R-HSA-435368/11.83/Up; Phosphate bond hydrolysis by NUDT proteins Homo sapiens R-HSA-2393930/11.83/Up; Cation-coUp; led Chloride cotransporters Homo sapiens R-HSA-426117/11.83/Up; Transmission across Chemical Synapses Homo sapiens R-HSA-112315/11.83/Up; Cocaine addiction Homo sapiens hsa05030/11.81/Up; Mitochondrial biogenesis Homo sapiens R-HSA-1592230/11.81/Up; TP53 Network Homo sapiens WP1742/11.59/Down; TP53 Regulates Transcription of Genes Involved in Cytochrome C Release Homo sapiens R-HSA-6803204/11.59/Down; Regulation of TP53 Activity through Methylation Homo sapiens R-HSA-6804760/11.59/Down; Ephrin signaling Homo sapiens R-HSA-3928664/11.59/Up; GABA synthesis, release, reUptake and degradation Homo sapiens R-HSA-888590/11.59/Up; Serotonin Receptor 4/6/7 and NR3C Signaling Homo sapiens WP734/11.59/Up; Phase 4-resting membrane potential Homo sapiens R-HSA-5576886/11.59/Up; MAPK Signaling Pathway Homo sapiens WP382/11.55/Down; Imatinib Resistance in Chronic Myeloid Leukemia Homo sapiens WP2946/11.34/Down; Nuclear Pore Complex (NPC) Disassembly Homo sapiens R-HSA-3301854/11.17/Up; p38 MAPK Signaling Pathway Homo sapiens WP400/11.17/Up; Transport of the SLBP dependent Mature mRNA Homo sapiens R-HSA-159230/11.17/Up; Platelet homeostasis Homo sapiens R-HSA-418346/11.14/Up; Mitotic Prophase Homo sapiens R-HSA-68875/11.08/Up; EGFR1 Signaling Pathway Mus musculus WP572/11.05/Down; Nuclear Envelope Breakdown Homo sapiens R-HSA-2980766/10.89/Up; G Protein Signaling Pathways Mus musculus WP232/10.85/Up; GABAergic synapse Homo sapiens hsa04727/10.85/Up; Adipogenesis Homo sapiens WP236/10.59/Up; Interactions of Rev with host cellular proteins Homo sapiens R-HSA-177243/10.56/Up; Interactions of Vpr with host cellular proteins Homo sapiens R-HSA-176033/10.56/Up; Serotonin Receptor 2 and ELK-SRF/GATA4 signaling Homo sapiens WP732/10.56/Up; Chondroitin sulfate biosynthesis Homo sapiens R-HSA-2022870/10.56/Up; TGF-beta Signaling Pathway Homo sapiens WP366/10.21/Up; Insulin Signaling Mus musculus WP65/10.19/Down; Fanconi anemia pathway Homo sapiens hsa03460/10.06/Down; TOR Signaling Homo sapiens WP1471/10.00/Up; Transmembrane transport of small molecules Homo sapiens R-HSA-382551/9.96/Up; Alcoholism Homo sapiens hsa05034/9.83/Up; Corticotropin-releasing hormone Homo sapiens WP2355/9.82/Up; Gastrin-CREB signaling pathway via PKC and MAPK Homo sapiens R-HSA-881907/9.75/Up; Interferon type I signaling pathways Homo sapiens WP585/9.67/Down; Copper homeostasis Homo sapiens WP3286/9.67/Down; IL-4 Signaling Pathway Homo sapiens WP395/9.67/Down; TP53 regulates transcription of additional cell cycle genes whose exact role in the p53 pathway remain uncertain Homo sapiens R-HSA-6804115/9.65/Down; NOTCH2 Activation and Transmission of Signal to the Nucleus Homo sapiens R-HSA-2979096/9.65/Down; Synthesis of very long-chain fatty acyl-CoAs Homo sapiens R-HSA-75876/9.65/Up; RHO GTPases activate PAKs Homo sapiens R-HSA-5627123/9.65/Up; Nuclear Receptors Mus musculus WP509/9.47/Up; Transport of Mature mRNA Derived from an Intronless Transcript Homo sapiens R-HSA-159231/9.47/Up; NAD Biosynthesis II (from tryptophan) Homo sapiens WP2485/9.47/Down; FTO Obesity Variant Mechanism Homo sapiens WP3407/9.47/Down; ABC transporter disorders Homo sapiens R-HSA-5619084/9.47/Down; BH3-only proteins associate with and inactivate anti-apoptotic BCL-2 members Homo sapiens R-HSA-111453/9.47/Down; Sema4D mediated inhibition of cell attachment and migration Homo sapiens R-HSA-416550/9.47/Down; RNF mutants show enhanced WNT signaling and proliferation Homo sapiens R-HSA-5340588/9.47/Down; Ketone body metabolism Homo sapiens R-HSA-74182/9.47/Down; Formyl peptide receptors bind formyl peptides and many other ligands Homo sapiens R-HSA-444473/9.47/Down; ACE Inhibitor Pathway Mus musculus WP396/9.47/Down; Synthesis of UDP-N-acetyl-glucosamine Homo sapiens R-HSA-446210/9.47/Up; Activation of PUMA and translocation to mitochondria Homo sapiens R-HSA-139915/9.47/Up; Linoleic acid (LA) metabolism Homo sapiens R-HSA-2046105/9.47/Up; NR1D1 (REV-ERBA) represses gene expression Homo sapiens R-HSA-136807/9.47/Up; Nicotinamide salvaging Homo sapiens R-HSA-197264/9.47/Up; Organic anion transporters Homo sapiens R-HSA-428643/9.47/Up; Vitamin C (ascorbate) metabolism Homo sapiens R-HSA-196836/9.47/Up;/9.47/Up; Orexin and neuropeptides FF and QRFP bind to their respective receptors Homo sapiens R-HSA-389397/9.47/Up; FTO Obesity Variant Mechanism Homo sapiens WP3407/9.47/Up; Wnt Signaling Pathway and Pluripotency Mus musculus WP723/9.35/Down; Basal cell carcinoma Homo sapiens hsa05217/9.31/Down; Ion transport by P-type ATPases Homo sapiens R-HSA-936837/9.31/Up; Insulin Signaling Homo sapiens WP481/9.26/Down; G Protein Signaling Pathways Homo sapiens WP35/9.13/Up; O-glycosylation of TSR domain-containing proteins Homo sapiens R-HSA-5173214/8.98/Up; Transport of Mature mRNAs Derived from Intronless Transcripts Homo sapiens R-HSA-159234/8.98/Up; ESC Pluripotency Pathways Mus musculus WP339/8.98/Down; Blood Clotting Cascade Homo sapiens WP272/8.86/Down; Nicotine Activity on Dopaminergic Neurons Homo sapiens WP1602/8.86/Down; Globo Sphingolipid Metabolism Homo sapiens WP1424/8.86/Down; Globo Sphingolipid Metabolism Homo sapiens WP1424/8.86/Up; Transcription factor regulation in adipogenesis Homo sapiens WP3599/8.86/Up; Toll-Like Receptors Cascades Homo sapiens R-HSA-168898/8.84/Up; TRIF-mediated TLR3/TLR4 signaling Homo sapiens R-HSA-937061/8.70/Up; Toll Like Receptor 3 (TLR3) Cascade Homo sapiens R-HSA-168164/8.70/Up; MyD88-independent TLR3/TLR4 cascade Homo sapiens R-HSA-166166/8.70/Up; Meiotic synapsis Homo sapiens R-HSA-1221632/8.64/Down; Wnt signaling pathway Homo sapiens hsa04310/8.53/Down; G alpha(s) signaling events Homo sapiens R-HSA-418555/8.53/Up; Neurotransmitter Receptor Binding And Downstream Transmission In The Postsynaptic Cell Homo sapiens R-HSA-112314/8.53/Up; PDGF Pathway Homo sapiens WP2526/8.53/Up; Neurotrophin signaling pathway Homo sapiens hsa04722/8.45/Up; Estrogen signaling pathway Homo sapiens hsa04915/8.29/Down; Biosynthesis of unsaturated fatty acids Homo sapiens hsa01040/8.16/Up; RAF-independent MAPK1/3 activation Homo sapiens R-HSA-112409/8.16/Up; NGF signaling via TRKA from the plasma membrane Homo sapiens R-HSA-187037/8.13/Up; Glycine, serine and threonine metabolism Homo sapiens hsa00260/8.11/Down; Nicotine addiction Homo sapiens hsa05033/8.11/Up; Apoptosis Mus musculus WP1254/8.06/Down; Preimplantation Embryo Homo sapiens WP3527/8.03/Down; Kit receptor signaling pathway Homo sapiens WP304/8.03/Up; MicroRNAs in cardiomyocyte hypertrophy Homo sapiens WP1544/7.92/Down; Heme Biosynthesis Mus musculus WP18/7.77/Down; Sperm Motility And Taxes Homo sapiens R-HSA-1300642/7.77/Down; Heme Biosynthesis Homo sapiens WP561/7.77/Down; Crosslinking of collagen fibrils Homo sapiens R-HSA-2243919/7.77/Down; RHO GTPases Activate Rhotekin and Rhophilins Homo sapiens R-HSA-5666185/7.77/Down; SHC-related events triggered by IGF1R Homo sapiens R-HSA-2428933/7.77/Down; Pyrimidine salvage reactions Homo sapiens R-HSA-73614/7.77/Down; Adrenoceptors Homo sapiens R-HSA-390696/7.77/Down; Transport of gamma-carboxylated protein precursors from the endoplasmic reticulum to the Golgi apparatus Homo sapiens R-HSA-159763/7.77/Down; AKT phosphorylates targets in the nucleus Homo sapiens R-HSA-198693/7.77/Down; Pregnenolone biosynthesis Homo sapiens R-HSA-196108/7.77/Up; EV release from cardiac cells and their functional effects Homo sapiens WP3297/7.77/Up; Heme Biosynthesis Mus musculus WP18/7.77/Up; Heme Biosynthesis Homo sapiens WP561/7.77/Up; AKT phosphorylates targets in the nucleus Homo sapiens R-HSA-198693/7.77/Up; Small interfering RNA (siRNA) biogenesis Homo sapiens R-HSA-426486/7.77/Up; Adrenoceptors Homo sapiens R-HSA-390696/7.77/Up; Mitochondrial iron-sulfur cluster biogenesis Homo sapiens R-HSA-1362409/7.77/Up; Pentose phosphate pathway (hexose monophosphate shunt) Homo sapiens R-HSA-71336/7.77/Up; MAP kinase activation in TLR cascade Homo sapiens R-HSA-450294/7.74/Down; Toll-like Receptor Signaling Pathway Homo sapiens WP75/7.73/Down; Pre-NOTCH Transcription and Translation Homo sapiens R-HSA-1912408/7.72/Down; Adrenergic signaling in cardiomyocytes Homo sapiens hsa04261/7.69/Up; Opioid signaling Homo sapiens R-HSA-111885/7.62/Up; IL-9 Signaling Pathway Mus musculus WP10/7.54/Down; Pyrimidine metabolism Homo sapiens R-HSA-73848/7.54/Down; DARPP-32 events Homo sapiens R-HSA-180024/7.54/Up; Insulin processing Homo sapiens R-HSA-264876/7.54/Up; Endogenous sterols Homo sapiens R-HSA-211976/7.54/Up; TP53 Regulates Transcription of DNA Repair Genes Homo sapiens R-HSA-6796648/7.47/Down; Ca2+ pathway Homo sapiens R-HSA-4086398/7.47/Up; Toll Like Receptor 10 (TLR10) Cascade Homo sapiens R-HSA-168142/7.41/Down; Toll Like Receptor 5 (TLR5) Cascade Homo sapiens R-HSA-168176/7.41/Down; MyD88 cascade initiated on plasma membrane Homo sapiens R-HSA-975871/7.41/Down; Viral Messenger RNA Synthesis Homo sapiens R-HSA-168325/7.35/Up; Cooperation of PDCL (PhLP1) and TRiC/CCT in G-protein beta folding Homo sapiens R-HSA-6814122/7.35/Up; Fatty Acyl-CoA Biosynthesis Homo sapiens R-HSA-75105/7.35/Up; Intrinsic Pathway for Apoptosis Homo sapiens R-HSA-109606/7.35/Up; Circadian rythm related genes Homo sapiens WP3594/7.32/Down; Adipogenesis genes Mus musculus WP447/7.21/Down; Toll-like receptor signaling pathway Homo sapiens hsa04620/7.06/Down; Wnt Signaling Pathway NetPath Mus musculus WP539/7.06/Down; Amine ligand-binding receptors Homo sapiens R-HSA-375280/7.00/Up; Metabolism of carbohydrates Homo sapiens R-HSA-71387/6.98/Up; Glycosylphosphatidylinositol (GPI)-anchor biosynthesis Homo sapiens hsa00563/6.98/Down; HDMs demethylate histones Homo sapiens R-HSA-3214842/6.98/Down; Synthesis of IP3 and IP4 in the cytosol Homo sapiens R-HSA-1855204/6.98/Down; ATF4 activates genes Homo sapiens R-HSA-380994/6.98/Down; Sulfur amino acid metabolism Homo sapiens R-HSA-1614635/6.98/Down; Glyoxylate metabolism and glycine degradation Homo sapiens R-HSA-389661/6.98/Down; Basigin interactions Homo sapiens R-HSA-210991/6.98/Up; Fatty acid elongation Homo sapiens hsa00062/6.98/Up; Nitric oxide stimulates guanylate cyclase Homo sapiens R-HSA-392154/6.98/Up; Synthesis of substrates in N-glycan biosythesis Homo sapiens R-HSA-446219/6.97/Up; Dual incision in TC-NER Homo sapiens R-HSA-6782135/6.97/Up; Adipogenesis Homo sapiens WP236/6.94/Down; signaling by NGF Homo sapiens R-HSA-166520/6.93/Up; Meiosis Homo sapiens R-HSA-1500620/6.83/Down; p53 signaling Mus musculus WP2902/6.73/Down; RNA Polymerase II Transcription Initiation Homo sapiens R-HSA-75953/6.68/Up; HIV Transcription Initiation Homo sapiens R-HSA-167161/6.68/Up; SUMOylation of DNA replication proteins Homo sapiens R-HSA-4615885/6.68/Up; Heart Development Mus musculus WP2067/6.68/Up; RNA Polymerase II Transcription Pre-Initiation And Promoter Opening Homo sapiens R-HSA-73779/6.68 Up; RNA Polymerase II Transcription Initiation And Promoter Clearance Homo sapiens R-HSA-76042/6.68/Up; RNA Polymerase II Promoter Escape Homo sapiens R-HSA-73776/6.68/Up; RNA Polymerase II HIV Promoter Escape Homo sapiens R-HSA-167162/6.68/Up; Apoptosis Homo sapiens WP254/6.64/Down; Creatine metabolism Homo sapiens R-HSA-71288/6.50/Down; Removal of aminoterminal propeptides from gamma-carboxylated proteins Homo sapiens R-HSA-159782/6.50/Down; Fatty acid oxidation Mus musculus WP2318/6.50/Down; Activation of the AP-1 family of transcription factors Homo sapiens R-HSA-45034/6.50/Down; IRAK2 mediated activation of TAK1 complex Homo sapiens R-HSA-937042/6.50/Down; IRAK2 mediated activation of TAK1 complex Up; on TLR7/8 or 9 stimulation Homo sapiens R-HSA-975163/6.50/Down; ATF6-alpha activates chaperone genes Homo sapiens R-HSA-381183/6.50/Down; Adenylate cyclase activating pathway Homo sapiens R-HSA-170660/6.50/Down; Sulfur relay system Homo sapiens hsa04122/6.50/Down; Synthesis and degradation of ketone bodies Homo sapiens hsa00072/6.50/Down; Gamma-carboxylation of protein precursors Homo sapiens R-HSA-159740/6.50/Down; Activation of Ca-permeable Kainate Receptor Homo sapiens R-HSA-451308/6.50/Up; Urea cycle Homo sapiens R-HSA-70635/6.50/Up; Calcitonin-like ligand receptors Homo sapiens R-HSA-419812/6.50/Up; Thyroxine biosynthesis Homo sapiens R-HSA-209968/6.50/Up; Tetrahydrobiopterin (BH4) synthesis, recycling, salvage and regulation Homo sapiens R-HSA-1474151/6.50/Up; IRAK1 recruits IKK complex Up; on TLR7/8 or 9 stimulation Homo sapiens R-HSA-975144/6.50/Up; IRAK1 recruits IKK complex Homo sapiens R-HSA-937039/6.50/Up; Signal attenuation Homo sapiens R-HSA-74749/6.50/Up; Removal of the Flap Intermediate from the C-strand Homo sapiens R-HSA-174437/6.50/Up; Activation of PPARGC1A (PGC-1alpha) by phosphorylation Homo sapiens R-HSA-2151209/6.50/Up; Reactions specific to the complex N-glycan synthesis pathway Homo sapiens R-HSA-975578/6.50/Up; Ionotropic activity of Kainate Receptors Homo sapiens R-HSA-451306/6.50/Up; Zinc influx into cells by the SLC39 gene family Homo sapiens R-HSA-442380/6.50/Up; Neurotoxicity of clostridium toxins Homo sapiens R-HSA-168799/6.50/Up; Leptin and adiponectin Mus musculus WP683/6.50/Up; Liver X Receptor Pathway Homo sapiens WP2874/6.50/Up; Synthesis and degradation of ketone bodies Homo sapiens hsa00072/6.50/Up; Glycosphingolipid biosynthesis-lacto and neolacto series Homo sapiens hsa00601/6.48/Down; Ligand-gated ion channel transport Homo sapiens R-HSA-975298/6.48/Down; Ligand-gated ion channel transport Homo sapiens R-HSA-975298/6.48/Up; Down; regulation of TGF-beta receptor signaling Homo sapiens R-HSA-2173788/6.48/Up; Calmodulin induced events Homo sapiens R-HSA-111933/6.48/Up; CaM pathway Homo sapiens R-HSA-111997/6.48/Up; N-glycan antennae elongation in the medial/trans-Golgi Homo sapiens R-HSA-975576/6.48/Up; PTK6 Regulates RHO GTPases, RAS GTPase and MAP kinases Homo sapiens R-HSA-8849471/6.48/Up; Metabolism of nucleotides Homo sapiens R-HSA-15869/6.47/Down; ErbB signaling pathway Homo sapiens hsa04012/6.47/Up; Integration of energy metabolism Homo sapiens R-HSA-163685/6.45/Up; O-linked glycosylation Homo sapiens R-HSA-5173105/6.45/Up; SUMOylation of RNA binding proteins Homo sapiens R-HSA-4570464/6.38/Up; Basal transcription factors Homo sapiens hsa03022/6.38/Up; Cholinergic synapse Homo sapiens hsa04725/6.31/Down; Cholinergic synapse Homo sapiens hsa04725/6.31/Up; Cardiac conduction Homo sapiens R-HSA-5576891/6.31/Up; Androgen receptor signaling pathway Homo sapiens WP138/6.13/Down; G alpha (z) signaling events Homo sapiens R-HSA-418597/6.10/Down; Selenium metabolism/Selenoproteins Mus musculus WP108/6.10/Up; DNA Damage Bypass Homo sapiens R-HSA-73893/6.10/Up; Kit Receptor Signaling Pathway Mus musculus WP407/6.08/Up; SLC-mediated transmembrane transport Homo sapiens R-HSA-425407/6.05/Up; CREB phosphorylation through the activation of Ras Homo sapiens R-HSA-442742/6.03/Up; Uptake and actions of bacterial toxins Homo sapiens R-HSA-5339562/6.03/Up; VEGFR2 mediated vascular permeability Homo sapiens R-HSA-5218920/6.03/Up; Keratan sulfate biosynthesis Homo sapiens R-HSA-2022854/6.03/Up; DNA Damage Response Homo sapiens WP707/5.89/Down; Interferon alpha/beta signaling Homo sapiens R-HSA-909733/5.89/Down; Nucleotide excision repair Homo sapiens hsa03420/5.83/Up; Selenium Metabolism and Selenoproteins Homo sapiens WP28/5.83/Up; Heart Development Homo sapiens WP1591/5.83/Up; Oxidative Stress Induced Senescence Homo sapiens R-HSA-2559580/5.82/Down; Morphine addiction Homo sapiens hsa05032/5.82/Down; Focal Adhesion Homo sapiens WP306/5.78/Down; AMPK Signaling Homo sapiens WP1403/5.70/Down; p53 signaling pathway Homo sapiens hsa04115/5.70/Down; Glyoxylate and dicarboxylate metabolism Homo sapiens hsa00630/5.62/Down; PERK regulates gene expression Homo sapiens R-HSA-381042/5.62/Down; Melatonin metabolism and effects Homo sapiens WP3298/5.62/Up; Ca-dependent events Homo sapiens R-HSA-111996/5.62/Up; Recognition of DNA damage by PCNA-containing replication complex Homo sapiens R-HSA-110314/5.62/Up; Butanoate metabolism Homo sapiens hsa00650/5.62/Up; cGMP-PKG signaling pathway Homo sapiens hsa04022/5.60/Up; Integrated Cancer Pathway Homo sapiens WP1971/5.58/Down; One carbon metabolism and related pathways Mus musculus WP1770/5.58/Down; TP53 Regulates Transcription of Cell Cycle Genes Homo sapiens R-HSA-6791312/5.58/Down; Type II diabetes mellitus Homo sapiens hsa04930/5.58/Down; Diurnally Regulated Genes with Circadian Orthologs Homo sapiens WP410/5.58/Up; Fatty acid metabolism Homo sapiens hsa01212/5.58/Up; Exercise-induced Circadian Regulation Mus musculus WP544/5.58/Up; G beta:gamma signaling through PI3Kgamma Homo sapiens R-HSA-392451/5.58/Up; Diurnally Regulated Genes with Circadian Orthologs Mus musculus WP1268/5.58/Up; Signaling by NOTCH3 Homo sapiens R-HSA-1980148/5.52/Down; Uptake and function of anthrax toxins Homo sapiens R-HSA-5210891/5.52/Down; Dermatan sulfate biosynthesis Homo sapiens R-HSA-2022923/5.52/Down; Interleukin-7 signaling Homo sapiens R-HSA-1266695/5.52/Down; Gamma-carboxylation, transport, and amino-terminal cleavage of proteins Homo sapiens R-HSA-159854/5.52/Down; Heme biosynthesis Homo sapiens R-HSA-189451/5.52/Down; Interleukin-6 signaling Homo sapiens R-HSA-1059683/5.52/Down; Gene regulatory network modelling somitogenesis Homo sapiens WP2854/5.52/Down; Signaling by NOTCH4 Homo sapiens R-HSA-1980150/5.52/Down; Tryptophan catabolismomo sapiens R-HSA-71240/5.52/Down; Uptake and function of anthrax toxins/5.52/Up; DSCAM interactions Homo sapiens R-HSA-Homo sapiens R-HSA-521089/376172/5.52/Up; Heme biosynthesis Homo sapiens R-HSA-189451/5.52/Up; Synthesis of IP2, IP, and Ins in the cytosol Homo sapiens R-HSA-1855183/5.52/Up; CLEC7A (Dectin-1) induces NFAT activation Homo sapiens R-HSA-5607763/5.52/Up; Interleukin-7 signaling Homo sapiens R-HSA-1266695/5.52/Up; Dermatan sulfate biosynthesis Homo sapiens R-HSA-2022923/5.52/Up; Processive synthesis on the C-strand of the telomere Homo sapiens R-HSA-174414/5.52/Up; Ubiquinone and other terpenoid-/5.52/Up; Taurine and hypotaurine quinone biosynthesis Homo sapiens hsa00130 metabolism Homo sapiens hsa00430/5.52/Up; Parkin-Ubiquitin Proteasomal System pathway Homo sapiens WP2359/5.52/Up; Neuroactive ligand-receptor interaction Homo sapiens hsa04080/5.47/Up; Longevity regulating pathway-mammal Homo sapiens hsa04211/5.39/Down; Endoderm Differentiation Homo sapiens WP2853/5.34/Up; Circadian entrainment Homo sapiens hsa04713/5.25/Up; Chaperonin-mediated protein folding Homo sapiens R-HSA-390466/5.25/Up; MAPK Cascade Homo sapiens WP422/5.25/Down; Thyroid cancer Homo sapiens hsa05216/5.25/Down; MAPK Cascade Mus musculus WP251/5.25/Down; Activation of BH3-only proteins Homo sapiens R-HSA-114452/5.25/Down; Interaction between L1 and Ankyrins Homo sapiens R-HSA-445095/5.25/Up; TRAF6 Mediated Induction of proinflammatory cytokines Homo sapiens R-HSA-168180/5.17/Down; Signaling by NOTCH1 Homo sapiens R-HSA-1980143/5.17/Up; Transcriptional regulation by small RNAs Homo sapiens R-HSA-5578749/5.17/Up; CAMP signaling pathway Homo sapiens hsa04024/5.15/Up; Imatinib Resistance in Chronic Myeloid Leukemia Homo sapiens WP2946/5.11/Up; Chondroitin sulfate/dermatan sulfate metabolism Homo sapiens R-HSA-1793185/5.11/Up; Metabolism of non-coding RNA Homo sapiens R-HSA-194441/5.11/Up; snRNP Assembly Homo sapiens R-HSA-191859/5.11/Up; MAPK signaling pathway Homo sapiens hsa04010/5.10/Down; Anchoring of the basal body to the plasma membrane Homo sapiens R-HSA-5620912/4.99/Up; IL-3 Signaling Pathway Mus musculus WP373/4.99/Up; HTLV-I infection Homo sapiens hsa05166/4.92/Down; MAPK targets/Nuclear events mediated by MAP kinases Homo sapiens R-HSA-450282/4.91/Down; Hypothetical Network for Drug Addiction Mus musculus WP1246/4.91/Up; RNA Polymerase I Promoter Escape Homo sapiens R-HSA-73772/4.91/Up; Tight junction interactions Homo sapiens R-HSA-420029/4.91/Up; Galactose metabolism Homo sapiens hsa00052/4.91/Up; Termination of translesion DNA synthesis Homo sapiens R-HSA-5656169/4.91/Up; Amyotrophic lateral sclerosis (ALS) Homo sapiens hsa05014/4.90/Down; Mineral absorption Homo sapiens hsa04978/4.90/Up; Neurotransmitter Release Cycle Homo sapiens R-HSA-112310/4.90/Up; mRNA 3′-end processing Homo sapiens R-HSA-72187/4.90/Up; Post-Elongation Processing of Intron-Containing pre-mRNA Homo sapiens R-HSA-112296/4.90/Up; G-protein beta:gamma signaling Homo sapiens R-HSA-397795/4.90/Up; Inflammatory mediator regulation of TRP channels Homo sapiens hsa04750/4.87/Down; Progesterone-mediated oocyte maturation Homo sapiens hsa04914/4.87/Down; AMPK signaling pathway Homo sapiens hsa04152/4.78/Down; RNA Polymerase II Transcription Homo sapiens R-HSA-73857/4.78/Up; Inhibition of replication initiation of damaged DNA by RB1/E2F1 Homo sapiens R-HSA-113501/4.75/Down; ATF6-alpha activates chaperones Homo sapiens R-HSA-381033/4.75/Down; MFAP5-mediated ovarian cancer cell motility and invasiveness Homo sapiens WP3301/4.75/Down; mRNA decay by 3′ to 5′ exoribonuclease Homo sapiens R-HSA-429958/4.75/Down; TP53 Regulates Transcription of Caspase Activators and Caspases Homo sapiens R-HSA-6803207/4.75/Down; TP53 Regulates Transcription of Death Receptors and Ligands Homo sapiens R-HSA-6803211/4.75/Down; Tandem pore domain potassium channels Homo sapiens R-HSA-1296346/4.75/Down; ERKs are inactivated Homo sapiens R-HSA-202670/4.75/Down; Physiological factors Homo sapiens R-HSA-5578768/4.75/Up; Peptide hormone biosynthesis Homo sapiens R-HSA-209952/4.75/Up; Serotonin receptors Homo sapiens R-HSA-390666/4.75/Up; Reduction of cytosolic Ca++ levels Homo sapiens R-HSA-418359/4.75/Up; Unwinding of DNA Homo sapiens R-HSA-176974/4.75/Up; mRNA decay by 3′ to 5′ exoribonuclease Homo sapiens R-HSA-429958/4.75/Up; Iron metabolism in placenta Homo sapiens WP2007/4.75/Up; Alanine and aspartate metabolism Homo sapiens WP106/4.75/Up; Alanine and aspartate metabolism Mus musculus WP240/4.75/Up; Potassium Channels Homo sapiens R-HSA-1296071/4.75/Down; Potassium Channels Homo sapiens R-HSA-1296071/4.75/Up; G alpha (12/13) signaling events Homo sapiens R-HSA-416482/4.71/Up; Nonhomologous End-Joining (NHEJ) Homo sapiens R-HSA-5693571/4.70/Down; Endometrial cancer Homo sapiens hsa05213/4.70/Down; Myometrial Relaxation and Contraction Pathways Mus musculus WP385/4.70/Down; Melanogenesis Homo sapiens hsa04916/4.64/Down; Gastric Cancer Network 2 Homo sapiens WP2363/4.61/Down; Ovarian Infertility Genes Mus musculus WP273/4.61/Down; Adherens junctions interactions Homo sapiens R-HSA-418990/4.61/Down; Inwardly rectifying K+ channels Homo sapiens R-HSA-1296065/4.61/Down; FGFR1 mutant receptor activation Homo sapiens R-HSA-1839124/4.61/Down; NOD1/2 Signaling Pathway Homo sapiens R-HSA-168638/4.61/Down; Other types of O-glycan biosynthesis Homo sapiens hsa00514/4.61/Down; HS-GAG biosynthesis Homo sapiens R-HSA-2022928/4.61/Up; Mucin type O-Glycan biosynthesis Homo sapiens hsa00512/4.61/Up; Other types of O-glycan biosynthesis Homo sapiens hsa00514/4.61/Up; Transcription-CoUp; led Nucleotide Excision Repair (TC-NER) Homo sapiens R-HSA-6781827/4.57/Up; Transcriptional misregulation in cancer Homo sapiens hsa05202/4.55/Up; Retrograde endocannabinoid signaling Homo sapiens hsa04723/4.52/Up; Glucagon signaling pathway Homo sapiens hsa04922/4.52/Up; Transport of glucose and other sugars, bile salts and organic acids, metal ions and amine compounds Homo sapiens R-HSA-425366/4.52/Up; Gene Silencing by RNA Homo sapiens R-HSA-211000/4.52/Up; Protein folding Homo sapiens R-HSA-391251/4.52/Up; GPVI-mediated activation cascade Homo sapiens R-HSA-114604/4.51/Down; Cardiac Progenitor Differentiation Homo sapiens WP2406/4.51/Up; Wnt Signaling Pathway and Pluripotency Homo sapiens WP399/4.41/Up; Late Phase of HIV Life Cycle Homo sapiens R-HSA-162599/4.40/Up; Signaling by NOTCH2 Homo sapiens R-HSA-1980145/4.33/Down; Ovarian Infertility Genes Homo sapiens WP34/4.33/Down; Type II interferon signaling (IFNG) Mus musculus WP1253/4.33/Down; Fructose and mannose metabolism Homo sapiens hsa00051/4.33/Down; Hypothetical Network for Drug Addiction Homo sapiens WP666/4.33/Up; Keratan sulfate/keratin metabolism Homo sapiens R-HSA-1638074/4.33/Up; RNA Polymerase I Transcription Termination Homo sapiens R-HSA-73863/4.33/Up; DNA strand elongation Homo sapiens R-HSA-69190/4.33/Up; Signaling by NOTCH2 Homo sapiens R-HSA-1980145/4.33/Up; Signaling by Robo receptor Homo sapiens R-HSA-376176/4.33/Up; DAG and IP3 signaling Homo sapiens R-HSA-1489509/4.33/Up; TGF-beta receptor signaling activates SMADs Homo sapiens R-HSA-2173789/4.33/Up; Myometrial Relaxation and Contraction Pathways Homo sapiens WP289/4.30/Down; Biosynthesis of the N-glycan precursor (dolichol lipid-linked oligosaccharide, LLO) and transfer to a nascent protein Homo sapiens R-HSA-446193/4.30/Up; Focal Adhesion Mus musculus WP85/4.20/Down; Apoptosis Mus musculus WP1254/4.17/Up; GABA receptor activation Homo sapiens R-HSA-977443/4.16/Down; RANKL/RANK Signaling Pathway Homo sapiens WP2018/4.16/Down; IL-1 signaling pathway Homo sapiens WP195/4.16/Down; GABA receptor activation Homo sapiens R-HSA-977443/4.16/Up; Cardiac Hypertrophic Response Homo sapiens WP2795/4.16/Up; Purine salvage Homo sapiens R-HSA-74217/4.14/Down; Apoptosis induced DNA fragmentation Homo sapiens R-HSA-140342/4.14/Down; Activation of DNA fragmentation factor Homo sapiens R-HSA-211227/4.14/Down; GABA A receptor activation Homo sapiens R-HSA-977441/4.14/Down; TP53 Regulates Transcription of Genes Involved in G1 Cell Cycle Arrest Homo sapiens R-HSA-6804116/4.14/Down; Signal regulatory protein (SIRP) family interactions Homo sapiens R-HSA-391160/4.14/Down; Serotonin and anxiety-related events Mus musculus WP2140/4.14/Down; Trafficking and processing of endosomal TLR Homo sapiens R-HSA-1679131/4.14/Down; Osteoclast Mus musculus WP454/4.14/Down; Estrogen Receptor Pathway Homo sapiens WP2881/4.14/Down; alpha-linolenic (omega3) and linoleic (omega6) acid metabolism Homo sapiens R-HSA-2046104/4.14/Up; Facilitative Na+-independent glucose transporters Homo sapiens R-HSA-428790/4.14/Up; Initiation of Nuclear Envelope Reformation Homo sapiens R-HSA-2995383/4.14/Up; Nuclear Envelope Reassembly Homo sapiens R-HSA-2995410/4.14/Up; POU5F1 (OCT4), SOX2, NANOG activate genes related to proliferation Homo sapiens R-HSA-2892247/4.14/Up; NICD traffics to nucleus Homo sapiens R-HSA-157052/4.14/Up; Serotonin and anxiety-related events Mus musculus WP2140/4.14/Up; Homologous recombination Mus musculus WP1258/4.14/Up; Homologous recombination Homo sapiens WP186/4.14/Up; Ganglio Sphingolipid Metabolism Homo sapiens WP1423/4.14/Up; Notch-HLH transcription pathway Homo sapiens R-HSA-350054/4.14/Up; p75NTR recruits signaling complexes Homo sapiens R-HSA-209543/4.14/Up; FGFRL1 modulation of FGFR1 signaling Homo sapiens R-HSA-5658623/4.14/Up; p38MAPK events Homo sapiens R-HSA-171007/4.14/Up; Advanced glycosylation endproduct receptor signaling Homo sapiens R-HSA-879415/4.14/Up; Synthesis, secretion, and inactivation of Glucose-dependent Insulinotropic Polypeptide (GIP) Homo sapiens R-HSA-400511/4.14/Up; Depolarization of the Presynaptic Terminal Triggers the Opening of Calcium Channels Homo sapiens R-HSA-112308/4.14/Up; Synthesis of PE Homo sapiens R-HSA-1483213/4.14/Up; alpha-linolenic acid (ALA) metabolism Homo sapiens R-HSA-2046106/4.14/Up; Senescence and Autophagy in Cancer Homo sapiens WP615/4.10/Down; Base excision repair Homo sapiens hsa03410/4.07/Down; Fluoropyrimidine Activity Homo sapiens WP1601/4.07/Down; E2F mediated regulation of DNA replication Homo sapiens R-HSA-113510/4.07/Down; Sialic acid metabolism Homo sapiens R-HSA-4085001/4.07/Up; CD28 co-stimulation Homo sapiens R-HSA-389356/4.07/Up; TGF-beta Signaling Pathway Homo sapiens WP366/4.06/Down; Osteoclast differentiation Homo sapiens hsa04380/4.06/Down; Regulation of insulin secretion Homo sapiens R-HSA-422356/4.05/Down; Non-small cell lung cancer Homo sapiens hsa05223/4.00/Down; FoxO signaling pathway Homo sapiens hsa04068/3.98/Down; Delta-Notch Signaling Pathway Mus musculus WP265/3.93/Up; Pre-NOTCH Expression and Processing Homo sapiens R-HSA-1912422/3.84/Down; tRNA processing in the nucleus Homo sapiens R-HSA-6784531/3.84/Up; Signaling by NOTCH1 HD+PEST/3.84/Up; Signaling by Domain Mutants in Cancer Homo sapiens R-HSA-2894858 NOTCH1 in Cancer Homo sapiens R-HSA-2644603/3.84/Up; Signaling by NOTCH1 PEST Domain Mutants in Cancer Homo sapiens R-HSA-2644602/3.84/Up; RNA Polymerase I Chain Elongation Homo sapiens R-HSA-73777/3.84/Up; Constitutive Signaling by NOTCH1 PEST Domain Mutants Homo sapiens R-HSA-2644606/3.84/Up; Constitutive Signaling by NOTCH1 HD+PEST Domain Mutants Homo sapiens R-HSA-2894862/3.84/Up; Metabolism of lipids and lipoproteins Homo sapiens R-HSA-556833/3.84/Up; Nuclear Pore Complex (NPC) Disassembly Homo sapiens R-HSA-3301854/3.83/Down; EGFR interacts with phospholipase C-gamma Homo sapiens R-HSA-212718/3.83/Up; PLC-gamma1 signaling Homo sapiens R-HSA-167021/3.83/Up; Signaling of Hepatocyte Growth Factor Receptor Homo sapiens WP313/3.83/Up; Signaling of Hepatocyte Growth Factor Receptor Mus musculus WP193/3.83/Up; Processing of Capped Intron-Containing Pre-mRNA Homo sapiens R-HSA-72203/3.72/Up; TRAF6 mediated induction of NFkB and MAP kinases Up; on TLR7/8 or 9 activation Homo sapiens R-HSA-975138/3.71/Down; Hypertrophic cardiomyopathy (HCM) Homo sapiens hsa05410/3.71/Up; Wnt Signaling Pathway Mus musculus WP403/3.70/Up; B—WICH complex positively regulates rRNA expression Homo sapiens R-HSA-5250924/3.70/Up; TNF signaling pathway Homo sapiens hsa04668/3.65/Down; Integration of energy metabolism Homo sapiens R-HSA-163685/3.65/Down; Kennedy pathway Mus musculus WP1771/3.63/Down; Metastatic brain tumor Homo sapiens WP2249/3.63/Down; Synthesis of Prostaglandins (PG) and Thromboxanes (TX) Homo sapiens R-HSA-2162123/3.63/Down; Receptor-ligand binding initiates the second proteolytic cleavage of Notch receptor Homo sapiens R-HSA-156988/3.63/Down; Regulation of IFNG signaling Homo sapiens R-HSA-877312/3.63/Down; Removal of the Flap Intermediate Homo sapiens R-HSA-69166/3.63/Down; Leading Strand Synthesis Homo sapiens R-HSA-69109/3.63/Down; Polymerase switching Homo sapiens R-HSA-69091/3.63/Down; Polymerase switching on the C-strand of the telomere Homo sapiens R-HSA-174411/3.63/Down; Regulation of TP53 Activity through Association with Co-factors Homo sapiens R-HSA-6804759/3.63/Down; SUMOylation of transcription factors Homo sapiens R-HSA-3232118/3.63/Down; Adenylate cyclase inhibitory pathway Homo sapiens R-HSA-170670/3.63/Down; Inhibition of adenylate cyclase pathway Homo sapiens R-HSA-997269/3.63/Down; TP53 regulates transcription of several additional cell death genes whose specific roles in p53-dependent apoptosis remain uncertain Homo sapiens R-HSA-6803205/3.63/Down; SEMA3A-Plexin repulsion signaling by inhibiting Integrin adhesion Homo sapiens R-HSA-399955/3.63/Down; Folate biosynthesis Homo sapiens hsa00790/3.63/Up; Biogenic Amine Synthesis Mus musculus WP522/3.63/Up; LGI-ADAM interactions Homo sapiens R-HSA-5682910/3.63/Up; Regulation of TP53 Activity through Association with Co-factors Homo sapiens R-HSA-6804759/3.63/Up; TP53 regulates transcription of several additional cell death genes whose specific roles in p53-dependent apoptosis remain uncertain Homo sapiens R-HSA-6803205/3.63/Up; Polymerase switching on the C-strand of the telomere Homo sapiens R-HSA-174411/3.63/Up; Polymerase switching Homo sapiens R-HSA-69091/3.63/Up; Leading Strand Synthesis Homo sapiens R-HSA-69109/3.63/Up; Mismatch repair (MMR) directed by MSH2:MSH3 (MutSbeta) Homo sapiens R-HSA-5358606/3.63/Up; Mismatch repair (MMR) directed by MSH2:MSH6 (MutSalpha) Homo sapiens R-HSA-5358565/3.63/Up; Removal of the Flap Intermediate Homo sapiens R-HSA-69166/3.63/Up; Golgi Cisternae Pericentriolar Stack Reorganization Homo sapiens R-HSA-162658/3.63/Up; Repression of WNT target genes Homo sapiens R-HSA-4641265/3.63/Up; Nicotinate metabolism Homo sapiens R-HSA-196807/3.63/Up; PluriNetWork Mus musculus WP1763/3.62/Up; Prion diseases Homo sapiens hsa05020/3.61/Down; Generation of second messenger molecules Homo sapiens R-HSA-202433/3.61/Up; SIRT1 negatively regulates rRNA Expression Homo sapiens R-HSA-427359/3.61/Up; Post NMDA receptor activation events Homo sapiens R-HSA-438064/3.61/Up; DNA Repair Homo sapiens R-HSA-73894/3.58/Down; SIDS Susceptibility Pathways Mus musculus WP1266/3.56/Down; DNA Damage/Telomere Stress Induced Senescence Homo sapiens R-HSA-2559586/3.56/Down; Glycerolipid metabolism Homo sapiens hsa00561/3.56/Up; Preimplantation Embryo Homo sapiens WP3527/3.56/Up; DNA Double Strand Break Response Homo sapiens R-HSA-5693606/3.56/Up; Muscle contraction Homo sapiens R-HSA-397014/3.55/Up; Toll Like Receptor 7/8 (TLR7/8) Cascade Homo sapiens R-HSA-168181/3.50/Down; MyD88 dependent cascade initiated on endosome Homo sapiens R-HSA-975155/3.50/Down; Ras signaling pathway Homo sapiens hsa04014/3.48/Up; Toll-Like Receptors Cascades Homo sapiens R-HSA-168898/3.46/Down; G1 to S cell cycle control Mus musculus WP413/3.43/Down; Cleavage of Growing Transcript in the Termination Region Homo sapiens R-HSA-109688/3.43/Up; RNA Polymerase II Transcription Termination Homo sapiens R-HSA-73856/3.43/Up; Post-Elongation Processing of the Transcript Homo sapiens R-HSA-76044/3.43/Up; VEGFA-VEGFR2 Pathway Homo sapiens R-HSA-4420097/3.42/Up; Pentose and glucuronate interconversions Homo sapiens hsa00040/3.41/Down; IL-1 Signaling Pathway Mus musculus WP37/3.41/Down; Regulation of mRNA stability by proteins that bind AU-rich elements Homo sapiens R-HSA-450531/3.40/Up; Apoptosis Homo sapiens WP254/3.40/Up; PPARA activates gene expression Homo sapiens R-HSA-1989781/3.40/Up; Regulation of toll-like receptor signaling pathway Homo sapiens WP1449/3.39/Down; HIV Life Cycle Homo sapiens R-HSA-162587/3.39/Up; Signaling pathways regulating pluripotency of stem cells Homo sapiens hsa04550/3.33/Down; Neurotransmitter Receptor Binding And Downstream Transmission In The Postsynaptic Cell Homo sapiens R-HSA-112314/3.33/Down; Glutamatergic synapse Homo sapiens hsa04724/3.32/Up; GPCRs, Class A Rhodopsin-like Mus musculus WP189/3.32/Up; miRNA Regulation of DNA Damage Response Homo sapiens WP1530/3.31/Down; Epigenetic regulation of gene expression Homo sapiens R-HSA-212165/3.25/Up; Primary immunodeficiency Homo sapiens hsa05340/3.22/Down; Platelet Aggregation (Plug Formation) Homo sapiens R-HSA-76009/3.22/Down; Defective B3GALTL causes Peters-plus syndrome (PpS) Homo sapiens R-HSA-5083635/3.22/Up; Zinc homeostasis Homo sapiens WP3529/3.22/Up; Oxidative Damage Mus musculus WP1496/3.22/Up; HDR through Single Strand Annealing (SSA) Homo sapiens R-HSA-5685938/3.22/Up; Translesion synthesis by Y family DNA polymerases bypasses lesions on DNA template Homo sapiens R-HSA-110313/3.22/Up; Apoptotic cleavage of cellular proteins Homo sapiens R-HSA-111465/3.22/Up; Toll Like Receptor 9 (TLR9) Cascade Homo sapiens R-HSA-168138/3.21/Down; G Protein Signaling Pathways Mus musculus WP232/3.21/Down; Glucuronidation Mus musculus WP1241/3.21/Down; Leptin Insulin Overlap Mus musculus WP578/3.21/Down; Disorders of transmembrane transporters Homo sapiens R-HSA-5619115/3.21/Down; Signaling by NOTCH1 HD Domain Mutants in Cancer Homo sapiens R-HSA-2691230/3.21/Down; Constitutive Signaling by NOTCH1 HD Domain Mutants Homo sapiens R-HSA-2691232/3.21/Down; Processive synthesis on the lagging strand Homo sapiens R-HSA-69183/3.21/Down; Regulation of innate immune responses to cytosolic DNA Homo sapiens R-HSA-3134975/3.21/Down; N-Glycan antennae elongation Homo sapiens R-HSA-975577/3.21/Down; Glycosphingolipid biosynthesis-ganglio series Homo sapiens hsa00604/3.21/Down; Glycosaminoglycan biosynthesis-keratan sulfate Homo sapiens hsa00533/3.21/Up; Glycogen synthesis Homo sapiens R-HSA-3322077/3.21/Up; Glucuronidation Mus musculus WP1241/3.21/Up; Biogenic Amine Synthesis Homo sapiens WP550/3.21/Up; CREB phosphorylation through the activation of CaMKII Homo sapiens R-HSA-442729/3.21/Up; p130Cas linkage to MAPK signaling for integrins Homo sapiens R-HSA-372708/3.21/Up; Glycogen breakdown (glycogenolysis) Homo sapiens R-HSA-70221/3.21/Up; Glycosphingolipid biosynthesis-ganglio series Homo sapiens hsa00604/3.21/Up; Processive synthesis on the lagging strand Homo sapiens R-HSA-69183/3.21/Up; Mismatch Repair Homo sapiens R-HSA-5358508/3.21/Up; Depolymerisation of the Nuclear Lamina Homo sapiens R-HSA-4419969/3.21/Up; N-Glycan antennae elongation Homo sapiens R-HSA-975577/3.21/Up; BDNF signaling pathway Homo sapiens WP2380/3.20/Down; Beta-catenin independent WNT signaling Homo sapiens R-HSA-3858494/3.20/Up; Proteoglycans in cancer Homo sapiens hsa05205/3.19/Down; Colorectal cancer Homo sapiens hsa05210/3.18/Down; Gap-filling DNA repair synthesis and ligation in TC-NER Homo sapiens R-HSA-6782210/3.18/Up; Post-translational protein modification Homo sapiens R-HSA-597592/3.18/Up; Regulation of lipid metabolism by Peroxisome proliferator-activated receptor alpha (PPARalpha) Homo sapiens R-HSA-400206/3.17/Up; DNA Double-Strand Break Repair Homo sapiens R-HSA-5693532/3.14/Down; DNA Double-Strand Break Repair Homo sapiens R-HSA-5693532/3.14/Up; Signaling by VEGF Homo sapiens R-HSA-194138/3.14/Up; Regulation of TP53 Activity through Phosphorylation Homo sapiens R-HSA-6804756/3.13/Up; Salivary secretion Homo sapiens hsa04970/3.13/Up; Influenza A Homo sapiens hsa05164/3.10/Down; Viral carcinogenesis Homo sapiens hsa05203/3.10/Down; Purine metabolism Homo sapiens hsa00230/3.05/Down; Signaling by FGFR1 in disease Homo sapiens R-HSA-5655302/3.05/Down; Transport of vitamins, nucleosides, and related molecules Homo sapiens R-HSA-425397/3.05/Down; Amyotrophic lateral sclerosis (ALS) Homo sapiens WP2447/3.05/Down; Retinoblastoma (RB) in Cancer Homo sapiens WP2446/3.04/Down; TCR Signaling Pathway Homo sapiens WP69/3.04/Up; Neuronal System Homo sapiens R-HSA-112316/2.98/Down; Spinal Cord Injury Homo sapiens WP2431/2.97/Down; miRNA regulation of DNA Damage Response Mus musculus WP2087/2.96/Down; RNA Polymerase II Pre-transcription Events Homo sapiens R-HSA-674695/2.96/Up; Neurotrophin signaling pathway Homo sapiens hsa04722/2.90/Down; TNF-alpha NF-kB Signaling Pathway Mus musculus WP246/2.90/Up; Activation of GABAB receptors Homo sapiens R-HSA-991365/2.89/Down; GABA B receptor activation Homo sapiens R-HSA-977444/2.89/Down; Activation of NMDA receptor Up; on glutamate binding and postsynaptic events Homo sapiens R-HSA-442755/2.89/Up; Association of TriC/CCT with target proteins during biosynthesis Homo sapiens R-HSA-390471/2.89/Up; Dual Incision in GG-NER Homo sapiens R-HSA-5696400/2.89/Up; Gamma carboxylation, hypusine formation and arylsulfatase activation Homo sapiens R-HSA-163841/2.89/Up; Regulation of autophagy Homo sapiens hsa04140/2.89/Up; Toll Like Receptor 2 (TLR2) Cascade Homo sapiens R-HSA-181438/2.88/Down; MyD88:Mal cascade initiated on plasma membrane Homo sapiens R-HSA-166058/2.88/Down; Toll Like Receptor TLR1:TLR2 Cascade Homo sapiens R-HSA-168179/2.88/Down; Toll Like Receptor TLR6:TLR2 Cascade Homo sapiens R-HSA-168188/2.88/Down; MAPK6/MAPK4 signaling Homo sapiens R-HSA-5687128/2.88/Up; Rap1 signaling Homo sapiens R-HSA-392517/2.86/Down; JNK (c-Jun kinases) phosphorylation and activation mediated by activated human TAK1 Homo sapiens R-HSA-450321/2.86/Down; Osteoclast Signaling Homo sapiens WP12/2.86/Down; ID signaling pathway Homo sapiens WP53/2.86/Down; Quercetin and Nf-KB/AP-1 Induced Cell Apoptosis Homo sapiens WP2435/2.86/Down; Deregulation of Rab and Rab Effector Genes in Bladder Cancer Homo sapiens WP2291/2.86/Down; RHO GTPases activate CIT Homo sapiens R-HSA-5625900/2.86/Down; PKA activation Homo sapiens R-HSA-163615/2.86/Down; Synthesis of glycosylphosphatidylinositol (GPI) Homo sapiens R-HSA-162710/2.86/Down; TRAF6 mediated induction of TAK1 complex Homo sapiens R-HSA-937072/2.86/Down; Synthesis of glycosylphosphatidylinositol (GPI) Homo sapiens R-HSA-162710/2.86/Up; Apoptosis Modulation by HSP70 Mus musculus WP166/2.86/Up; TGF-beta receptor signaling in EMT (epithelial to mesenchymal transition) Homo sapiens R-HSA-2173791/2.86/Up; Regulation of KIT signaling Homo sapiens R-HSA-1433559/2.86/Up; MAP3K8 (TPL2)-dependent MAPK1/3 activation Homo sapiens R-HSA-5684264/2.86/Up; JNK (c-Jun kinases) phosphorylation and activation mediated by activated human TAK1 Homo sapiens R-HSA-450321/2.86/Up; p75NTR signals via NF-kB Homo sapiens R-HSA-193639/2.86/Up; Interactome of polycomb repressive complex 2 (PRC2) Homo sapiens WP2916/2.86/Up; Transcriptional misregulation in cancer Homo sapiens hsa05202/2.85/Down; Inflammatory bowel disease (IBD) Homo sapiens hsa05321/2.85/Down; Glioma Homo sapiens hsa05214/2.85/Down; Regulation of TP53 Activity Homo sapiens R-HSA-5633007/2.80/Up; Interferon gamma signaling Homo sapiens R-HSA-877300/2.80/Down; Signaling by NOTCH Homo sapiens R-HSA-157118/2.77/Down; Signaling by NOTCH Homo sapiens R-HSA-157118/2.77/Up; Triglyceride Biosynthesis Homo sapiens R-HSA-75109/2.75/Up; O-linked glycosylation of mucins Homo sapiens R-HSA-913709/2.75/Up; Renal cell carcinoma Homo sapiens hsa05211/2.75/Up; ATM Signaling Pathway Homo sapiens WP2516/2.74/Down; IL-5 Signaling Pathway Homo sapiens WP127/2.74/Down; Nicotine addiction Homo sapiens hsa05033/2.74/Down; Negative regulation of MAPK pathway Homo sapiens R-HSA-5675221/2.74/Up; PRC2 methylates histones and DNA Homo sapiens R-HSA-212300/2.74/Up; Wnt Signaling Pathway and Pluripotency Mus musculus WP723/2.72/Up; C-type lectin receptors (CLRs) Homo sapiens R-HSA-5621481/2.71/Up; Semaphorin interactions Homo sapiens R-HSA-373755/2.66/Down; Amphetamine addiction Homo sapiens hsa05031/2.66/Up; Signaling by PTK6 Homo sapiens R-HSA-8848021/2.66/Up; Wnt Signaling Pathway Homo sapiens WP428/2.66/Up; Transcription of the HIV genome Homo sapiens R-HSA-167172/2.66/Up; AMPK signaling pathway Homo sapiens hsa04152/2.65/Up; G Protein Signaling Pathways Homo sapiens WP35/2.65/Down; Striated Muscle Contraction Mus musculus WP216/2.60/Down; Condensation of Prophase Chromosomes Homo sapiens R-HSA-2299718/2.60/Up; Eukaryotic Transcription Initiation Mus musculus WP567/2.60/Up; Fatty acid, triacylglycerol, and ketone body metabolism Homo sapiens R-HSA-535734/2.59/Up; Spinal Cord Injury Mus musculus WP2432/2.58/Down; SUMO E3 ligases SUMOylate target proteins Homo sapiens R-HSA-3108232/2.58/Down; L1CAM interactions Homo sapiens R-HSA-373760/2.58/Up; G1 to S cell cycle control Homo sapiens WP45/2.56/Down; NOTCH1 regulation of human endothelial cell calcification Homo sapiens WP3413/2.56/Down; ACE Inhibitor Pathway Homo sapiens WP554/2.56/Down; IL-9 Signaling Pathway Homo sapiens WP22/2.56/Down; Zinc transporters Homo sapiens R-HSA-435354/2.56/Down; KSRP (KHSRP) binds and destabilizes mRNA Homo sapiens R-HSA-450604/2.56/Down; Butyrate Response Factor 1 (BRF1) binds and destabilizes mRNA Homo sapiens R-HSA-450385/2.56/Down; Tristetraprolin (TTP, ZFP36) binds and destabilizes mRNA Homo sapiens R-HSA-450513/2.56/Down; PKA activation in glucagon signaling Homo sapiens R-HSA-164378/2.56/Down; PKA-mediated phosphorylation of CREB Homo sapiens R-HSA-111931/2.56/Down; G1/S-Specific Transcription Homo sapiens R-HSA-69205/2.56/Down; Mitochondrial Fatty Acid Beta-Oxidation Homo sapiens R-HSA-77289/2.56/Down; Metabolism of porphyrins Homo sapiens R-HSA-189445/2.56/Down; Ras activation upon Ca2+ influx through NMDA receptor Homo sapiens R-HSA-442982/2.56/Up; Synthesis, secretion, and deacylation of Ghrelin Homo sapiens R-HSA-422085/2.56/Up; activated TAK1 mediates p38 MAPK activation Homo sapiens R-HSA-450302/2.56/Up; Hyaluronan metabolism Homo sapiens R-HSA-2142845/2.56/Up; Serotonin and anxiety Mus musculus WP2141/2.56/Up; Statin Pathway Mus musculus WP1/2.56/Up; KSRP (KHSRP) binds and destabilizes mRNA Homo sapiens R-HSA-450604/2.56/Up; Digestion of dietary lipid Homo sapiens R-HSA-192456/2.56/Up; NOTCH1 regulation of human endothelial cell calcification Homo sapiens WP3413/2.56/Up; Unblocking of NMDA receptor, glutamate binding and activation Homo sapiens R-HSA-438066/2.56/Up; SRF and miRs in Smooth Muscle Differentiation and Proliferation Homo sapiens WP199/2.56/Up; Acyl chain remodelling of PG Homo sapiens R-HSA-1482925/2.56/Up; Metabolism of porphyrins Homo sapiens R-HSA-189445/2.56/Up; TRIF-mediated TLR3/TLR4 signaling Homo sapiens R-HSA-937061/2.51/Down; Toll Like Receptor 3 (TLR3) Cascade Homo sapiens R-HSA-168164/2.51/Down; MyD88-independent TLR3/TLR4 cascade Homo sapiens R-HSA-166166/2.51/Down; IL-3 Signaling Pathway Mus musculus WP373/2.51/Down; IL-6 signaling Pathway Mus musculus WP387/2.51/Down; B Cell Receptor Signaling Pathway Homo sapiens WP23/2.51/Up; MAPK family signaling cascades Homo sapiens R-HSA-5683057/2.49/Up; mRNA Processing Homo sapiens WP411/2.48/Up; AMPK Signaling Homo sapiens WP1403/2.48/Up; EPHB-mediated forward signaling Homo sapiens R-HSA-3928662/2.47/Down; Glucagon-like Peptide-1 (GLP1) regulates insulin secretion Homo sapiens R-HSA-381676/2.47/Down; Intrinsic Pathway for Apoptosis Homo sapiens R-HSA-109606/2.47/Down; DNA Repair Homo sapiens R-HSA-73894/2.46/Up; Purine metabolism Mus musculus WP2185/2.45/Down; Oxytocin signaling pathway Homo sapiens hsa04921/2.45/Up; signaling to ERKs Homo sapiens R-HSA-187687/2.45/Up; Integrin-mediated Cell Adhesion Mus musculus WP6/2.38/Down; Hedgehog ‘off’ state Homo sapiens R-HSA-5610787/2.38/Down; Integrin-mediated Cell Adhesion Mus musculus WP6/2.38/Up; Transport of inorganic cations/anions and amino acids/oligopeptides Homo sapiens R-HSA-425393/2.38/Up; Dopaminergic synapse Homo sapiens hsa04728/2.38/Up; SIDS Susceptibility Pathways Homo sapiens WP706/2.37/Down; Voltage gated Potassium channels Homo sapiens R-HSA-1296072/2.35/Down; TP53 Regulates Transcription of Cell Death Genes Homo sapiens R-HSA-5633008/2.35/Down; ERCC6 (CSB) and EHMT2 (G9a) positively regulate rRNA expression Homo sapiens R-HSA-427389/2.35/Down; IL-7 Signaling Pathway Mus musculus WP297/2.35/Down; IL-7 Signaling Pathway Mus musculus WP297/2.35/Up; TP53 Regulates Transcription of Cell Death Genes Homo sapiens R-HSA-5633008/2.35/Up; Formation of Incision Complex in GG-NER Homo sapiens R-HSA-5696395/2.35/Up; Voltage gated Potassium channels Homo sapiens R-HSA-1296072/2.35/Up; Iron Uptake and transport Homo sapiens R-HSA-917937/2.35/Up; FCERI mediated MAPK activation Homo sapiens R-HSA-2871796/2.34/Up; Cellular Senescence Homo sapiens R-HSA-2559583/2.32/Down; Innate Immune System Homo sapiens R-HSA-168249/2.31/Up; Melanoma Homo sapiens hsa05218/2.31/Down; Thyroid hormone synthesis Homo sapiens hsa04918/2.31/Down; Inositol phosphate metabolism Homo sapiens hsa00562/2.31/Down; Regulation of signaling by CBL Homo sapiens R-HSA-912631/2.31/Down; cGMP effects Homo sapiens R-HSA-418457/2.31/Down; Signaling by cytosolic FGFR1 fusion mutants Homo sapiens R-HSA-1839117/2.31/Down; Miscellaneous transport and binding events Homo sapiens R-HSA-5223345/2.31/Down; Other glycan degradation Homo sapiens hsa00511/2.31/Down; Eicosanoid Synthesis Mus musculus WP318/2.31/Down; Sulfation Biotransformation Reaction Homo sapiens WP692/2.31/Up; Amine-derived hormones Homo sapiens R-HSA-209776/2.31/Up; Serotonin Neurotransmitter Release Cycle Homo sapiens R-HSA-181429/2.31/Up; Norepinephrine Neurotransmitter Release Cycle Homo sapiens R-HSA-181430/2.31/Up; Pre-NOTCH Processing in Golgi Homo sapiens R-HSA-1912420/2.31/Up; Signaling by cytosolic FGFR1 fusion mutants Homo sapiens R-HSA-1839117/2.31/Up; Regulation of signaling by CBL Homo sapiens R-HSA-912631/2.31/Up; Synthesis of PIPs at the Golgi membrane Homo sapiens R-HSA-1660514/2.31/Up; Cytosolic sulfonation of small molecules Homo sapiens R-HSA-156584/2.31/Up; Miscellaneous transport and binding events Homo sapiens R-HSA-5223345/2.31/Up; XPodNet-protein-protein interactions in the podocyte expanded by STRING Mus musculus WP2309/2.30/Up; SUMOylation Homo sapiens R-HSA-2990846/2.26/Down; AGE-RAGE signaling pathway in diabetic complications Homo sapiens hsa04933/2.26/Down; Integrin-mediated Cell Adhesion Homo sapiens WP185/2.26/Down; Retrograde endocannabinoid signaling Homo sapiens hsa04723/2.26/Down; Integrin-mediated Cell Adhesion Homo sapiens WP185/2.26/Up; EGF/EGFR Signaling Pathway Homo sapiens WP437/2.24/Down; Apoptosis Homo sapiens R-HSA-109581/2.24/Up; Prolactin signaling pathway Homo sapiens hsa04917/2.23/Down; Cell Cycle, Mitotic Homo sapiens R-HSA-69278/2.23/Up; Interleukin-1 signaling Homo sapiens R-HSA-446652/2.23/Down; Inositol phosphate metabolism Homo sapiens R-HSA-1483249/2.23/Down; PLC beta mediated events Homo sapiens R-HSA-112043/2.23/Up; Interleukin-1 signaling Homo sapiens R-HSA-446652/2.23/Up; Eukaryotic Transcription Initiation Homo sapiens WP405/2.23/Up; Phospholipase C-mediated cascade; FGFR3 Homo sapiens R-HSA-5654227/2.23/Up; Phase 0-rapid depolarisation Homo sapiens R-HSA-5576892/2.23/Up; Metapathway biotransformation Mus musculus WP1251/2.23/Up; Osteoclast differentiation Homo sapiens hsa04380/2.23/Up; Interferon Signaling Homo sapiens R-HSA-913531/2.19/Down; Positive epigenetic regulation of rRNA expression Homo sapiens R-HSA-5250913/2.16/Down; Leishmaniasis Homo sapiens hsa05140/2.16/Down; Signaling by TGF-beta Receptor Complex Homo sapiens R-HSA-170834/2.16/Up; Positive epigenetic regulation of rRNA expression Homo sapiens R-HSA-5250913/2.16/Up; NoRC negatively regulates rRNA expression Homo sapiens R-HSA-427413/2.16/Up; Chemokine signaling pathway Mus musculus WP2292/2.16/Up; SUMOylation of RNA binding proteins Homo sapiens R-HSA-4570464/2.12/Down; Ether lipid metabolism Homo sapiens hsa00565/2.12/Down; ABC-family proteins mediated transport Homo sapiens R-HSA-382556/2.12/Down; G-protein mediated events Homo sapiens R-HSA-112040/2.12/Up; Programmed Cell Death Homo sapiens R-HSA-5357801/2.12/Up; Arrhythmogenic right ventricular cardiomyopathy (ARVC) Homo sapiens hsa05412/2.09/Down; Signaling by NODAL Homo sapiens R-HSA-1181150/2.09/Down; Ephrin signaling Homo sapiens R-HSA-3928664/2.09/Down; Overview of nanoparticle effects Homo sapiens WP3287/2.09/Down; Blood Clotting Cascade Mus musculus WP460/2.09/Down; Serotonin Receptor 4/6/7 and NR3C Signaling Homo sapiens WP734/2.09/Down; Phase 4-resting membrane potential Homo sapiens R-HSA-5576886/2.09/Down; Gap junction assembly Homo sapiens R-HSA-190861/2.09/Up; TP53 Regulates Transcription of Genes Involved in Cytochrome C Release Homo sapiens R-HSA-6803204/2.09/Up; PCNA-Dependent Long Patch Base Excision Repair Homo sapiens R-HSA-5651801/2.09/Up; Small Ligand GPCRs Homo sapiens WP247/2.09/Up; Farnesoid X Receptor Pathway Homo sapiens WP2879/2.09/Up; Apoptosis Modulation by HSP70 Homo sapiens WP384/2.09/Up; Cellular responses to stress Homo sapiens R-HSA-2262752/2.07/Up; Measles Homo sapiens hsa05162/2.05/Down; Pyrimidine metabolism Homo sapiens hsa00240/2.04/Down; Phospholipase C-mediated cascade; FGFR4 Homo sapiens R-HSA-5654228/2.02/Up; Notch Signaling Pathway Homo sapiens WP268/2.02/Up; Mecp2 and Associated Rett Syndrome Mus musculus WP2910/2.02/Up; Notch Signaling Pathway Mus musculus WP29/2.02/Up; and Arrhythmogenic Right Ventricular Cardiomyopathy Homo sapiens WP2118/2.02/Down.

### Upregulated Kinase Activity in GSE88773 by Combined Score

DCLK1; MAPK6; OXSR1; STK11; MAP3K12; CDK19; PLK2; BRSK2; STK39; CAMKK2; STK24; CCNB1; MAPK8; STK3; MAP3K11; CDK8; IRAK2; MAP3K2; IRAK4; SRPK1; GSK3B; AKT1; MAPK4; MAP3K1; IRAK1; CSNK2A2; ABL2; CDK1; MAPK10; PRKAA2; BRSK1; ADRBK2; CDK5; MAPK3; PRKACA; NEK6; NEK2; MAP2K4; NTRK2; UHMK1; FGFR3; CAMKK1; RPS6KB1; PRKACG; CDK15; MAPK1; CDK18; PAK3; CDK11A; CDK2; CSNK1E; CDK14; RPS6KA1; ZAP70; IKBKE; CAMK2B; PDPK1; CAMK2A; PRKCA; MAPKAPK2; RPS6KA3; MAP3K5; TBK1; PRKCZ; MARK2; CSNK1G2; AKT2; CAMK2D; PRKAA1; CAMK2G; MAPK9; MINK1; PDK1; GSK3A; LYN; CHEK1; PRKCQ; CSNKIA1; AURKA; INSR; CSK; PRKC1; PAK2; MAPK12; MAPK11; MAPK14; MAPK7; SGK1; FGFR1; PLK3; EGFR; PAK1; CDK7; PRKDC; FYN; CSNK2A1; CHUK; CHEK2; ADRBK1; IKBKB; RPS6KA5; ATM; PRKCD; PRKG1; PRKACB; JAK2; MTOR; PRKCG; PRKCB; SYK; SRC; IGF1R; PRKCE; ABL1; TRIB1 human kinase ARCHS4 coexpression; EIF2AK3 human kinase ARCHS4 coexpression; SIK1 human kinase ARCHS4 coexpression; RNASEL human kinase ARCHS4 coexpression; STK38L human kinase ARCHS4 coexpression; ACVR1C human kinase ARCHS4 coexpression; CHUK human kinase ARCHS4 coexpression; UHMK1 human kinase ARCHS4 coexpression; PRKAA1 human kinase ARCHS4 coexpression; CLK1 human kinase ARCHS4 coexpression; MAP3K2 human kinase ARCHS4 coexpression; PDK4 human kinase ARCHS4 coexpression; DYRK1A human kinase ARCHS4 coexpression; PKN2 human kinase ARCHS4 coexpression; PIK3CA human kinase ARCHS4 coexpression; RPS6KA3 human kinase ARCHS4 coexpression; MET human kinase ARCHS4 coexpression; OXSR1 human kinase ARCHS4 coexpression; PRKD3 human kinase ARCHS4 coexpression; DYRK3 human kinase ARCHS4 coexpression; PRKACB human kinase ARCHS4 coexpression; MAPK6 human kinase ARCHS4 coexpression; NEK7 human kinase ARCHS4 coexpression; CDC42BPA human kinase ARCHS4 coexpression; ALK human kinase ARCHS4 coexpression; CDK17 human kinase ARCHS4 coexpression; PRPF4B human kinase ARCHS4 coexpression; STK24 human kinase ARCHS4 coexpression; ATR human kinase ARCHS4 coexpression; PRKCE human kinase ARCHS4 coexpression; NLK human kinase ARCHS4 coexpression; MYO3A human kinase ARCHS4 coexpression; CDK14 human kinase ARCHS4 coexpression; ROCK2 human kinase ARCHS4 coexpression; SLK human kinase ARCHS4 coexpression; EPHA1 human kinase ARCHS4 coexpression; EPHA2 human kinase ARCHS4 coexpression; ROR1 human kinase ARCHS4 coexpression; PLK3 human kinase ARCHS4 coexpression; CDK8 human kinase ARCHS4 coexpression; MAP2K4 human kinase ARCHS4 coexpression; MAP3K8 human kinase ARCHS4 coexpression; ABL2 human kinase ARCHS4 coexpression; RYK human kinase ARCHS4 coexpression; PIK3R4 human kinase ARCHS4 coexpression; TLK2 human kinase ARCHS4 coexpression; CDK12 human kinase ARCHS4 coexpression; SGK1 human kinase ARCHS4 coexpression; TGFBR1 human kinase ARCHS4 coexpression; MAP4K3 human kinase ARCHS4 coexpression; ROCK1 human kinase ARCHS4 coexpression; CAMK4 human kinase ARCHS4 coexpression; DCLK1 human kinase ARCHS4 coexpression; CDK1 human kinase ARCHS4 coexpression; CDK13 human kinase ARCHS4 coexpression; STK26 human kinase ARCHS4 coexpression; RIPK2 human kinase ARCHS4 coexpression; YES1 human kinase ARCHS4 coexpression; CSNK2A1 human kinase ARCHS4 coexpression; PLK2 human kinase ARCHS4 coexpression; SCYL2 human kinase ARCHS4 coexpression; MASTL human kinase ARCHS4 coexpression; LATS1 human kinase ARCHS4 coexpression; AKT3 human kinase ARCHS4 coexpression; PLK4 human kinase ARCHS4 coexpression; CSNK2A2 human kinase ARCHS4 coexpression; BMP2K human kinase ARCHS4 coexpression; PDGFRA human kinase ARCHS4 coexpression; FLT1 human kinase ARCHS4 coexpression; AXL human kinase ARCHS4 coexpression; KSR1 human kinase ARCHS4 coexpression; IRAK2 human kinase ARCHS4 coexpression; BRAF human kinase ARCHS4 coexpression; MAP3K4 human kinase ARCHS4 coexpression; MAPK1 human kinase ARCHS4 coexpression; TTBK2 human kinase ARCHS4 coexpression; PIM3 human kinase ARCHS4 coexpression; MKNK2 human kinase ARCHS4 coexpression; MELK human kinase ARCHS4 coexpression; MAPKAPK2 human kinase ARCHS4 coexpression; WEE2 human kinase ARCHS4 coexpression; ATM human kinase ARCHS4 coexpression; TTK human kinase ARCHS4 coexpression; RET human kinase ARCHS4 coexpression; DDR2 human kinase ARCHS4 coexpression; PRKCA human kinase ARCHS4 coexpression; CIT human kinase ARCHS4 coexpression; NTRK3 human kinase ARCHS4 coexpression; CAMK1G human kinase ARCHS4 coexpression; PRKC1 human kinase ARCHS4 coexpression; HIPK1 human kinase ARCHS4 coexpression; PBK human kinase ARCHS4 coexpression; PAK6 human kinase ARCHS4 coexpression; PAK1 human kinase ARCHS4 coexpression; CAMK2A human kinase ARCHS4 coexpression; CAMK2B human kinase ARCHS4 coexpression; CHEK 1 human kinase ARCHS4 coexpression; PIM1 human kinase ARCHS4 coexpression; SNRK human kinase ARCHS4 coexpression; TSSK4 human kinase ARCHS4 coexpression; CDK15 human kinase ARCHS4 coexpression; GSK3B human kinase ARCHS4 coexpression; MAP3K9 human kinase ARCHS4 coexpression; MAP2K1 human kinase ARCHS4 coexpression; MAP3K1 human kinase ARCHS4 coexpression; VRK1 human kinase ARCHS4 coexpression; CSNK1A1 human kinase ARCHS4 coexpression; DYRK2 human kinase ARCHS4 coexpression; NEK3 human kinase ARCHS4 coexpression; SIK3 human kinase ARCHS4 coexpression; MAPK9 human kinase ARCHS4 coexpression; PNCK human kinase ARCHS4 coexpression; PRKCZ human kinase ARCHS4 coexpression; PRKCG human kinase ARCHS4 coexpression; LATS2 human kinase ARCHS4 coexpression; GRK3 human kinase ARCHS4 coexpression; MYLK human kinase ARCHS4 coexpression; TNIK human kinase ARCHS4 coexpression; BMPR2 human kinase ARCHS4 coexpression; RAF1 human kinase ARCHS4 coexpression; EPHA6 human kinase ARCHS4 coexpression; EPHB6 human kinase ARCHS4 coexpression; JAK1 human kinase ARCHS4 coexpression; LTK human kinase ARCHS4 coexpression; BUB1 human kinase ARCHS4 coexpression; BUB1B human kinase ARCHS4 coexpression; CAMKK1 human kinase ARCHS4 coexpression; STK35 human kinase ARCHS4 coexpression; KALRN human kinase ARCHS4 coexpression; KDR human kinase ARCHS4 coexpression; FYN human kinase ARCHS4 coexpression; CSK human kinase ARCHS4 coexpression; TNNI3K human kinase ARCHS4 coexpression; LRRK2 human kinase ARCHS4 coexpression; ARAF human kinase ARCHS4 coexpression; STK39 human kinase ARCHS4 coexpression; PAK3 human kinase ARCHS4 coexpression; MAP3K7 human kinase ARCHS4 coexpression; MAP3K3 human kinase ARCHS4 coexpression; MAP3K11 human kinase ARCHS4 coexpression; MAP2K5 human kinase ARCHS4 coexpression; NEK2 human kinase ARCHS4 coexpression; ICK human kinase ARCHS4 coexpression; HIPK4 human kinase ARCHS4 coexpression; HIPK2 human kinase ARCHS4 coexpression; CDK19 human kinase ARCHS4 coexpression; MARK3 human kinase ARCHS4 coexpression; MARK2 human kinase ARCHS4 coexpression; KIT human kinase ARCHS4 coexpression; LMTK2 human kinase ARCHS4 coexpression; MUSK human kinase ARCHS4 coexpression; PTK2 human kinase ARCHS4 coexpression; SRC human kinase ARCHS4 coexpression; TYRO3 human kinase ARCHS4 coexpression; AURKA human kinase ARCHS4 coexpression; CDC7 human kinase ARCHS4 coexpression; EIF2AK2 human kinase ARCHS4 coexpression; MOS human kinase ARCHS4 coexpression; SBK3 human kinase ARCHS4 coexpression; WEE1 human kinase ARCHS4 coexpression; PRKDC human kinase ARCHS4 coexpression; SMG1 human kinase ARCHS4 coexpression; RIOK3 human kinase ARCHS4 coexpression; STK32B human kinase ARCHS4 coexpression; TYK2 human kinase ARCHS4 coexpression; GSG2 human kinase ARCHS4 coexpression; PLK5 human kinase ARCHS4 coexpression; STK32A human kinase ARCHS4 coexpression; CAMKK2 human kinase ARCHS4 coexpression; GRK6 human kinase ARCHS4 coexpression; TRPM7 human kinase ARCHS4 coexpression; RIOK1 human kinase ARCHS4 coexpression; RIOK2 human kinase ARCHS4 coexpression; CDC42BPG human kinase ARCHS4 coexpression; ROS1 human kinase ARCHS4 coexpression; ULK1 human kinase ARCHS4 coexpression; NTRK1 human kinase ARCHS4 coexpression; ITK human kinase ARCHS4 coexpression; FGFR3 human kinase ARCHS4 coexpression; STK40 human kinase ARCHS4 coexpression; PSKH2 human kinase ARCHS4 coexpression; PRKD2 human kinase ARCHS4 coexpression; NUAK2 human kinase ARCHS4 coexpression; DCLK3 human kinase ARCHS4 coexpression; CHEK2 human kinase ARCHS4 coexpression; TRIB2 human kinase ARCHS4 coexpression; CAMK2D human kinase ARCHS4 coexpression; BRSK2 human kinase ARCHS4 coexpression; BRSK1 human kinase ARCHS4 coexpression; PRKCB human kinase ARCHS4 coexpression; PRKACA human kinase ARCHS4 coexpression; PDPK1 human kinase ARCHS4 coexpression; MAST1 human kinase ARCHS4 coexpression; CAMK1D human kinase ARCHS4 coexpression; CSNKIG2 human kinase ARCHS4 coexpression; TTBK1 human kinase ARCHS4 coexpression; CDK20 human kinase ARCHS4 coexpression; AATK human kinase ARCHS4 coexpression; TGFBR2 human kinase ARCHS4 coexpression; TESK2 human kinase ARCHS4 coexpression; RIPK4 human kinase ARCHS4 coexpression; ILK human kinase ARCHS4 coexpression; ACVR1 human kinase ARCHS4 coexpression; MAP3K5 human kinase ARCHS4 coexpression; MAP2K7 human kinase ARCHS4 coexpression; MAP2K3 human kinase ARCHS4 coexpression; NEK4 human kinase ARCHS4 coexpression; SRPK2 human kinase ARCHS4 coexpression; MAPK4 human kinase ARCHS4 coexpression; CDKL2 human kinase ARCHS4 coexpression; CDK9 human kinase ARCHS4 coexpression; CDK6 human kinase ARCHS4 coexpression; GRK2 human kinase ARCHS4 coexpression; MAP3K14 human kinase ARCHS4 coexpression; PAK5 human kinase ARCHS4 coexpression; STK11 human kinase ARCHS4 coexpression; STK17B human kinase ARCHS4 coexpression; CSNKIG3 human kinase ARCHS4 coexpression; RPS6KC1 human kinase ARCHS4 coexpression; PINK1 human kinase ARCHS4 coexpression; GAK human kinase ARCHS4 coexpression; STYK1 human kinase ARCHS4 coexpression; SRMS human kinase ARCHS4 coexpression; PDGFRB human kinase ARCHS4 coexpression; JAK2 human kinase ARCHS4 coexpression; EPHA5 human kinase ARCHS4 coexpression; EPHA10 human kinase ARCHS4 coexpression; LIMK2 human kinase ARCHS4 coexpression; KSR2 human kinase ARCHS4 coexpression; STK4 human kinase ARCHS4 coexpression; CDK11B human kinase ARCHS4 coexpression; CDK18 human kinase ARCHS4 coexpression; CDK3 human kinase ARCHS4 coexpression; CDKL5 human kinase ARCHS4 coexpression; MAPK10 human kinase ARCHS4 coexpression; MAPK14 human kinase ARCHS4 coexpression; SRPK1 human kinase ARCHS4 coexpression; NEK9 human kinase ARCHS4 coexpression; MAP3K10 human kinase ARCHS4 coexpression; MAP3K12 human kinase ARCHS4 coexpression; NRK human kinase ARCHS4 coexpression; PAK2 human kinase ARCHS4 coexpression; NIM1K human kinase ARCHS4 coexpression; PIM2 human kinase ARCHS4 coexpression; MAPKAPK5 human kinase ARCHS4 coexpression; MAST3 human kinase ARCHS4 coexpression; PRKCD human kinase ARCHS4 coexpression; STK10 human kinase ARCHS4 coexpression; WNK3 human kinase ARCHS4 coexpression; WNK1 human kinase ARCHS4 coexpression; ULK2 human kinase ARCHS4 coexpression; STK36 human kinase ARCHS4 coexpression; STK16 human kinase ARCHS4 coexpression; SCYL3 human kinase ARCHS4 coexpression; MARK1 human kinase ARCHS4 coexpression; SBK1 human kinase ARCHS4 coexpression; PRKCH human kinase ARCHS4 coexpression; RPS6KA1 human kinase ARCHS4 coexpression; RPS6KA6 human kinase ARCHS4 coexpression; CAMKV human kinase ARCHS4 coexpression; CASK human kinase ARCHS4 coexpression; MAPKAPK3 human kinase ARCHS4 coexpression; ACVR2A human kinase ARCHS4 coexpression; ACVR1B human kinase ARCHS4 coexpression; TAOK1 human kinase ARCHS4 coexpression; STRADB human kinase ARCHS4 coexpression; MAP2K2 human kinase ARCHS4 coexpression; NEK6 human kinase ARCHS4 coexpression; MAPK8 human kinase ARCHS4 coexpression; MAPK7 human kinase ARCHS4 coexpression; MAPK3 human kinase ARCHS4 coexpression; HIPK3 human kinase ARCHS4 coexpression; CLK2 human kinase ARCHS4 coexpression; CDK7 human kinase ARCHS4 coexpression; ACVR2B human kinase ARCHS4 coexpression; LRRK1 human kinase ARCHS4 coexpression; RIPK1 human kinase ARCHS4 coexpression; RIPK3 human kinase ARCHS4 coexpression; TBK1 human kinase ARCHS4 coexpression; ERN1 human kinase ARCHS4 coexpression; AURKB human kinase ARCHS4 coexpression; ZAP70 human kinase ARCHS4 coexpression; ROR2 human kinase ARCHS4 coexpression; LMTK3 human kinase ARCHS4 coexpression; CDK2 human kinase ARCHS4 coexpression; IGFIR human kinase ARCHS4 coexpression; FGR human kinase ARCHS4 coexpression; FGFR4 human kinase ARCHS4 coexpression; EPHB2 human kinase ARCHS4 coexpression; EPHA8 human kinase ARCHS4 coexpression; EGFR human kinase ARCHS4 coexpression; BLK human kinase ARCHS4 coexpression; HCK human kinase ARCHS4 coexpression; VRK2 human kinase ARCHS4 coexpression; SIK2 human kinase ARCHS4 coexpression; NUAK1 human kinase ARCHS4 coexpression; ADCK2 human kinase ARCHS4 coexpression; CAMK2G human kinase ARCHS4 coexpression; RPS6KA4 human kinase ARCHS4 coexpression; GRK4 human kinase ARCHS4 coexpression; ALPK2 human kinase ARCHS4 coexpression; IRAK3 human kinase ARCHS4 coexpression; IRAK4 human kinase ARCHS4 coexpression; BMPR1B human kinase ARCHS4 coexpression; BMPR1A human kinase ARCHS4 coexpression; TESK1 human kinase ARCHS4 coexpression; BMX human kinase ARCHS4 coexpression; LYN human kinase ARCHS4 coexpression; EPHA7 human kinase ARCHS4 coexpression; AAK1 human kinase ARCHS4 coexpression; AURKC human kinase ARCHS4 coexpression; PDIK1L human kinase ARCHS4 coexpression; PEAK1 human kinase ARCHS4 coexpression; PLK1 human kinase ARCHS4 coexpression; RPS6KL1 human kinase ARCHS4 coexpression; TLK1 human kinase ARCHS4 coexpression; WNK2 human kinase ARCHS4 coexpression; ADCK1 human kinase ARCHS4 coexpression; EPHA3 human kinase ARCHS4 coexpression; ALPK1 human kinase ARCHS4 coexpression; TEC human kinase ARCHS4 coexpression; PTK2B human kinase ARCHS4 coexpression; MST1R human kinase ARCHS4 coexpression; INSR human kinase ARCHS4 coexpression; FGFR1 human kinase ARCHS4 coexpression; FER human kinase ARCHS4 coexpression; ERBB4 human kinase ARCHS4 coexpression; EPHB1 human kinase ARCHS4 coexpression; EEF2K human kinase ARCHS4 coexpression; MAP4K5 human kinase ARCHS4 coexpression; MAP4K4 human kinase ARCHS4 coexpression; MAP3K19 human kinase ARCHS4 coexpression; NEK11 human kinase ARCHS4 coexpression; MAPK15 human kinase ARCHS4 coexpression; MAPK11 human kinase ARCHS4 coexpression; CSNK1G1 human kinase ARCHS4 coexpression; CSNK1D human kinase ARCHS4 coexpression; STK33 human kinase ARCHS4 coexpression; OBSCN human kinase ARCHS4 coexpression; MYLK3 human kinase ARCHS4 coexpression; MYLK2 human kinase ARCHS4 coexpression; MARK4 human kinase ARCHS4 coexpression; DCLK2 human kinase ARCHS4 coexpression; PRKX human kinase ARCHS4 coexpression; GRK1 human kinase ARCHS4 coexpression; TNK2 human kinase ARCHS4 coexpression; TNK1 human kinase ARCHS4 coexpression; FRK human kinase ARCHS4 coexpression; INSRR human kinase ARCHS4 coexpression; FLT3 human kinase ARCHS4 coexpression; ERBB3 human kinase ARCHS4 coexpression; EPHB3 human kinase ARCHS4 coexpression; EPHA4 human kinase ARCHS4 coexpression; IRAK1 human kinase ARCHS4 coexpression; AMHR2 human kinase ARCHS4 coexpression; TAOK3 human kinase ARCHS4 coexpression; STRADA human kinase ARCHS4 coexpression; STK3 human kinase ARCHS4 coexpression; MAP4K2 human kinase ARCHS4 coexpression; MAP3K13 human kinase ARCHS4 coexpression; NEK5 human kinase ARCHS4 coexpression; NEK10 human kinase ARCHS4 coexpression; MAPK13 human kinase ARCHS4 coexpression; GSK3A human kinase ARCHS4 coexpression; DYRK1B human kinase ARCHS4 coexpression; NTRK2 human kinase ARCHS4 coexpression; PTK7 human kinase ARCHS4 coexpression; TEK human kinase ARCHS4 coexpression; TXK human kinase ARCHS4 coexpression; MTOR human kinase ARCHS4 coexpression; PDK3 human kinase ARCHS4 coexpression; PDK2 human kinase ARCHS4 coexpression; PDK1 human kinase ARCHS4 coexpression; FASTK human kinase ARCHS4 coexpression; ULK3 human kinase ARCHS4 coexpression; TP53RK human kinase ARCHS4 coexpression; TBCK human kinase ARCHS4 coexpression; CLK4 human kinase ARCHS4 coexpression; SGK494 human kinase ARCHS4 coexpression; PXK human kinase ARCHS4 coexpression; PKDCC human kinase ARCHS4 coexpression; PAN3 human kinase ARCHS4 coexpression; NRBP2 human kinase ARCHS4 coexpression; IKBKB human kinase ARCHS4 coexpression; ERN2 human kinase ARCHS4 coexpression; EIF2AK4 human kinase ARCHS4 coexpression; DSTYK human kinase ARCHS4 coexpression; SCYL1 human kinase ARCHS4 coexpression; CDK10 human kinase ARCHS4 coexpression; CDKL1 human kinase ARCHS4 coexpression; TRIB3 human kinase ARCHS4 coexpression; TRIO human kinase ARCHS4 coexpression; GRK5 human kinase ARCHS4 coexpression; PDPK2P human kinase ARCHS4 coexpression; PKN1 human kinase ARCHS4 coexpression; PRKY human kinase ARCHS4 coexpression; PRKG1 human kinase ARCHS4 coexpression; STK38 human kinase ARCHS4 coexpression; DAPK3 human kinase ARCHS4 coexpression; HUNK human kinase ARCHS4 coexpression; PRKAA2 human kinase ARCHS4 coexpression; SPEG human kinase ARCHS4 coexpression; RPS6KB1 human kinase ARCHS4 coexpression; EIF2AK1 human kinase ARCHS4 coexpression; MAP2K6 human kinase ARCHS4 coexpression; TSSK3 human kinase ARCHS4 coexpression; STK17A human kinase ARCHS4 coexpression; PASK human kinase ARCHS4 coexpression; MKNK1 human kinase ARCHS4 coexpression; DAPK1 human kinase ARCHS4 coexpression; CAMK1 human kinase ARCHS4 coexpression; SGK3 human kinase ARCHS4 coexpression; SGK2 human kinase ARCHS4 coexpression; CSNKIAIL human kinase ARCHS4 coexpression; RPS6KB2 human kinase ARCHS4 coexpression; RPS6KA2 human kinase ARCHS4 coexpression; MAST4 human kinase ARCHS4 coexpression; MAST2 human kinase ARCHS4 coexpression; AKT2 human kinase ARCHS4 coexpression; AKT1 human kinase ARCHS4 coexpression; PKMYT1 human kinase ARCHS4 coexpression; POMK human kinase ARCHS4 coexpression; STKLD1 human kinase ARCHS4 coexpression; RPS6KA5 human kinase ARCHS4 coexpression; ALPK3 human kinase ARCHS4 coexpression; CSNK1E human kinase ARCHS4 coexpression; CDK5 human kinase ARCHS4 coexpression; TIE1 human kinase ARCHS4 coexpression; SYK human kinase ARCHS4 coexpression; PTK6 human kinase ARCHS4 coexpression; JAK3 human kinase ARCHS4 coexpression; FLT4 human kinase ARCHS4 coexpression; FES human kinase ARCHS4 coexpression; DDR1 human kinase ARCHS4 coexpression; CSF1R human kinase ARCHS4 coexpression; VRK3 human kinase ARCHS4 coexpression; BTK human kinase ARCHS4 coexpression; LIMK1 human kinase ARCHS4 coexpression; ANKK1 human kinase ARCHS4 coexpression; MYO3B human kinase ARCHS4 coexpression; IKBKE human kinase ARCHS4 coexpression; NEK8 human kinase ARCHS4 coexpression; NEK1 human kinase ARCHS4 coexpression; SRPK3 human kinase ARCHS4 coexpression; CDKL4 human kinase ARCHS4 coexpression; ABL1 human kinase ARCHS4 coexpression; PIK3CG human kinase ARCHS4 coexpression; CDK4 human kinase ARCHS4 coexpression; DAPK2 human kinase ARCHS4 coexpression; MINK1 human kinase ARCHS4 coexpression; MAP4K1 human kinase ARCHS4 coexpression; MAP3K6 human kinase ARCHS4 coexpression; MAP3K15 human kinase ARCHS4 coexpression; MOK human kinase ARCHS4 coexpression; MAPK12 human kinase ARCHS4 coexpression; DYRK4 human kinase ARCHS4 coexpression; CLK3 human kinase ARCHS4 coexpression; CDKL3 human kinase ARCHS4 coexpression; CDK11A human kinase ARCHS4 coexpression; TTN human kinase ARCHS4 coexpression; TSSK6 human kinase ARCHS4 coexpression; TSSK2 human kinase ARCHS4 coexpression; TSSK1B human kinase ARCHS4 coexpression; PSKH1 human kinase ARCHS4 coexpression; PRKD1 human kinase ARCHS4 coexpression; PHKG1 human kinase ARCHS4 coexpression; PKN3 human kinase ARCHS4 coexpression; PRKCQ human kinase ARCHS4 coexpression; PRKG2 human kinase ARCHS4 coexpression; STK25 human kinase ARCHS4 coexpression; MYLK4 human kinase ARCHS4 coexpression; TAOK2 human kinase ARCHS4 coexpression; ERBB2 human kinase ARCHS4 coexpression; ADCK5 human kinase ARCHS4 coexpression; EPHB4 human kinase ARCHS4 coexpression; TEX14 human kinase ARCHS4 coexpression; STK32C human kinase ARCHS4 coexpression; SBK2 human kinase ARCHS4 coexpression; WNK4 human kinase ARCHS4 coexpression; MLKL human kinase ARCHS4 coexpression; MERTK human kinase ARCHS4 coexpression; MATK human kinase ARCHS4 coexpression; FGFR2 human kinase ARCHS4 coexpression; NRBP1 human kinase ARCHS4 coexpression; BCKDK human kinase ARCHS4 coexpression; TRPM6 human kinase ARCHS4 coexpression; ULK4 human kinase ARCHS4 coexpression; CSNK2A3 human kinase ARCHS4 coexpression; LCK human kinase ARCHS4 coexpression; MAK human kinase ARCHS4 coexpression; CDK16 human kinase ARCHS4 coexpression; PHKG2 human kinase ARCHS4 coexpression; DMPK human kinase ARCHS4 coexpression; CDC42BPB human kinase ARCHS4 coexpression

### Downregulated Kinase Activity in GSE88773 by Combined Score

EPHB3; STK17A; TP53RK; GRK4; NEK7; CDK20; MAPK15; TAF1; SMG1; VRK2; VRK1; GRK5; TAOK2; ICK; NEK9; NUAK1; HIPK2; ADRBK1; IKBKB; PRKAA2; ADRBK2; MAPKAPK5; GRK6; GRK1; PLK3; MAP2K6; RPS6KB1; CDK7; FER; DYRK1B; MAP2K2; CHUK; JAK3; MELK; DAPK1; STK11; CSNK1A1L; IKBKE; CSNK1G3; STK4; SGK2; CDC42BPA; TBK1; EIF2AK2; MAP3K5; CDK9; CSNK1G1; PRKCH; DYRK2; CSNK1G2; SGK494; SGK3; SGK223; CDK3; GSK3A; JAK2; ATR; DAPK3; CSNK1A1; IGF1R; MAPK3; PAK2; RET; MAPK1; MAPK7; PRKD1; BTK; PIM1; DYRK1A; PRKACA; PRKCD; MAP3K8; MAP2K1; MAPKAPK2; MTOR; PRKAA1; CHEK2; SRC; ROCK2; LCK; CDK5; PRKACG; CAMK2D; CSNK2A2; PDK1; MAPK4; ROCK1; PRKCA; AURKA; CDK15; CDK18; CDK11A; PRKCB; CSNK1E; HCK; MAPK9; CDK14; PRKDC; ABL1; MAPK10; GSK3B; AKT1; ATM; PRKACB; INSR; FYN; MAPK8; RPS6KA1; CSNK2A1; SGK1; MAPK14; CSNK1D; CHEK1; AURKB; LYN; RPS6KA3; CDK2; CDK1; IKBKB human kinase ARCHS4 coexpression; STK17B human kinase ARCHS4 coexpression; EEF2K human kinase ARCHS4 coexpression; PLK2 human kinase ARCHS4 coexpression; TXK human kinase ARCHS4 coexpression; FGFR2 human kinase ARCHS4 coexpression; STK4 human kinase ARCHS4 coexpression; MAP4K1 human kinase ARCHS4 coexpression; CLK1 human kinase ARCHS4 coexpression; STK17A human kinase ARCHS4 coexpression; PRKD2 human kinase ARCHS4 coexpression; PRKCQ human kinase ARCHS4 coexpression; PRKCD human kinase ARCHS4 coexpression; PRKD1 human kinase ARCHS4 coexpression; RIPK1 human kinase ARCHS4 coexpression; CSFIR human kinase ARCHS4 coexpression; IKBKE human kinase ARCHS4 coexpression; TESK2 human kinase ARCHS4 coexpression; GRK2 human kinase ARCHS4 coexpression; ITK human kinase ARCHS4 coexpression; PTK2B human kinase ARCHS4 coexpression; PDK3 human kinase ARCHS4 coexpression; STK36 human kinase ARCHS4 coexpression; ERN1 human kinase ARCHS4 coexpression; JAK1 human kinase ARCHS4 coexpression; MLKL human kinase ARCHS4 coexpression; PRKCH human kinase ARCHS4 coexpression; FYN human kinase ARCHS4 coexpression; CSK human kinase ARCHS4 coexpression; NRK human kinase ARCHS4 coexpression; GRK6 human kinase ARCHS4 coexpression; IRAK4 human kinase ARCHS4 coexpression; MYLK human kinase ARCHS4 coexpression; NUAK2 human kinase ARCHS4 coexpression; PIM1 human kinase ARCHS4 coexpression; TRIB1 human kinase ARCHS4 coexpression; SNRK human kinase ARCHS4 coexpression; MAPK7 human kinase ARCHS4 coexpression; NEK9 human kinase ARCHS4 coexpression; MAP3K8 human kinase ARCHS4 coexpression; MAPK12 human kinase ARCHS4 coexpression; RIPK3 human kinase ARCHS4 coexpression; TGFBR2 human kinase ARCHS4 coexpression; EPHA7 human kinase ARCHS4 coexpression; EPHB2 human kinase ARCHS4 coexpression; FES human kinase ARCHS4 coexpression; LYN human kinase ARCHS4 coexpression; LTK human kinase ARCHS4 coexpression; PDGFRA human kinase ARCHS4 coexpression; PLK3 human kinase ARCHS4 coexpression; ALPK3 human kinase ARCHS4 coexpression; ATM human kinase ARCHS4 coexpression; IGF1R human kinase ARCHS4 coexpression; MYLK4 human kinase ARCHS4 coexpression; OBSCN human kinase ARCHS4 coexpression; AMHR2 human kinase ARCHS4 coexpression; ACVR1 human kinase ARCHS4 coexpression; MAP4K2 human kinase ARCHS4 coexpression; MAP3K14 human kinase ARCHS4 coexpression; MAP2K5 human kinase ARCHS4 coexpression; STK40 human kinase ARCHS4 coexpression; SIK1 human kinase ARCHS4 coexpression; PRKCB human kinase ARCHS4 coexpression; RPS6KA1 human kinase ARCHS4 coexpression; STK38 human kinase ARCHS4 coexpression; MKNK1 human kinase ARCHS4 coexpression; MYLK2 human kinase ARCHS4 coexpression; TTN human kinase ARCHS4 coexpression; MAP3K5 human kinase ARCHS4 coexpression; MAP3K3 human kinase ARCHS4 coexpression; STRADA human kinase ARCHS4 coexpression; ILK human kinase ARCHS4 coexpression; RAF1 human kinase ARCHS4 coexpression; TNNI3K human kinase ARCHS4 coexpression; BTK human kinase ARCHS4 coexpression; HCK human kinase ARCHS4 coexpression; MAP3K1 human kinase ARCHS4 coexpression; DDR2 human kinase ARCHS4 coexpression; MAP2K3 human kinase ARCHS4 coexpression; SRPK3 human kinase ARCHS4 coexpression; MAPK15 human kinase ARCHS4 coexpression; MAPK14 human kinase ARCHS4 coexpression; CSNK1G2 human kinase ARCHS4 coexpression; TSSK4 human kinase ARCHS4 coexpression; SPEG human kinase ARCHS4 coexpression; GRK3 human kinase ARCHS4 coexpression; FGR human kinase ARCHS4 coexpression; PHKG1 human kinase ARCHS4 coexpression; JAK3 human kinase ARCHS4 coexpression; PRKX human kinase ARCHS4 coexpression; PIK3CG human kinase ARCHS4 coexpression; PDK2 human kinase ARCHS4 coexpression; ALPK2 human kinase ARCHS4 coexpression; ZAP70 human kinase ARCHS4 coexpression; TYK2 human kinase ARCHS4 coexpression; TEK human kinase ARCHS4 coexpression; TEC human kinase ARCHS4 coexpression; SYK human kinase ARCHS4 coexpression; SRC human kinase ARCHS4 coexpression; MAP3K11 human kinase ARCHS4 coexpression; NEK5 human kinase ARCHS4 coexpression; MAPK13 human kinase ARCHS4 coexpression; DYRK2 human kinase ARCHS4 coexpression; PIM3 human kinase ARCHS4 coexpression; PRKAA2 human kinase ARCHS4 coexpression; STRADB human kinase ARCHS4 coexpression; PIM2 human kinase ARCHS4 coexpression; CLK4 human kinase ARCHS4 coexpression; ARAF human kinase ARCHS4 coexpression; LIMK2 human kinase ARCHS4 coexpression; IRAK2 human kinase ARCHS4 coexpression; IRAK3 human kinase ARCHS4 coexpression; MKNK2 human kinase ARCHS4 coexpression; LRRK2 human kinase ARCHS4 coexpression; ABL2 human kinase ARCHS4 coexpression; ERBB3 human kinase ARCHS4 coexpression; FGFR1 human kinase ARCHS4 coexpression; JAK2 human kinase ARCHS4 coexpression; MERTK human kinase ARCHS4 coexpression; PDGFRB human kinase ARCHS4 coexpression; BRAF human kinase ARCHS4 coexpression; MAPKAPK3 human kinase ARCHS4 coexpression; MAST3 human kinase ARCHS4 coexpression; SGK1 human kinase ARCHS4 coexpression; DMPK human kinase ARCHS4 coexpression; LATS2 human kinase ARCHS4 coexpression; MOS human kinase ARCHS4 coexpression; PRKG1 human kinase ARCHS4 coexpression; CASK human kinase ARCHS4 coexpression; PXK human kinase ARCHS4 coexpression; CDK17 human kinase ARCHS4 coexpression; MARK3 human kinase ARCHS4 coexpression; MARK2 human kinase ARCHS4 coexpression; PDK4 human kinase ARCHS4 coexpression; GAK human kinase ARCHS4 coexpression; TIE1 human kinase ARCHS4 coexpression; ROR2 human kinase ARCHS4 coexpression; RET human kinase ARCHS4 coexpression; FASTK human kinase ARCHS4 coexpression; BCKDK human kinase ARCHS4 coexpression; RIOK3 human kinase ARCHS4 coexpression; MATK human kinase ARCHS4 coexpression; LCK human kinase ARCHS4 coexpression; FLT4 human kinase ARCHS4 coexpression; FLT1 human kinase ARCHS4 coexpression; FGFR4 human kinase ARCHS4 coexpression; EPHB3 human kinase ARCHS4 coexpression; SCYL3 human kinase ARCHS4 coexpression; KSR1 human kinase ARCHS4 coexpression; EPHA10 human kinase ARCHS4 coexpression; STK10 human kinase ARCHS4 coexpression; MAPKAPK2 human kinase ARCHS4 coexpression; DAPK1 human kinase ARCHS4 coexpression; RPS6KB1 human kinase ARCHS4 coexpression; PRKACA human kinase ARCHS4 coexpression; MAST2 human kinase ARCHS4 coexpression; GRK5 human kinase ARCHS4 coexpression; GRK1 human kinase ARCHS4 coexpression; ANKK1 human kinase ARCHS4 coexpression; PASK human kinase ARCHS4 coexpression; SIK3 human kinase ARCHS4 coexpression; CDK15 human kinase ARCHS4 coexpression; DYRK1B human kinase ARCHS4 coexpression; ICK human kinase ARCHS4 coexpression; NEK7 human kinase ARCHS4 coexpression; MAP3K19 human kinase ARCHS4 coexpression; WNK4 human kinase ARCHS4 coexpression; RIPK2 human kinase ARCHS4 coexpression; ALK human kinase ARCHS4 coexpression; BLK human kinase ARCHS4 coexpression; BMX human kinase ARCHS4 coexpression; DDR1 human kinase ARCHS4 coexpression; PHKG2 human kinase ARCHS4 coexpression; EPHA1 human kinase ARCHS4 coexpression; EPHA4 human kinase ARCHS4 coexpression; EPHB1 human kinase ARCHS4 coexpression; FLT3 human kinase ARCHS4 coexpression; INSR human kinase ARCHS4 coexpression; ACVRL1 human kinase ARCHS4 coexpression; MYO3A human kinase ARCHS4 coexpression; PTK7 human kinase ARCHS4 coexpression; NEK8 human kinase ARCHS4 coexpression; CAMK2D human kinase ARCHS4 coexpression; PRKACB human kinase ARCHS4 coexpression; PRKY human kinase ARCHS4 coexpression; RPS6KA2 human kinase ARCHS4 coexpression; SGK3 human kinase ARCHS4 coexpression; CAMK1 human kinase ARCHS4 coexpression; NEK6 human kinase ARCHS4 coexpression; NEK10 human kinase ARCHS4 coexpression; DYRK3 human kinase ARCHS4 coexpression; VRK3 human kinase ARCHS4 coexpression; STK11 human kinase ARCHS4 coexpression; MAP3K6 human kinase ARCHS4 coexpression; SRMS human kinase ARCHS4 coexpression; PINK1 human kinase ARCHS4 coexpression; TNK2 human kinase ARCHS4 coexpression; PKDCC human kinase ARCHS4 coexpression; SBK1 human kinase ARCHS4 coexpression; SGK494 human kinase ARCHS4 coexpression; STK16 human kinase ARCHS4 coexpression; WEE2 human kinase ARCHS4 coexpression; STKLD1 human kinase ARCHS4 coexpression; BMPR1B human kinase ARCHS4 coexpression; TAOK3 human kinase ARCHS4 coexpression; ACVR2B human kinase ARCHS4 coexpression; LRRK1 human kinase ARCHS4 coexpression; EPHA2 human kinase ARCHS4 coexpression; EPHA6 human kinase ARCHS4 coexpression; EPHA8 human kinase ARCHS4 coexpression; STK25 human kinase ARCHS4 coexpression; NTRK1 human kinase ARCHS4 coexpression; STYK1 human kinase ARCHS4 coexpression; TNK1 human kinase ARCHS4 coexpression; BMP2K human kinase ARCHS4 coexpression; STK32A human kinase ARCHS4 coexpression; TLK1 human kinase ARCHS4 coexpression; ULK3 human kinase ARCHS4 coexpression; ROR1 human kinase ARCHS4 coexpression; PAK4 human kinase ARCHS4 coexpression; MYO3B human kinase ARCHS4 coexpression; GRK7 human kinase ARCHS4 coexpression; MAST4 human kinase ARCHS4 coexpression; PKN1 human kinase ARCHS4 coexpression; PAK2 human kinase ARCHS4 coexpression; SGK2 human kinase ARCHS4 coexpression; NIM1K human kinase ARCHS4 coexpression; CSNK1A1 human kinase ARCHS4 coexpression; CDKL1 human kinase ARCHS4 coexpression; MAK human kinase ARCHS4 coexpression; SRPK2 human kinase ARCHS4 coexpression; NEK11 human kinase ARCHS4 coexpression; DAPK2 human kinase ARCHS4 coexpression; NUAK1 human kinase ARCHS4 coexpression; PRKC1 human kinase ARCHS4 coexpression; RPS6KA3 human kinase ARCHS4 coexpression; RPS6KA4 human kinase ARCHS4 coexpression; CIT human kinase ARCHS4 coexpression; TRIB3 human kinase ARCHS4 coexpression; RPS6KA6 human kinase ARCHS4 coexpression; CAMK4 human kinase ARCHS4 coexpression; CDK11A human kinase ARCHS4 coexpression; DAPK3 human kinase ARCHS4 coexpression; HUNK human kinase ARCHS4 coexpression; PSKH1 human kinase ARCHS4 coexpression; SIK2 human kinase ARCHS4 coexpression; TTBK2 human kinase ARCHS4 coexpression; CDK3 human kinase ARCHS4 coexpression; CDK9 human kinase ARCHS4 coexpression; CAMKV human kinase ARCHS4 coexpression; CDKL5 human kinase ARCHS4 coexpression; DYRK1A human kinase ARCHS4 coexpression; MAP3K2 human kinase ARCHS4 coexpression; MAPK8 human kinase ARCHS4 coexpression; ADCK1 human kinase ARCHS4 coexpression; ADCK2 human kinase ARCHS4 coexpression; SMG1 human kinase ARCHS4 coexpression; SCYL2 human kinase ARCHS4 coexpression; MAPK10 human kinase ARCHS4 coexpression; SCYL1 human kinase ARCHS4 coexpression; PAN3 human kinase ARCHS4 coexpression; MOK human kinase ARCHS4 coexpression; MAP2K2 human kinase ARCHS4 coexpression; MAP2K4 human kinase ARCHS4 coexpression; MAP2K7 human kinase ARCHS4 coexpression; PAK1 human kinase ARCHS4 coexpression; PAK5 human kinase ARCHS4 coexpression; ACVR1C human kinase ARCHS4 coexpression; ACVR2A human kinase ARCHS4 coexpression; PDIK1L human kinase ARCHS4 coexpression; BMPR1A human kinase ARCHS4 coexpression; AXL human kinase ARCHS4 coexpression; EPHA3 human kinase ARCHS4 coexpression; FER human kinase ARCHS4 coexpression; KDR human kinase ARCHS4 coexpression; ROS1 human kinase ARCHS4 coexpression; AURKC human kinase ARCHS4 coexpression; DSTYK human kinase ARCHS4 coexpression; EIF2AK2 human kinase ARCHS4 coexpression; ABL1 human kinase ARCHS4 coexpression; POMK human kinase ARCHS4 coexpression; CSNK1E human kinase ARCHS4 coexpression; CSNK1G1 human kinase ARCHS4 coexpression; VRK2 human kinase ARCHS4 coexpression; TBCK human kinase ARCHS4 coexpression; ULK1 human kinase ARCHS4 coexpression; ULK4 human kinase ARCHS4 coexpression; WNK2 human kinase ARCHS4 coexpression; WNK3 human kinase ARCHS4 coexpression; CDK18 human kinase ARCHS4 coexpression; CDK19 human kinase ARCHS4 coexpression; CDK20 human kinase ARCHS4 coexpression; CDK4 human kinase ARCHS4 coexpression; CDK6 human kinase ARCHS4 coexpression; GSK3A human kinase ARCHS4 coexpression; HIPK1 human kinase ARCHS4 coexpression; NLK human kinase ARCHS4 coexpression; NEK4 human kinase ARCHS4 coexpression; MAP2K6 human kinase ARCHS4 coexpression; MAP3K13 human kinase ARCHS4 coexpression; MAP3K9 human kinase ARCHS4 coexpression; MAP4K3 human kinase ARCHS4 coexpression; MINK1 human kinase ARCHS4 coexpression; TNIK human kinase ARCHS4 coexpression; TSSK6 human kinase ARCHS4 coexpression; TSSK2 human kinase ARCHS4 coexpression; TSSK1B human kinase ARCHS4 coexpression; STK33 human kinase ARCHS4 coexpression; PLK5 human kinase ARCHS4 coexpression; ERN2 human kinase ARCHS4 coexpression; EIF2AK4 human kinase ARCHS4 coexpression; EIF2AK1 human kinase ARCHS4 coexpression; CSNK2A2 human kinase ARCHS4 coexpression; CAMKK1 human kinase ARCHS4 coexpression; AURKA human kinase ARCHS4 coexpression; AAK1 human kinase ARCHS4 coexpression; PTK2 human kinase ARCHS4 coexpression; KIT human kinase ARCHS4 coexpression; ERBB2 human kinase ARCHS4 coexpression; SBK3 human kinase ARCHS4 coexpression; EPHA5 human kinase ARCHS4 coexpression; TESK1 human kinase ARCHS4 coexpression; RIPK4 human kinase ARCHS4 coexpression; AKT2 human kinase ARCHS4 coexpression; CDC42BPG human kinase ARCHS4 coexpression; PRKCE human kinase ARCHS4 coexpression; ROCK1 human kinase ARCHS4 coexpression; CAMKIG human kinase ARCHS4 coexpression; CAMK2A human kinase ARCHS4 coexpression; CHEK1 human kinase ARCHS4 coexpression; DCLK1 human kinase ARCHS4 coexpression; PNCK human kinase ARCHS4 coexpression; EGFR human kinase ARCHS4 coexpression; EPHB6 human kinase ARCHS4 coexpression; BMPR2 human kinase ARCHS4 coexpression; ACVR1B human kinase ARCHS4 coexpression; TP53RK human kinase ARCHS4 coexpression; SBK2 human kinase ARCHS4 coexpression; STK31 human kinase ARCHS4 coexpression; RPS6KL1 human kinase ARCHS4 coexpression; STK32C human kinase ARCHS4 coexpression; ULK2 human kinase ARCHS4 coexpression; RIOK1 human kinase ARCHS4 coexpression; TRPM7 human kinase ARCHS4 coexpression; PDK1 human kinase ARCHS4 coexpression; PIK3CA human kinase ARCHS4 coexpression; RNASEL human kinase ARCHS4 coexpression; TAOK1 human kinase ARCHS4 coexpression; STK24 human kinase ARCHS4 coexpression; WNK1 human kinase ARCHS4 coexpression; PEAK1 human kinase ARCHS4 coexpression; NTRK3 human kinase ARCHS4 coexpression; CDC7 human kinase ARCHS4 coexpression; SLK human kinase ARCHS4 coexpression; LIMK1 human kinase ARCHS4 coexpression; AATK human kinase ARCHS4 coexpression; EPHB4 human kinase ARCHS4 coexpression; FGFR3 human kinase ARCHS4 coexpression; INSRR human kinase ARCHS4 coexpression; EIF2AK3 human kinase ARCHS4 coexpression; KSR2 human kinase ARCHS4 coexpression; MSTIR human kinase ARCHS4 coexpression; MUSK human kinase ARCHS4 coexpression; RYK human kinase ARCHS4 coexpression; TYRO3 human kinase ARCHS4 coexpression; YES1 human kinase ARCHS4 coexpression; CAMKK2 human kinase ARCHS4 coexpression; MET human kinase ARCHS4 coexpression; PBK human kinase ARCHS4 coexpression; CAMK2B human kinase ARCHS4 coexpression; MAP4K5 human kinase ARCHS4 coexpression; CDK12 human kinase ARCHS4 coexpression; CDK10 human kinase ARCHS4 coexpression; CDK1 human kinase ARCHS4 coexpression; CSNK1D human kinase ARCHS4 coexpression; TRIB2 human kinase ARCHS4 coexpression; PSKH2 human kinase ARCHS4 coexpression; CDK13 human kinase ARCHS4 coexpression; PRKAA1 human kinase ARCHS4 coexpression; MARK4 human kinase ARCHS4 coexpression; MARK1 human kinase ARCHS4 coexpression; MAPKAPK5 human kinase ARCHS4 coexpression; KALRN human kinase ARCHS4 coexpression; DCLK3 human kinase ARCHS4 coexpression; CAMK2G human kinase ARCHS4 coexpression; MYLK3 human kinase ARCHS4 coexpression; PAK3 human kinase ARCHS4 coexpression; RPS6KB2 human kinase ARCHS4 coexpression; PRKCG human kinase ARCHS4 coexpression; MAP2K1 human kinase ARCHS4 coexpression; MAPK9 human kinase ARCHS4 coexpression; MAPK6 human kinase ARCHS4 coexpression; CAMKID human kinase ARCHS4 coexpression; HIPK4 human kinase ARCHS4 coexpression; HIPK3 human kinase ARCHS4 coexpression; RPS6KA5 human kinase ARCHS4 coexpression; CLK3 human kinase ARCHS4 coexpression; AKT3 human kinase ARCHS4 coexpression; CDC42BPA human kinase ARCHS4 coexpression; PDPK1 human kinase ARCHS4 coexpression; PDPK2P human kinase ARCHS4 coexpression; PKN2 human kinase ARCHS4 coexpression; PRKCA human kinase ARCHS4 coexpression; CDK14 human kinase ARCHS4 coexpression; CDKL4 human kinase ARCHS4 coexpression; NEK1 human kinase ARCHS4 coexpression; SRPK1 human kinase ARCHS4 coexpression; PRPF4B human kinase ARCHS4 coexpression; MAPK3 human kinase ARCHS4 coexpression; MAPK1 human kinase ARCHS4 coexpression; HIPK2 human kinase ARCHS4 coexpression; CLK2 human kinase ARCHS4 coexpression; CDKL3 human kinase ARCHS4 coexpression; CDK7 human kinase ARCHS4 coexpression; CDK5 human kinase ARCHS4 coexpression; CDK2 human kinase ARCHS4 coexpression; CDK11B human kinase ARCHS4 coexpression; TTBK1 human kinase ARCHS4 coexpression; CSNK1G3 human kinase ARCHS4 coexpression; CSNK1A1L human kinase ARCHS4 coexpression; TSSK3 human kinase ARCHS4 coexpression; TRIO human kinase ARCHS4 coexpression; MELK human kinase ARCHS4 coexpression; BRSK1 human kinase ARCHS4 coexpression; STK38L human kinase ARCHS4 coexpression; NEK3 human kinase ARCHS4 coexpression; MAP3K15 human kinase ARCHS4 coexpression; MAP3K4 human kinase ARCHS4 coexpression; MAP4K4 human kinase ARCHS4 coexpression; WEE1 human kinase ARCHS4 coexpression; TTK human kinase ARCHS4 coexpression; STK35 human kinase ARCHS4 coexpression; PLK4 human kinase ARCHS4 coexpression; PLK1 human kinase ARCHS4 coexpression; PKMYT1 human kinase ARCHS4 coexpression; NRBP1 human kinase ARCHS4 coexpression; GSG2 human kinase ARCHS4 coexpression; CSNK2A3 human kinase ARCHS4 coexpression; CSNK2A1 human kinase ARCHS4 coexpression; BUB1B human kinase ARCHS4 coexpression; PTK6 human kinase ARCHS4 coexpression; NTRK2 human kinase ARCHS4 coexpression; LMTK2 human kinase ARCHS4 coexpression; FRK human kinase ARCHS4 coexpression; ERBB4 human kinase ARCHS4 coexpression; TGFBR1 human kinase ARCHS4 coexpression; IRAK1 human kinase ARCHS4 coexpression; STK39 human kinase ARCHS4 coexpression; STK3 human kinase ARCHS4 coexpression; PAK6 human kinase ARCHS4 coexpression; ROCK2 human kinase ARCHS4 coexpression; PRKCZ human kinase ARCHS4 coexpression; PKN3 human kinase ARCHS4 coexpression; MASTL human kinase ARCHS4 coexpression; PRKDC human kinase ARCHS4 coexpression; LATS1 human kinase ARCHS4 coexpression; ALPK1 human kinase ARCHS4 coexpression; MTOR human kinase ARCHS4 coexpression; LMTK3 human kinase ARCHS4 coexpression; TAOK2 human kinase ARCHS4 coexpression; STK26 human kinase ARCHS4 coexpression; OXSR1 human kinase ARCHS4 coexpression; MAP3K7 human kinase ARCHS4 coexpression; MAP3K10 human kinase ARCHS4 coexpression; NEK2 human kinase ARCHS4 coexpression; MAPK4 human kinase ARCHS4 coexpression; MAPK11 human kinase ARCHS4 coexpression; TRPM6 human kinase ARCHS4 coexpression; AURKB human kinase ARCHS4 coexpression; BUB1 human kinase ARCHS4 coexpression; PIK3R4 human kinase ARCHS4 coexpression; RPS6KC1 human kinase ARCHS4 coexpression; STK32B human kinase ARCHS4 coexpression; TBK1 human kinase ARCHS4 coexpression; TEX14 human kinase ARCHS4 coexpression; TLK2 human kinase ARCHS4 coexpression; UHMK1 human kinase ARCHS4 coexpression; GSK3B human kinase ARCHS4 coexpression; DYRK4 human kinase ARCHS4 coexpression; CDK8 human kinase ARCHS4 coexpression; ATR human kinase ARCHS4 coexpression; AKT1 human kinase ARCHS4 coexpression; CDC42BPB human kinase ARCHS4 coexpression; GRK4 human kinase ARCHS4 coexpression; MAST1 human kinase ARCHS4 coexpression; BRSK2 human kinase ARCHS4 coexpression; CHEK2 human kinase ARCHS4 coexpression; DCLK2 human kinase ARCHS4 coexpression; PRKD3 human kinase ARCHS4 coexpression; VRK1 human kinase ARCHS4 coexpression; CDKL2 human kinase ARCHS4 coexpression; RIOK2 human kinase ARCHS4 coexpression.

### Upregulated Transcription Factor Target Activity in GSE88773 by Combined Score

NR4A2 human tf ARCHS4 coexpression; GZF1 human tf ARCHS4 coexpression; FOSB human tf ARCHS4 coexpression; JUN human tf ARCHS4 coexpression; CREB1 23762244 ChIP-Seq HIPPOCAMPUS Rat; CREM 20920259 ChIP-Seq GC1-SPG Mouse; CREB1 20920259 ChIP-Seq GC1-SPG Mouse; CHD1 human tf ARCHS4 coexpression; ZNF800 human tf ARCHS4 coexpression; KLF4 human tf ARCHS4 coexpression; ETV3 human tf ARCHS4 coexpression; CREB1 K562 hg19; TIPARP human tf ARCHS4 coexpression; KLF10 human tf ARCHS4 coexpression; NR4A3 human tf ARCHS4 coexpression; HIFIA human tf ARCHS4 coexpression; AHR human tf ARCHS4 coexpression; KDM2B 26808549 Chip-Seq K562 Human; SUZ12 20075857 ChIP-Seq MESCs Mouse; CREB1 ECC-1 hg19; SUZ12 18555785 ChIP-Seq MESCs Mouse; KDM2B 26808549 Chip-Seq SUP—B15 Human; GLIS3 human tf ARCHS4 coexpression; ATF3 human tf ARCHS4 coexpression; CLOCK 20551151 ChIP-Seq 293T Human; JUN HepG2 hg19; RARB 27405468 Chip-Seq BRAIN Mouse; ZFP91 human tf ARCHS4 coexpression; MEF2D human tf ARCHS4 coexpression; ZNF267 human tf ARCHS4 coexpression; ZBTB38 human tf ARCHS4 coexpression; NR4A1 human tf ARCHS4 coexpression; PRDM4 human tf ARCHS4 coexpression; ZBTB1 human tf ARCHS4 coexpression; KLF6 26769127 Chip-Seq PDAC-Cell line Human; CHD1 IMR-90 hg19; MTF2 20144788 ChIP-Seq MESCs Mouse; SA1 22415368 ChIP-Seq MEFs Mouse; KDM2B 26808549 Chip-Seq SIL-ALL Human; KDM2B 26808549 Chip-Seq JURKAT Human; PAX6 human tf ARCHS4 coexpression; KLF6 human tf ARCHS4 coexpression; SP3 human tf ARCHS4 coexpression; ZBTB24 human tf ARCHS4 coexpression; NKX2-2 human tf ARCHS4 coexpression; ZNF331 human tf ARCHS4 coexpression; TCF3 myocyte mm9; KDM2B 26808549 Chip-Seq HPB-ALL Human; RINGIB 27294783 Chip-Seq NPCs Mouse; WTI 20215353 ChIP-ChIP NEPHRON PROGENITOR Mouse; SUZ12 18974828 ChIP-Seq MESCs Mouse; SMAD4 21799915 ChIP-Seq A2780 Human; SUZ12 18692474 ChIP-Seq MESCs Mouse; ZNF217 24962896 ChIP-Seq MCF-7 Human; GATA3 SK—N—SH hg19; SREBF1 HepG2 hg19; SUZ12 18692474 ChIP-Seq MEFs Mouse; CJUN 26792858 Chip-Seq BT549 Human; KDM2B 26808549 Chip-Seq DND41 Human; EGR4 human tf ARCHS4 coexpression; MYSM1 human tf ARCHS4 coexpression; RBM27 human tf ARCHS4 coexpression; IRX2 human tf ARCHS4 coexpression; JUND human tf ARCHS4 coexpression; MET human tf ARCHS4 coexpression; NR3C1 human tf ARCHS4 coexpression; NROB1 human tf ARCHS4 coexpression; PRB4 human tf ARCHS4 coexpression; CEBPD 21427703 ChIP-Seq 3T3-L1 Mouse; PPARD 21283829 ChIP-Seq MYOFIBROBLAST Human; ETS1 MEL cell line mm9; TCF7 22412390 ChIP-Seq EML Mouse; KLF4 26769127 Chip-Seq PDAC-Cell line Human; PIAS1 25552417 ChIP-Seq VCAP Human; WT1 19549856 ChIP-ChIP CCG9911 Human; NR3C1 23031785 ChIP-Seq PC12 Mouse; JUN H1-hESC hg19; FOSL1 K562 hg19; NFIC SK—N—SH hg19; TP53 18474530 ChIP-ChIP U2OS Human; JUND 26020271 ChIP-Seq SMOOTH MUSCLE Human; E2F1 HeLa—S3 hg19; KCMF1 human tf ARCHS4 coexpression; PRB3 human tf ARCHS4 coexpression; CUL4B human tf ARCHS4 coexpression; SMAD7 human tf ARCHS4 coexpression; EGR1 human tf ARCHS4 coexpression; EGR2 human tf ARCHS4 coexpression; RBAK human tf ARCHS4 coexpression; ZNF281 human tf ARCHS4 coexpression; FOSL1 human tf ARCHS4 coexpression; ZMAT4 human tf ARCHS4 coexpression; RELA 24523406 ChIP-Seq FIBROSARCOMA Human; OLIG2 23332759 ChIP-Seq OLIGODENDROCYTES Mouse; RFX5 SK—N—SH hg19; RUNX1 21571218 ChIP-Seq MEGAKARYOCYTES Human; VDR 24763502 ChIP-Seq THP-1 Human; GATA3 27048872 Chip-Seq THYMUS Human; E2F4 myocyte mm9; FLI1 21867929 ChIP-Seq TH2 Mouse; SMRT Chip-Seq Bcells Human; 27268052 SMARCA4 23332759 ChIP-Seq OLIGODENDROCYTES Mouse; NOTCH1 21737748 ChIP-Seq TLL Human; UBTF MEL cell line mm9; REST myocyte mm9; SMC3 SK—N—SH hg19; JUN 26020271 ChIP-Seq SMOOTH MUSCLE Human; FOSL2 SK—N—SH hg19; TCF3 18467660 ChIP-ChIP MESCs Mouse; RNF2 18974828 ChIP-Seq MESCs Mouse; EZH2 18974828 ChIP-Seq MESCs Mouse; NKX6-1 human tf ARCHS4 coexpression; ZFP36 human tf ARCHS4 coexpression; RFX6 human tf ARCHS4 coexpression; RGS7 human tf ARCHS4 coexpression; ZC3H11A human tf ARCHS4 coexpression; ZXDB human tf ARCHS4 coexpression; ZNF317 human tf ARCHS4 coexpression; DBX1 human tf ARCHS4 coexpression; JUNB human tf ARCHS4 coexpression; ATF6 human tf ARCHS4 coexpression; PRDM2 human tf ARCHS4 coexpression; NFE2L3 human tf ARCHS4 coexpression; ARNTL2 human tf ARCHS4 coexpression; KLF3 human tf ARCHS4 coexpression; CTBP2 H1-hESC hg19; RACK7 27058665 Chip-Seq MCF-7 Human; CTCF 27219007 Chip-Seq ERYTHROID Human; JUN K562 hg19; MITF 21258399 ChIP-Seq MELANOMA Human; WT1 25993318 ChIP-Seq PODOCYTE Human; JUND MEL cell line mm9; E2F4 21247883 ChIP-Seq LYMPHOBLASTOID Human; EGR1 HCT116 hg19; POLR2A liver mm9; CEBPB 21427703 ChIP-Seq 3T3-L1 Mouse; CEBPB IMR-90 hg19; SMARCB1 HeLa—S3 hg19; JUND HeLa—S3 hg19; REST HepG2 hg19; JUND HepG2 hg19; EGR1 19374776 ChIP-ChIP THP-1 Human; FOXO3 23340844 ChIP-Seq DLD1 Human; JUN endothelial cell of umbilical vein hg19; EZH2 27294783 Chip-Seq ESCs Mouse; NCOR 22465074 ChIP-Seq MACROPHAGES Mouse; SMC1 22415368 ChIP-Seq MEFs Mouse; SUZ12 27294783 Chip-Seq ESCs Mouse; TAL1 20566737 ChIP-Seq PRIMARY FETAL LIVER ERYTHROID Mouse; KDM5B 21448134 ChIP-Seq MESCs Mouse; JUN HeLa—S3 hg19; ATF3 K562 hg19; PPARG 20887899 ChIP-Seq 3T3-L1 Mouse; RCOR1 human tf ARCHS4 coexpression; KLF9 human tf ARCHS4 coexpression; EN2 human tf ARCHS4 coexpression; MKX human tf ARCHS4 coexpression; ZNF697 human tf ARCHS4 coexpression; ZNF365 human tf ARCHS4 coexpression; SATB1 human tf ARCHS4 coexpression; ZEB2 human tf ARCHS4 coexpression; SMAD3 human tf ARCHS4 coexpression; ELK4 human tf ARCHS4 coexpression; BACH1 human tf ARCHS4 coexpression; ISL1 human tf ARCHS4 coexpression; JAZF1 human tf ARCHS4 coexpression; HIVEP1 human tf ARCHS4 coexpression; NFKB1 human tf ARCHS4 coexpression; ELK3 human tf ARCHS4 coexpression; BCLAF1 human tf ARCHS4 coexpression; AHCTF1 human tf ARCHS4 coexpression; ZXDA human tf ARCHS4 coexpression; NCOA2 human tf ARCHS4 coexpression; AFF1 human tf ARCHS4 coexpression; RREB1 human tf ARCHS4 coexpression; LARP4 human tf ARCHS4 coexpression; MBNL1 human tf ARCHS4 coexpression; SLC4A10 human tf ARCHS4 coexpression; CHD2 human tf ARCHS4 coexpression; POLR2A bone marrow macrophage mm9; SOX2 27498859 Chip-Seq STOMACH Mouse; CEBPB HepG2 hg19; CREB1 HepG2 hg19; TEAD4 SK—N—SH hg19; ATF3 23680149 ChIP-Seq GBM1-GSC Human; CEBPB ECC-1 hg19; AR 22383394 ChIP-Seq PROSTATE CANCER Human; TAF7 H1-hESC hg19; CREB1 H1-hESC hg19; PRDM5 23873026 ChIP-Seq MEFs Mouse; SMARCCI HeLa—S3 hg19; POLR2A olfactory bulb mm9; E2F4 K562 hg19; SMRT 22465074 ChIP-Seq MACROPHAGES Mouse; ZFP281 27345836 Chip-Seq ESCs Mouse; P300 19829295 ChIP-Seq ESCs Human; CEBPB A549 hg19; TEAD4 A549 hg19; PKCTHETA 26484144 Chip-Seq BREAST Human; RAD21 SK—N—SH hg19; CTCF SK—N—SH hg19; YAP1 20516196 ChIP-Seq MESCs Mouse; CEBPB 26923725 Chip-Seq MESODERM Mouse; RNF2 27304074 Chip-Seq ESCs Mouse; JUND SK—N—SH hg19; EZH2 B cell hg19; CTCF C2C12 mm9; RING1B 27294783 Chip-Seq ESCs Mouse; ELF3 26769127 Chip-Seq PDAC-Cell line Human; GATA3 24758297 ChIP-Seq MCF-7 Human; SMC3 22415368 ChIP-Seq MEFs Mouse; ELK3 25401928 ChIP-Seq HUVEC Human; RUNX2 24764292 ChIP-Seq MC3T3 Mouse; CHD1 CH12.LX mm9; P300 27058665 Chip-Seq ZR-75-30cells Human; ZNF263 K562 hg19; DMRT1 23473982 ChIP-Seq TESTES Mouse; TAF1 H1-hESC hg19; ARNT 22903824 ChIP-Seq MCF-7 Human; REST PFSK-1 hg19; SALL4 18804426 ChIP-ChIP MESCs Mouse; POLR2A endothelial cell of umbilical vein hg19; TEAD4 ECC-1 hg19; AR 21909140 ChIP-Seq LNCAP Human; MYODI myocyte mm9; BRD4 25478319 ChIP-Seq HGPS Human; JARID2 20075857 ChIP-Seq MESCs Mouse; HAND2 human tf ARCHS4 coexpression; FOXJ2 human tf ARCHS4 coexpression; REST human tf ARCHS4 coexpression; EZH2 human tf ARCHS4 coexpression; SNAIL human tf ARCHS4 coexpression; PLAGL2 human tf ARCHS4 coexpression; BCL11B human tf ARCHS4 coexpression; IRF4 human tf ARCHS4 coexpression; CEBPG human tf ARCHS4 coexpression; RUNX1 human tf ARCHS4 coexpression; FOS human tf ARCHS4 coexpression; ZNF395 human tf ARCHS4 coexpression; FOSL2 human tf ARCHS4 coexpression; LIN28B human tf ARCHS4 coexpression; ATMIN human tf ARCHS4 coexpression; MBNL2 human tf ARCHS4 coexpression; SMARCA1 human tf ARCHS4 coexpression; DEPDC1 human tf ARCHS4 coexpression; TERF1 human tf ARCHS4 coexpression; AFF4 human tf ARCHS4 coexpression; ZBTB10 human tf ARCHS4 coexpression; ZMAT3 human tf ARCHS4 coexpression; HLF human tf ARCHS4 coexpression; ADNP2 human tf ARCHS4 coexpression; ZNF207 human tf ARCHS4 coexpression; PAX5 GM12892 hg19; TAF15 26573619 Chip-Seq HEK293 Human; FOXP2 21765815 ChIP-ChIP NEURO2A Mouse; EZH2 27304074 Chip-Seq ESCs Mouse; RAD21 GM12878 hg19; BHLHE40 MEL cell line mm9; TCF7L2 HEK293 hg19; BCL6 25482012 ChIP-Seq CML-JURL-MK1 Human; E2A 27217539 Chip-Seq RAMOS-Cell line Human; TP63 23658742 ChIP-Seq EP156T Human; CEBPB MCF-7 hg19; ATF2 H1-hESC hg19; SCL 19346495 ChIP-Seq HPC-7 Human; SUZ12 NT2-D1 hg19; TCF3 GM12878 hg19; PPAR 26484153 Chip-Seq NCI-H1993 Human; E2F6 K562 hg19; JUND GM12878 hg19; RAD21 A549 hg19; PRDM14 20953172 ChIP-Seq ESCs Human; ATF3 27146783 Chip-Seq COLON Human; CREB1 26743006 Chip-Seq LNCaP-abl Human; POLR2A small intestine mm9; POLR2A kidney mm9; IRF8 27001747 Chip-Seq BMDM Mouse; DACH1 20351289 ChIP-Seq MDA-MB-231 Human; PHC1 16625203 ChIP-ChIP MESCs Mouse; FOXO1 23066095 ChIP-Seq LIVER Mouse; CTNNB1 20460455 ChIP-Seq HCT116 Human; CTBP2 25329375 ChIP-Seq LNCAP Human; NR3C1 A549 hg19; TET1 21490601 ChIP-Seq MESCs Mouse; HIFIA 21447827 ChIP-Seq MCF-7 Human; EGR1 ECC-1 hg19; ZEBI GM12878 hg19; SOX2 21211035 ChIP-Seq LN229 Gbm; UBF1/2 26484160 Chip-Seq FIBROBLAST Human; UBF1/2 26484160 Chip-Seq HMEC-DERIVED Human; CTCF embryonic fibroblast mm9; POLR2A HCT116 hg19; RFX5 H1-hESC hg19; CREB1 26743006 Chip-Seq LNCaP Human; OCT1 27270436 Chip-Seq PROSTATE Human; ATF1 K562 hg19; REST neural cell hg19; REST HCT116 hg19; TRIM28 21343339 ChIP-Seq HEK293 Human; CEBPB K562 hg19; ZHX1 human tf ARCHS4 coexpression; ETS2 human tf ARCHS4 coexpression; ZHX2 human tf ARCHS4 coexpression; FOXO4 human tf ARCHS4 coexpression; SP1 human tf ARCHS4 coexpression; RORB human tf ARCHS4 coexpression; MIER3 human tf ARCHS4 coexpression; INSM1 human tf ARCHS4 coexpression; NEUROD1 human tf ARCHS4 coexpression; SKIL human tf ARCHS4 coexpression; THRB human tf ARCHS4 coexpression; ZIC1 human tf ARCHS4 coexpression; ZNF589 human tf ARCHS4 coexpression; EBF3 human tf ARCHS4 coexpression; ZNF318 human tf ARCHS4 coexpression; ARX human tf ARCHS4 coexpression; TFAP2B human tf ARCHS4 coexpression; CREM human tf ARCHS4 coexpression; SUZ12 human tf ARCHS4 coexpression; NOC3L human tf ARCHS4 coexpression; E2F7 human tf ARCHS4 coexpression; ATXN7 human tf ARCHS4 coexpression; TOX2 human tf ARCHS4 coexpression; SIM1 human tf ARCHS4 coexpression; ZBTB2 human tf ARCHS4 coexpression; CTCF human tf ARCHS4 coexpression; ZFP42 human tf ARCHS4 coexpression; FOXC2 human tf ARCHS4 coexpression; NFIL3 human tf ARCHS4 coexpression; SALL4 18804426 ChIP-ChIP XEN Mouse; SIN3A HCT116 hg19; KLFI 20508144 ChIP-Seq FETAL-LIVER-ERYTHROID Mouse; RFX5 IMR-90 hg19; STAT3 24763339 ChIP-Seq IMN-ESCs Mouse; HA-E2F1 HeLa—S3 hg19; eGFP-FOS K562 hg19; NFYA HeLa—S3 hg19; FOS endothelial cell of umbilical vein hg19; POLR2AphosphoS2 GM12878 hg19; CTCF small intestine mm9; GTF2B K562 hg19; FOXO1 25302145 ChIP-Seq T-LYMPHOCYTE Mouse; TCF21 26020271 ChIP-Seq SMOOTH MUSCLE Human; TAL1 21186366 ChIP-Seq BM-HSCs Mouse; PU 27001747 Chip-Seq BMDM Mouse; LXR 22158963 ChIP-Seq LIVER Mouse; GATA1 21571218 ChIP-Seq MEGAKARYOCYTES Human; REST HeLa—S3 hg19; FOXM1 26100407 CHIP-SEQ Hek293 flp-in Human; NFIB 24661679 ChIP-Seq LUNG Mouse; ELF5 23300383 ChIP-Seq T47D Human; THAPI K562 hg19; STAT3 23295773 ChIP-Seq U87 Human; EOMES 21245162 ChIP-Seq HESCs Human; REST U-87 MG hg19; GATA3 MCF-7 hg19; EGR1 MCF-7 hg19; RUNX1 27514584 Chip-Seq MCF-7 Human; MAF 26560356 Chip-Seq TH2 Human; TBX20 22328084 ChIP-Seq HEART Mouse; TBX20 22080862 ChIP-Seq HEART Mouse; CTCF 21964334 Chip-Seq Bcells Human; SMAD2/3 21741376 ChIP-Seq EPCs Human; ZNF384 CH12.LX mm9; CEBPB C2C12 mm9; SREBF2 GM12878 hg19; KAT2A HeLa—S3 hg19; SOX9 24532713 ChIP-Seq HFSC Mouse; CEBPD HepG2 hg19; REST Panc1 hg19; JARID2 20064375 ChIP-Seq MESCs Mouse; CHD1 MEL cell line mm9; POLR2A HepG2 hg19; BMI1 23680149 ChIP-Seq NPCS Mouse; TRIM28 19339689 ChIP-ChIP MESCs Mouse; NR1H3 23393188 ChIP-Seq ATHEROSCLEROTIC-FOAM Human; KLF4 19030024 ChIP-ChIP MESCs Mouse; Nerf2 26677805 Chip-Seq MACROPHAGESS Mouse; EGR1 20690147 ChIP-Seq ERYTHROLEUKEMIA Human; PAX3-FKHR 20663909 ChIP-Seq RHABDOMYOSARCOMA Human; IKZF1 GM12878 hg19; UBF1/2 26484160 Chip-Seq HMECs Human; TCF4 22108803 ChIP-Seq LS180 Human; CTCF 21964334 ChIP-Seq BJAB-B Human; NF1 21473784 ChIP-Seq ESCs Mouse; FOXA1 25552417 ChIP-Seq VCAP Human; GATA2 endothelial cell of umbilical vein hg19; TAL1 K562 hg19; BRCA1 H1-hESC hg19; MAF 26560356 Chip-Seq THI Human; CEBPB H1-hESC hg19; NR3C1 21868756 ChIP-Seq MCF10A Human; CREB1 GM12878 hg19; RAD21 MCF-7 hg19; CREB1 A549 hg19; MBD2 human tf ARCHS4 coexpression; RAPGEF5 human tf ARCHS4 coexpression; HMGA2 human tf ARCHS4 coexpression; FBN1 human tf ARCHS4 coexpression; SLC39A10 human tf ARCHS4 coexpression; CBFB human tf ARCHS4 coexpression; CUL2 human tf ARCHS4 coexpression; YOD1 human tf ARCHS4 coexpression; RBM22 human tf ARCHS4 coexpression; NFRKB human tf ARCHS4 coexpression; HBP1 human tf ARCHS4 coexpression; CEBPZ human tf ARCHS4 coexpression; KLF12 human tf ARCHS4 coexpression; ONECUT2 human tf ARCHS4 coexpression; RBPJL human tf ARCHS4 coexpression; NRID1 human tf ARCHS4 coexpression; UBP1 human tf ARCHS4 coexpression; PHOX2A human tf ARCHS4 coexpression; ZNF202 human tf ARCHS4 coexpression; MTA3 human tf ARCHS4 coexpression; ETV5 human tf ARCHS4 coexpression; IKZF2 human tf ARCHS4 coexpression; RLF human tf ARCHS4 coexpression; TBR1 human tf ARCHS4 coexpression; GBX2 human tf ARCHS4 coexpression; ZBTB41 human tf ARCHS4 coexpression; ZFP36L1 human tf ARCHS4 coexpression; FOXJ3 human tf ARCHS4 coexpression; BMP2 human tf ARCHS4 coexpression; PAWR human tf ARCHS4 coexpression; MAFG human tf ARCHS4 coexpression; VDR 24787735 ChIP-Seq THP-1 Human; FOSL2 MCF-7 hg19; POLR2AphosphoS5 H1-hESC hg19; ZFP281 18358816 ChIP-ChIP MESCs Mouse; POLR2A A549 hg19; PAX5 GM12891 hg19; GATA3 A549 hg19; REST GM12878 hg19; REST MCF-7 hg19; RUNX 20019798 ChIP-Seq JUKART Human; CTCF T47D hg19; TAF1 GM12891 hg19; ZC3H11A CH12.LX mm9; TAL1 20887958 ChIP-Seq HPC-7 Mouse; STAT3 18555785 ChIP-Seq MESCs Mouse; AR 21572438 ChIP-Seq LNCaP Human; FOXA1 26769127 Chip-Seq PDAC-Cell line Human; NCOR1 26117541 ChIP-Seq K562 Human; NFYB HeLa—S3 hg19; NRF1 K562 hg19; RAD21 HeLa—S3 hg19; MAX endothelial cell of umbilical vein hg19; BHLHE40 A549 hg19; EP300 MEL cell line mm9; POLR2A thymus mm9; USF1 C2C12 mm9; POLR2A heart mm9; ZNF143 H1-hESC hg19; NUCKS1 24931609 ChIP-Seq HEPATOCYTES Mouse; POLR2A MCF-7 hg19; PML MCF-7 hg19; POU2F2 GM12878 hg19; POU2F2 GM12891 hg19; SOX2 18555785 ChIP-Seq MESCs Mouse; FOS HeLa—S3 hg19; FOXP2 23625967 ChIP-Seq PFSK-1 AND SK—N-MC Human; SOX2 19030024 ChIP-ChIP MESCs Mouse; EP300 HeLa—S3 hg19; SMAD4 19686287 ChIP-ChIP HaCaT Human; FOXMI 25889361 ChIP-Seq OE33 AND U2OS Human; MYC 22102868 ChIP-Seq BL Human; POLR2AphosphoS5 GM12892 hg19; RUNX2 22187159 ChIP-Seq PCA Human; SRF myocyte mm9; USF1 SK—N—SH hg19; GATA2 21571218 ChIP-Seq MEGAKARYOCYTES Human; ZMIZ1 MEL cell line mm9; FOXA1 21915096 ChIP-Seq LNCaP-IF5 Human; NRF1 SK—N—SH hg19; BRD4 27068464 Chip-Seq AML-cells Mouse; SA1 27219007 Chip-Seq ERYTHROID Human; USF1 myocyte mm9; SMC3 CH12.LX mm9; NFYB K562 hg19; CHD2 H1-hESC hg19; MAZ IMR-90 hg19; RFX5 K562 hg19; POLR2A embryonic fibroblast mm9; CHD1 H1-hESC hg19; SMAD3 21741376 ChIP-Seq EPCs Human; HDAC2 MCF-7 hg19; ESR1 21235772 ChIP-Seq MCF-7 Human; HNF4A 19822575 ChIP-Seq HepG2 Human; ZFP57 27257070 Chip-Seq ESCs Mouse; MYC 18940864 ChIP-ChIP HL60 Human; NRF2 20460467 ChIP-Seq MEFs Mouse; NFE2L2 20460467 ChIP-Seq MEFs Mouse; NANOG 18347094 ChIP-ChIP MESCs Mouse; FOSL1 C2C12 mm9; MYB 21317192 ChIP-Seq ERMYB Mouse; SIN3A PFSK-1 hg19; HNF4A HepG2 hg19; USF2 MEL cell line mm9; CTCF MCF-7 hg19; CTCFL K562 hg19; ESR2 21235772 ChIP-Seq MCF-7 Human; POU3F1 26484290 ChIP-Seq ESCss Mouse; FOXM1 26456572 ChIP-Seq MCF-7 Human; FOXA1 26743006 Chip-Seq LNCaP-abl Human; POLR2A ES-Bruce4 mm9; NELFE MEL cell line mm9; GATA2 21186366 ChIP-Seq BM-HSCs Mouse; ZNF384 K562 hg19; BACH1 K562 hg19; RNF2 K562 hg19; POLR2A limb mm9; MAFK IMR-90 hg19; CTCF limb mm9; CHD4 K562 hg19; POLR2AphosphoS2 myocyte mm9; MEIS1 26253404 ChIP-Seq OPTIC CUPS Mouse; AHR 22903824 ChIP-Seq MCF-7 Human; RXRA H1-hESC hg19; LMO2 26923725 Chip-Seq HEMANGIOBLAST Mouse; FOSL2 HepG2 hg19; GATA1 22025678 ChIP-Seq K562 Human; STAT3 22323479 ChIP-Seq MACROPHAGE Mouse; MAX HepG2 hg19; MXD1 human tf ARCHS4 coexpression; PRDM1 human tf ARCHS4 coexpression; NFKB2 human tf ARCHS4 coexpression; NFAT5 human tf ARCHS4 coexpression; IKZF4 human tf ARCHS4 coexpression; NANOG human tf ARCHS4 coexpression; HMGB1 human tf ARCHS4 coexpression; PRMT3 human tf ARCHS4 coexpression; KNTC1 human tf ARCHS4 coexpression; SF3A3 human tf ARCHS4 coexpression; TTF1 human tf ARCHS4 coexpression; ZC3H13 human tf ARCHS4 coexpression; TRIM23 human tf ARCHS4 coexpression; RAPGEF4 human tf ARCHS4 coexpression; UBE2K human tf ARCHS4 coexpression; BAZ2A human tf ARCHS4 coexpression; ZFR human tf ARCHS4 coexpression; PRDM12 human tf ARCHS4 coexpression; ZNF710 human tf ARCHS4 coexpression; ZNF385B human tf ARCHS4 coexpression; PRDM14 human tf ARCHS4 coexpression; PRDM8 human tf ARCHS4 coexpression; OLIG3 human tf ARCHS4 coexpression; MYTIL human tf ARCHS4 coexpression; NPAS4 human tf ARCHS4 coexpression; MBNL3 human tf ARCHS4 coexpression; NPAS3 human tf ARCHS4 coexpression; ID4 human tf ARCHS4 coexpression; ZBED2 human tf ARCHS4 coexpression; ZNF770 human tf ARCHS4 coexpression; ZNF655 human tf ARCHS4 coexpression; HOXA13 human tf ARCHS4 coexpression; PRRX1 human tf ARCHS4 coexpression; MYCN human tf ARCHS4 coexpression; TCF7L1 human tf ARCHS4 coexpression; FOXF1 human tf ARCHS4 coexpression; ZNF143 human tf ARCHS4 coexpression; GSX1 human tf ARCHS4 coexpression; NPAS2 human tf ARCHS4 coexpression; KLF11 human tf ARCHS4 coexpression; TGIF1 human tf ARCHS4 coexpression; ZBTB33 human tf ARCHS4 coexpression; MAF human tf ARCHS4 coexpression; NEUROG1 human tf ARCHS4 coexpression; CREB3L2 human tf ARCHS4 coexpression; POU3F2 human tf ARCHS4 coexpression; PBX1 human tf ARCHS4 coexpression; MTF1 human tf ARCHS4 coexpression; SALL4 human tf ARCHS4 coexpression; ZBTB11 human tf ARCHS4 coexpression; SALL2 human tf ARCHS4 coexpression; STAT3 HeLa—S3 hg19; ISL1 27105846 Chip-Seq CPCs Mouse; RNF2 16625203 ChIP-ChIP MESCs Mouse; AR 25329375 ChIP-Seq VCAP Human; NR2F2 HepG2 hg19; POLR2A ECC-1 hg19; VDR 23401126 ChIP-Seq LCL-AND-THP1 Human; MYOG C2C12 mm9; TCF3/E2A 22897851 ChIP-Seq JUKARTE6-1 Human; MYC 20876797 ChIP-ChIP MEDULLOBLASTOMA Human; TP53 20018659 ChIP-ChIP RIE Mouse; JUND MCF-7 hg19; BHLHE40 HepG2 hg19; HNF4G HepG2 hg19; POLR2A cerebellum mm9; HDAC6 H1-hESC hg19; CEBPB HeLa—S3 hg19; POLR2A IMR-90 hg19; SIN3A GM12878 hg19; POLR2A HEK293 hg19; CTCF IMR-90 hg19; ZMIZ1 CH12.LX mm9; CTCF brain mm9; E2F4 MCF 10A hg19; NRF1 H1-hESC hg19; STAT1 GM12878 hg19; CTCF spleen mm9; MXII H1-hESC hg19; POLR2A brain mm9; ZNF384 MEL cell line mm9; TBLI 22424771 ChIP-Seq 293T Human; PHF8 20622853 ChIP-Seq HELA Human; CEBPB 20513432 ChIP-Seq MACROPHAGES Mouse; NELFA 20434984 ChIP-Seq ESCs Mouse; CDX2 22108803 ChIP-Seq LS180 Human; TEAD4 HepG2 hg19; SMARCC2 HeLa—S3 hg19; TAF1 ECC-1 hg19; TFAP2C 20629094 ChIP-Seq MCF-7 Human; EZH2 22144423 ChIP-Seq EOC Human; RCOR1 IMR-90 hg19; POLR2AphosphoS5 endothelial cell of umbilical vein hg19; EP300 GM12878 hg19; POLR2A GM12878 hg19; FUS 26573619 Chip-Seq HEK293 Human; RAD21 HCT116 hg19; MYOG myocyte mm9; CTCF H1-hESC hg19; SMAD 19615063 ChIP-ChIP OVARY Human; USF1 GM12878 hg19; TCF12 SK—N—SH hg19; PRDM14 21183938 ChIP-Seq MESCs Mouse; GABPA MEL cell line mm9; ELF1 K562 hg19; FOS MCF 10A hg19; NROB1 18358816 ChIP-ChIP MESCs Mouse; TBLIXR1 K562 hg19; YY1 HCT116 hg19; RAD21 K562 hg19; USF1 HCT116 hg19; EED 16625203 ChIP-ChIP MESCs Mouse; CEBPB 20176806 ChIP-Seq THIOMACROPHAGE Mouse; FOXA2 19822575 ChIP-Seq HepG2 Human; JUND A549 hg19; PPARG 20176806 ChIP-Seq MACROPHAGES Mouse; CTCF WI38 hg19; PHF8 H1-hESC hg19; BHLHE40 CH12.LX mm9; UBTF CH12.LX mm9; CHD1 26751641 Chip-Seq LNCaP Human; CTCF skeletal muscle myoblast hg19; CTCF 27219007 Chip-Seq Bcells Human; MYCN 27167114 Chip-Seq NEUROBLASTOMA Human; LM02 26923725 Chip-Seq HEMOGENIC-ENDOTHELIUM Mouse; TOP2B 26459242 ChIP-Seq MCF-7 Human; SOX6 21985497 ChIP-Seq MYOTUBES Mouse; NFYA 21822215 ChIP-Seq K562 Human; KLF4 19829295 ChIP-Seq ESCs Human; POLR2AphosphoS2 HepG2 hg19; MAFK H1-hESC hg19; TAL1 26923725 Chip-Seq HEMANGIOBLAST Mouse; SOX2 20726797 ChIP-Seq SW620 Human; REST 21632747 ChIP-Seq MESCs Mouse; GATA2 21666600 ChIP-Seq HMVEC Human; HDAC2 HepG2 hg19; SUZ12 16625203 ChIP-ChIP MESCs Mouse; BCL11B 21912641 ChIP-Seq STHDH STRIUM Mouse; ESR1 T47D hg19; EP300 20729851 ChIP-Seq FORBRAIN MIDBRAIN LIMB HEART Mouse; PPARD 23176727 ChIP-Seq KERATINOCYTES Mouse; POLR2A H1-hESC hg19; GFIIB 20887958 ChIP-Seq HPC-7 Mouse; EP300 HepG2 hg19; SRY 25088423 ChIP-ChIP EMBRYONIC GONADS Mouse; TCF4 23295773 ChIP-Seq U87 Human; ESR1 22446102 ChIP-Seq UTERUS Mouse; CTCF endothelial cell of umbilical TEAD4 vein hg19; 22529382 ChIP-Seq TROPHECTODERM Mouse; RAD21 ECC-1 hg19; JUN CH12.LX mm9; JUN 21703547 ChIP-Seq K562 Human; KLF5 18264089 ChIP-ChIP MESCs Mouse; KLF2 18264089 ChIP-ChIP MESCs Mouse; ERA 27197147 Chip-Seq ENDOMETRIOID-ADENOCARCINOMA Human; PADI4 21655091 ChIP-ChIP MCF-7 Human; NFIC ECC-1 hg19; ZIC3 20872845 ChIP-ChIP MESCs Mouse; TFAP2C HeLa—S3 hg19; RFX5 GM12878 hg19; CTCF medulloblastoma hg19; eGFP-JUNB K562 hg19; RELA GM10847 hg19; RELA GM12891 hg19; CTCF T-cell acute lymphoblastic leukemia hg19; NRF1 HepG2 hg19; USF2 H1-hESC hg19; GTF2F1 H1-hESC hg19; CTCF olfactory bulb mm9; TCF12/HEB 22897851 ChIP-Seq JUKARTE6-1 Human; CEBPB 20176806 ChIP-Seq MACROPHAGES Mouse; CBP 21632823 ChIP-Seq H3396 Human; AR 21915096 ChIP-Seq LNCaP-1F5 Human; RXR 22158963 ChIP-Seq LIVER Mouse; CBX2 22325352 ChIP-Seq 293T-Rex Human; GATA1 22383799 ChIP-Seq G1ME Mouse; RUNX1 22412390 ChIP-Seq EML Mouse; MYB 26560356 Chip-Seq THI Human; MYC 27129775 Chip-Seq CORNEA Mouse; POU3F2 20337985 ChIP-ChIP 501MEL Human; CEBPD 23245923 ChIP-Seq MEFs Mouse; KLF4 18264089 ChIP-ChIP MESCs Mouse; FOXA1 ECC-1 hg19; ZNF81 human tf ARCHS4 coexpression; ZBTB34 human tf ARCHS4 coexpression; ZNF131 human tf ARCHS4 coexpression; CAMTA1 human tf ARCHS4 coexpression; ZBTB39 human tf ARCHS4 coexpression; FOXQ1 human tf ARCHS4 coexpression; ZIC3 human tf ARCHS4 coexpression; ZNF385D human tf ARCHS4 coexpression; FOXO1 human tf ARCHS4 coexpression; ZNF189 human tf ARCHS4 coexpression; ZSCAN10 human tf ARCHS4 coexpression; CAMTA2 human tf ARCHS4 coexpression; ZNF516 human tf ARCHS4 coexpression; IKZF5 human tf ARCHS4 coexpression; LHX5 human tf ARCHS4 coexpression; ZNF491 human tf ARCHS4 coexpression; ZNF586 human tf ARCHS4 coexpression; ZNF551 human tf ARCHS4 coexpression; MAFB human tf ARCHS4 coexpression; MYT1 human tf ARCHS4 coexpression; PAX5 human tf ARCHS4 coexpression; SP6 human tf ARCHS4 coexpression; RXRA human tf ARCHS4 coexpression; NR2F2 human tf ARCHS4 coexpression; EGR3 human tf ARCHS4 coexpression; NR2F1 human tf ARCHS4 coexpression; NRID2 human tf ARCHS4 coexpression; GLIS1 human tf ARCHS4 coexpression; ZNF24 human tf ARCHS4 coexpression; ARNT2 human tf ARCHS4 coexpression; FOXN2 human tf ARCHS4 coexpression; PRDM6 human tf ARCHS4 coexpression; ZNF746 human tf ARCHS4 coexpression; FOXL2 human tf ARCHS4 coexpression; DBX2 human tf ARCHS4 coexpression; ZNF805 human tf ARCHS4 coexpression; GTF2E2 human tf ARCHS4 coexpression; PSMD12 human tf ARCHS4 coexpression; H1FOO human tf ARCHS4 coexpression; RAG1 human tf ARCHS4 coexpression; LARP6 human tf ARCHS4 coexpression; SORBS2 human tf ARCHS4 coexpression; MKRN1 human tf ARCHS4 coexpression; DEK human tf ARCHS4 coexpression; HIST1H1A human tf ARCHS4 coexpression; EEA1 human tf ARCHS4 coexpression; PLEK2 human tf ARCHS4 coexpression; TRIT1 human tf ARCHS4 coexpression; ZNF75D human tf ARCHS4 coexpression; ZNF107 human tf ARCHS4 coexpression; ZBTB44 human tf ARCHS4 coexpression; ZNF335 human tf ARCHS4 coexpression; ZNF649 human tf ARCHS4 coexpression; ZNF134 human tf ARCHS4 coexpression; KLF13 human tf ARCHS4 coexpression; ZNF407 human tf ARCHS4 coexpression; UNKL human tf ARCHS4 coexpression; NCOA1 human tf ARCHS4 coexpression; PLEK human tf ARCHS4 coexpression; ZNF367 human tf ARCHS4 coexpression; NFXL1 human tf ARCHS4 coexpression; CUL3 human tf ARCHS4 coexpression; SLC30A9 human tf ARCHS4 coexpression; EOMES human tf ARCHS4 coexpression; STAT3 human tf ARCHS4 coexpression; ZBTB7B human tf ARCHS4 coexpression; HIVEP2 human tf ARCHS4 coexpression; POU2F2 human tf ARCHS4 coexpression; MEF2A human tf ARCHS4 coexpression; RORA human tf ARCHS4 coexpression; TBX5 human tf ARCHS4 coexpression; TFAM human tf ARCHS4 coexpression; ELF2 human tf ARCHS4 coexpression; E2F3 human tf ARCHS4 coexpression; HAND1 human tf ARCHS4 coexpression; HES1 human tf ARCHS4 coexpression; ZNF644 human tf ARCHS4 coexpression; BARHL1 human tf ARCHS4 coexpression; ETS1 human tf ARCHS4 coexpression; TBX3 human tf ARCHS4 coexpression; POU2F1 human tf ARCHS4 coexpression; GABPA human tf ARCHS4 coexpression; SMAD5 human tf ARCHS4 coexpression; FOXH1 human tf ARCHS4 coexpression; MEF2A GM12878 hg19; EZH2 27294783 Chip-Seq NPCs Mouse; DNAJC2 21179169 ChIP-ChIP NT2 Human; SMARCA4 20176728 ChIP-ChIP TSCs Mouse; POLR2A K562 hg19; E2F7 22180533 ChIP-Seq HELA Human; MYC MCF-7 hg19; SOX9 26525672 Chip-Seq Limbbuds Mouse; ATF2 GM12878 hg19; USF1 ECC-1 hg19; JUND H1-hESC hg19; CEBPB 26923725 Chip-Seq HEMANGIOBLAST Mouse; RCOR3 21632747 ChIP-Seq MESCs Mouse; CTCF LNCaP clone FGC hg19; CEBPB HCT116 hg19; FOSL1 H1-hESC hg19; RNF2 27304074 Chip-Seq NSC Mouse; POLR2A HeLa—S3 hg19; PAX5 GM12878 hg19; IRF1 K562 hg19; STAT1 HeLa—S3 hg19; REST ECC-1 hg19; POUSF1 16153702 ChIP-ChIP HESCs Human; GATA2 19941826 ChIP-Seq K562 Human; ESRRA GM12878 hg19; TCF3 18347094 ChIP-ChIP MESCs Mouse; TFAP2A HeLa—S3 hg19; CTCF GM12867 hg19; MAFK ES-E14 mm9; TAF1 HeLa—S3 hg19; MYOD1 C2C12 mm9; SIN3A K562 hg19; KLF4 25985364 ChIP-Seq ATHEROSCLEROSIS LESION Mouse; CTCF GM12891 hg19; SOX3 22085726 ChIP-Seq NPCs Mouse; MYB 26560356 Chip-Seq TH2 Human; KDM2B 26808549 Chip-Seq REH Human; TBLIXRI GM12878 hg19; BACH1 H1-hESC hg19; POLR2A erythroblast hg19; POLR2A HGPS cell hg19; SUZ12 K562 hg19; DPY 21335234 ChIP-Seq ESCs Mouse; NFYB 21822215 ChIP-Seq K562 Human; NMYC 18555785 Chip-Seq ESCs Mouse; GATA1 19941827 ChIP-Seq MEL86 Mouse; HNFA 21074721 ChIP-Seq CACO-2 Human; SMAD3 21741376 ChIP-Seq ESCs Human; SMAD4 21741376 ChIP-Seq EPCs Human; EP300 H1-hESC hg19; CTCF A549 hg19; USF1 K562 hg19; FLI1 21571218 ChIP-Seq MEGAKARYOCYTES Human; CEBPB 23403033 ChIP-Seq LIVER Mouse; POLR2AphosphoS5 SK—N—SH hg19; CTCF K562 hg19; JUND HCT116 hg19; SMAD2 18955504 ChIP-ChIP HaCaT Human; SMAD3 18955504 ChIP-ChIP HaCaT Human; ZBTB33 HCT116 hg19; TCF12 myocyte mm9; EWS 26573619 Chip-Seq HEK293 Human; NFE2L2 22581777 ChIP-Seq LYMPHOBLASTOID Human; POLR2A GM12891 hg19; GFIB 26923725 Chip-Seq HPCs Mouse; MEIS1 20887958 ChIP-Seq HPC-7 Mouse; RELA GM12892 hg19; TP63 19390658 ChIP-ChIP HaCaT Human; RAD21 H1-hESC hg19; EZH2 23942234 ChIP-Seq MYOBLASTS AND MYOTUBES Mouse; MEIS1 26923725 Chip-Seq HEMOGENIC-ENDOTHELIUM Mouse; POLR2AphosphoS5 HL-60 hg19; POLR2AphosphoS5 K562 hg19; HSF1 HepG2 hg19; EP300 SK—N—SH hg19; P53 22127205 ChIP-Seq FIBROBLAST Human; RARG 19884340 ChIP-ChIP MEFs Mouse; SETDB1 K562 hg19; ETS2 20176728 ChIP-ChIP TROPHOBLAST STEM CELLS Mouse; CTCF fibroblast of foreskin hg19; ESRRA HepG2 hg19; ERG 21242973 ChIP-ChIP JURKAT Human; TAF1 SK—N—SH hg19; TAF1 K562 hg19; POLR2AphosphoS5 GM12891 hg19; IRF3 HeLa—S3 hg19; TAF1 MCF-7 hg19; SMAD4 21741376 ChIP-Seq ESCs Human; P63 20808887 ChIP-Seq KERATINOCYTES Human; CEBPA 20513432 ChIP-Seq MACROPHAGES Mouse; TCFCP2L1 18555785 Chip-Seq ESCs Mouse; GATA3 22897851 ChIP-Seq JUKARTE6-1 Human; TAL1 22897851 ChIP-Seq JUKARTE6-1 Human; CTCF lung mm9; ZNF384 ES-E14 mm9; NCORI K562 hg19; RAD21 CH12.LX mm9; MXII K562 hg19; CHD2 HepG2 hg19; WHSC1 K562 hg19; CTCF GM13976 hg19; NFYB GM12878 hg19; MAFK HeLa—S3 hg19; CBX8 K562 hg19; KDM5B H1-hESC hg19; NKX2-5 21415370 ChIP-Seq HL-1 Mouse; BCAT 22108803 ChIP-Seq LS180 Human; POLR2AphosphoS5 MEL cell line mm9; BCL11A H1-hESC hg19; PML K562 hg19; CTCF retinal pigment epithelial cell hg19; MYC 19030024 ChIP-ChIP MESCs Mouse; TCF4 18268006 ChIP-ChIP LS174T Human; REST HL-60 hg19; STAT6 20620947 ChIP-Seq CD4 POS T Human; TCF7L2 HeLa—S3 hg19; FOXP3 17237761 ChIP-ChIP TREG Mouse; USF1 A549 hg19; PBX3 A549 hg19; CTCF ECC-1 hg19; EGR1 GM12878 hg19; GTF3C2 K562 hg19; HDAC2 H1-hESC hg19; MYCN 18555785 ChIP-Seq MESCs Mouse; SRF HepG2 hg19; POLR2AphosphoS5 SK—N-MC hg19; EP300 T47D hg19; GATA1 19941826 ChIP-Seq K562 Human; RXR 22108803 ChIP-Seq LS180 Human; PRDM16 22522345 ChIP-ChIP PALATE MESENCHYMAL Mouse; POLR2A cortical plate mm9; CEBPB 22108803 ChIP-Seq LS180 Human; ETS1 21867929 ChIP-Seq TH2 Mouse; OCT4 21477851 ChIP-Seq ESCs Mouse; ETV1 20927104 ChIP-Seq GIST48 Human; LUZP1 20508642 ChIP-Seq ESCs Mouse; OCT4 19829295 ChIP-Seq ESCs Human; P300 18555785 Chip-Seq ESCs Mouse; GATA3 26560356 Chip-Seq TH2 Human; MYC A549 hg19; PU1 27457419 Chip-Seq LIVER Mouse; ZKSCANI HeLa—S3 hg19; GTF2F1 HeLa—S3 hg19; HCFC1 K562 hg19; BRCA1 GM12878 hg19; CTCF testis mm9; HDAC6 K562 hg19; POLR2A NB4 hg19; GTF2F1 K562 hg19; NFATC1 human tf ARCHS4 coexpression; NFE2L2 human tf ARCHS4 coexpression; ELF1 human tf ARCHS4 coexpression; NFYB human tf ARCHS4 coexpression; PAX1 human tf ARCHS4 coexpression; TCF15 human tf ARCHS4 coexpression; HIVEP3 human tf ARCHS4 coexpression; KLF2 human tf ARCHS4 coexpression; ZNF12 human tf ARCHS4 coexpression; T human tf ARCHS4 coexpression; BCL6B human tf ARCHS4 coexpression; TCF7L2 human tf ARCHS4 coexpression; NR6A1 human tf ARCHS4 coexpression; CREB5 human tf ARCHS4 coexpression; ARNT human tf ARCHS4 coexpression; PDX1 human tf ARCHS4 coexpression; MYC human tf ARCHS4 coexpression; HEY2 human tf ARCHS4 coexpression; MEIS2 human tf ARCHS4 coexpression; NR1H2 human tf ARCHS4 coexpression; ZNF215 human tf ARCHS4 coexpression; NR2E1 human tf ARCHS4 coexpression; PHOX2B human tf ARCHS4 coexpression; GMEB2 human tf ARCHS4 coexpression; ATF4 human tf ARCHS4 coexpression; ZFX human tf ARCHS4 coexpression; HOXC8 human tf ARCHS4 coexpression; MNT human tf ARCHS4 coexpression; MEF2C human tf ARCHS4 coexpression; TCFL5 human tf ARCHS4 coexpression; ZNF155 human tf ARCHS4 coexpression; ZNF709 human tf ARCHS4 coexpression; ZFP62 human tf ARCHS4 coexpression; FOXE3 human tf ARCHS4 coexpression; ZBTB40 human tf ARCHS4 coexpression; FOXL1 human tf ARCHS4 coexpression; ZNF483 human tf ARCHS4 coexpression; ZNF57 human tf ARCHS4 coexpression; NHLH1 human tf ARCHS4 coexpression; ZNF524 human tf ARCHS4 coexpression; ZNF592 human tf ARCHS4 coexpression; NKRF human tf ARCHS4 coexpression; UNCX human tf ARCHS4 coexpression; SALL3 human tf ARCHS4 coexpression; FOXF2 human tf ARCHS4 coexpression; ZNF304 human tf ARCHS4 coexpression; ZNF227 human tf ARCHS4 coexpression; MNX1 human tf ARCHS4 coexpression; KLF7 human tf ARCHS4 coexpression; ZNF324 human tf ARCHS4 coexpression; ZNF263 human tf ARCHS4 coexpression; TFDP1 human tf ARCHS4 coexpression; FOXD3 human tf ARCHS4 coexpression; TAL1 human tf ARCHS4 coexpression; ZHX3 human tf ARCHS4 coexpression; DNAJC2 human tf ARCHS4 coexpression; ZNF831 human tf ARCHS4 coexpression; LGR4 human tf ARCHS4 coexpression; HELZ human tf ARCHS4 coexpression; GTF2F2 human tf ARCHS4 coexpression; HIST1HIC human tf ARCHS4 coexpression; HIST1HIB human tf ARCHS4 coexpression; TIGD3 human tf ARCHS4 coexpression; SMARCC1 human tf ARCHS4 coexpression; COPS2 human tf ARCHS4 coexpression; BRPF1 human tf ARCHS4 coexpression; SMARCA5 human tf ARCHS4 coexpression; POLE3 human tf ARCHS4 coexpression; NCOA3 human tf ARCHS4 coexpression; SSH2 human tf ARCHS4 coexpression; SSB human tf ARCHS4 coexpression; SMAD6 human tf ARCHS4 coexpression; PLXNC1 human tf ARCHS4 coexpression; DHX34 human tf ARCHS4 coexpression; RNF138 human tf ARCHS4 coexpression; KIAA1683 human tf ARCHS4 coexpression; TGIF2 human tf ARCHS4 coexpression; HMGB3 human tf ARCHS4 coexpression; WDHD1 human tf ARCHS4 coexpression; FOXI2 human tf ARCHS4 coexpression; THAP1 human tf ARCHS4 coexpression; CUL4A human tf ARCHS4 coexpression; ZBTB4 human tf ARCHS4 coexpression; ZNF689 human tf ARCHS4 coexpression; ZNF274 human tf ARCHS4 coexpression; RELA human tf ARCHS4 coexpression; ZNF35 human tf ARCHS4 coexpression; PRRX2 human tf ARCHS4 SOHLH1 human tf ARCHS4 coexpression; ZNF22 human tf ARCHS4 coexpression; coexpression; PDX1 19855005 ChIP-ChIP MIN6 Mouse; MAX myocyte mm9; CDKN2AIP 20523734 ChIP-Seq CORTICAL Neurons; POU5F1 26923725 Chip-Seq MESODERM Mouse; TEAD4 26923725 Chip-Seq HEMANGIOBLAST Mouse; TCFAP2C 20176728 ChIP-ChIP TROPHOBLAST STEM CELLS Mouse; CTCF cardiac muscle cell hg19; MYC K562 hg19; EP300 21415370 ChIP-Seq HL-1 Mouse; SRF HCT116 hg19; ASH2L 23239880 ChIP-Seq MESCs Mouse; TTF2 22483619 ChIP-Seq HELA Human; FOXM1 23109430 ChIP-Seq U2OS Human; CTCF mammary epithelial cell hg19; USF1 H1-hESC hg19; SREBP2 21459322 ChIP-Seq LIVER Mouse; POU5F1 18358816 ChIP-ChIP MESCs Mouse; SMAD3 22036565 ChIP-Seq ESCs Mouse; CTNNB1 24651522 ChIP-Seq LGR5+ INTESTINAL STEM Human; CTCF HeLa—S3 hg19; DROSHA 22980978 ChIP-Seq HELA Human; GATA1 19941827 ChIP-Seq MEL Mouse; STATI K562 hg19; POLR2A MEL cell line mm9; REST H1-hESC hg19; ZC3H11A ES-E14 mm9; FOXA2 A549 hg19; PPARG 20176806 ChIP-Seq THIOMACROPHAGE Mouse; XRN2 22483619 ChIP-Seq HELA Human; USF1 HepG2 hg19; CIITA 18437201 ChIP-ChIP Raji B and iDC Human; EWS-FLI1 20517297 ChIP-Seq SK—N-MC Human; MAX A549 hg19; NRF1 HeLa—S3 hg19; JARID1B-DAIN 22020125 ChIP-Seq ESCs Mouse; TBP HeLa—S3 hg19; NRF1 GM12878 hg19; MXII IMR-90 hg19; TBP HepG2 hg19; CTCF GM20000 hg19; USF2 HepG2 hg19; POLR2A testis mm9; eGFP-JUND K562 hg19; FOS K562 hg19; ELKI GM12878 hg19; GATA6 25053715 ChIP-Seq YYC3 Human; SOX2 18555785 Chip-Seq ESCs Mouse; PU.1 20513432 ChIP-Seq MACROPHAGES Mouse; CDX2 21402776 ChIP-Seq INTESTINAL-VILLUS Mouse; GATA3 21867929 ChIP-Seq THI Mouse; LXR 22292898 ChIP-Seq THP-1 Human; ELKI HeLa—S3 hg19; CHD1 GM12878 hg19; USF2 GM12878 hg19; CTCF GM19240 hg19; POLR2A CH12.LX mm9; CTCF skin fibroblast hg19; BRCA1 HepG2 hg19; SAP30 H1-hESC hg19; E2F4 GM12878 hg19; POLR2AphosphoS2 CH12.LX mm9; BHLHE40 GM12878 hg19; MAX NB4 hg19; FOXA2 HepG2 hg19; SPII 20176806 ChIP-Seq THIOMACROPHAGE Mouse; ER 23166858 ChIP-Seq MCF-7 Human; CEBPD K562 hg19; SUZ12 H1-hESC hg19; NR2F2 MCF-7 hg19; ATF3 H1-hESC hg19; TAF1 GM12892 hg19; AUTS2 25519132 ChIP-Seq 293T-REX Human; TDRD3 21172665 ChIP-Seq MCF-7 Human; BCL3 GM12878 hg19; ELK4 26923725 Chip-Seq MESODERM Mouse; REST K562 hg19; CTCF epithelial cell of proximal tubule hg19; SPI1 23127762 ChIP-Seq K562 Human; MEF2A K562 hg19; KLF4 18555785 ChIP-Seq MESCs Mouse; BACH1 22875853 ChIP-PCR HELA AND SCP4 Human; GTF3C2 HeLa—S3 hg19; EOMES 20176728 ChIP-ChIP TSCs Mouse; TCF7L2 HepG2 hg19; NOTCH1 17114293 ChIP-ChIP T-ALL Human; LMO2 26923725 Chip-Seq MACROPHAGESS Mouse; EP300 A549 hg19; TAF1 A549 hg19; CBX3 HCT116 hg19; SMAD4 21741376 ChIP-Seq HESCs Human; GATA1 erythroblast hg19; CTCF GM12865 hg19; E2F6 H1-hESC hg19; NR112 20693526 ChIP-Seq LIVER Mouse; SOX2 18358816 ChIP-ChIP MESCs Mouse; RAD21 21589869 ChIP-Seq MESCs Mouse; ATF3 HepG2 hg19; ZBTB7A K562 hg19; PPARD 23208498 ChIP-Seq MDA-MB-231 Human; RCOR2 21632747 ChIP-Seq MESCs Mouse; CTCF GM19238 hg19; FOXH1 21741376 ChIP-Seq ESCs Human; CDX2 21074721 ChIP-Seq CACO-2 Mouse; CMYC 18555785 Chip-Seq ESCs Mouse; E2F1 18555785 Chip-Seq ESCs Mouse; NANOG 18555785 Chip-Seq ESCs Mouse; CTCF GM13977 hg19; HMGN3 K562 hg19; RAD21 IMR-90 hg19; CTCF liver mm9; TCF7L2 HCT116 hg19; CTCF GM19239 hg19; TBP K562 hg19; CTCF GM12892 hg19; MAZ MEL cell line mm9; NFE2 GM12878 hg19; TBP H1-hESC hg19; GATA4 25053715 ChIP-Seq YYC3 Human; POLR2A H54 hg19; EBF1 22473956 ChIP-Seq BONE MARROW Mouse; FOXA1 27197147 Chip-Seq ENDOMETRIOID-ADENOCARCINOMA Human; PPARG 20176806 ChIP-Seq 3T3-L1 Mouse; ZFP281 18757296 ChIP-ChIP E14 Mouse; E2F6 HeLa—S3 hg19; KAT2A GM12878 hg19; ESR1 ECC-1 hg19; GATA1 megakaryocyte mm9; CTBP1 25329375 ChIP-Seq LNCAP Human; SP1 HCT116 hg19; RXRA SK—N—SH hg19; SFPI1 20887958 ChIP-Seq HPC-7 Mouse; CEBPB myocyte mm9; ETS1 K562 hg19; TAF1 PFSK-1 hg19; CTCF BE2C hg19; SRF H1-hESC hg19; PRDMI HeLa—S3 hg19; CTCF fibroblast of pedal digit skin hg19; CREB1 15753290 ChIP-ChIP HEK293T Human; TP53 16413492 ChIP-PET HCT116 Human; THRA 23701648 ChIP-Seq CEREBELLUM Mouse; NANOG 21062744 ChIP-ChIP HESCs Human; EWS-ERG 20517297 ChIP-Seq CADO-ESI Human; GF1 26923725 Chip-Seq HPCs Mouse; CDX2 19796622 ChIP-Seq MESCs Mouse; POLR2AphosphoS5 neural cell hg19; MAX MCF-7 hg19; TCF12 MCF-7 hg19; ATF3 GM12878 hg19; MAX HCT116 hg19; TCFCP2L1 18555785 ChIP-Seq MESCs Mouse; CHD1 19587682 ChIP-ChIP MESCs Mouse; ESRRB 18555785 ChIP-Seq MESCs Mouse; RUNX1 20887958 ChIP-Seq HPC-7 Mouse; TEAD4 K562 hg19; CTCF WERI-Rb-1 hg19; EGR1 K562 hg19; NACC1 18358816 ChIP-ChIP MESCs Mouse; FOXO3 human tf ARCHS4 coexpression; GSC human tf ARCHS4 coexpression; CLOCK human tf ARCHS4 coexpression; DMTF1 human tf ARCHS4 coexpression; GATA4 human tf ARCHS4 coexpression; LEF1 human tf ARCHS4 coexpression; LHX9 human tf ARCHS4 coexpression; NKX6-2 human tf ARCHS4 coexpression; FEV human tf ARCHS4 coexpression; RARA human tf ARCHS4 coexpression; HESX1 human tf ARCHS4 coexpression; HEY1 human tf ARCHS4 coexpression; OTX2 human tf ARCHS4 coexpression; STAT6 human tf ARCHS4 coexpression; NKX3-1 human tf ARCHS4 coexpression; PBX3 human tf ARCHS4 coexpression; SP2 human tf ARCHS4 coexpression; ZNF146 human tf ARCHS4 coexpression; RBPJ human tf ARCHS4 coexpression; HEYL human tf ARCHS4 coexpression; ZNF236 human tf ARCHS4 coexpression; NRL human tf ARCHS4 coexpression; ID1 human tf ARCHS4 coexpression; ZNF121 human tf ARCHS4 coexpression; ZFP28 human tf ARCHS4 coexpression; ZNF699 human tf ARCHS4 coexpression; HES4 human tf ARCHS4 coexpression; ZFP3 human tf ARCHS4 coexpression; ZNF135 human tf ARCHS4 coexpression; ZNF654 human tf ARCHS4 coexpression; ETV3L human tf ARCHS4 coexpression; TCF7 human tf ARCHS4 coexpression; PGR human tf ARCHS4 coexpression; ELF4 human tf ARCHS4 coexpression; BACH2 human tf ARCHS4 coexpression; BCL6 human tf ARCHS4 coexpression; MSX2 human tf ARCHS4 coexpression; TSHZ3 human tf ARCHS4 coexpression; LHX4 human tf ARCHS4 coexpression; SOX9 human tf ARCHS4 coexpression; PAX8 human tf ARCHS4 coexpression; FOXA2 human tf ARCHS4 coexpression; ZFPM2 human tf ARCHS4 coexpression; IRX1 human tf ARCHS4 coexpression; SIX2 human tf ARCHS4 coexpression; SCRT1 human tf ARCHS4 coexpression; ADNP human tf ARCHS4 coexpression; PBX4 human tf ARCHS4 coexpression; ZNF85 human tf ARCHS4 coexpression; RCOR3 human tf ARCHS4 coexpression; BCL11A human tf ARCHS4 coexpression; ZSCAN18 human tf ARCHS4 coexpression; BATF3 human tf ARCHS4 coexpression; ZNF484 human tf ARCHS4 coexpression; ZNF337 human tf ARCHS4 coexpression; CASZ1 human tf ARCHS4 coexpression; LHX1 human tf ARCHS4 coexpression; ZNF670 human tf ARCHS4 coexpression; AEBP2 human tf ARCHS4 coexpression; ZIC5 human tf ARCHS4 coexpression; ZSCAN29 human tf ARCHS4 coexpression; ZNF687 human tf ARCHS4 coexpression; ZNF92 human tf ARCHS4 coexpression; ZNF761 human tf ARCHS4 coexpression; EN1 human tf ARCHS4 coexpression; ZXDC human tf ARCHS4 coexpression; ZNF560 human tf ARCHS4 coexpression; FOXN3 human tf ARCHS4 coexpression; ZNF195 human tf ARCHS4 coexpression; CEBPB human tf ARCHS4 coexpression; STAT5B human tf ARCHS4 coexpression; ETV4 human tf ARCHS4 coexpression; HIC1 human tf ARCHS4 coexpression; NR2C2 human tf ARCHS4 coexpression; ZBTB7A human tf ARCHS4 coexpression; SOX2 human tf ARCHS4 coexpression; POU6F1 human tf ARCHS4 coexpression; SIX6 human tf ARCHS4 coexpression; MITF human tf ARCHS4 coexpression; MIER1 human tf ARCHS4 coexpression; MAFK human tf ARCHS4 coexpression; ZNF540 human tf ARCHS4 coexpression; ZBTB26 human tf ARCHS4 coexpression; ZNF701 human tf ARCHS4 coexpression; ZNF319 human tf ARCHS4 coexpression; TBX20 human tf ARCHS4 coexpression; POU3F3 human tf ARCHS4 coexpression; ZBTB48 human tf ARCHS4 coexpression; ZBTB32 human tf ARCHS4 coexpression; ZNF607 human tf ARCHS4 coexpression; USF1 human tf ARCHS4 coexpression; MATR3 human tf ARCHS4 coexpression; CD36 human tf ARCHS4 coexpression; DZIP1 human tf ARCHS4 coexpression; ZC3H7A human tf ARCHS4 coexpression; FOXD4L6 human tf ARCHS4 coexpression; AKAP8 human tf ARCHS4 coexpression; PMS1 human tf ARCHS4 coexpression; RIOK2 human tf ARCHS4 coexpression; RAD51 human tf ARCHS4 coexpression; ZNF316 human tf ARCHS4 coexpression; PHF21A human tf ARCHS4 coexpression; HES3 human tf ARCHS4 coexpression; KIAA1549 human tf ARCHS4 coexpression; TOX3 human tf ARCHS4 coexpression; ZNF821 human tf ARCHS4 coexpression; SOHLH2 human tf ARCHS4 coexpression; RC3H2 human tf ARCHS4 coexpression; PHB2 human tf ARCHS4 coexpression; PCGF6 human tf ARCHS4 coexpression; ARID3A human tf ARCHS4 coexpression; ARID5B human tf ARCHS4 coexpression; E2F8 human tf ARCHS4 coexpression; RBM26 human tf ARCHS4 coexpression; TRIM3 human tf ARCHS4 coexpression; FMNL2 human tf ARCHS4 coexpression; ADAMTS19 human tf ARCHS4 coexpression; PPP1R10 human tf ARCHS4 coexpression; RPA4 human tf ARCHS4 coexpression; GPATCH8 human tf ARCHS4 coexpression; ZNF485 human tf ARCHS4 coexpression; TERF2IP human tf ARCHS4 coexpression; HMGB2 human tf ARCHS4 coexpression; FBXO41 human tf ARCHS4 coexpression; CSDC2 human tf ARCHS4 coexpression; RNF166 human tf ARCHS4 coexpression; CTCF HCT116 hg19; ZNF217 MCF-7 hg19; P300 27268052 Chip-Seq Bcells Human; CEBPB 26923725 Chip-Seq HEMOGENIC-ENDOTHELIUM Mouse; P53 22387025 ChIP-Seq ESCs Mouse; SOX3 22085726 ChIP-Seq MUSCLE Mouse; ZFX 18555785 Chip-Seq ESCs Mouse; CTCF 18555785 Chip-Seq ESCs Mouse; POLR2A lung mm9; CTCF kidney hg19; EP300 osteoblast hg19; SAP30 K562 hg19; POLR2A bone marrow mm9; TBP GM12878 hg19; EZH2 endothelial cell of umbilical vein hg19; POLR2A spleen mm9; MAZ HepG2 hg19; BCL6 27268052 Chip-Seq Bcells Human; RUNX1 27457419 Chip-Seq LIVER Mouse; SMC3 K562 hg19; NFE2 27457419 Chip-Seq LIVER Mouse; ZNF274 K562 hg19; RAD21 HepG2 hg19; EBF1 GM12878 hg19; BCL3 23251550 ChIP-Seq MUSCLE Mouse; TAL1 MEL cell line mm9; HOXC9 25013753 ChIP-Seq NEUROBLASTOMA BE2-C Human; MYC 19079543 ChIP-ChIP MESCs Mouse; UBTF K562 hg19; CCND1 20090754 ChIP-ChIP RETINA Mouse; ZEB1 HepG2 hg19; SOX9 26525672 Chip-Seq HEART Mouse; ZNF263 19887448 ChIP-Seq K562 Human; TAF1 GM12878 hg19; E2F1 21310950 ChIP-Seq MCF-7 Human; TEAD4 MCF-7 hg19; SIN3A 21632747 ChIP-Seq MESCs Mouse; MAX H1-hESC hg19; CNOT3 19339689 ChIP-ChIP MESCs Mouse; NANOG 18692474 ChIP-Seq MEFs Mouse; POU5F1 18347094 ChIP-ChIP MESCs Mouse; STAT2 K562 hg19; VDR 23849224 ChIP-Seq CD4+ Human; ATF3 A549 hg19; MECOM 23826213 ChIP-Seq KASUMI Mouse; CTCF GM12869 hg19; POLR3A K562 hg19; POLR2A MCF 10A hg19; FLI1 27457419 Chip-Seq LIVER Mouse; AR 19668381 ChIP-Seq PC3 Human; GABP 19822575 ChIP-Seq HepG2 Human; RELA GM19193 hg19; POLR2A GM19099 hg19; JUND K562 hg19; RXRA 24833708 ChIP-Seq LIVER Mouse; OLIG2 26023283 ChIP-Seq AINV15 Mouse; MNX1 26342078 ChIP-Seq MIN6-4N Mouse; KLF4 18555785 Chip-Seq ESCs Mouse; SUZ12 18555785 Chip-Seq ESCs Mouse; ESET 19884257 ChIP-Seq ESCs Mouse; CRX 20693478 ChIP-Seq RETINA Mouse; SOX2 22085726 ChIP-Seq NPCs Mouse; ETS1 22383799 ChIP-Seq GIME Mouse; CTCF kidney mm9; TCF7L2 MCF-7 hg19; CTCF GM10248 hg19; USF2 CH12.LX mm9; NFYA K562 hg19; CTCF ES-Bruce4 mm9; CTCF myotube hg19; CTCF astrocyte hg19; MAX CH12.LX mm9; CTCF bone marrow mm9; RELA GM19099 hg19; FOXP1 21924763 ChIP-Seq HESCs Human; POLR3A HeLa—S3 hg19; CTCF GM12875 hg19; PPARG 23326641 ChIP-Seq C3H10T1-2 Mouse; ZKSCAN1 CH12.LX mm9; CBX2 27304074 Chip-Seq ESCs Mouse; EGR1 19032775 ChIP-ChIP M12 Human; E2F4 17652178 ChIP-ChIP JURKAT Human; ERG 20887958 ChIP-Seq HPC-7 Mouse; RCOR1 MEL cell line mm9; RXRA GM12878 hg19; EGR1 H1-hESC hg19; POLR2AphosphoS2 K562 hg19; FOSL1 HCT116 hg19; NFATC1 GM12878 hg19; CTCF foreskin fibroblast hg19; KDM5A 27292631 Chip-Seq BREAST Human; TAL1 G1E-ER4 mm9; CTCF bronchial epithelial cell hg19; LMO2 20887958 ChIP-Seq HPC-7 Mouse; CTCF GM12878 hg19; SCL 21571218 ChIP-Seq MEGAKARYOCYTES Human; TCF12 A549 hg19; SPI1 23547873 ChIP-Seq NB4 Human; TAF1 neural cell hg19; SOX2 18692474 ChIP-Seq MEFs Mouse; ERG 20517297 ChIP-Seq VCAP Human; CTCF GM12868 hg19; PCGF2 27294783 Chip-Seq NPCs Mouse; CTCF GM06990 hg19; SREBP1 19666523 ChIP-Seq LIVER Mouse; TAL1 G1E mm9; POLR2A GM10847 hg19; MAZ HeLa—S3 hg19; MYC HepG2 hg19; MXI1 HeLa—S3 hg19; SETDBI U20S hg19; MXII GM12878 hg19; RCOR1 HepG2 hg19; CTCF thymus mm9; ZNF143 GM12878 hg19; CHD2 K562 hg19; KLF5 20875108 ChIP-Seq MESCs Mouse; UTX 26944678 Chip-Seq JUKART Human; GATA2 22383799 ChIP-Seq G1ME Mouse; RBPJ 21746931 ChIP-Seq IB4 Human; MAZ GM12878 hg19; MAFK CH12.LX mm9; HOXB7 26014856 ChIP-Seq BT474 Human; GATA2 SH-SY5Y hg19; ETS1 CH12.LX mm9; TBP CH12.LX mm9; MYC endothelial cell of umbilical vein hg19; CTCF HGPS cell hg19; GATA3 SH-SY5Y hg19; EP300 ES-Bruce4 mm9; SIRT6 K562 hg19; CHD7 H1-hESC hg19; FOXP2 PFSK-1 hg19; TBX3 20139965 ChIP-Seq MESCs Mouse; TBX3 20139965 ChIP-Seq ESCs Mouse; ZBTB33 SK—N—SH hg19; MYC MCF 10A hg19; JUND T47D hg19; TAL1 megakaryocyte mm9; TAF1 HepG2 hg19; CTCF MEL cell line mm9; NELFE K562 hg19; CIITA 25753668 ChIP-Seq RAJ1Human; YY1 SK—N—SH hg19; FOS GM12878 hg19; TCF12 ECC-1 hg19; CTCF HEK293 hg19; LYL1 20887958 ChIP-Seq HPC-7 Mouse; CDX2 20551321 ChIP-Seq CACO-2 Human; CBP 20019798 ChIP-Seq JUKART Human; IRF4 20064451 ChIP-Seq CD4+T Mouse; POLR2AphosphoS5 Panc1 hg19; NR3C1HepG2 hg19; CTCF kidney epithelial cell hg19; GABPA K562 hg19; CTCF HepG2 hg19; RELA GM18526 hg19; FOXA1 HepG2 hg19; TP63 17297297 ChIP-ChIP HaCaT Human; CTCF astrocyte of the cerebellum hg19; ZBTB33 HepG2 hg19; SMAD3 21741376 ChIP-Seq HESCs Human; CTCF fibroblast of upper leg skin hg19; SOX17 human tf ARCHS4 coexpression; PPARG human tf ARCHS4 coexpression; SOX5 human tf ARCHS4 coexpression; HLX human tf ARCHS4 coexpression; NR5A1 human tf ARCHS4 coexpression; DMRT1 human tf ARCHS4 coexpression; TFAP2A human tf ARCHS4 coexpression; TCF12 human tf ARCHS4 coexpression; IRF8 human tf ARCHS4 coexpression; TEF human tf ARCHS4 coexpression; SMAD4 human tf ARCHS4 coexpression; ZNF182 human tf ARCHS4 coexpression; ST18 human tf ARCHS4 coexpression; NR3C2 human tf ARCHS4 coexpression; TSC22D3 human tf ARCHS4 coexpression; CREB3L1 human tf ARCHS4 coexpression; PKNOX1 human tf ARCHS4 coexpression; ZNF385A human tf ARCHS4 coexpression; MSX1 human tf ARCHS4 coexpression; TLX3 human tf ARCHS4 coexpression; BARHL2 human tf ARCHS4 coexpression; SIM2 human tf ARCHS4 coexpression; ZNF229 human tf ARCHS4 coexpression; NRF1 human tf ARCHS4 coexpression; DEAF1 human tf ARCHS4 coexpression; ZNF260 human tf ARCHS4 coexpression; MSC human tf ARCHS4 coexpression; HOXC9 human tf ARCHS4 coexpression; SOX1 human tf ARCHS4 coexpression; MAFF human tf ARCHS4 coexpression; IRF7 human tf ARCHS4 coexpression; MYBL1 human tf ARCHS4 coexpression; TEAD1 human tf ARCHS4 coexpression; TCF4 human tf ARCHS4 coexpression; ZNF398 human tf ARCHS4 coexpression; PBX2 human tf ARCHS4 coexpression; POU5F1 human tf ARCHS4 coexpression; ZBED1 human tf ARCHS4 coexpression; DDIT3 human tf ARCHS4 coexpression; ZNF582 human tf ARCHS4 coexpression; ZNF93 human tf ARCHS4 coexpression; ZNF416 human tf ARCHS4 coexpression; ZBED4 human tf ARCHS4 coexpression; ZNF90 human tf ARCHS4 coexpression; PRDM13 human tf ARCHS4 coexpression; GON4L human tf ARCHS4 coexpression; ZNF436 human tf ARCHS4 coexpression; ZBTB46 human tf ARCHS4 coexpression; ZNF414 human tf ARCHS4 coexpression; ZNF740 human tf ARCHS4 coexpression; JDP2 human tf ARCHS4 coexpression; ZNF41 human tf ARCHS4 coexpression; ZNF222 human tf ARCHS4 coexpression; ZNF394 human tf ARCHS4 coexpression; ZNF510 human tf ARCHS4 coexpression; ZNF622 human tf ARCHS4 coexpression; ZNF350 human tf ARCHS4 coexpression; HOXD4 human tf ARCHS4 coexpression; TCF20 human tf ARCHS4 coexpression; ZNF251 human tf ARCHS4 coexpression; ZNF71 human tf ARCHS4 coexpression; ZNF311 human tf ARCHS4 coexpression; ZNF239 human tf ARCHS4 coexpression; ZNF569 human tf ARCHS4 coexpression; ZNF573 human tf ARCHS4 coexpression; ZBTB6 human tf ARCHS4 coexpression; ZNF696 human tf ARCHS4 coexpression; SNAI3 human tf ARCHS4 coexpression; RFX7 human tf ARCHS4 coexpression; ZNF354C human tf ARCHS4 coexpression; BNC2 human tf ARCHS4 coexpression; ZNF217 human tf ARCHS4 coexpression; ZIK1 human tf ARCHS4 coexpression; OTP human tf ARCHS4 coexpression; ZNF672 human tf ARCHS4 coexpression; PTF1A human tf ARCHS4 coexpression; BATF2 human tf ARCHS4 coexpression; STAT5A human tf ARCHS4 coexpression; CUX1 human tf ARCHS4 coexpression; ZNF75A human tf ARCHS4 coexpression; LHX8 human tf ARCHS4 coexpression; AFF3 human tf ARCHS4 coexpression; ZNF326 human tf ARCHS4 coexpression; PKNOX2 human tf ARCHS4 coexpression; ZSCAN21 human tf ARCHS4 coexpression; TSC22D4 human tf ARCHS4 coexpression; ZNF232 human tf ARCHS4 coexpression; ZBTB43 human tf ARCHS4 coexpression; PRDM10 human tf ARCHS4 coexpression; ZNF496 human tf ARCHS4 coexpression; SOX21 human tf ARCHS4 coexpression; ZC3H15 human tf ARCHS4 coexpression; ZNFX1 human tf ARCHS4 coexpression; HIF3A human tf ARCHS4 coexpression; TCF19 human tf ARCHS4 coexpression; HOXA11 human tf ARCHS4 coexpression; E2F2 human tf ARCHS4 coexpression; MXI1 human tf ARCHS4 coexpression; MYB human tf ARCHS4 coexpression; FOXP1 human tf ARCHS4 coexpression; TFEB human tf ARCHS4 coexpression; EZH1 human tf ARCHS4 coexpression; USF2 human tf ARCHS4 coexpression; DBP human tf ARCHS4 coexpression; MEIS3 human tf ARCHS4 coexpression; TRPS1 human tf ARCHS4 coexpression; NFATC4 human tf ARCHS4 coexpression; SIX4 human tf ARCHS4 coexpression; GLI2 human tf ARCHS4 coexpression; NFATC3 human tf ARCHS4 coexpression; ZFY human tf ARCHS4 coexpression; DMRT3 human tf ARCHS4 coexpression; FOXC1 human tf ARCHS4 coexpression; SNAI2 human tf ARCHS4 coexpression; NFIX human tf ARCHS4 coexpression; CUX2 human tf ARCHS4 coexpression; ZMAT2 human tf ARCHS4 coexpression; ZNF776 human tf ARCHS4 coexpression; TCF23 human tf ARCHS4 coexpression; NKX6-3 human tf ARCHS4 coexpression; ZNF717 human tf ARCHS4 coexpression; ZNF836 human tf ARCHS4 coexpression; ZNF782 human tf ARCHS4 coexpression; ZNF808 human tf ARCHS4 coexpression; CSDE1 human tf ARCHS4 coexpression; RGS11 human tf ARCHS4 coexpression; PLXNA2 human tf ARCHS4 coexpression; POGK human tf ARCHS4 coexpression; PLEKHA4 human tf ARCHS4 coexpression; NRK human tf ARCHS4 coexpression; UBR4 human tf ARCHS4 coexpression; HP1BP3 human tf ARCHS4 coexpression; MACF1 human tf ARCHS4 coexpression; TIGD7 human tf ARCHS4 coexpression; SLC22A4 human tf ARCHS4 coexpression; ARID2 human tf ARCHS4 coexpression; LENG9 human tf ARCHS4 coexpression; TIGD2 human tf ARCHS4 coexpression; LARP7 human tf ARCHS4 coexpression; ZMAT1 human tf ARCHS4 coexpression; FARSB human tf ARCHS4 coexpression; THAP10 human tf ARCHS4 coexpression; ZNF428 human tf ARCHS4 coexpression; COPS3 human tf ARCHS4 coexpression; EP300 ECC-1 hg19; RCOR1 K562 hg19; ETS1 GM12878 hg19; ELK1 22589737 ChIP-Seq MCF10A Human; TEAD4 H1-hESC hg19; IGF1R 20145208 ChIP-Seq DFB Human; FOXA1 21572438 ChIP-Seq LNCaP Human; POLR2AphosphoS2 A549 hg19; CTCF GM12801 hg19; CTCF HL-60 hg19; CTCF GM10266 hg19; SIN3A MEL cell line mm9; eGFP-HDAC8 K562 hg19; CTCF heart mm9; POLR2AphosphoS2 HeLa—S3 hg19; EZH2 HeLa—S3 hg19; NELFE CH12.LX mm9; HA-E2F1 MCF-7 hg19; RELA GM15510 hg19; RCOR1 HeLa—S3 hg19; USF2 K562 hg19; KLF5 25053715 ChIP-Seq YYC3 Human; RARA 24833708 ChIP-Seq LIVER Mouse; SMAD1 18555785 Chip-Seq ESCs Mouse; STAT3 18555785 Chip-Seq ESCs Mouse; EBF1 22473956 ChIP-Seq LYMPHODE Mouse; NCOR 22424771 ChIP-Seq 293T Human; PCGF4 22325352 ChIP-Seq 293T-Rex Human; SOX11 22085726 ChIP-Seq ESNs Mouse; SMC4 20622854 ChIP-Seq HELA Human; OCT4 20526341 ChIP-Seq ESCs Human; ESRRB 18555785 Chip-Seq ESCs Mouse; TET1 21451524 ChIP-Seq MESCs Mouse; CTCF astrocyte of the spinal cord hg19; CTCF GM12870 hg19; DMRT1 21621532 ChIP-ChIP FETAL Ovary; GATA1 erythroblast mm9; SRF K562 hg19; CTCF keratinocyte hg19; TCF12 GM12878 hg19; CTCF GM12873 hg19; SIN3A H1-hESC hg19; GATA2 26923725 Chip-Seq HEMANGIOBLAST Mouse; REST A549 hg19; FOXP3 21729870 ChIP-Seq TREG Human; STAT5 23275557 ChIP-Seq MAMMARY-EPITHELIUM Mouse; CBX3 K562 hg19; TCF12 H1-hESC hg19; CTCF GM12872 hg19; SOX17 20123909 ChIP-Seq XEN Mouse; HOXB4 20404135 ChIP-ChIP EML Mouse; GATA2 K562 hg19; CTCF fibroblast of gingiva hg19; STAT3 1855785 ChIP-Seq MESCs Mouse; CTCF GM12871 hg19; SMARCD1 25818293 ChIP-Seq ESCs Mouse; FOSL2 A549 hg19; POLR2AphosphoS5 HepG2 hg19; CSB 26484114 Chip-Seq FIBROBLAST Human; STAT1 17558387 ChIP-Seq HELA Human; OCT4 18555785 Chip-Seq ESCs Mouse; SOX2 19829295 ChIP-Seq ESCs Human; FLI1 26923725 Chip-Seq MACROPHAGESS Mouse; GATA6 21074721 ChIP-Seq CACO-2 Mouse; GATA6 21074721 ChIP-Seq CACO-2 Human; NANOG 19829295 ChIP-Seq ESCs Human; NFYA GM12878 hg19; BHLHE40 K562 hg19; CHD2 HeLa—S3 hg19; TCF7L2 Panc1 hg19; EZH2 HepG2 hg19; CTCF NB4 hg19; HCFC1 ES-E14 mm9; POLR2A GM18505 hg19; CTCF lung hg19; E2F1 18555785 ChIP-Seq MESCs Mouse; E2F6 A549 hg19; BCL3 A549 hg19; SPI1 22790984 ChIP-Seq ERYTHROLEUKEMIA Mouse; SIN3A MCF-7 hg19; SUZ12 27294783 Chip-Seq NPCs Mouse; POLR2AphosphoS5 PFSK-1 hg19; SOX11 23321250 ChIP-ChIP Z138-A519-JVM2 Human; CHD7 19251738 ChIP-ChIP MESCs Mouse; GATA2 G1E-ER4 mm9; IRF3 HepG2 hg19; CTCF fibroblast of pulmonary artery hg19; TAL1 erythroblast mm9; SETDB1 19884255 ChIP-Seq MESCs Mouse; PBX3 SK—N—SH hg19; BATF GM12878 hg19; CTCF GM12874 hg19; CEBPA 23403033 ChIP-Seq LIVER Mouse; SIN3A Panc1 hg19; SPI1GM12878 hg19; TFEB 21752829 ChIP-Seq HELA Human; MAX K562 hg19; MYCN 21190229 ChIP-Seq SHEP-21N Human; TCF7L2 21901280 ChIP-Seq H4IIE Rat; KLF4 18358816 ChIP-ChIP MESCs Mouse; FLI1 megakaryocyte mm9; MAFK GM12878 hg19; MYC NB4 hg19; RELA GM18505 hg19; PU.1 20176806 ChIP-Seq MACROPHAGES Mouse; MAFK K562 hg19; ARID3A K562 hg19; CTCF bone marrow macrophage mm9; MXII SK—N—SH hg19; POLR2A GM18526 hg19; PHF8 K562 hg19; CTCF H54 hg19; CTCF CD14-positive monocyte hg19; MXI1 MEL cell line mm9; CTCF fibroblast of dermis hg19; POLR2A myocyte mm9; RUNX1 26923725 Chip-Seq HPCs Mouse; SPI1 GM12891 hg19; PCGF2 27294783 Chip-Seq ESCs Mouse; GATA2 G1E mm9; STAT3 19079543 ChIP-ChIP MESCs Mouse; SALL4 22934838 ChIP-ChIP CD34+ Human; STAT3 MCF 10A hg19; CTCF epithelial cell of esophagus hg19; GRHL2 25758223 ChIP-Seq PLACENTA Mouse; SRF MCF-7 hg19; MBD4 HepG2 hg19; SP1 H1-hESC hg19; CTCF fibroblast of skin of abdomen hg19; TP53 22573176 ChIP-Seq HFKS Human; IKZF1 21737484 ChIP-ChIP HCT116 Human; GPR155 human tf ARCHS4 coexpression; ADAR human tf ARCHS4 coexpression; LARP1 human tf ARCHS4 coexpression; SSRP1 human tf ARCHS4 coexpression; GTF2H2 human tf ARCHS4 coexpression; GATAD2B human tf ARCHS4 coexpression; MBD1 human tf ARCHS4 coexpression; MBD4 human tf ARCHS4 coexpression; YEATS4 human tf ARCHS4 coexpression; HIST1H1D human tf ARCHS4 coexpression; CASP8AP2 human tf ARCHS4 coexpression; RPA2 human tf ARCHS4 coexpression; PSMD11 human tf ARCHS4 coexpression; DOT1L human tf ARCHS4 coexpression; CHRAC1 human tf ARCHS4 coexpression; PARP12 human tf ARCHS4 coexpression; CUL1 human tf ARCHS4 coexpression; RELL2 human tf ARCHS4 coexpression; HIST1H1E human tf ARCHS4 coexpression; USP39 human tf ARCHS4 coexpression; KAT5 human tf ARCHS4 coexpression; RBM5 human tf ARCHS4 coexpression; REPIN1 human tf ARCHS4 coexpression; TOX human tf ARCHS4 coexpression; SEMA4A human tf ARCHS4 coexpression; PLXNA4 human tf ARCHS4 coexpression; RGS6 human tf ARCHS4 coexpression; HLA-DQB1 human tf ARCHS4 coexpression; CHD9 human tf ARCHS4 coexpression; ZSCAN5B human tf ARCHS4 coexpression; ZNF518B human tf ARCHS4 coexpression; DNAJC21 human tf ARCHS4 coexpression; SETBP1 human tf ARCHS4 coexpression; ZNF787 human tf ARCHS4 coexpression; SCAPER human tf ARCHS4 coexpression; ZBED3 human tf ARCHS4 coexpression; FEZF1 human tf ARCHS4 coexpression; ZNF391 human tf ARCHS4 coexpression; ZNF346 human tf ARCHS4 coexpression; ZNF772 human tf ARCHS4 coexpression; ZC3H3 human tf ARCHS4 coexpression; SCRT2 human tf ARCHS4 coexpression; ZNF425 human tf ARCHS4 coexpression; ZNF468 human tf ARCHS4 coexpression; ZBTB22 human tf ARCHS4 coexpression; ZNF521 human tf ARCHS4 coexpression; ZNF280B human tf ARCHS4 coexpression; ZNF460 human tf ARCHS4 coexpression; GABPB1 human tf ARCHS4 coexpression; ZNF343 human tf ARCHS4 coexpression; MYNN human tf ARCHS4 coexpression; ZZZ3 human tf ARCHS4 coexpression; IRF9 human tf ARCHS4 coexpression; MXD3 human tf ARCHS4 coexpression; SP5 human tf ARCHS4 coexpression; PAX9 human tf ARCHS4 coexpression; YY2 human tf ARCHS4 coexpression; ZNF43 human tf ARCHS4 coexpression; TEAD4 human tf ARCHS4 coexpression; POU3F4 human tf ARCHS4 coexpression; ESRRG human tf ARCHS4 coexpression; LCOR human tf ARCHS4 coexpression; PPARA human tf ARCHS4 coexpression; SRY human tf ARCHS4 coexpression; NKX2-5 human tf ARCHS4 coexpression; MAFA human tf ARCHS4 coexpression; PLAG1 human tf ARCHS4 coexpression; GSX2 human tf ARCHS4 coexpression; ZFPM1 human tf ARCHS4 coexpression; FOXE1 human tf ARCHS4 coexpression; SOX12 human tf ARCHS4 coexpression; HOXD8 human tf ARCHS4 coexpression; ZFHX4 human tf ARCHS4 coexpression; RHOXF1 human tf ARCHS4 coexpression; ZNF574 human tf ARCHS4 coexpression; NEUROD4 human tf ARCHS4 coexpression; FOXS1 human tf ARCHS4 coexpression; NHLH2 human tf ARCHS4 coexpression; ZNF77 human tf ARCHS4 coexpression; MGA human tf ARCHS4 coexpression; ZNF449 human tf ARCHS4 coexpression; OVOL1 human tf ARCHS4 coexpression; MLLT3 human tf ARCHS4 coexpression; ZNF561 human tf ARCHS4 coexpression; ZNF439 human tf ARCHS4 coexpression; ZBTB5 human tf ARCHS4 coexpression; ZNF25 human tf ARCHS4 coexpression; ZNF597 human tf ARCHS4 coexpression; ZNF219 human tf ARCHS4 coexpression; ZNF488 human tf ARCHS4 coexpression; HDX human tf ARCHS4 coexpression; ZNF225 human tf ARCHS4 coexpression; ZNF276 human tf ARCHS4 coexpression; ZNF618 human tf ARCHS4 coexpression; ZSCAN1 human tf ARCHS4 coexpression; ZNF711 human tf ARCHS4 coexpression; EMX1 human tf ARCHS4 coexpression; TOE1 human tf ARCHS4 coexpression; ZNF133 human tf ARCHS4 coexpression; ZNF646 human tf ARCHS4 coexpression; NEUROD2 human tf ARCHS4 coexpression; HIC2 human tf ARCHS4 coexpression; ATF5 human tf ARCHS4 coexpression; HNF1B human tf ARCHS4 coexpression; CREB3 human tf ARCHS4 coexpression; LHX6 human tf ARCHS4 coexpression; LHX2 human tf ARCHS4 coexpression; GLI3 human tf ARCHS4 coexpression; HOXA3 human tf ARCHS4 coexpression; HOXA2 human tf ARCHS4 coexpression; DLX5 human tf ARCHS4 coexpression; RELB human tf ARCHS4 coexpression; SALL1 human tf ARCHS4 coexpression; YY1 human tf ARCHS4 coexpression; CREBL2 human tf ARCHS4 coexpression; XBP1 human tf ARCHS4 coexpression; NFX1 human tf ARCHS4 coexpression; RFX3 human tf ARCHS4 coexpression; TP73 human tf ARCHS4 coexpression; RARB human tf ARCHS4 coexpression; TFE3 human tf ARCHS4 coexpression; SPI1 human tf ARCHS4 coexpression; POU1F1 human tf ARCHS4 coexpression; MEF2B human tf ARCHS4 coexpression; ZIC2 human tf ARCHS4 coexpression; TEAD3 human tf ARCHS4 coexpression; ETV1 human tf ARCHS4 coexpression; SOX10 human tf ARCHS4 coexpression; ZNF648 human tf ARCHS4 coexpression; SRF human tf ARCHS4 coexpression; HOXB5 human tf ARCHS4 coexpression; E2F6 human tf ARCHS4 coexpression; ZNF30 human tf ARCHS4 coexpression; RCOR2 human tf ARCHS4 coexpression; ZNF23 human tf ARCHS4 coexpression; DACH1 human tf ARCHS4 coexpression; NEUROD6 human tf ARCHS4 coexpression; LMX1A human tf ARCHS4 coexpression; NFIA human tf ARCHS4 coexpression; ISL2 human tf ARCHS4 coexpression; ERG human tf ARCHS4 coexpression; NFIB human tf ARCHS4 coexpression; STAT1 human tf ARCHS4 coexpression; ESR2 human tf ARCHS4 coexpression; TFCP2 human tf ARCHS4 coexpression; ARNTL human tf ARCHS4 coexpression; NPAS1 human tf ARCHS4 coexpression; FOXA1 human tf ARCHS4 coexpression; FOXP2 human tf ARCHS4 coexpression; DACH2 human tf ARCHS4 coexpression; THRA human tf ARCHS4 coexpression; TRERF1 human tf ARCHS4 coexpression; ATF1 human tf ARCHS4 coexpression; TWIST1 human tf ARCHS4 coexpression; HOXB1 human tf ARCHS4 coexpression; LMX1B human tf ARCHS4 coexpression; TSHZ1 human tf ARCHS4 coexpression; ZNF621 human tf ARCHS4 coexpression; ZNF467 human tf ARCHS4 coexpression; ZNF48 human tf ARCHS4 coexpression; ZNF418 human tf ARCHS4 coexpression; ZNF703 human tf ARCHS4 coexpression; ZNF695 human tf ARCHS4 coexpression; ZNF165 human tf ARCHS4 coexpression; ZNF679 human tf ARCHS4 coexpression; ZNF441 human tf ARCHS4 coexpression; ZKSCAN4 human tf ARCHS4 coexpression; ZNF471 human tf ARCHS4 coexpression; CTCF BJ hg19; OCT4 18692474 ChIP-Seq MEFs Mouse; VDR 20736230 ChIP-Seq LYMPHOBLASTOID Human; VDR 21846776 ChIP-Seq THP-1 Human; TBP 23326641 ChIP-Seq C3H10T1-2 Mouse; ZFX 18555785 ChIP-Seq MESCs Mouse; GATA4 21415370 ChIP-Seq HL-1 Mouse; GATA3 T47D hg19; NR4A2 19515692 ChIP-ChIP MN9D Mouse; NANOG 18358816 ChIP-ChIP MESCs Mouse; SA1 27219007 Chip-Seq Bcells Human; GATA3 26560356 Chip-Seq TH1 Human; RUNX1 22897851 ChIP-Seq JUKARTE6-1 Human; GATA3 21878914 ChIP-Seq MCF-7 Human; FOXH1 21741376 ChIP-Seq EPCs Human; ERA 21632823 ChIP-Seq H3396 Human; LDB1 21186366 ChIP-Seq BM-HSCs Mouse; EBNA1 20929547 Chip-Seq RAJI-cells Human; ELK1 K562 hg19; KDM5A H1-hESC hg19; ELK4 HEK293 hg19; TRIM28 HEK293 hg19; POLR2A Raji hg19; HDAC1 K562 hg19; ZC3H11A K562 hg19; CBX2 K562 hg19; MAX GM12878 hg19; EP300 CH12.LX mm9; RFX5 HepG2 hg19; FOXM1 ECC-1 hg19; NANOG 18692474 ChIP-Seq MESCs Mouse; RBPJ 21746931 ChIP-Seq IB4-LCL Human; YY1 23942234 ChIP-Seq MYOBLASTS AND MYOTUBES Mouse; NANOG H1-hESC hg19; E2F1 17053090 ChIP-ChIP MCF-7 Human; ESR1 20079471 ChIP-ChIP T-47D Human; GATA1 K562 hg19; HNF4A 19761587 ChIP-ChIP CACO-2 Human; TFAP2A 17053090 ChIP-ChIP MCF-7 Human; E2F4 HeLa—S3 hg19; FOXM1 MCF-7 hg19; CTCF fibroblast of the aortic adventitia hg19; ZFP42 18358816 ChIP-ChIP MESCs Mouse; TAF7L 23326641 ChIP-Seq C3H10T1-2 Mouse; PAX6 23342162 ChIP-ChIP BETA-FORBRAIN-LENS Mouse; SIN3B 21632747 ChIP-Seq MESCs Mouse; ZNF263 HEK293 hg19; MAX MEL cell line mm9; KDM4A H1-hESC hg19; CHD1 K562 hg19; MAFF HepG2 hg19; NEUROD2 26341353 ChIP-Seq CORTEX Mouse; SMC3 GM12878 hg19; RBBP5 K562 hg19; CTCF cortical plate mm9; MXI1 CH12.LX mm9; SPI1 26923725 Chip-Seq MACROPHAGESS Mouse; P63 26484246 Chip-Seq KERATINOCYTES Human; SOX2 21211035 ChIP-Seq LN229 Human; CTCF 20526341 ChIP-Seq ESCs Human; TAF2 19829295 ChIP-Seq ESCs Human; RARB 24833708 ChIP-Seq LIVER Mouse; MAZ K562 hg19; CEBPB 26923725 Chip-Seq MACROPHAGESS Mouse; CTCF fibroblast of villous mesenchyme hg19; POLR2AphosphoS5 U-87 MG hg19; RELA GM12878 hg19; SUPT20H HeLa—S3 hg19; GATA2 20887958 ChIP-Seq HPC-7 Mouse; EBNA2 21746931 ChIP-Seq IB4-LCL Human; SREBF1 GM12878 hg19; TCF3 18692474 ChIP-Seq MEFs Mouse; SIN3A SK—N—SH hg19; GATA1 26923725 Chip-Seq HPCs Mouse; EP300 MCF-7 hg19; SMAD1 18555785 ChIP-Seq MESCs Mouse; FOXP2 SK—N-MC hg19; ELF1 HepG2 hg19; ZNF274 21170338 ChIP-Seq K562 Hela; ELF1 20517297 ChIP-Seq JURKAT Human; CTCF Caco-2 hg19; STAT4 19710469 ChIP-ChIP TH1 Mouse; POLR2AphosphoS5 G1E-ER4 mm9; SPI1 20517297 ChIP-Seq HL60 Human; STAT3 20064451 ChIP-Seq CD4+T Mouse; WDR5 24793694 ChIP-Seq LNCAP Human; REST SK—N—SH hg19; MYCN 19997598 ChIP-ChIP NEUROBLASTOMA Human; RXRA HepG2 hg19; ASXL1 24218140 ChIP-Seq BMDM Mouse; GBX2 23144817 ChIP-Seq PC3 Human; SP4 H1-hESC hg19; EZH2 keratinocyte hg19; MAX C2C12 mm9; CTCF osteoblast hg19; WRNIP1 GM12878 hg19; EZH2 GM12878 hg19; MXII HepG2 hg19; EZH2 skeletal muscle myoblast hg19; KDM1A K562 hg19; E2F4 CH12.LX mm9; ZNF143 HeLa—S3 hg19; CTCF spleen hg19; FOXO3 22982991 ChIP-Seq MACROPHAGES Mouse; FOXA1 25329375 ChIP-Seq VCAP Human; PU.1 20513432 ChIP-Seq Bcells Mouse; FLI1 21867929 ChIP-Seq CD8 Mouse; PPARA 22158963 ChIP-Seq LIVER Mouse; FOXA1 27270436 Chip-Seq PROSTATE Human; KAP1 27257070 Chip-Seq ESCs Mouse; SOX2 18692474 ChIP-Seq MESCs Mouse; TP53 23651856 ChIP-Seq MEFs Mouse; NFE2 K562 hg19; YY1 A549 hg19; POLR2A GM12892 hg19; NR3C1 ECC-1 hg19; TCF3 18692474 ChIP-Seq MESCs Mouse; CTCF brain microvascular endothelial cell hg19; RUNX1 26923725 Chip-Seq MACROPHAGESS Mouse; SETDB1 19884257 ChIP-Seq MESCs Mouse; VDR 22108803 ChIP-Seq LS180 Human; MYC HeLa—S3 hg19; CTCF fibroblast of mammary gland hg19; NANOG 18700969 ChIP-ChIP MESCs Mouse; SP2 H1-hESC hg19; ESR1 17901129 ChIP-ChIP LIVER Mouse; NANOG 18555785 ChIP-Seq MESCs Mouse; JUND CH12.LX mm9; SOX2 16153702 ChIP-ChIP HESCs Human; YY1 HepG2 hg19; FLI1 20887958 ChIP-Seq HPC-7 Mouse; EKLF 21900194 ChIP-Seq ERYTHROCYTE Mouse; FOXD4 human tf ARCHS4 coexpression; ZNF212 human tf ARCHS4 coexpression; ZNF415 human tf ARCHS4 coexpression; ZNF556 human tf ARCHS4 coexpression; ZNF554 human tf ARCHS4 coexpression; ZNF596 human tf ARCHS4 coexpression; ZNF680 human tf ARCHS4 coexpression; ZIC4 human tf ARCHS4 coexpression; ISX human tf ARCHS4 coexpression; ZNF620 human tf ARCHS4 coexpression; ZNF366 human tf ARCHS4 coexpression; INSM2 human tf ARCHS4 coexpression; ZNF543 human tf ARCHS4 coexpression; ZNF664 human tf ARCHS4 coexpression; FIZ1 human tf ARCHS4 coexpression; ZNF609 human tf ARCHS4 coexpression; ZNF443 human tf ARCHS4 coexpression; ZBTB20 human tf ARCHS4 coexpression; ZBTB7C human tf ARCHS4 coexpression; ZNF74 human tf ARCHS4 coexpression; ZBTB37 human tf ARCHS4 coexpression; ZNF749 human tf ARCHS4 coexpression; ZNF17 human tf ARCHS4 coexpression; ZNF555 human tf ARCHS4 coexpression; ZNF354B human tf ARCHS4 coexpression; ZNF286A human tf ARCHS4 coexpression; ZNF503 human tf ARCHS4 coexpression; ZNF474 human tf ARCHS4 coexpression; DPF1 human tf ARCHS4 coexpression; ZNF280C human tf ARCHS4 coexpression; ZFAT human tf ARCHS4 coexpression; ZNF302 human tf ARCHS4 coexpression; ZNF423 human tf ARCHS4 coexpression; ZKSCAN1 human tf ARCHS4 coexpression; TBX18 human tf ARCHS4 coexpression; BBX human tf ARCHS4 coexpression; DR1 human tf ARCHS4 coexpression; ZNF639 human tf ARCHS4 coexpression; ZNF45 human tf ARCHS4 coexpression; DRGX human tf ARCHS4 coexpression; NKX2-4 human tf ARCHS4 coexpression; ZNF132 human tf ARCHS4 coexpression; NKX2-8 human tf ARCHS4 coexpression; VEZF1 human tf ARCHS4 coexpression; ZFP37 human tf ARCHS4 coexpression; ZNF750 human tf ARCHS4 coexpression; OSR1 human tf ARCHS4 coexpression; ZNF462 human tf ARCHS4 coexpression; ZNF256 human tf ARCHS4 coexpression; ZNF18 human tf ARCHS4 ZNF714 human tf ARCHS4 coexpression; ZNF691 human tf ARCHS4 coexpression; coexpression; GLIS2 human tf ARCHS4 coexpression; ZNF292 human tf ARCHS4 coexpression; ZNF34 human tf ARCHS4 coexpression; ZKSCAN5 human tf ARCHS4 coexpression; ZC3HAV1 human tf ARCHS4 coexpression; EXOC2 human tf ARCHS4 coexpression; TRIM32 human tf ARCHS4 coexpression; CENPB human tf ARCHS4 coexpression; SLC26A10 human tf ARCHS4 coexpression; RC3H1 human tf ARCHS4 coexpression; NCOR1 human tf ARCHS4 coexpression; FAM171B human tf ARCHS4 coexpression; HES6 human tf ARCHS4 coexpression; U2AF1 human tf ARCHS4 coexpression; BPNT1 human tf ARCHS4 coexpression; FGD1 human tf ARCHS4 coexpression; MST1R human tf ARCHS4 coexpression; MECP2 human tf ARCHS4 coexpression; RNASE2 human tf ARCHS4 coexpression; CCDC71 human tf ARCHS4 coexpression; TCEAL8 human tf ARCHS4 coexpression; EWSR1 human tf ARCHS4 coexpression; H1FX human tf ARCHS4 coexpression; CPSF4L human tf ARCHS4 coexpression; ZDHHC11 human tf ARCHS4 coexpression; PLXNB3 human tf ARCHS4 coexpression; RBM20 human tf ARCHS4 coexpression; PKHDIL1 human tf ARCHS4 coexpression; TIGD6 human tf ARCHS4 coexpression; DEPDC5 human tf ARCHS4 coexpression; DEPDC1B human tf ARCHS4 coexpression; BRD9 human tf ARCHS4 coexpression; ZNF615 human tf ARCHS4 coexpression; ZNF461 human tf ARCHS4 coexpression; ZNF512 human tf ARCHS4 coexpression; ZFP57 human tf ARCHS4 coexpression; LEUTX human tf ARCHS4 coexpression; ZNF718 human tf ARCHS4 coexpression; PREB human tf ARCHS4 coexpression; CEBPA human tf ARCHS4 coexpression; TOX4 human tf ARCHS4 coexpression; TAXIBP1 human tf ARCHS4 coexpression; AKNA human tf ARCHS4 coexpression; HMGA1 human tf ARCHS4 coexpression; ZNF827 human tf ARCHS4 coexpression; ZNF837 human tf ARCHS4 coexpression; FEZF2 human tf ARCHS4 coexpression; ZNF362 human tf ARCHS4 coexpression; THAP9 human tf ARCHS4 coexpression; ATOH8 human tf ARCHS4 coexpression; ZNF804A human tf ARCHS4 coexpression; THAP2 human tf ARCHS4 coexpression; ZNF788 human tf ARCHS4 coexpression; ZNF775 human tf ARCHS4 coexpression; ZNF720 human tf ARCHS4 coexpression; ZNF845 human tf ARCHS4 coexpression; MEOX1 human tf ARCHS4 coexpression; ZNF254 human tf ARCHS4 coexpression; ZFYVE26 human tf ARCHS4 coexpression; E2F4 human tf ARCHS4 coexpression; TFAP2D human tf ARCHS4 coexpression; RUNX3 human tf ARCHS4 coexpression; YBX1 human tf ARCHS4 coexpression; MYBL2 human tf ARCHS4 coexpression; E2F1 human tf ARCHS4 coexpression; GATA1 human tf ARCHS4 coexpression; LYL1 human tf ARCHS4 coexpression; ZNF175 human tf ARCHS4 coexpression; POU6F2 human tf ARCHS4 coexpression; MLX human tf ARCHS4 coexpression; MYF5 human tf ARCHS4 coexpression; GLI1 human tf ARCHS4 coexpression; PPARD human tf ARCHS4 coexpression; IRX4 human tf ARCHS4 coexpression; TLX2 human tf ARCHS4 coexpression; OTX1 human tf ARCHS4 coexpression; OLIG2 human tf ARCHS4 coexpression; DLX2 human tf ARCHS4 coexpression; TIGD4 human tf ARCHS4 coexpression; ASHIL human tf ARCHS4 coexpression; ETV6 human tf ARCHS4 coexpression; ASCL1 human tf ARCHS4 coexpression; TBX19 human tf ARCHS4 coexpression; VAX1 human tf ARCHS4 coexpression; POU4F1 human tf ARCHS4 coexpression; HOXA10 human tf ARCHS4 coexpression; BATF human tf ARCHS4 coexpression; IKZF3 human tf ARCHS4 coexpression; ZNF2 human tf ARCHS4 coexpression; PITX2 human tf ARCHS4 coexpression; HNF4G human tf ARCHS4 coexpression; CREBZF human tf ARCHS4 coexpression; SPIC human tf ARCHS4 coexpression; AR human tf ARCHS4 coexpression; BNC1 human tf ARCHS4 coexpression; EMX2 human tf ARCHS4 coexpression; MLXIP human tf ARCHS4 coexpression; IRX3 human tf ARCHS4 coexpression; ASCL2 human tf ARCHS4 coexpression; OLIG1 human tf ARCHS4 coexpression; IKZF1 human tf ARCHS4 coexpression; SHOX human tf ARCHS4 coexpression; ZNF181 human tf ARCHS4 coexpression; VAX2 human tf ARCHS4 coexpression; SOX14 human tf ARCHS4 coexpression; GCM1 human tf ARCHS4 coexpression; ONECUT1 human tf ARCHS4 coexpression; ID3 human tf ARCHS4 coexpression; ZBTB17 human tf ARCHS4 coexpression; PAX3 human tf ARCHS4 coexpression; E2F5 human tf ARCHS4 coexpression; KLF16 human tf ARCHS4 coexpression; HOXD13 human tf ARCHS4 coexpression; SOX15 human tf ARCHS4 coexpression; HOXD10 human tf ARCHS4 coexpression; IRF3 human tf ARCHS4 coexpression; MAX human tf ARCHS4 coexpression; PLAGL1 human tf ARCHS4 coexpression; CREB1 human tf ARCHS4 coexpression; HOXD11 human tf ARCHS4 coexpression; MYB MEL cell line mm9; GATA3 20176728 ChIP-ChIP TSCs Mouse; PBX3 GM12878 hg19; SRF ECC-1 hg19; MAX ECC-1 hg19; CEBPB 24764292 ChIP-Seq MC3T3 Mouse; ZMIZ1 K562 hg19; FLI1 26923725 Chip-Seq HEMOGENIC-ENDOTHELIUM Mouse; PBX 27287812 Chip-Seq EMBYONIC-LIMB Mouse; SMC3 HeLa—S3 hg19; CCNT2 K562 hg19; CTCF pancreas hg19; ARID3A HepG2 hg19; RELA GM18951 hg19; RCOR1 GM12878 hg19; YY1 NT2-D1 hg19; YY1 GM12878 hg19; MYC 19829295 ChIP-Seq ESCs Human; HOXD13 18407260 ChIP-ChIP DEVELOPING-LIMBS Mouse; MAFK HepG2 hg19; ELF1 GM12878 hg19; MAX SK—N—SH hg19; SRY 22984422 ChIP-ChIP TESTIS Rat; HCFC1 20581084 ChIP-Seq MESCs Mouse; STAT1 20625510 ChIP-Seq HELA Human; TRP63 18441228 ChIP-ChIP KERATINOCYTES Mouse; CTCF CH12.LX mm9; BMI1 19503595 ChIP-Seq MEFsC Mouse; IRF8 22096565 ChIP-ChIP GC-B Human; CTCF GM12864 hg19; ZBTB33 A549 hg19; PML GM12878 hg19; ZBTB7A ECC-1 hg19; SPI1 22096565 ChIP-ChIP GC-B Mouse; CTCF GM12866 hg19; CTCF fibroblast of lung hg19; ZBTB33 K562 hg19; RUNX1 17652178 ChIP-ChIP JURKAT Human; YY1 H1-hESC hg19; YY1 GM12891 hg19; POLR2AphosphoS2 MEL cell line mm9; POLR2A GM19193 hg19; EZH2 astrocyte hg19; SMC3 MEL cell line mm9; MAFF K562 hg19; ZNF143 K562 hg19; USF2 HeLa—S3 hg19; HCFC1 MEL cell line mm9; PHF8 20622854 ChIP-Seq HELA Human; P68 20966046 ChIP-Seq HELA Human; KDM5B K562 hg19; CTCF B cell hg19; CREBBP K562 hg19; TBP MEL cell line mm9; IRF8 22096565 ChIP-ChIP GC-B Mouse; SP1 A549 hg19; CHD2 MEL cell line mm9; ELK1 19687146 ChIP-ChIP HELA Human; MTA3 GM12878 hg19; CTCF choroid plexus epithelial cell hg19; MYC 18555785 ChIP-Seq MESCs Mouse; ELF1 A549 hg19; MYC H1-hESC hg19; POLR2AphosphoS5 GM12878 hg19; SP1 K562 hg19; ZBTB7A HepG2 hg19; NR2C2 K562 hg19; TRIM28 K562 hg19; MEF2C GM12878 hg19; MAFK MEL cell line mm9; TP63 22573176 ChIP-Seq HFKS Human; SRF GM12878 hg19; ZFP322A 24550733 ChIP-Seq MESCs Mouse; RUNX3 GM12878 hg19; SP1 GM12878 hg19; POLR2AphosphoS5 G1E mm9; TAF7 K562 hg19; REST 18959480 ChIP-ChIP MESCs Mouse; ELF1 MCF-7 hg19; MYBL1 21750041 ChIP-ChIP SPERMATOCYTES Mouse; E2F4 MEL cell line mm9; TRIM28 U20S hg19; EP300 heart mm9; SMC3 HepG2 hg19; POLR2A GM18951 hg19; RFX5 HeLa—S3 hg19; EZH2 H1-hESC hg19; CTCF cerebellum mm9; NANOG 20526341 ChIP-Seq ESCs Human; FOXM1 GM12878 hg19; DCP1A 22483619 ChIP-Seq HELA Human; YY1 22570637 ChIP-Seq MALME-3M Human; IRF1 21803131 ChIP-Seq MONOCYTES Human; SIN3A HepG2 hg19; RAD21 MEL cell line mm9; YY1 ECC-1 hg19; CTCF 18555785 ChIP-Seq MESCs Mouse; eGFP-NR4A1 K562 hg19; ESR1 15608294 ChIP-ChIP MCF-7 Human; CRX 20693478 ChIP-Seq ADULT RETINA Mouse; TBX5 21415370 ChIP-Seq HL-1 Mouse; REST 19997604 ChIP-ChIP NEURONS Mouse; ZFP1 human tf ARCHS4 coexpression; TSHZ2 human tf ARCHS4 coexpression; ZSCAN22 human tf ARCHS4 coexpression; ZNF572 human tf ARCHS4 coexpression; ZSCAN4 human tf ARCHS4 coexpression; ZNF571 human tf ARCHS4 coexpression; LBX2 human tf ARCHS4 coexpression; TPRX1 human tf ARCHS4 coexpression; IRX5 human tf ARCHS4 coexpression; FOXR1 human tf ARCHS4 coexpression; FOXD4L1 human tf ARCHS4 coexpression; TAL2 human tf ARCHS4 coexpression; ZNF70 human tf ARCHS4 coexpression; ZSCAN23 human tf ARCHS4 coexpression; ZNF283 human tf ARCHS4 coexpression; HMX3 human tf ARCHS4 coexpression; ZNF600 human tf ARCHS4 coexpression; ZNF79 human tf ARCHS4 coexpression; ZNF681 human tf ARCHS4 ZNF565 human tf ARCHS4 coexpression; ZNF493 human tf ARCHS4 coexpression; coexpression; ZNF431 human tf ARCHS4 coexpression; ZNF700 human tf ARCHS4 coexpression; ZSCAN16 human tf ARCHS4 coexpression; ZNF138 human tf ARCHS4 coexpression; ZNF100 human tf ARCHS4 coexpression; ZNF501 human tf ARCHS4 coexpression; ZNF613 human tf ARCHS4 coexpression; ZNF608 human tf ARCHS4 coexpression; ZNF562 human tf ARCHS4 coexpression; TBX22 human tf ARCHS4 coexpression; ZSCAN20 human tf ARCHS4 coexpression; ZFP30 human tf ARCHS4 coexpression; ZNF142 human tf ARCHS4 coexpression; ZBTB47 human tf ARCHS4 coexpression; ZBTB16 human tf ARCHS4 coexpression; OVOL2 human tf ARCHS4 coexpression; ZNF419 human tf ARCHS4 coexpression; PATZ1 human tf ARCHS4 coexpression; ZNF8 human tf ARCHS4 coexpression; ZNF532 human tf ARCHS4 coexpression; ZNF275 human tf ARCHS4 coexpression; HMX1 human tf ARCHS4 coexpression; RXRB human tf ARCHS4 coexpression; ZNF500 human tf ARCHS4 coexpression; FOXJ1 human tf ARCHS4 coexpression; ZNF20 human tf ARCHS4 coexpression; ZFHX2 human tf ARCHS4 coexpression; ZNF280A human tf ARCHS4 coexpression; ZNF354A human tf ARCHS4 coexpression; ZNF688 human tf ARCHS4 coexpression; ZNF83 human tf ARCHS4 coexpression; PRDM9 human tf ARCHS4 coexpression; ZNF513 human tf ARCHS4 coexpression; GFI1 human tf ARCHS4 coexpression; ZNF333 human tf ARCHS4 coexpression; ZNF486 human tf ARCHS4 coexpression; ZNF233 human tf ARCHS4 coexpression; ZKSCAN2 human tf ARCHS4 coexpression; VENTX human tf ARCHS4 coexpression; HMBOX1 human tf ARCHS4 coexpression; ZNF660 human tf ARCHS4 coexpression; FOXN4 human tf ARCHS4 coexpression; ZNF519 human tf ARCHS4 coexpression; ZNF628 human tf ARCHS4 coexpression; ZNF84 human tf ARCHS4 coexpression; ZNF595 human tf ARCHS4 coexpression; SETDB1 human tf ARCHS4 coexpression; RNF113B human tf ARCHS4 coexpression; TRAFD1 human tf ARCHS4 coexpression; ZC3H4 human tf ARCHS4 coexpression; AHDC1 human tf ARCHS4 coexpression; GTF2F1 human tf ARCHS4 coexpression; DZIP1L human tf ARCHS4 coexpression; SLC2A4RG human tf ARCHS4 coexpression; DEPDC7 human tf ARCHS4 coexpression; RGS9 human tf ARCHS4 coexpression; DVL1 human tf ARCHS4 coexpression; CBLL1 human tf ARCHS4 coexpression; SSH1 human tf ARCHS4 coexpression; PDS5B human tf ARCHS4 coexpression; ZBP1 human tf ARCHS4 coexpression; ZC3H18 human tf ARCHS4 coexpression; DHX57 human tf ARCHS4 coexpression; PBRM1 human tf ARCHS4 coexpression; IGHM human tf ARCHS4 coexpression; ZGPAT human tf ARCHS4 coexpression; OTOP3 human tf ARCHS4 coexpression; TIGD5 human tf ARCHS4 coexpression; MKRN3 human tf ARCHS4 coexpression; ZNF738 human tf ARCHS4 coexpression; ZNF528 human tf ARCHS4 coexpression; GATAD2A human tf ARCHS4 coexpression; CUL5 human tf ARCHS4 coexpression; NUFIP1 human tf ARCHS4 coexpression; DUSP12 human tf ARCHS4 coexpression; MKRN2 human tf ARCHS4 coexpression; MBD3 human tf ARCHS4 coexpression; ZNF705D human tf ARCHS4 coexpression; HOMEZ human tf ARCHS4 coexpression; ZNF580 human tf ARCHS4 coexpression; ONECUT3 human tf ARCHS4 coexpression; ZNF783 human tf ARCHS4 coexpression; ZNF616 human tf ARCHS4 coexpression; DUXA human tf ARCHS4 coexpression; ZNF358 human tf ARCHS4 coexpression; ZNF652 human tf ARCHS4 coexpression; ZNF69 human tf ARCHS4 coexpression; ZNF334 human tf ARCHS4 coexpression; ZNF248 human tf ARCHS4 coexpression; ZNF667 human tf ARCHS4 coexpression; SOX18 human tf ARCHS4 coexpression; HES5 human tf ARCHS4 coexpression; ZNF579 human tf ARCHS4 coexpression; ZNF433 human tf ARCHS4 coexpression; CXXC1 human tf ARCHS4 coexpression; SP100 human tf ARCHS4 coexpression; ZNF790 human tf ARCHS4 coexpression; ZNF780A human tf ARCHS4 coexpression; ZNF841 human tf ARCHS4 coexpression; ZNF98 human tf ARCHS4 coexpression; ZNF763 human tf ARCHS4 coexpression; HMX2 human tf ARCHS4 coexpression; ZNF385C human tf ARCHS4 coexpression; ZNF792 human tf ARCHS4 coexpression; NEUROG2 human tf ARCHS4 coexpression; BOLA1 human tf ARCHS4 coexpression; THAP11 human tf ARCHS4 coexpression; ZNF598 human tf ARCHS4 coexpression; ANKZF1 human tf ARCHS4 coexpression; ZUFSP human tf ARCHS4 coexpression; ZNF780B human tf ARCHS4 coexpression; TUB human tf ARCHS4 coexpression; HOXC4 human tf ARCHS4 coexpression; ATOH7 human tf ARCHS4 coexpression; HOXC6 human tf ARCHS4 coexpression; PITX1 human tf ARCHS4 coexpression; ZNF76 human tf ARCHS4 coexpression; BARX2 human tf ARCHS4 coexpression; NR2E3 human tf ARCHS4 coexpression; ZNF200 human tf ARCHS4 coexpression; DLX6 human tf ARCHS4 coexpression; SOX8 human tf ARCHS4 coexpression; TEAD2 human tf ARCHS4 coexpression; ZNF37A human tf ARCHS4 coexpression; GRHL2 human tf ARCHS4 coexpression; RFX2 human tf ARCHS4 coexpression; ZNF148 human tf ARCHS4 coexpression; ELF3 human tf ARCHS4 coexpression; REL human tf ARCHS4 coexpression; NR2F6 human tf ARCHS4 coexpression; HOXB13 human tf ARCHS4 coexpression; ALX3 human tf ARCHS4 coexpression; CDX2 human tf ARCHS4 coexpression; FLI1 human tf ARCHS4 coexpression; DLX1 human tf ARCHS4 coexpression; SOX13 human tf ARCHS4 coexpression; RFX5 human tf ARCHS4 coexpression; RXRG human tf ARCHS4 coexpression; PRDM5 human tf ARCHS4 coexpression; LBX1 human tf ARCHS4 coexpression; ZEB1 human tf ARCHS4 coexpression; SIX3 human tf ARCHS4 coexpression; SNAPC4 human tf ARCHS4 coexpression; ZNF180 human tf ARCHS4 coexpression; ZNF197 human tf ARCHS4 coexpression; POU3F1 human tf ARCHS4 coexpression; WT1 human tf ARCHS4 coexpression; HOXC10 human tf ARCHS4 coexpression; ZSCAN2 human tf ARCHS4 coexpression; ZNF91 human tf ARCHS4 coexpression; FOXG1 human tf ARCHS4 coexpression; HOXC5 human tf ARCHS4 coexpression; SOX7 human tf ARCHS4 coexpression; ZNF160 human tf ARCHS4 coexpression; STAT2 human tf ARCHS4 coexpression; ZNF444 human tf ARCHS4 coexpression; ESRRA human tf ARCHS4 coexpression; ZNF44 human tf ARCHS4 coexpression; NKX2-1 human tf ARCHS4 coexpression; HOXD9 human tf ARCHS4 coexpression; EPAS1 human tf ARCHS4 coexpression; ATF2 human tf ARCHS4 coexpression; CDX1 human tf ARCHS4 coexpression; VDR human tf ARCHS4 coexpression; UBTF human tf ARCHS4 coexpression; TLX1 human tf ARCHS4 coexpression; NR5A2 human tf ARCHS4 coexpression; LHX3 human tf ARCHS4 coexpression; SP4 human tf ARCHS4 coexpression; TSC22D1 human tf ARCHS4 coexpression; KLF5 human tf ARCHS4 coexpression; NFATC2 human tf ARCHS4 coexpression; ZNF10 human tf ARCHS4 coexpression; TFAP4 human tf ARCHS4 coexpression; HOXA5 human tf ARCHS4 coexpression; SOX3 human tf ARCHS4 coexpression; IRF6 human tf ARCHS4 coexpression; TCF21 human tf ARCHS4 coexpression; HOXD3 human tf ARCHS4 coexpression; HOXD1 human tf ARCHS4 coexpression; IRF5 human tf ARCHS4 coexpression; PRDM7 human tf ARCHS4 coexpression; IRF1 human tf ARCHS4 coexpression; GCM2 human tf ARCHS4 coexpression; PROX1 human tf ARCHS4 coexpression; SPDEF human tf ARCHS4 coexpression; HOXC11 human tf ARCHS4 coexpression; HOXC13 human tf ARCHS4 coexpression; HOXA7 human tf ARCHS4 coexpression; ZNF205 human tf ARCHS4 coexpression; NR2C1 human tf ARCHS4 coexpression; SATB2 human tf ARCHS4 coexpression; NFE2 human tf ARCHS4 coexpression; MAX HeLa—S3 hg19; ZBTB33 GM12878 hg19; IRF3 GM12878 hg19; BRCA1 HeLa—S3 hg19; KAT2B K562 hg19; SIN3A CH12.LX mm9; CHD2 GM12878 hg19; CHD7 K562 hg19; RCOR1 CH12.LX mm9; SIRT6 H1-hESC hg19; MYC CH12.LX mm9; CTCF 26484167 Chip-Seq Bcells Mouse; BCOR 27268052 Chip-Seq Bcells Human; AR 20517297 ChIP-Seq VCAP Human; YY1 K562 hg19; POU5F1 18692474 ChIP-Seq MESCs Mouse; POU5F1 16518401 ChIP-PET MESCs Mouse; SALL1 21062744 ChIP-ChIP HESCs Human; SP2 K562 hg19; FOXA1 T47D hg19; KDM6A 18722178 ChIP-ChIP U937 AND SAOS2 Human; CUX1 19635798 ChIP-ChIP MULTIPLE HUMAN CANCER TYPES Human; STAT5A GM12878 hg19; SPI1HL-60 hg19; MYC 18358816 ChIP-ChIP MESCs Mouse; HDAC2 K562 hg19; SOX9 25088423 ChIP-ChIP EMBRYONIC GONADS Mouse; NR2F2 K562 hg19; BCLAF1 K562 hg19; IRF8 21731497 ChIP-ChIP J774 Mouse; CTCF G1E-ER4 mm9; MYC MEL cell line mm9; MYC GM12878 hg19; CHD2 CH12.LX mm9; CTCF cardiac fibroblast hg19; EZH2 myotube hg19; POLR2A GM15510 hg19; CEBPA 26348894 ChIP-Seq LIVER Mouse; ELK4 HeLa—S3 hg19; GABPA H1-hESC hg19; POU5F1 18700969 ChIP-ChIP MESCs Mouse; GATA1 G1E-ER4 mm9; MYC 19915707 ChIP-ChIP AK7 Human; PPARG 19300518 ChIP-PET 3T3-L1 Mouse; BCL11A GM12878 hg19; NR2C2 HepG2 hg19; EZH2 fibroblast of lung hg19; CUX1 GM12878 hg19; KAT2A CH12.LX mm9; E2F1 20622854 ChIP-Seq HELA Human; SIN3A A549 hg19; MEF2A 21415370 ChIP-Seq HL-1 Mouse; GABPA HepG2 hg19; CEBPB GM12878 hg19; STAT5A K562 hg19; CEBPZ HepG2 hg19; TP53 22127205 ChIP-Seq IMR90 Human; HCFC1 HeLa—S3 hg19; RBBP5 H1-hESC hg19; HCFC1 CH12.LX mm9; EZH2 fibroblast of dermis hg19; CUX1 K562 hg19; P53 21459846 ChIP-Seq SAOS-2 Human; RAC3 21632823 ChIP-Seq H3396 Human; SUPT20H GM12878 hg19; ZNF671 human tf ARCHS4 coexpression; ZSCAN5A human tf ARCHS4 coexpression; ZNF280D human tf ARCHS4 coexpression; FOXB1 human tf ARCHS4 coexpression; ZIM3 human tf ARCHS4 coexpression; PRDM15 human tf ARCHS4 coexpression; ATF7 human tf ARCHS4 coexpression; SP7 human tf ARCHS4 coexpression; ZNF282 human tf ARCHS4 coexpression; ZNF32 human tf ARCHS4 coexpression; SHOX2 human tf ARCHS4 coexpression; IRF2 human tf ARCHS4 coexpression; FOXI1 human tf ARCHS4 coexpression; ZNF473 human tf ARCHS4 coexpression; RERE human tf ARCHS4 coexpression; GFI1B human tf ARCHS4 coexpression; EBF1 human tf ARCHS4 coexpression; KLF15 human tf ARCHS4 coexpression; AIRE human tf ARCHS4 coexpression; ZNF19 human tf ARCHS4 coexpression; ZNF341 human tf ARCHS4 coexpression; ZNF426 human tf ARCHS4 coexpression; ZNF557 human tf ARCHS4 coexpression; ATOH1 human tf ARCHS4 coexpression; PHF20 human tf ARCHS4 coexpression; ZNF839 human tf ARCHS4 coexpression; ZNF28 human tf ARCHS4 coexpression; ZNF26 human tf ARCHS4 coexpression; HOXA4 human tf ARCHS4 coexpression; MESP2 human tf ARCHS4 coexpression; MIXL1 human tf ARCHS4 coexpression; ALX1 human tf ARCHS4 coexpression; LCORL human tf ARCHS4 coexpression; ZNF213 human tf ARCHS4 coexpression; DMRT2 human tf ARCHS4 coexpression; HHEX human tf ARCHS4 coexpression; ZBTB25 human tf ARCHS4 coexpression; TBX15 human tf ARCHS4 coexpression; ZNF184 human tf ARCHS4 coexpression; ZNF629 human tf ARCHS4 coexpression; ZNF550 human tf ARCHS4 coexpression; NKX3-2 human tf ARCHS4 coexpression; RFX4 human tf ARCHS4 coexpression; ESX1 human tf ARCHS4 coexpression; RARG human tf ARCHS4 coexpression; POU4F2 human tf ARCHS4 coexpression; CDC5L human tf ARCHS4 coexpression; MEIS1 human tf ARCHS4 coexpression; ELK1 human tf ARCHS4 coexpression; ZNF384 human tf ARCHS4 coexpression; MXD4 human tf ARCHS4 coexpression; HOXC12 human tf ARCHS4 coexpression; NEUROG3 human tf ARCHS4 coexpression; MYOG human tf ARCHS4 coexpression; TBX4 human tf ARCHS4 coexpression; TBX2 human tf ARCHS4 coexpression; SMAD9 human tf ARCHS4 coexpression; SIX1 human tf ARCHS4 coexpression; HOXB8 human tf ARCHS4 coexpression; ZNF410 human tf ARCHS4 coexpression; VSX2 human tf ARCHS4 coexpression; ID2 human tf ARCHS4 coexpression; TFDP2 human tf ARCHS4 coexpression; ZNF451 human tf ARCHS4 coexpression; FOXM1 human tf ARCHS4 coexpression; MYF6 human tf ARCHS4 coexpression; WHSC1 human tf ARCHS4 coexpression; HOXB6 human tf ARCHS4 coexpression; NKX2-3 human tf ARCHS4 coexpression; MYOD1 human tf ARCHS4 coexpression; GATA5 human tf ARCHS4 coexpression; GRHL1 human tf ARCHS4 coexpression; RORC human tf ARCHS4 coexpression; SPIB human tf ARCHS4 coexpression; NFIC human tf ARCHS4 coexpression; FOXK2 human tf ARCHS4 coexpression; TP53 human tf ARCHS4 coexpression; GATA6 human tf ARCHS4 coexpression; HNF4A human tf ARCHS4 coexpression; KLF8 human tf ARCHS4 coexpression; ZNF174 human tf ARCHS4 coexpression; MAZ human tf ARCHS4 coexpression; TFEC human tf ARCHS4 coexpression; HOXA6 human tf ARCHS4 coexpression; MEOX2 human tf ARCHS4 coexpression; GATA3 human tf ARCHS4 coexpression; PRDM16 human tf ARCHS4 coexpression; ZFHX3 human tf ARCHS4 coexpression; STAT4 human tf ARCHS4 coexpression; ELF5 human tf ARCHS4 coexpression; EHF human tf ARCHS4 coexpression; HNF1A human tf ARCHS4 coexpression; RAX human tf ARCHS4 coexpression; TBX6 human tf ARCHS4 coexpression; DMRTB1 human tf ARCHS4 coexpression; KLF17 human tf ARCHS4 coexpression; ZNF300 human tf ARCHS4 coexpression; PPP1R13L human tf ARCHS4 coexpression; ZC3H14 human tf ARCHS4 coexpression; RAPGEF3 human tf ARCHS4 coexpression; SP140 human tf ARCHS4 coexpression; WNT8B human tf ARCHS4 coexpression; RBM6 human tf ARCHS4 coexpression; ZNF506 human tf ARCHS4 coexpression; ZNF429 human tf ARCHS4 coexpression; ZNF846 human tf ARCHS4 coexpression; ZNF781 human tf ARCHS4 coexpression; ZFP92 human tf ARCHS4 coexpression; ZNF793 human tf ARCHS4 coexpression; ZFR2 human tf ARCHS4 coexpression; TCF24 human tf ARCHS4 coexpression; ZNF784 human tf ARCHS4 coexpression; THAP6 human tf ARCHS4 coexpression; ZNF768 human tf ARCHS4 coexpression; ZNF764 human tf ARCHS4 coexpression; FOXK1 human tf ARCHS4 coexpression; THAP8 human tf ARCHS4 coexpression; GATAD1 human tf ARCHS4 coexpression; ZNF773 human tf ARCHS4 coexpression; ZCCHC11 human tf ARCHS4 coexpression; ZNF835 human tf ARCHS4 coexpression; ZCCHC6 human tf ARCHS4 coexpression; TCF25 human tf ARCHS4 coexpression; ZNF804B human tf ARCHS4 coexpression; ZNF564 human tf ARCHS4 coexpression; MIER2 human tf ARCHS4 coexpression; CHD6 human tf ARCHS4 coexpression; HLA-DRB3 human tf ARCHS4 coexpression; FOXD4L5 human tf ARCHS4 coexpression; RHOXF2B human tf ARCHS4 coexpression; INF2 human tf ARCHS4 coexpression; KRTAP5-9 human tf ARCHS4 coexpression; GTF2IRD2 human tf ARCHS4 coexpression; HILS1 human tf ARCHS4 coexpression; EP400 human tf ARCHS4 coexpression; FARSA human tf ARCHS4 coexpression; DMAP1 human tf ARCHS4 coexpression; ANAPC2 human tf ARCHS4 coexpression; CBX2 human tf ARCHS4 coexpression; BAZ2B human tf ARCHS4 coexpression; SSH3 human tf ARCHS4 coexpression; PPP2R3B human tf ARCHS4 coexpression; KIN human tf ARCHS4 coexpression; TUT1 human tf ARCHS4 coexpression; SHPRH human tf ARCHS4 coexpression; BDP1 human tf ARCHS4 coexpression; ZC3H8 human tf ARCHS4 coexpression; COPS4 human tf ARCHS4 coexpression; XPA human tf ARCHS4 coexpression; NUPL2 human tf ARCHS4 coexpression; SETDB2 human tf ARCHS4 coexpression; NROB2 human tf ARCHS4 coexpression; RNF113A human tf ARCHS4 coexpression; CHD7 human tf ARCHS4 coexpression; ZNF253 human tf ARCHS4 coexpression; ZBTB9 human tf ARCHS4 coexpression; ZNF625 human tf ARCHS4 coexpression; ZNF566 human tf ARCHS4 coexpression; ZNF445 human tf ARCHS4 coexpression; ZNF530 human tf ARCHS4 coexpression; ZNF623 human tf ARCHS4 coexpression; ZNF320 human tf ARCHS4 coexpression; ZNF721 human tf ARCHS4 coexpression; ZNF329 human tf ARCHS4 coexpression; ZNF678 human tf ARCHS4 coexpression; ZNF552 human tf ARCHS4 coexpression; ZNF114 human tf ARCHS4 coexpression; ZNF518A human tf ARCHS4 coexpression; ZNF575 human tf ARCHS4 coexpression; ZNF224 human tf ARCHS4 coexpression; ZNF404 human tf ARCHS4 coexpression; CEBPE human tf ARCHS4 coexpression; ZNF296 human tf ARCHS4 coexpression; ZNF507 human tf ARCHS4 coexpression; ZNF668 human tf ARCHS4 coexpression; ZNF226 human tf ARCHS4 coexpression; SP8 human tf ARCHS4 coexpression; ZNF653 human tf ARCHS4 coexpression; ZNF577 human tf ARCHS4 coexpression; ZNF208 human tf ARCHS4 coexpression; ZSCAN12 human tf ARCHS4 coexpression; ZNF268 human tf ARCHS4 coexpression; ZNF214 human tf ARCHS4 coexpression; ZNF611 human tf ARCHS4 coexpression; ZNF284 human tf ARCHS4 coexpression; ARGFX human tf ARCHS4 coexpression; FOXD2 human tf ARCHS4 coexpression; DPRX human tf ARCHS4 coexpression; ZFP2 human tf ARCHS4 coexpression; ZNF658 human tf ARCHS4 coexpression; ZNF627 human tf ARCHS4 coexpression; ZNF587 human tf ARCHS4 coexpression; ZNF480 human tf ARCHS4 coexpression; ZNF568 human tf ARCHS4 coexpression; ZNF583 human tf ARCHS4 coexpression; ZIM2 human tf ARCHS4 coexpression; ZNF347 human tf ARCHS4 coexpression; ZNF823 human tf ARCHS4 coexpression; ZNF517 human tf ARCHS4 coexpression; ZNF257 human tf ARCHS4 coexpression; ZNF470 human tf ARCHS4 coexpression; ZNF705A human tf ARCHS4 coexpression; ZNF33B human tf ARCHS4 coexpression; ZNF502 human tf ARCHS4 coexpression; ZNF136 human tf ARCHS4 coexpression; ZNF605 human tf ARCHS4 coexpression; TSC22D2 human tf ARCHS4 ZKSCAN3 human tf ARCHS4 coexpression; ZNF33A human tf ARCHS4 coexpression; coexpression; ZNF567 human tf ARCHS4 coexpression; ZNF563 human tf ARCHS4 coexpression; ZNF548 human tf ARCHS4 coexpression; ZNF177 human tf ARCHS4 coexpression; ZNF669 human tf ARCHS4 coexpression; ZNF514 human tf ARCHS4 coexpression; ESR1 human tf ARCHS4 coexpression; OSR2 human tf ARCHS4 coexpression; TBX21 human tf ARCHS4 coexpression; NFE2L1 human tf ARCHS4 coexpression; TP63 human tf ARCHS4 coexpression; TCF3 human tf ARCHS4 coexpression; NFYC human tf ARCHS4 coexpression; DLX3 human tf ARCHS4 coexpression; SOX30 human tf ARCHS4 coexpression; NR1H3 human tf ARCHS4 coexpression; ETV7 human tf ARCHS4 coexpression; NFYA human tf ARCHS4 coexpression; ZRSR1 human tf ARCHS4 coexpression; ZNF578 human tf ARCHS4 coexpression; ZNF774 human tf ARCHS4 coexpression; RCOR1 19997604 ChIP-ChIP NEURONS Mouse; ZC3H11A MEL cell line mm9; POU5F1 18555785 ChIP-Seq MESCs Mouse; SPI1 K562 hg19; MYBL2 HepG2 hg19; HTT 18923047 ChIP-ChIP STHdh Human; SPI126923725 Chip-Seq HPCs Mouse; GABPA CH12.LX mm9; EZH2 mammary epithelial cell hg19; IRF4 GM12878 hg19; THAP11 20581084 ChIP-Seq MESCs Mouse; POLR2AphosphoS5 HCT116 hg19; NANOG 16153702 ChIP-ChIP HESCs Human; YY1 21170310 ChIP-Seq MESCs Mouse; GABPA HeLa—S3 hg19; YY1 GM12892 hg19; SP2 HepG2 hg19; GATA3 21867929 ChIP-Seq CD8 Mouse; ZNF384 GM12878 hg19; MAZ CH12.LX mm9; GABPA MCF-7 hg19; ETV2 25802403 ChIP-Seq MESCs Mouse; GATA1 MEL cell line mm9; GABPA GM12878 hg19; EZH2 T-cell acute lymphoblastic leukemia hg19; eGFP-GATA2 K562 hg19; SIX5 H1-hESC hg19; IRF1 19129219 ChIP-ChIP H3396 Human; ZZZ3 GM12878 hg19; ELF1 SK—N—SH hg19; NR2C2 HeLa—S3 hg19; GABP 17652178 ChIP-ChIP JURKAT Human; TFAP2C human tf ARCHS4 coexpression; CIC human tf ARCHS4 coexpression; ZNF777 human tf ARCHS4 coexpression; ZNF99 human tf ARCHS4 coexpression; CENPT human tf ARCHS4 coexpression; TERF2 human tf ARCHS4 coexpression; BOLA3 human tf ARCHS4 coexpression; YEATS2 human tf ARCHS4 coexpression; MESP1 human tf ARCHS4 coexpression; ZNF778 human tf ARCHS4 coexpression; ZNF791 human tf ARCHS4 coexpression; THAP4 human tf ARCHS4 coexpression; THAP5 human tf ARCHS4 coexpression; ZNF536 human tf ARCHS4 coexpression; ZNF264 human tf ARCHS4 coexpression; EVX1 human tf ARCHS4 coexpression; ZNF541 human tf ARCHS4 coexpression; RHOXF2 human tf ARCHS4 coexpression; SP110 human tf ARCHS4 coexpression; ZFP14 human tf ARCHS4 coexpression; DMRTA2 human tf ARCHS4 coexpression; ZNF547 human tf ARCHS4 coexpression; ZFP36L2 human tf ARCHS4 coexpression; ZNF117 human tf ARCHS4 coexpression; EBF4 human tf ARCHS4 coexpression; ZNF599 human tf ARCHS4 coexpression; ZNF511 human tf ARCHS4 coexpression; ZNF585B human tf ARCHS4 coexpression; AKAP8L human tf ARCHS4 coexpression; KRTAP5-1 human tf ARCHS4 coexpression; DPF3 human tf ARCHS4 coexpression; PRR3 human tf ARCHS4 coexpression; FOXO6 human tf ARCHS4 coexpression; PLXNB2 human tf ARCHS4 coexpression; ZNF544 human tf ARCHS4 coexpression; H1F0 human tf ARCHS4 coexpression; HIST1HIT human tf ARCHS4 coexpression; JRKL human tf ARCHS4 coexpression; EIF3K human tf ARCHS4 coexpression; POLR2L human tf ARCHS4 coexpression; HMGB4 human tf ARCHS4 coexpression; ZRSR2 human tf ARCHS4 coexpression; PLXNB1 human tf ARCHS4 coexpression; ZDHHC19 human tf ARCHS4 coexpression; ZNF124 human tf ARCHS4 coexpression; ZNF624 human tf ARCHS4 coexpression; ZC3H6 human tf ARCHS4 coexpression; ZNF234 human tf ARCHS4 coexpression; ZNF704 human tf ARCHS4 coexpression; MTA2 human tf ARCHS4 coexpression; MRRF human tf ARCHS4 coexpression; DUS3L human tf ARCHS4 coexpression; MINK1 human tf ARCHS4 coexpression; PCSK6 human tf ARCHS4 coexpression; APTX human tf ARCHS4 coexpression; ZC3H10 human tf ARCHS4 coexpression; UNK human tf ARCHS4 coexpression; PLXNA3 human tf ARCHS4 coexpression; RNF114 human tf ARCHS4 coexpression; CPSF4 human tf ARCHS4 coexpression; POLE4 human tf ARCHS4 coexpression; PLXNA1 human tf ARCHS4 coexpression; GRM6 human tf ARCHS4 coexpression; TRMT1 human tf ARCHS4 coexpression; RNF125 human tf ARCHS4 coexpression; DSP human tf ARCHS4 coexpression; MRPL28 human tf ARCHS4 coexpression; SRCAP human tf ARCHS4 coexpression; ZNF638 human tf ARCHS4 coexpression; SMARCEL human tf ARCHS4 coexpression; HMG20B human tf ARCHS4 coexpression; ZNF382 human tf ARCHS4 coexpression; DVL3 human tf ARCHS4 coexpression; PLXND1 human tf ARCHS4 coexpression; ZNF641 human tf ARCHS4 coexpression; ZNF345 human tf ARCHS4 coexpression; ZNF581 human tf ARCHS4 coexpression; ZNF80 human tf ARCHS4 coexpression; EVX2 human tf ARCHS4 coexpression; ZNF497 human tf ARCHS4 coexpression; ZNF266 human tf ARCHS4 coexpression; DRAP1 human tf ARCHS4 coexpression; ZNF169 human tf ARCHS4 coexpression; ZNF683 human tf ARCHS4 coexpression; DVL2 human tf ARCHS4 coexpression; DMRTA1 human tf ARCHS4 coexpression; ZNF154 human tf ARCHS4 coexpression; ZNF707 human tf ARCHS4 coexpression; ZNF662 human tf ARCHS4 coexpression; ZNF594 human tf ARCHS4 coexpression; THAP7 human tf ARCHS4 coexpression; ZNF676 human tf ARCHS4 coexpression; coexpression; ZNF766 human tf ARCHS4 coexpression; ZNF799 human tf ARCHS4 coexpression; ZNF786 human tf ARCHS4 coexpression; ZNF789 human tf ARCHS4 coexpression; ZNF619 human tf ARCHS4 coexpression; RUNX2 human tf ARCHS4 coexpression; CDX4 human tf ARCHS4 coexpression; RFX1 human tf ARCHS4 coexpression; POU2F3 human tf ARCHS4 coexpression; ZNF157 human tf ARCHS4 coexpression; GRHL3 human tf ARCHS4 coexpression; ZNF230 human tf ARCHS4 coexpression; GMEB1 human tf ARCHS4 coexpression; E4F1 human tf ARCHS4 coexpression; SMAD1 human tf ARCHS4 coexpression; SOX4 human tf ARCHS4 coexpression; FOXA3 human tf ARCHS4 coexpression; HOXB9 human tf ARCHS4 coexpression; SMAD2 human tf ARCHS4 coexpression; HSF5 human tf ARCHS4 coexpression; SOX11 human tf ARCHS4 coexpression; HES7 human tf ARCHS4 coexpression; ZNF16 human tf ARCHS4 coexpression; ZNF708 human tf ARCHS4 coexpression; CTCFL human tf ARCHS4 coexpression; SOX6 human tf ARCHS4 coexpression; PAX2 human tf ARCHS4 coexpression; SREBF1 human tf ARCHS4 coexpression; ALX4 human tf ARCHS4 coexpression; NR1H4 human tf ARCHS4 coexpression; PAX7 human tf ARCHS4 coexpression; HSF4 human tf ARCHS4 coexpression; KLF1 human tf ARCHS4 coexpression; ETV2 human tf ARCHS4 coexpression; ZNF527 human tf ARCHS4 coexpression; ZNF383 human tf ARCHS4 coexpression; TFCP2L1 human tf ARCHS4 coexpression; ZNF479 human tf ARCHS4 coexpression; TFDP3 human tf ARCHS4 coexpression; ZNF585A human tf ARCHS4 coexpression; ZNF420 human tf ARCHS4 coexpression; ZNF682 human tf ARCHS4 coexpression; ZNF675 human tf ARCHS4 coexpression; HOXA1 human tf ARCHS4 coexpression; PAX4 human tf ARCHS4 coexpression; PITX3 human tf ARCHS4 coexpression; DLX4 human tf ARCHS4 coexpression; FOXN1 human tf ARCHS4 coexpression; ZNF438 human tf ARCHS4 coexpression; TBX1 human tf ARCHS4 coexpression; GATA2 human tf ARCHS4 coexpression; ZNF273 human tf ARCHS4 coexpression; ZNF396 human tf ARCHS4 coexpression; YBX2 human tf ARCHS4 coexpression; ZFP64 human tf ARCHS4 coexpression; BCLAFI GM12878 hg19; NR2C2 GM12878 hg19; NFIC HepG2 hg19; SMAD2/3 21741376 ChIP-Seq ESCs Human; MYBL2 22936984 ChIP-ChIP MESCs Mouse; PBX1 22567123 ChIP-ChIP OVCAR3 Human; ETS1 20019798 ChIP-Seq JURKAT Human; ELF1 HCT116 hg19; GABPA A549 hg19; GABPA SK—N—SH hg19; TAL1 26923725 Chip-Seq HPCs Mouse; STAT3 GM12878 hg19; SIX5 GM12878 hg19; EGR1 23403033 ChIP-Seq LIVER Mouse; CTNNB1 20615089 ChIP-ChIP FETAL BRAIN Human; EP300 K562 hg19; SIX5 A549 hg19; ZKSCAN1 MEL cell line mm9; HSF1 23293686 ChIP-Seq STHDH STRIATAL Mouse; NANOG 16518401 ChIP-PET MESCs Mouse; ETS1 A549 hg19; CTCF G1E mm9; KAT2A MEL cell line mm9; DMBX1 human tf ARCHS4 coexpression; ZNF665 human tf ARCHS4 coexpression; ZNF140 human tf ARCHS4 coexpression; ZNF250 human tf ARCHS4 coexpression; ZNF626 human tf ARCHS4 coexpression; ZNF490 human tf ARCHS4 coexpression; ZNF546 human tf ARCHS4 coexpression; ZNF529 human tf ARCHS4 coexpression; ZBTB3 human tf ARCHS4 coexpression; ZNF101 human tf ARCHS4 coexpression; ZNF442 human tf ARCHS4 coexpression; HKR1 human tf ARCHS4 coexpression; ZFP82 human tf ARCHS4 coexpression; ZNF771 human tf ARCHS4 coexpression; ZNF223 human tf ARCHS4 coexpression; ZNF408 human tf ARCHS4 coexpression; KLF14 human tf ARCHS4 coexpression; ZNF417 human tf ARCHS4 coexpression; ZNF570 human tf ARCHS4 coexpression; ZNF440 human tf ARCHS4 coexpression; ZNF692 human tf ARCHS4 coexpression; ZFP41 human tf ARCHS4 coexpression; ZNF277 human tf ARCHS4 coexpression; ZBTB12 human tf ARCHS4 coexpression; IFI16 human tf ARCHS4 coexpression; GTF2IRD2B human tf ARCHS4 coexpression; PKHD1 human tf ARCHS4 coexpression; VPS72 human tf ARCHS4 coexpression; U2AF1L4 human tf ARCHS4 coexpression; SCML4 human tf ARCHS4 coexpression; POGZ human tf ARCHS4 coexpression; ADAMTS17 human tf ARCHS4 coexpression; HMG20A human tf ARCHS4 coexpression; SMARCC2 human tf ARCHS4 coexpression; DNAJC1 human tf ARCHS4 coexpression; PRR12 human tf ARCHS4 coexpression; GTF3A human tf ARCHS4 coexpression; EDF1 human tf ARCHS4 coexpression; SF3A2 human tf ARCHS4 coexpression; ZMAT5 human tf ARCHS4 coexpression; ZNF829 human tf ARCHS4 coexpression; NOC4L human tf ARCHS4 coexpression; ZNF716 human tf ARCHS4 coexpression; ZNF487 human tf ARCHS4 coexpression; ZNF843 human tf ARCHS4 coexpression; CARHSP1 human tf ARCHS4 coexpression; ZNF747 human tf ARCHS4 coexpression; ZNF674 human tf ARCHS4 coexpression; ZNF558 human tf ARCHS4 coexpression; GBX1 human tf ARCHS4 coexpression; MAEL human tf ARCHS4 coexpression; DPF2 human tf ARCHS4 coexpression; BARX1 human tf ARCHS4 coexpression; HOXB3 human tf ARCHS4 coexpression; HOXB7 human tf ARCHS4 coexpression; ESRRB human tf ARCHS4 coexpression; PROX2 human tf ARCHS4 coexpression; TFAP2E human tf ARCHS4 coexpression; ERF human tf ARCHS4 coexpression; NR113 human tf ARCHS4 coexpression; CRX human tf ARCHS4 coexpression; MZF1 human tf ARCHS4 coexpression; HOXA9 human tf ARCHS4 coexpression; HSF2 human tf ARCHS4 coexpression; MLXIPL human tf ARCHS4 coexpression; MTA1 human tf ARCHS4 coexpression; NCOR2 human tf ARCHS4 coexpression; ZNF765 human tf ARCHS4 coexpression; HLA-DQB2 human tf ARCHS4 coexpression; VSX1 human tf ARCHS4 coexpression; ZNF526 human tf ARCHS4 coexpression; ZNF7 human tf ARCHS4 coexpression; ZNF446 human tf ARCHS4 coexpression; ZNF432 human tf ARCHS4 coexpression; DMRTC2 human tf ARCHS4 coexpression; ZNF287 human tf ARCHS4 coexpression; FOXP4 human tf ARCHS4 coexpression; MLLT1 human tf ARCHS4 coexpression; ZNF576 human tf ARCHS4 coexpression; ZNF211 human tf ARCHS4 coexpression; ZBTB45 human tf ARCHS4 coexpression; ZNF430 human tf ARCHS4 coexpression; SK1 human tf ARCHS4 coexpression; RBM10 human tf ARCHS4 coexpression; FOXP3 human tf ARCHS4 coexpression; ZNF221 human tf ARCHS4 coexpression; WIZ human tf ARCHS4 coexpression; NOTO human tf ARCHS4 coexpression; HSF1 human tf ARCHS4 coexpression; HOXB4 human tf ARCHS4 coexpression; SIX5 human tf ARCHS4 coexpression; TBX10 human tf ARCHS4 coexpression; ZNF3 human tf ARCHS4 coexpression; IRX6 human tf ARCHS4 coexpression; CREB3L3 human tf ARCHS4 coexpression; EST1 17652178 ChIP-ChIP JURKAT Human; KAPI 22055183 ChIP-Seq ESCs Mouse; EZH2 K562 hg19; NFIC GM12878 hg19; SRF 21415370 ChIP-Seq HL-1 Mouse; JARIDIA 20064375 ChIP-Seq MESCs Mouse; GABPA HL-60 hg19; SIX5 K562 hg19; TRIM28 17542650 ChIP-ChIP NTERA2 Human

### Downregulated Transcription Factor Target Activity in GSE88773 by Combined Score

FOSB human tf ARCHS4 coexpression; NR4A2 human tf ARCHS4 coexpression; ZFP36 human tf ARCHS4 coexpression; JUN human tf ARCHS4 coexpression; KLF6 human tf ARCHS4 coexpression; NR4A1 human tf ARCHS4 coexpression; JUNB human tf ARCHS4 coexpression; SATB1 human tf ARCHS4 coexpression; TRIM28 21343339 ChIP-Seq HEK293 Human; ZFP36L1 human tf ARCHS4 coexpression; RACK7 27058665 Chip-Seq MCF-7 Human; ZNF775 human tf ARCHS4 coexpression; IRX5 human tf ARCHS4 coexpression; JUND human tf ARCHS4 coexpression; JARID2 20075857 ChIP-Seq MESCs Mouse; CREB1 26743006 Chip-Seq LNCaP Human; ETS1 MEL cell line mm9; NUCKS1 24931609 ChIP-Seq HEPATOCYTES Mouse; TBX21 human tf ARCHS4 coexpression; PAX8 human tf ARCHS4 coexpression; STAT5A human tf ARCHS4 coexpression; NFATC1 human tf ARCHS4 coexpression; TBX18 human tf ARCHS4 coexpression; EZH2 27304074 Chip-Seq ESCs Mouse; NR1H3 23393188 ChIP-Seq ATHEROSCLEROTIC-FOAM Human; RAD21 MCF-7 hg19; BCL11B human tf ARCHS4 coexpression; HOXA3 human tf ARCHS4 coexpression; RELB human tf ARCHS4 coexpression; SALL1 human tf ARCHS4 coexpression; ZBTB16 human tf ARCHS4 coexpression; NPAS4 human tf ARCHS4 coexpression; SMC3 SK—N—SH hg19; WT1 19549856 ChIP-ChIP CCG9911 Human; FOXA1 T47D hg19; ESR2 21235772 ChIP-Seq MCF-7 Human; SUZ12 18692474 ChIP-Seq MEFs Mouse; EZH2 18974828 ChIP-Seq MESCs Mouse; RNF2 18974828 ChIP-Seq MESCs Mouse; MEF2A K562 hg19; RFX5 SK—N—SH hg19; GATA2 26923725 Chip-Seq HEMANGIOBLAST Mouse; KDM2B 26808549 Chip-Seq SUP—B15 Human; FOXM1 25889361 ChIP-Seq OE33 AND U2OS Human; SP140 human tf ARCHS4 coexpression; FOS human tf ARCHS4 coexpression; ATF3 human tf ARCHS4 coexpression; USF1 human tf ARCHS4 coexpression; TSC22D3 human tf ARCHS4 coexpression; IRF3 human tf ARCHS4 coexpression; RBPJL human tf ARCHS4 coexpression; STAT1 human tf ARCHS4 coexpression; HBP1 human tf ARCHS4 coexpression; GLI2 human tf ARCHS4 coexpression; MEF2A human tf ARCHS4 coexpression; ATXN7 human tf ARCHS4 coexpression; ZNF827 human tf ARCHS4 coexpression; ZNF800 human tf ARCHS4 coexpression; ZNF655 human tf ARCHS4 coexpression; GFI1 human tf ARCHS4 coexpression; KLF3 human tf ARCHS4 coexpression; FOXN3 human tf ARCHS4 coexpression; IRF9 human tf ARCHS4 coexpression; STAT2 human tf ARCHS4 coexpression; FOXJ2 human tf ARCHS4 coexpression; RUNX3 human tf ARCHS4 coexpression; NOTCH1 21737748 ChIP-Seq TLL Human; RUNX1 26923725 Chip-Seq MACROPHAGESS Mouse; RFX5 IMR-90 hg19; BRD4 27068464 Chip-Seq AML-cells Mouse; SUZ12 27294783 Chip-Seq ESCs Mouse; KDM2B 26808549 Chip-Seq JURKAT Human; RELA 24523406 ChIP-Seq FIBROSARCOMA Human; JUN endothelial cell of umbilical vein hg19; TEAD4 SK—N—SH hg19; FOXA1 21915096 ChIP-Seq LNCaP-1F5 Human; SUZ12 18692474 ChIP-Seq MESCs Mouse; JARID2 20064375 ChIP-Seq MESCs Mouse; TAL1 20566737 ChIP-Seq PRIMARY FETAL LIVER ERYTHROID Mouse; RNF2 16625203 ChIP-ChIP MESCs Mouse; SUZ12 20075857 ChIP-Seq MESCs Mouse; EP300 T47D hg19; TET1 21490601 ChIP-Seq MESCs Mouse; SMC3 22415368 ChIP-Seq MEFs Mouse; CREB1 26743006 Chip-Seq LNCaP-abl Human; KDM2B 26808549 Chip-Seq SIL-ALL Human; SMC1 22415368 ChIP-Seq MEFs Mouse; ZBTB7A ECC-1 hg19; SUZ12 18974828 ChIP-Seq MESCs Mouse; IKZF1 21737484 ChIP-ChIP HCT116 Human; MEF2C GM12878 hg19; WT1 25993318 ChIP-Seq PODOCYTE Human; RAD21 ECC-1 hg19; PKHDIL1 human tf ARCHS4 coexpression; CHD7 human tf ARCHS4 coexpression; SNAI2 human tf ARCHS4 coexpression; PHF21A human tf ARCHS4 coexpression; NRK human tf ARCHS4 coexpression; ZGPAT human tf ARCHS4 coexpression; SSH1 human tf ARCHS4 coexpression; FGD1 human tf ARCHS4 coexpression; FLI1 human tf ARCHS4 coexpression; LEF1 human tf ARCHS4 coexpression; KLF4 human tf ARCHS4 coexpression; ELF5 human tf ARCHS4 coexpression; FOXA1 human tf ARCHS4 coexpression; IRF1 human tf ARCHS4 coexpression; SOX21 human tf ARCHS4 coexpression; ZNFX1 human tf ARCHS4 coexpression; TSHZ3 human tf ARCHS4 coexpression; EGR1 human tf ARCHS4 coexpression; NKX2-3 human tf ARCHS4 coexpression; KLF12 human tf ARCHS4 coexpression; LHX6 human tf ARCHS4 coexpression; GLI3 human tf ARCHS4 coexpression; NFATC2 human tf ARCHS4 coexpression; NFATC4 human tf ARCHS4 coexpression; MEF2C human tf ARCHS4 coexpression; NFATC3 human tf ARCHS4 coexpression; RORA human tf ARCHS4 coexpression; HOXC8 human tf ARCHS4 coexpression; IKZF3 human tf ARCHS4 coexpression; HAND2 human tf ARCHS4 coexpression; ZBTB7B human tf ARCHS4 coexpression; HOXD8 human tf ARCHS4 coexpression; PARP12 human tf ARCHS4 coexpression; ZNF831 human tf ARCHS4 coexpression; SP100 human tf ARCHS4 coexpression; IFI16 human tf ARCHS4 coexpression; OTX2 human tf ARCHS4 coexpression; ZNF319 human tf ARCHS4 coexpression; ZNF503 human tf ARCHS4 coexpression; ZNF641 human tf ARCHS4 coexpression; ZFP36L2 human tf ARCHS4 coexpression; POU3F1 human tf ARCHS4 coexpression; NR2F2 human tf ARCHS4 coexpression; WT1 human tf ARCHS4 coexpression; ZHX2 human tf ARCHS4 coexpression; ZNF618 human tf ARCHS4 coexpression; TCF12 MCF-7 hg19; RAD21 SK—N—SH hg19; TEAD4 HepG2 hg19; PIAS1 25552417 ChIP-Seq VCAP Human; TCF12 myocyte mm9; TP53 20018659 ChIP-ChIP RIE Mouse; MNX1 26342078 ChIP-Seq MIN6-4N Mouse; MXI1 IMR-90 hg19; CTCF 27219007 Chip-Seq ERYTHROID Human; NCOR1 K562 hg19; FOXM1 26100407 CHIP-SEQ Hek293 flp-in Human; E2F1 17053090 ChIP-ChIP MCF-7 Human; RAD21 A549 hg19; MTF2 20144788 ChIP-Seq MESCs Mouse; PPARG 20176806 ChIP-Seq THIOMACROPHAGE Mouse; GF1B 26923725 Chip-Seq HPCs Mouse; RING1B 27294783 Chip-Seq NPCs Mouse; UBF1/2 26484160 Chip-Seq HMECs Human; POLR2AphosphoS5 GM12878 hg19; FOXA2 HepG2 hg19; SRF HCT116 hg19; JUND GM12878 hg19; RAD21 GM12878 hg19; EP300 CH12.LX mm9; FOXA1 26743006 Chip-Seq LNCaP-abl Human; FOXM1 23109430 ChIP-Seq U2OS Human; GATA1 22025678 ChIP-Seq K562 Human; STAT3 22323479 ChIP-Seq MACROPHAGE Mouse; FOXF2 human tf ARCHS4 coexpression; SP110 human tf ARCHS4 coexpression; ZBTB46 human tf ARCHS4 coexpression; DBX1 human tf ARCHS4 coexpression; PATZ1 human tf ARCHS4 coexpression; TBX15 human tf ARCHS4 coexpression; FOXP3 human tf ARCHS4 coexpression; ZBTB48 human tf ARCHS4 coexpression; IKZF1 human tf ARCHS4 coexpression; MEF2D human tf ARCHS4 coexpression; NR4A3 human tf ARCHS4 coexpression; TWIST1 human tf ARCHS4 coexpression; EGR2 human tf ARCHS4 coexpression; KLF2 human tf ARCHS4 coexpression; MYOD1 human tf ARCHS4 coexpression; ARNTL human tf ARCHS4 coexpression; MEIS2 human tf ARCHS4 coexpression; RAX human tf ARCHS4 coexpression; HLX human tf ARCHS4 coexpression; ETV6 human tf ARCHS4 coexpression; RORC human tf ARCHS4 coexpression; CREB3L4 human tf ARCHS4 coexpression; MTF1 human tf ARCHS4 coexpression; BATF human tf ARCHS4 coexpression; SALL2 human tf ARCHS4 coexpression; STAT6 human tf ARCHS4 coexpression; SP2 human tf ARCHS4 coexpression; AR human tf ARCHS4 coexpression; BNC1 human tf ARCHS4 coexpression; STAT5B human tf ARCHS4 coexpression; IRX3 human tf ARCHS4 coexpression; MSC human tf ARCHS4 coexpression; FOXE1 human tf ARCHS4 coexpression; ZFPM1 human tf ARCHS4 coexpression; SOX2 human tf ARCHS4 coexpression; IRF7 human tf ARCHS4 coexpression; CREB3L1 human tf ARCHS4 coexpression; HMGA2 human tf ARCHS4 coexpression; MBNL1 human tf ARCHS4 coexpression; RNF166 human tf ARCHS4 coexpression; ADAR human tf ARCHS4 coexpression; MKRN3 human tf ARCHS4 coexpression; ZDHHC11 human tf ARCHS4 coexpression; PLXNC1 human tf ARCHS4 coexpression; TRAFD1 human tf ARCHS4 coexpression; ZBP1 human tf ARCHS4 coexpression; ZNF710 human tf ARCHS4 coexpression; WNT8B human tf ARCHS4 coexpression; ZNF784 human tf ARCHS4 coexpression; NEUROG2 human tf ARCHS4 coexpression; ZNF837 human tf ARCHS4 coexpression; ZNF165 human tf ARCHS4 coexpression; ZNF48 human tf ARCHS4 coexpression; MKRN1 human tf ARCHS4 coexpression; FOXL1 human tf ARCHS4 coexpression; ZNF394 human tf ARCHS4 coexpression; ZKSCAN2 human tf ARCHS4 coexpression; SALL3 human tf ARCHS4 coexpression; OSR1 human tf ARCHS4 coexpression; ZNF608 human tf ARCHS4 coexpression; FOXO1 human tf ARCHS4 coexpression; HES1 human tf ARCHS4 coexpression; TBX5 human tf ARCHS4 coexpression; PGR human tf ARCHS4 coexpression; NFKB2 human tf ARCHS4 coexpression; SPI1 human tf ARCHS4 coexpression; NR1H3 human tf ARCHS4 coexpression; CREBL2 human tf ARCHS4 coexpression; TFEB human tf ARCHS4 coexpression; TFEC human tf ARCHS4 coexpression; MYF6 human tf ARCHS4 coexpression; E2F6 A549 hg19; TP63 23658742 ChIP-Seq EP156T Human; SUZ12 H1-hESC hg19; CTCF ECC-1 hg19; GATA3 A549 hg19; TP63 19390658 ChIP-ChIP HaCaT Human; EP300 ECC-1 hg19; TEAD4 MCF-7 hg19; CTCF astrocyte of the cerebellum hg19; HNF4G HepG2 hg19; KDM2B 26808549 Chip-Seq DND41 Human; KDM2B 26808549 Chip-Seq K562 Human; CTCF 27219007 Chip-Seq Bcells Human; CUX1 GM12878 hg19; EZH2 skeletal muscle myoblast hg19; TBP HeLa—S3 hg19; E2F4 MCF 10A hg19; SMARCC2 HeLa—S3 hg19; JUND CH12.LX mm9; JUND SK—N—SH hg19; PHC1 16625203 ChIP-ChIP MESCs Mouse; PML MCF-7 hg19; EGR1 19374776 ChIP-ChIP THP-1 Human; EOMES 21245162 ChIP-Seq HESCs Human; CTCF T47D hg19; ZEB1 HepG2 hg19; EED 16625203 ChIP-ChIP MESCs Mouse; E2F6 H1-hESC hg19; GATA1 K562 hg19; RING1B 27294783 Chip-Seq ESCs Mouse; SMRT 27268052 Chip-Seq Bcells Human; P300 27058665 Chip-Seq ZR-75-30cells Human; SMRT 22465074 ChIP-Seq MACROPHAGES Mouse; KLF4 26769127 Chip-Seq PDAC-Cell line Human; MYB 26560356 Chip-Seq TH1 Human; FOXO1 25302145 ChIP-Seq T-LYMPHOCYTE Mouse; KDM2B 26808549 Chip-Seq HPB-ALL Human; EZH2 T-cell acute lymphoblastic leukemia hg19; EZH2 myotube hg19; TRIM28 U2OS hg19; ZNF217 24962896 ChIP-Seq MCF-7 Human; NFIC SK—N—SH hg19; DROSHA 22980978 ChIP-Seq HELA Human; EGR1 ECC-1 hg19; HDAC2 MCF-7 hg19; RARG 19884340 ChIP-ChIP MEFs Mouse; SUZ12 18555785 ChIP-Seq MESCs Mouse; CIITA 25753668 ChIP-Seq RAJI Human; SUZ12 NT2-D1 hg19; SPI1 23127762 ChIP-Seq K562 Human; PPARD 23176727 ChIP-Seq KERATINOCYTES Mouse; FOS GM12878 hg19; PAX3-FKHR 20663909 ChIP-Seq RHABDOMYOSARCOMA Human; RNF2 27304074 Chip-Seq ESCs Mouse; CTNNB1 20615089 ChIP-ChIP FETAL BRAIN Human; ESR1 ECC-1 hg19; ESR1 21235772 ChIP-Seq MCF-7 Human; ZC3H11A MEL cell line mm9; CTCFL K562 hg19; CTCF WI38 hg19; TTF2 22483619 ChIP-Seq HELA Human; POLR2A ECC-1 hg19; ERA 27197147 Chip-Seq ENDOMETRIOID-ADENOCARCINOMA Human; POLR2AphosphoS5 HCT116 hg19; FOS K562 hg19; POLR2A H54 hg19; TCF7 22412390 ChIP-Seq EML Mouse; TBX20 22328084 ChIP-Seq HEART Mouse; TBX20 22080862 ChIP-Seq HEART Mouse; MAZ IMR-90 hg19; FOXA1 ECC-1 hg19; FOSL2 human tf ARCHS4 coexpression; DACH1 human tf ARCHS4 coexpression; ZNF202 human tf ARCHS4 coexpression; PRRX2 human tf ARCHS4 coexpression; RCOR2 human tf ARCHS4 coexpression; IRF2 human tf ARCHS4 coexpression; KLF13 human tf ARCHS4 coexpression; DDIT3 human tf ARCHS4 coexpression; HOXC10 human tf ARCHS4 coexpression; SOX1 human tf ARCHS4 coexpression; POU3F2 human tf ARCHS4 coexpression; PHOX2A human tf ARCHS4 coexpression; ZNF395 human tf ARCHS4 coexpression; ESRRG human tf ARCHS4 coexpression; HOXC6 human tf ARCHS4 coexpression; SOX18 human tf ARCHS4 coexpression; MXD3 human tf ARCHS4 coexpression; ZBED1 human tf ARCHS4 coexpression; ZNF324 human tf ARCHS4 coexpression; FOXO4 human tf ARCHS4 coexpression; EBF1 human tf ARCHS4 coexpression; NFIA human tf ARCHS4 coexpression; ZFP30 human tf ARCHS4 coexpression; BCL6 human tf ARCHS4 coexpression; OTX1 human tf ARCHS4 coexpression; PRRX1 human tf ARCHS4 coexpression; PRDM2 human tf ARCHS4 coexpression; TCF21 human tf ARCHS4 coexpression; KLF9 human tf ARCHS4 coexpression; ONECUT2 human tf ARCHS4 coexpression; VSX2 human tf ARCHS4 coexpression; PITX2 human tf ARCHS4 coexpression; TRERF1 human tf ARCHS4 coexpression; NR1H2 human tf ARCHS4 coexpression; RARA human tf ARCHS4 coexpression; ETS1 human tf ARCHS4 coexpression; HIVEP2 human tf ARCHS4 coexpression; TBX3 human tf ARCHS4 coexpression; LBX1 human tf ARCHS4 coexpression; ZEB1 human tf ARCHS4 coexpression; ETS2 human tf ARCHS4 coexpression; HIVEP3 human tf ARCHS4 coexpression; NEUROD4 human tf ARCHS4 coexpression; ZBTB1 human tf ARCHS4 coexpression; SOX6 human tf ARCHS4 coexpression; MATR3 human tf ARCHS4 coexpression; ZFYVE26 human tf ARCHS4 coexpression; AKNA human tf ARCHS4 coexpression; ANKZF1 human tf ARCHS4 coexpression; ATOH8 human tf ARCHS4 coexpression; ID4 human tf ARCHS4 coexpression; SMAD7 human tf ARCHS4 coexpression; ZC3HAV1 human tf ARCHS4 coexpression; CEBPA human tf ARCHS4 coexpression; MBD2 human tf ARCHS4 coexpression; DNAJC1 human tf ARCHS4 coexpression; SSH2 human tf ARCHS4 coexpression; ZC3H8 human tf ARCHS4 coexpression; FBN1 human tf ARCHS4 coexpression; PPP2R3B human tf ARCHS4 coexpression; TIGD3 human tf ARCHS4 coexpression; PRB3 human tf ARCHS4 coexpression; RBM20 human tf ARCHS4 coexpression; PRB4 human tf ARCHS4 coexpression; ARID5B human tf ARCHS4 coexpression; ZNF783 human tf ARCHS4 coexpression; ZNF239 human tf ARCHS4 coexpression; FOXJ1 human tf ARCHS4 coexpression; PRDM14 human tf ARCHS4 coexpression; MKX human tf ARCHS4 coexpression; ZNF276 human tf ARCHS4 coexpression; ZNF653 human tf ARCHS4 coexpression; ZNF474 human tf ARCHS4 coexpression; SP8 human tf ARCHS4 coexpression; GBX1 human tf ARCHS4 coexpression; OSR2 human tf ARCHS4 coexpression; TSC22D4 human tf ARCHS4 coexpression; ZNF212 human tf ARCHS4 coexpression; OTP human tf ARCHS4 coexpression; ETV3L human tf ARCHS4 coexpression; NHLH2 human tf ARCHS4 coexpression; ZNF619 human tf ARCHS4 coexpression; ZNF114 human tf ARCHS4 coexpression; ZBTB2 human tf ARCHS4 coexpression; SNAI3 human tf ARCHS4 coexpression; ZKSCAN4 human tf ARCHS4 coexpression; ZNF133 human tf ARCHS4 coexpression; PITX3 human tf ARCHS4 coexpression; HNF1A human tf ARCHS4 coexpression; HOXA9 human tf ARCHS4 coexpression; TCF7 human tf ARCHS4 coexpression; GRHL2 human tf ARCHS4 coexpression; SP4 human tf ARCHS4 coexpression; MEF2A GM12878 hg19; LMO2 26923725 Chip-Seq MACROPHAGESS Mouse; EP300 20729851 ChIP-Seq FORBRAIN MIDBRAIN LIMB HEART Mouse; FOXA2 19822575 ChIP-Seq HepG2 Human; SOX9 24532713 ChIP-Seq HFSC Mouse; STAT1 20625510 ChIP-Seq HELA Human; HCFC1 20581084 ChIP-Seq MESCs Mouse; PPARG 19300518 ChIP-PET 3T3-L1 Mouse; EZH2 keratinocyte hg19; ARID3A K562 hg19; POLR2A cerebellum mm9; POU3F1 26484290 ChIP-Seq ESCss Mouse; EZH2 27294783 Chip-Seq ESCs Mouse; CHD7 H1-hESC hg19; GTF2B K562 hg19; CTCF cerebellum mm9; NANOG 18700969 ChIP-ChIP MESCs Mouse; POLR3A HeLa—S3 hg19; EP300 MCF-7 hg19; POLR2A H1-hESC hg19; TRP63 18441228 ChIP-ChIP KERATINOCYTES Mouse; CTCF SK—N—SH hg19; PBX3 GM12878 hg19; ZNF652 21678463 ChIP-ChIP ZR75-1 Human; PBX3 A549 hg19; THRA 23701648 ChIP-Seq CEREBELLUM Mouse; NANOG H1-hESC hg19; FOXP2 SK—N-MC hg19; SUZ12 16625203 ChIP-ChIP MESCs Mouse; HSF1 HepG2 hg19; ZBTB7A K562 hg19; JUN K562 hg19; MYC MCF 10A hg19; IRF3 HepG2 hg19; YY1 21170310 ChIP-Seq MESCs Mouse; NFIB 24661679 ChIP-Seq LUNG Mouse; ZC3H11A CH12.LX mm9; NFYA K562 hg19; CTBP2 H1-hESC hg19; POLR2A HCT116 hg19; CTCF GM12801 hg19; E2F4 GM12878 hg19; FLI1 21867929 ChIP-Seq CD8 Mouse; POLR2A MCF 10A hg19; EBF1 GM12878 hg19; DCP1A 22483619 ChIP-Seq HELA Human; GATA3 20176728 ChIP-ChIP TSCs Mouse; HNF4A HepG2 hg19; SP2 K562 hg19; USF1 ECC-1 hg19; RUNX 20019798 ChIP-Seq JUKART Human; RELA GM18526 hg19; MYOD1 C2C12 mm9; CBX2 27304074 Chip-Seq ESCs Mouse; TDRD3 21172665 ChIP-Seq MCF-7 Human; USF1 A549 hg19; FOXA2 A549 hg19; MAX K562 hg19; GATA2 19941826 ChIP-Seq K562 Human; GATA2 G1E-ER4 mm9; FOSL2 A549 hg19; TAL1 G1E-ER4 mm9; XRN2 22483619 ChIP-Seq HELA Human; GTF3C2 K562 hg19; SREBF2 GM12878 hg19; EWS 26573619 Chip-Seq HEK293 Human; KLF6 26769127 Chip-Seq PDAC-Cell line Human; GATA2 22383799 ChIP-Seq GIME Mouse; UTX 26944678 Chip-Seq JUKART Human; P300 27268052 Chip-Seq Bcells Human; HA-E2F1 MCF-7 hg19; NFYA GM12878 hg19; TCF7L2 HEK293 hg19; MXII HepG2 hg19; POLR2A bone marrow mm9; RFX5 H1-hESC hg19; IRF3 GM12878 hg19; EZH2 B cell hg19; ELK3 25401928 ChIP-Seq HUVEC Human; MYCN 27167114 Chip-Seq NEUROBLASTOMA Human; BCL6B human tf ARCHS4 coexpression; NKX6-1 human tf ARCHS4 coexpression; FOXH1 human tf ARCHS4 coexpression; RUNX1 human tf ARCHS4 coexpression; KLF10 human tf ARCHS4 coexpression; HHEX human tf ARCHS4 coexpression; ZNF182 human tf ARCHS4 coexpression; ZNF157 human tf ARCHS4 coexpression; ARNT human tf ARCHS4 coexpression; POU2F1 human tf ARCHS4 coexpression; RXRG human tf ARCHS4 coexpression; IRF8 human tf ARCHS4 coexpression; IRF4 human tf ARCHS4 coexpression; IRF5 human tf ARCHS4 coexpression; SIX1 human tf ARCHS4 coexpression; MAX human tf ARCHS4 coexpression; RREB1 human tf ARCHS4 coexpression; ELK4 human tf ARCHS4 coexpression; HEYL human tf ARCHS4 coexpression; SMAD1 human tf ARCHS4 coexpression; NKX3-1 human tf ARCHS4 coexpression; ZNF582 human tf ARCHS4 coexpression; MEOX1 human tf ARCHS4 coexpression; MAFB human tf ARCHS4 coexpression; YY2 human tf ARCHS4 coexpression; MIER1 human tf ARCHS4 coexpression; POU3F4 human tf ARCHS4 coexpression; PPARA human tf ARCHS4 coexpression; RXRA human tf ARCHS4 coexpression; ASCL2 human tf ARCHS4 coexpression; NKX2-5 human tf ARCHS4 coexpression; MAFA human tf ARCHS4 coexpression; SOX17 human tf ARCHS4 coexpression; NEUROG1 human tf ARCHS4 coexpression; HIC1 human tf ARCHS4 coexpression; NR2F1 human tf ARCHS4 coexpression; GLIS1 human tf ARCHS4 coexpression; RARG human tf ARCHS4 coexpression; CEBPB human tf ARCHS4 coexpression; FOXB1 human tf ARCHS4 coexpression; IRX2 human tf ARCHS4 coexpression; NFE2 human tf ARCHS4 coexpression; STAT3 human tf ARCHS4 coexpression; PTF1A human tf ARCHS4 coexpression; BATF2 human tf ARCHS4 coexpression; SOX12 human tf ARCHS4 coexpression; MYOG human tf ARCHS4 coexpression; HOXA5 human tf ARCHS4 coexpression; EPAS1 human tf ARCHS4 coexpression; ZXDC human tf ARCHS4 coexpression; CUX2 human tf ARCHS4 coexpression; MYF5 human tf ARCHS4 coexpression; ELF2 human tf ARCHS4 coexpression; NFKB1 human tf ARCHS4 coexpression; EZH1 human tf ARCHS4 coexpression; HOXA6 human tf ARCHS4 coexpression; TRPS1 human tf ARCHS4 coexpression; HIFIA human tf ARCHS4 coexpression; XBP1 human tf ARCHS4 coexpression; TEAD2 human tf ARCHS4 coexpression; TP63 human tf ARCHS4 coexpression; ZNF76 human tf ARCHS4 coexpression; MXD1 human tf ARCHS4 coexpression; POU2F2 human tf ARCHS4 coexpression; PAX7 human tf ARCHS4 coexpression; TBX2 human tf ARCHS4 coexpression; ZNF532 human tf ARCHS4 coexpression; ZNF462 human tf ARCHS4 coexpression; FOXF1 human tf ARCHS4 coexpression; ZNF586 human tf ARCHS4 coexpression; ZNF649 human tf ARCHS4 coexpression; ZNF335 human tf ARCHS4 coexpression; ZNF595 human tf ARCHS4 coexpression; HES4 human tf ARCHS4 coexpression; ZNF467 human tf ARCHS4 coexpression; FOXC2 human tf ARCHS4 coexpression; ZNF135 human tf ARCHS4 coexpression; OVOL1 human tf ARCHS4 coexpression; ZNF131 human tf ARCHS4 coexpression; NHLH1 human tf ARCHS4 coexpression; ZNF672 human tf ARCHS4 coexpression; ZNF521 human tf ARCHS4 coexpression; ZNF592 human tf ARCHS4 coexpression; ZNF750 human tf ARCHS4 coexpression; ZSCAN29 human tf ARCHS4 coexpression; FOXN4 human tf ARCHS4 coexpression; ZNF189 human tf ARCHS4 coexpression; EGR4 human tf ARCHS4 coexpression; ZNF317 human tf ARCHS4 coexpression; ZSCAN10 human tf ARCHS4 coexpression; OVOL2 human tf ARCHS4 coexpression; NKX2-2 human tf ARCHS4 coexpression; GZF1 human tf ARCHS4 coexpression; RCOR3 human tf ARCHS4 coexpression; ZNF574 human tf ARCHS4 coexpression; TBX20 human tf ARCHS4 coexpression; ZNF468 human tf ARCHS4 coexpression; OLIG3 human tf ARCHS4 coexpression; SCRT2 human tf ARCHS4 coexpression; DPF3 human tf ARCHS4 coexpression; SLC22A4 human tf ARCHS4 coexpression; ZBTB10 human tf ARCHS4 coexpression; PLXNA4 human tf ARCHS4 coexpression; RFX6 human tf ARCHS4 coexpression; DMAP1 human tf ARCHS4 coexpression; TIGD4 human tf ARCHS4 coexpression; BRPF1 human tf ARCHS4 coexpression; SCML4 human tf ARCHS4 coexpression; SEMA4A human tf ARCHS4 coexpression; CHD6 human tf ARCHS4 coexpression; UNKL human tf ARCHS4 coexpression; NCOA1 human tf ARCHS4 coexpression; ZNF362 human tf ARCHS4 coexpression; TCF23 human tf ARCHS4 coexpression; ADAMTS19 human tf ARCHS4 coexpression; MBNL2 human tf ARCHS4 coexpression; AHDC1 human tf ARCHS4 coexpression; POLR3A K562 hg19; EGR1 H1-hESC hg19; ZFP281 18757296 ChIP-ChIP E14 Mouse; TEAD4 A549 hg19; ELF5 23300383 ChIP-Seq T47D Human; SOX2 16153702 ChIP-ChIP HESCs Human; NR3C1 A549 hg19; WT1 20215353 ChIP-ChIP NEPHRON PROGENITOR Mouse; SMAD 19615063 ChIP-ChIP OVARY Human; RAD21 H1-hESC hg19; SPI1 HL-60 hg19; ELF1 K562 hg19; IRF1 19129219 ChIP-ChIP H3396 Human; BMI1 23680149 ChIP-Seq NPCS Mouse; GATA1 21571218 ChIP-Seq MEGAKARYOCYTES Human; MYBL1 21750041 ChIP-ChIP SPERMATOCYTES Mouse; AR 25329375 ChIP-Seq VCAP Human; POU5F1 18700969 ChIP-ChIP MESCs Mouse; NFIC ECC-1 hg19; REST MCF-7 hg19; SRF ECC-1 hg19; MECOM 23826213 ChIP-Seq KASUMI Mouse; POLR2A A549 hg19; CTNNB1 20460455 ChIP-Seq HCT116 Human; MYOD1 myocyte mm9; GATA2 K562 hg19; NR3C1 ECC-1 hg19; FOXA1 HepG2 hg19; POLR2A liver mm9; KDM5B H1-hESC hg19; KDM1A K562 hg19; SMC3 HepG2 hg19; TRIM28 HEK293 hg19; SMC3 MEL cell line mm9; ARID3A HepG2 hg19; JUN H1-hESC hg19; RNF2 K562 hg19; USF2 K562 hg19; OLIG2 26023283 ChIP-Seq AINV15 Mouse; GATA1 19941827 ChIP-Seq MEL86 Mouse; RXR 22158963 ChIP-Seq LIVER Mouse; GATA3 27048872 Chip-Seq THYMUS Human; RUNX1 27514584 Chip-Seq MCF-7 Human; OLIG2 23332759 ChIP-Seq OLIGODENDROCYTES Mouse; TBLIXR1 K562 hg19; VDR 24787735 ChIP-Seq THP-1 Human; SP1 HCT116 hg19; VDR 22108803 ChIP-Seq LS180 Human; TAF1 MCF-7 hg19; FOSL1 H1-hESC hg19; FOXM1 ECC-1 hg19; RCOR1 IMR-90 hg19; MYB 21317192 ChIP-Seq ERMYB Mouse; JUN CH12.LX mm9; ELK4 26923725 Chip-Seq MESODERM Mouse; REST HepG2 hg19; SOX2 20726797 ChIP-Seq SW620 Human; PKCTHETA 26484144 26923725 Chip-Seq Human; TAL1 Chip-Seq MACROPHAGESS Mouse; LMO2 26923725 Chip-Seq HEMANGIOBLAST Mouse; RXRA GM12878 hg19; SOX9 25088423 ChIP-ChIP EMBRYONIC GONADS Mouse; SREBP1 19666523 ChIP-Seq LIVER Mouse; ELF1 17652178 ChIP-ChIP JURKAT Human; GATA2 20887958 ChIP-Seq HPC-7 Mouse; TFAP2C 20629094 ChIP-Seq MCF-7 Human; MITF 21258399 ChIP-Seq MELANOMA Human; CTCF fibroblast of foreskin hg19; E2F4 MEL cell line mm9; EZH2 23942234 ChIP-Seq MYOBLASTS AND MYOTUBES Mouse; ERG 21242973 ChIP-ChIP JURKAT Human; NR3C1 21868756 ChIP-Seq MCF10A Human; DNAJC2 21179169 ChIP-ChIP NT2 Human; EGR1 K562 hg19; TAF1 HeLa—S3 hg19; PPARG 20176806 ChIP-Seq MACROPHAGES Mouse; ZBTB7A HepG2 hg19; RXR 22108803 ChIP-Seq LS180 Human; CTCF 21964334 Chip-Seq Bcells Human; BACH1 H1-hESC hg19; SAI 27219007 Chip-Seq ERYTHROID Human; SA1 27219007 Chip-Seq Bcells Human; FOXA1 26769127 Chip-Seq PDAC-Cell line Human; MAF 26560356 Chip-Seq TH2 Human; GATA4 25053715 ChIP-Seq YYC3 Human; NCOR1 26117541 ChIP-Seq K562 Human; FOXA1 25552417 ChIP-Seq VCAP Human; GATA6 25053715 ChIP-Seq YYC3 Human; ZNF143 H1-hESC hg19; RCOR1 CH12.LX mm9; NFYA HeLa—S3 hg19; POLR2A spleen mm9; ZNF143 K562 hg19; SUZ12 K562 hg19; SMARCC1 HeLa—S3 hg19; EP300 MEL cell line mm9; TP53 18474530 ChIP-ChIP U2OS Human; SP1 H1-hESC hg19; CEBPZ HepG2 hg19; TCF12 GM12878 hg19; SMAD4 19686287 ChIP-ChIP HaCaT Human; SMARCA4 23332759 ChIP-Seq OLIGODENDROCYTES Mouse; CTCF G1E mm9; POLR2AphosphoS5 K562 hg19; RXRA SK—N—SH hg19; CTCF MCF-7 hg19; ETS1 GM12878 hg19; CTCF cardiac muscle cell hg19; HOXC9 25013753 ChIP-Seq NEUROBLASTOMA BE2-C Human; WDR5 24793694 ChIP-Seq LNCAP Human; SRY 25088423 ChIP-ChIP EMBRYONIC GONADS Mouse; POLR2AphosphoS5 PFSK-1 hg19; HDAC2 HepG2 hg19; FOXP1 21924763 ChIP-Seq HESCs Human; CTCF GM12868 hg19; REST HCT116 hg19; CHD7 19251738 ChIP-ChIP MESCs Mouse; TFAP2A 17053090 ChIP-ChIP MCF-7 Human; E4F1 26484288 ChIP-Seq MOUSE EMBRYONIC FIBROBLAST Mouse; GF1 26923725 Chip-Seq HPCs Mouse; CDX2 19796622 ChIP-Seq MESCs Mouse; NELFE K562 hg19; MAX MCF-7 hg19; JUND MEL cell line mm9; GTF3C2 HeLa—S3 hg19; CTCF A549 hg19; SIN3A PFSK-1 hg19; REST Panc1 hg19; TAL1 erythroblast mm9; SALL1 21062744 ChIP-ChIP HESCs Human; SALL4 18804426 ChIP-ChIP XEN Mouse; SRF HepG2 hg19; CEBPD K562 hg19; CREB1 23762244 ChIP-Seq HIPPOCAMPUS Rat; ZNF169 human tf ARCHS4 coexpression; ISX human tf ARCHS4 coexpression; ZNF483 human tf ARCHS4 coexpression; ZNF524 human tf ARCHS4 coexpression; ZNF219 human tf ARCHS4 coexpression; ZNF214 human tf ARCHS4 coexpression; ZNF687 human tf ARCHS4 coexpression; ZNF432 human tf ARCHS4 coexpression; ZIC5 human tf ARCHS4 coexpression; ZNF740 human tf ARCHS4 coexpression; VEZF1 human tf ARCHS4 coexpression; POU5F1 human tf ARCHS4 coexpression; GLIS2 human tf ARCHS4 coexpression; ZNF436 human tf ARCHS4 coexpression; KLF7 human tf ARCHS4 coexpression; GON4L human tf ARCHS4 coexpression; ZBTB47 human tf ARCHS4 coexpression; ZNF423 human tf ARCHS4 coexpression; LHX5 human tf ARCHS4 coexpression; ZNF343 human tf ARCHS4 coexpression; ZFP64 human tf ARCHS4 coexpression; ZBTB32 human tf ARCHS4 coexpression; FOXS1 human tf ARCHS4 coexpression; SP5 human tf ARCHS4 coexpression; PRDM12 human tf ARCHS4 coexpression; ZNF648 human tf ARCHS4 coexpression; ZBED3 human tf ARCHS4 coexpression; ZNF703 human tf ARCHS4 coexpression; RNF114 human tf ARCHS4 coexpression; EDF1 human tf ARCHS4 coexpression; AKAP8 human tf ARCHS4 coexpression; RBM22 human tf ARCHS4 coexpression; PLXNA2 human tf ARCHS4 coexpression; MTA3 human tf ARCHS4 coexpression; HMGB3 human tf ARCHS4 coexpression; ZNF841 human tf ARCHS4 coexpression; ZNF782 human tf ARCHS4 coexpression; ZNF781 human tf ARCHS4 coexpression; THAP5 human tf ARCHS4 coexpression; HES3 human tf ARCHS4 coexpression; ZNF746 human tf ARCHS4 coexpression; ZNF764 human tf ARCHS4 coexpression; CARHSP1 human tf ARCHS4 coexpression; SETBP1 human tf ARCHS4 coexpression; PBX2 human tf ARCHS4 coexpression; TOX3 human tf ARCHS4 coexpression; ZNF564 human tf ARCHS4 coexpression; ZNF580 human tf ARCHS4 coexpression; FOXD4L6 human tf ARCHS4 coexpression; ZNF568 human tf ARCHS4 coexpression; ZXDA human tf ARCHS4 coexpression; ZNF248 human tf ARCHS4 coexpression; TSC22D2 human tf ARCHS4 coexpression; ZBTB6 human tf ARCHS4 coexpression; FEZF2 human tf ARCHS4 coexpression; ZNF26 human tf ARCHS4 coexpression; TBX1 human tf ARCHS4 coexpression; MYT1 human tf ARCHS4 coexpression; SPDEF human tf ARCHS4 coexpression; HOXC11 human tf ARCHS4 coexpression; TBX4 human tf ARCHS4 coexpression; ELF1 human tf ARCHS4 coexpression; ETV3 human tf ARCHS4 coexpression; ZBTB17 human tf ARCHS4 coexpression; NFE2L2 human tf ARCHS4 coexpression; TFCP2L1 human tf ARCHS4 coexpression; FOXP1 human tf ARCHS4 coexpression; HNF1B human tf ARCHS4 coexpression; ELF4 human tf ARCHS4 coexpression; RFX2 human tf ARCHS4 coexpression; MNT human tf ARCHS4 coexpression; PITX1 human tf ARCHS4 coexpression; SOX9 human tf ARCHS4 coexpression; NFYC human tf ARCHS4 coexpression; ISL1 human tf ARCHS4 coexpression; TFAP2B human tf ARCHS4 coexpression; ZFX human tf ARCHS4 coexpression; HOXA11 human tf ARCHS4 coexpression; PLXNA3 human tf ARCHS4 coexpression; CD36 human tf ARCHS4 coexpression; SETDB2 human tf ARCHS4 coexpression; MBD1 human tf ARCHS4 coexpression; VPS72 human tf ARCHS4 coexpression; TIPARP human tf ARCHS4 coexpression; CUL5 human tf ARCHS4 coexpression; H1FOO human tf ARCHS4 coexpression; LIN28B human tf ARCHS4 coexpression; HLA-DRB3 human tf ARCHS4 coexpression; FOXC1 human tf ARCHS4 coexpression; HOXA4 human tf ARCHS4 coexpression; HOXD9 human tf ARCHS4 coexpression; PAX3 human tf ARCHS4 coexpression; TEAD1 human tf ARCHS4 coexpression; HIST1H1A human tf ARCHS4 coexpression; SHOX human tf ARCHS4 coexpression; ZNF267 human tf ARCHS4 coexpression; MYBL1 human tf ARCHS4 coexpression; SIX5 human tf ARCHS4 coexpression; MLXIP human tf ARCHS4 coexpression; INSM1 human tf ARCHS4 coexpression; DMRT2 human tf ARCHS4 coexpression; SOX7 human tf ARCHS4 coexpression; ZNF350 human tf ARCHS4 coexpression; FOXA3 human tf ARCHS4 coexpression; IRX1 human tf ARCHS4 coexpression; SP7 human tf ARCHS4 coexpression; MYCN human tf ARCHS4 coexpression; ONECUT1 human tf ARCHS4 coexpression; ZNF23 human tf ARCHS4 coexpression; TLX3 human tf ARCHS4 coexpression; ZNF148 human tf ARCHS4 coexpression; NEUROD1 human tf ARCHS4 coexpression; ZNF385A human tf ARCHS4 coexpression; GRHL3 human tf ARCHS4 coexpression; TCF7L1 human tf ARCHS4 coexpression; NKX6-2 human tf ARCHS4 coexpression; NFIB human tf ARCHS4 coexpression; ST18 human tf ARCHS4 coexpression; CREB5 human tf ARCHS4 coexpression; ASCL1 human tf ARCHS4 coexpression; TFAP2A human tf ARCHS4 coexpression; NR5A1 human tf ARCHS4 coexpression; SOX14 human tf ARCHS4 coexpression; GFI1B human tf ARCHS4 coexpression; ZC3H4 human tf ARCHS4 coexpression; YY1 GM12892 hg19; FOXP3 21729870 ChIP-Seq TREG Human; LXR 22158963 ChIP-Seq LIVER Mouse; SA1 22415368 ChIP-Seq MEFs Mouse; ZFP281 27345836 Chip-Seq ESCs Mouse; RUNX2 24764292 ChIP-Seq MC3T3 Mouse; MAFK CH12.LX mm9; RARB 27405468 Chip-Seq BRAIN Mouse; CTCF small intestine mm9; ELF3 26769127 Chip-Seq PDAC-Cell line Human; CHD1 K562 hg19; RAD21 IMR-90 hg19; CEBPB H1-hESC hg19; CHD4 K562 hg19; EZH2 fibroblast of dermis hg19; CTCF medulloblastoma hg19; FOS endothelial cell of umbilical vein hg19; CTCF kidney hg19; TBP K562 hg19; E2A 27217539 Chip-Seq RAMOS-Cell line Human; REST U-87 MG hg19; REST SK—N—SH hg19; MYOG C2C12 mm9; ZBTB33 A549 hg19; YY1 A549 hg19; ERG 20887958 ChIP-Seq HPC-7 Mouse; JUND HeLa—S3 hg19; SIX5 GM12878 hg19; SMAD3 22036565 ChIP-Seq ESCs Mouse; AR 21572438 ChIP-Seq LNCaP Human; FOXP2 23625967 ChIP-Seq PFSK-1 AND SK—N-MC Human; KLF4 18358816 ChIP-ChIP MESCs Mouse; ETS1 K562 hg19; REST HeLa—S3 hg19; CEBPB A549 hg19; STAT3 19079543 ChIP-ChIP MESCs Mouse; TCF3 myocyte mm9; CTCF epithelial cell of esophagus hg19; NR2F2 K562 hg19; LYL1 20887958 ChIP-Seq HPC-7 Mouse; TAF1 A549 hg19; RAD21 21589869 ChIP-Seq MESCs Mouse; VDR 21846776 ChIP-Seq THP-1 Human; CTCF GM12870 hg19; NCOR 22465074 ChIP-Seq MACROPHAGES Mouse; NFYB 21822215 ChIP-Seq K562 Human; RCOR1 HeLa—S3 hg19; P63 26484246 Chip-Seq KERATINOCYTES Human; MYB 26560356 Chip-Seq TH2 Human; MXII K562 hg19; GATA3 26560356 Chip-Seq TH2 Human; RFX5 K562 hg19; EP300 osteoblast hg19; BHLHE40 A549 hg19; POLR2A Raji hg19; POLR2A bone marrow macrophage mm9; MAX NB4 hg19; PBX 27287812 Chip-Seq EMBYONIC-LIMB Mouse; SPI1 26923725 Chip-Seq HPCs Mouse; MAF 26560356 Chip-Seq TH1 Human; POLR2A kidney mm9; ZNF384 K562 hg19; ZNF263 HEK293 hg19; MAX C2C12 mm9; POLR2A heart mm9; UBTF CH12.LX mm9; MXII HeLa—S3 hg19; MAZ K562 hg19; MXI1 H1-hESC hg19; EZH2 HepG2 hg19; USF2 HeLa—S3 hg19; RBBP5 K562 hg19; ZNF384 GM12878 hg19; eGFP-HDAC8 K562 hg19; EZH2 endothelial cell of umbilical vein hg19; EZH2 fibroblast of lung hg19; BACH1 K562 hg19; TCF12 H1-hESC hg19; ETV2 25802403 ChIP-Seq MESCs Mouse; CTCF H1-hESC hg19; NANOG 21062744 ChIP-ChIP HESCs Human; P53 22127205 ChIP-Seq FIBROBLAST Human; EP300 A549 hg19; EGR1 19032775 ChIP-ChIP M12 Human; CEBPB GM12878 hg19; HOXD13 18407260 ChIP-ChIP DEVELOPING-LIMBS Mouse; RCOR1 MEL cell line mm9; CTCF fibroblast of mammary gland hg19; TEAD4 ECC-1 hg19; SRY 22984422 ChIP-ChIP TESTIS Rat; CTCF BE2C hg19; RAD21 HCT116 hg19; RCOR1 K562 hg19; TAF1H1-hESC hg19; MAX H1-hESC hg19; BCL11A H1-hESC hg19; FOSL1 K562 hg19; SMAD4 21799915 ChIP-Seq A2780 Human; JUND MCF-7 hg19; GATA1 erythroblast hg19; JUND HepG2 hg19; AHR 22903824 ChIP-Seq MCF-7 Human; SPI1 GM12878 hg19; POLR2A MEL cell line mm9; TAF1 neural cell hg19; TP53 16413492 ChIP-PET HCT116 Human; SP2 HepG2 hg19; BCL3 23251550 ChIP-Seq MUSCLE Mouse; TFAP2A HeLa—S3 hg19; RUNX1 26923725 Chip-Seq HPCs Mouse; CTNNB1 24651522 ChIP-Seq LGR5+ INTESTINAL STEM Human; USF1 H1-hESC hg19; TAL1 G1E mm9; JUND A549 hg19; KAP1 27257070 Chip-Seq ESCs Mouse; OCT1 27270436 Chip-Seq PROSTATE Human; TCF12/HEB 22897851 ChIP-Seq JUKARTE6-1 Human; FOXM1 26456572 ChIP-Seq MCF-7 Human; TOP2B 26459242 ChIP-Seq MCF-7 Human; NFYA 21822215 ChIP-Seq K562 Human; PU 27001747 Chip-Seq BMDM Mouse; SMC3 K562 hg19; POLR2A HEK293 hg19; ZMIZ1 CH12.LX mm9; CTCF bone marrow mm9; USF2 GM12878 hg19; GATA2 endothelial cell of umbilical vein hg19; TCF7L2 Panc1 hg19; POLR2A brain mm9; NFYB HeLa—S3 hg19; MYC NB4 hg19; WRNIP1 GM12878 hg19; CTCF spleen hg19; CTCF H54 hg19; ZNF384 ES-E14 mm9; GTF2F1 H1-hESC hg19; ZMIZ1 MEL cell line mm9; FOXP2 PFSK-1 hg19; SOX2 19030024 ChIP-ChIP MESCs Mouse; TAF15 26573619 Chip-Seq HEK293 Human; ZFP281 18358816 ChIP-ChIP MESCs Mouse; ARNT 22903824 ChIP-Seq MCF-7 Human; BMI1 19503595 ChIP-Seq MEFsC Mouse; SOX2 18555785 ChIP-Seq MESCs Mouse; ZC3H11A ES-E14 mm9; HOXA2 22223247 ChIP-Seq E11.5 EMBRYO Mouse; CDKN2AIP 20523734 ChIP-Seq CORTICAL Neurons; JUND T47D hg19; SPI1 GM12891 hg19; STAT5 23275557 ChIP-Seq MAMMARY-EPITHELIUM Mouse; CTCF LNCaP clone FGC hg19; IRF3 HeLa—S3 hg19; CTCF fibroblast of villous mesenchyme hg19; CBX3 HCT116 hg19; ZZZ3 GM12878 hg19; SREBF1 HepG2 hg19; SCL 19346495 ChIP-Seq HPC-7 Human; GABPA MEL cell line mm9; THAP1 K562 hg19; TCF12 ECC-1 hg19; BCL3 GM12878 hg19; CTCF HEK293 hg19; FOXM1 MCF-7 hg19; POLR2AphosphoS5 U-87 MG hg19; GRHL2 25758223 ChIP-Seq PLACENTA Mouse; NR3C1 23031785 ChIP-Seq PC12 Mouse; POLR2AphosphoS5 Panc1 hg19; MEIS1 20887958 ChIP-Seq HPC-7 Mouse; DACH1 20351289 ChIP-Seq MDA-MB-231 Human; ISL1 27105846 Chip-Seq CPCs Mouse; SP1 K562 hg19; SOX17 20123909 ChIP-Seq XEN Mouse; TP63 17297297 ChIP-ChIP HaCaT Human; SMAD3 21741376 ChIP-Seq HESCs Human; ZNF709 human tf ARCHS4 coexpression; FOXD2 human tf ARCHS4 coexpression; ZNF75D human tf ARCHS4 coexpression; ZNF749 human tf ARCHS4 coexpression; ZBTB40 human tf ARCHS4 coexpression; FOXL2 human tf ARCHS4 coexpression; ZNF438 human tf ARCHS4 coexpression; ZNF354C human tf ARCHS4 coexpression; ZFP28 human tf ARCHS4 coexpression; ZBTB34 human tf ARCHS4 coexpression; BNC2 human tf ARCHS4 coexpression; ZNF217 human tf ARCHS4 coexpression; MLLT3 human tf ARCHS4 coexpression; ZNF540 human tf ARCHS4 coexpression; ZBTB26 human tf ARCHS4 coexpression; FOXD4 human tf ARCHS4 coexpression; ZNF747 human tf ARCHS4 coexpression; ZBTB39 human tf ARCHS4 coexpression; ZNF497 human tf ARCHS4 coexpression; HES5 human tf ARCHS4 coexpression; ZNF536 human tf ARCHS4 coexpression; ZNF425 human tf ARCHS4 coexpression; DHX34 human tf ARCHS4 coexpression; KIAA1683 human tf ARCHS4 coexpression; MACF1 human tf ARCHS4 coexpression; ZC3H7A human tf ARCHS4 coexpression; TRIM32 human tf ARCHS4 coexpression; RNF125 human tf ARCHS4 coexpression; ZNF786 human tf ARCHS4 coexpression; THAP7 human tf ARCHS4 coexpression; THAP11 human tf ARCHS4 coexpression; MESP1 human tf ARCHS4 coexpression; ZCCHC11 human tf ARCHS4 coexpression; FEZF1 human tf ARCHS4 coexpression; CENPT human tf ARCHS4 coexpression; TOX4 human tf ARCHS4 coexpression; ARID3A human tf ARCHS4 coexpression; SIM2 human tf ARCHS4 coexpression; TCF25 human tf ARCHS4 coexpression; ZNF99 human tf ARCHS4 coexpression; LEUTX human tf ARCHS4 coexpression; ZSCAN21 human tf ARCHS4 coexpression; DZIP1 human tf ARCHS4 coexpression; ZNF488 human tf ARCHS4 coexpression; ZNF589 human tf ARCHS4 coexpression; EMX2 human tf ARCHS4 coexpression; NPAS2 human tf ARCHS4 coexpression; SIX2 human tf ARCHS4 coexpression; ATOH1 human tf ARCHS4 coexpression; RELA human tf ARCHS4 coexpression; ESRRA human tf ARCHS4 coexpression; ETV4 human tf ARCHS4 coexpression; FOXG1 human tf ARCHS4 coexpression; ZNF32 human tf ARCHS4 coexpression; ATOH7 human tf ARCHS4 coexpression; SRY human tf ARCHS4 coexpression; PBX1 human tf ARCHS4 coexpression; MITF human tf ARCHS4 coexpression; MESP2 human tf ARCHS4 coexpression; SP6 human tf ARCHS4 coexpression; TCF4 human tf ARCHS4 coexpression; PAX9 human tf ARCHS4 coexpression; RXRB human tf ARCHS4 coexpression; PLAG1 human tf ARCHS4 coexpression; HIC2 human tf ARCHS4 coexpression; SMAD3 human tf ARCHS4 coexpression; ZBED4 human tf ARCHS4 coexpression; EN1 human tf ARCHS4 coexpression; LHX8 human tf ARCHS4 coexpression; TAL1 human tf ARCHS4 coexpression; CUX1 human tf ARCHS4 coexpression; ZIC3 human tf ARCHS4 coexpression; ZNF41 human tf ARCHS4 coexpression; ZNF385B human tf ARCHS4 coexpression; PRDM16 human tf ARCHS4 coexpression; ZNF287 human tf ARCHS4 coexpression; NKX2-8 human tf ARCHS4 coexpression; LHX1 human tf ARCHS4 coexpression; ZNF132 human tf ARCHS4 coexpression; FOXO3 human tf ARCHS4 coexpression; RFX4 human tf ARCHS4 coexpression; NKX3-2 human tf ARCHS4 coexpression; HLF human tf ARCHS4 coexpression; PBX4 human tf ARCHS4 coexpression; ZNF419 human tf ARCHS4 coexpression; SALL4 human tf ARCHS4 coexpression; EN2 human tf ARCHS4 coexpression; CUL4A human tf ARCHS4 coexpression; GATAD2B human tf ARCHS4 coexpression; TEAD3 human tf ARCHS4 coexpression; MLXIPL human tf ARCHS4 coexpression; PRDM1 human tf ARCHS4 coexpression; RFX3 human tf ARCHS4 coexpression; NFE2L1 human tf ARCHS4 coexpression; TFAP2C human tf ARCHS4 coexpression; CREM human tf ARCHS4 coexpression; MZF1 human tf ARCHS4 coexpression; PAX6 human tf ARCHS4 coexpression; HNF4A human tf ARCHS4 coexpression; MEIS3 human tf ARCHS4 coexpression; KLF1 human tf ARCHS4 coexpression; USF2 human tf ARCHS4 coexpression; HOXA13 human tf ARCHS4 coexpression; PAX4 human tf ARCHS4 coexpression; MEOX2 human tf ARCHS4 coexpression; GATA3 human tf ARCHS4 coexpression; PHOX2B human tf ARCHS4 coexpression; LYL1 human tf ARCHS4 coexpression; IKZF2 human tf ARCHS4 coexpression; GLI1 human tf ARCHS4 coexpression; PCSK6 human tf ARCHS4 coexpression; ELK3 human tf ARCHS4 coexpression; NR2E1 human tf ARCHS4 coexpression; ZNF215 human tf ARCHS4 coexpression; THRB human tf ARCHS4 coexpression; HOXA10 human tf ARCHS4 coexpression; ZNF281 human tf ARCHS4 coexpression; MSX1 human tf ARCHS4 coexpression; KLF15 human tf ARCHS4 coexpression; NEUROD6 human tf ARCHS4 coexpression; HNF4G human tf ARCHS4 coexpression; TCF7L2 human tf ARCHS4 coexpression; SPIC human tf ARCHS4 coexpression; HIST1H1T human tf ARCHS4 coexpression; LENG9 human tf ARCHS4 coexpression; RGS6 human tf ARCHS4 coexpression; HLA-DQB1 human tf ARCHS4 coexpression; KAT5 human tf ARCHS4 coexpression; DEPDC4 human tf ARCHS4 coexpression; KIN human tf ARCHS4 coexpression; HES6 human tf ARCHS4 coexpression; IGHM human tf ARCHS4 coexpression; LHX9 human tf ARCHS4 coexpression; TBX19 human tf ARCHS4 coexpression; ZNF593 human tf ARCHS4 coexpression; HAND1 human tf ARCHS4 coexpression; IRX4 human tf ARCHS4 coexpression; ID3 human tf ARCHS4 coexpression; PROX1 human tf ARCHS4 coexpression; MXI1 human tf ARCHS4 coexpression; HOXB5 human tf ARCHS4 coexpression; HOXC13 human tf ARCHS4 coexpression; PAX1 human tf ARCHS4 coexpression; ID1 human tf ARCHS4 coexpression; NRL human tf ARCHS4 coexpression; GATA5 human tf ARCHS4 coexpression; GRHL1 human tf ARCHS4 coexpression; SOX3 human tf ARCHS4 coexpression; NKX2-1 human tf ARCHS4 coexpression; GATA4 human tf ARCHS4 coexpression; LMX1B human tf ARCHS4 coexpression; STAT4 human tf ARCHS4 coexpression; ZFHX3 human tf ARCHS4 coexpression; NFIC human tf ARCHS4 coexpression; BACH2 human tf ARCHS4 coexpression; IRF6 human tf ARCHS4 coexpression; ZNF302 human tf ARCHS4 coexpression; E2F6 K562 hg19; GATA1 19941826 ChIP-Seq K562 Human; POLR2AphosphoS5 neural cell hg19; EP300 H1-hESC hg19; STAT6 21828071 ChIP-Seq BEAS2B Human; RELA GM12892 hg19; POU5F1 18555785 ChIP-Seq MESCs Mouse; POLR2A cortical plate mm9; EZH2 mammary epithelial cell hg19; POLR2A ES-Bruce4 mm9; CTCF bone marrow macrophage mm9; ZKSCAN1 HeLa—S3 hg19; TBP CH12.LX mm9; GTF2F1 HeLa—S3 hg19; HCFC1 K562 hg19; RBBP5 H1-hESC hg19; EZH2 GM12878 hg19; ZNF384 MEL cell line mm9; GTF2F1 K562 hg19; GATA1 22383799 ChIP-Seq G1ME Mouse; ATF3 27146783 Chip-Seq COLON Human; FLI1 26923725 Chip-Seq HEMOGENIC-ENDOTHELIUM Mouse; TBL1 22424771 ChIP-Seq 293T Human; HA-E2F1 HeLa—S3 hg19; LXR 22292898 ChIP-Seq THP-1 Human; P68 20966046 ChIP-Seq HELA Human; SMC4 20622854 ChIP-Seq HELA Human; PHF8 20622854 ChIP-Seq HELA Human; BCOR 27268052 Chip-Seq Bcells Human; TCFCP2L1 18555785 Chip-Seq ESCs Mouse; TCF21 26020271 ChIP-Seq SMOOTH MUSCLE Human; MAX MEL cell line mm9; PPARA 22158963 ChIP-Seq LIVER Mouse; STAT5A K562 hg19; CEBPD HepG2 hg19; ZNF274 K562 hg19; CTCF astrocyte of the spinal cord hg19; REST ECC-1 hg19; TAL1 MEL cell line mm9; YY1 H1-hESC hg19; PPARD 23208498 ChIP-Seq MDA-MB-231 Human; CTCF HCT116 hg19; SP2 H1-hESC hg19; CTCF G1E-ER4 mm9; GATA3 T47D hg19; CREB1 A549 hg19; DMRT1 21621532 ChIP-ChIP FETAL Ovary; SOX9 26525672 Chip-Seq Limbbuds Mouse; RAD21 MEL cell line mm9; SIN3A H1-hESC hg19; REST GM12878 hg19; ZNF263 K562 hg19; CTCF fibroblast of pulmonary artery hg19; MYB MEL cell line mm9; BCL11B 21912641 ChIP-Seq STHDH STRIUM Mouse; GATA2 21571218 ChIP-Seq MEGAKARYOCYTES Human; MAX SK—N—SH hg19; SMAD1 18555785 ChIP-Seq MESCs Mouse; FOX03 23340844 ChIP-Seq DLD1 Human; VDR 20736230 ChIP-Seq LYMPHOBLASTOID Human; SMAD3 21741376 ChIP-Seq EPCs Human; ZKSCAN1 CH12.LX mm9; MYOG myocyte mm9; ZFP322A 24550733 ChIP-Seq MESCs Mouse; EP300 SK—N—SH hg19; NFE2L2 22581777 ChIP-Seq LYMPHOBLASTOID Human; LMO2 20887958 ChIP-Seq HPC-7 Mouse; SMARCA4 20176728 ChIP-ChIP TSCs Mouse; ATF3 A549 hg19; TAF1 GM12892 hg19; PADI4 21655091 ChIP-ChIP MCF-7 Human; VDR 24763502 ChIP-Seq THP-1 Human; CTCF GM06990 hg19; SOX2 18358816 ChIP-ChIP MESCs Mouse; RELA GM12878 hg19; GATA4 21415370 ChIP-Seq HL-1 Mouse; GATA1 19941827 ChIP-Seq MEL Mouse; UBF1/2 26484160 Chip-Seq HMEC-DERIVED Human; CSB 26484114 Chip-Seq FIBROBLAST Human; CTCF 21964334 ChIP-Seq BJAB-B Human; SMAD4 21741376 ChIP-Seq EPCs Human; SMAD4 21741376 ChIP-Seq ESCs Human; LMO2 26923725 Chip-Seq HEMOGENIC-ENDOTHELIUM Mouse; P300 19829295 ChIP-Seq ESCs Human; MEIS1 26253404 ChIP-Seq OPTIC CUPS Mouse; CEBPB 24764292 ChIP-Seq MC3T3 Mouse; TAL1 26923725 Chip-Seq HPCs Mouse; KLF5 20875108 ChIP-Seq MESCs Mouse; SOX2 27498859 Chip-Seq STOMACH Mouse; MAFF HepG2 hg19; E2F1 HeLa—S3 hg19; MAFK HeLa—S3 hg19; SAP30 H1-hESC hg19; MAZ HepG2 hg19; CEBPB IMR-90 hg19; POLR2A thymus mm9; TFAP2C HeLa—S3 hg19; STAT1 GM12878 hg19; JUND K562 hg19; BRCA1 HepG2 hg19; BHLHE40 GM12878 hg19; GATA2 SH-SY5Y hg19; POLR2A small intestine mm9; MXII CH12.LX mm9; MYC endothelial cell of umbilical vein hg19; eGFP-JUND K562 hg19; RAD21 CH12.LX mm9; eGFP-FOS K562 hg19; POLR2A GM18526 hg19; ZNF143 GM12878 hg19; BHLHE40 HepG2 hg19; TCF7L2 HeLa—S3 hg19; CTCF foreskin fibroblast hg19; SPI1 22790984 ChIP-Seq ERYTHROLEUKEMIA Mouse; PPARG 20887899 ChIP-Seq 3T3-L1 Mouse; POU5F1 16153702 ChIP-ChIP HESCs Human; CLOCK 20551151 ChIP-Seq 293T Human; PPARD 21283829 ChIP-Seq MYOFIBROBLAST Human; CTCF epithelial cell of proximal tubule hg19; HIFIA 21447827 ChIP-Seq MCF-7 Human; TAF1 GM12878 hg19; PDX1 19855005 ChIP-ChIP MIN6 Mouse; RNF2 27304074 Chip-Seq NSC Mouse; CTCF GM12873 hg19; SRF MCF-7 hg19; POU2F2 GM12891 hg19; MAX A549 hg19; FUS 26573619 Chip-Seq HEK293 Human; NANOG 16153702 ChIP-ChIP HESCs Human; HDAC2 H1-hESC hg19; BACH1 22875853 ChIP-PCR HELA AND SCP4 Human; JUND H1-hESC hg19; POU2F2 GM12878 hg19; EGR1 HCT116 hg19; FOXM1 GM12878 hg19; RXRA HepG2 hg19; AR 22383394 ChIP-Seq PROSTATE CANCER Human; POLR2A HeLa—S3 hg19; SIN3A Panc1 hg19; TFEB 21752829 ChIP-Seq HELA Human; MTA3 GM12878 hg19; TCF7L2 21901280 ChIP-Seq H4IIE Rat; SMAD4 21741376 ChIP-Seq HESCs Human; E2F6 HeLa—S3 hg19; VDR 23401126 ChIP-Seq LCL-AND-THP1 Human; SRF K562 hg19; NACC1 18358816 ChIP-ChIP MESCs Mouse; EBNA2 21746931 ChIP-Seq IB4-LCL Human; SOX9 26525672 Chip-Seq HEART Mouse; CTCF keratinocyte hg19; CEBPB myocyte mm9; TCF12 SK—N—SH hg19; CTCF fibroblast of the aortic adventitia hg19; HDAC2 K562 hg19; EP300 HepG2 hg19; POLR2A NB4 hg19; JUN HepG2 hg19; CTCF liver mm9; CTCF olfactory bulb mm9; CEBPB 26923725 Chip-Seq HEMOGENIC-ENDOTHELIUM Mouse; BRCA1 HeLa—S3 hg19; FLI1 21867929 ChIP-Seq TH2 Mouse; eGFP-JUNB K562 hg19; ZC3H11A K562 hg19; EZH2 H1-hESC hg19; CTCF heart mm9; IKZF1 GM12878 hg19; E2F4 K562 hg19; RAD21 HeLa—S3 hg19; SIN3A GM12878 hg19; CUX1 K562 hg19; YY1 NT2-D1 hg19; TP63 22573176 ChIP-Seq HFKS Human; TAL1 26923725 Chip-Seq HEMANGIOBLAST Mouse; PRDM1 HeLa—S3 hg19; GATA2 G1E mm9; SIN3A HCT116 hg19; RUNX1 20887958 ChIP-Seq HPC-7 Mouse; SIX5 A549 hg19; SALL4 22934838 ChIP-ChIP CD34+ Human; TCF4 18268006 ChIP-ChIP LS174T Human; ZEB1 GM12878 hg19; GATA1 erythroblast mm9; RBPJ 21746931 ChIP-Seq IB4-LCL Human; MAFK MEL cell line mm9; CBX3 K562 hg19; KLF5 18264089 ChIP-ChIP MESCs Mouse; KLF2 18264089 ChIP-ChIP MESCs Mouse; FOSL1 C2C12 mm9; RCOR3 21632747 ChIP-Seq MESCs Mouse; CEBPB C2C12 mm9; CTCF GM12865 hg19; MBD4 HepG2 hg19; E2F4 HeLa—S3 hg19; CEBPB MCF-7 hg19; SMAD2 18955504 ChIP-ChIP HaCaT Human; SMAD3 18955504 ChIP-ChIP HaCaT Human; CTCF mammary epithelial cell hg19; KLF4 18264089 ChIP-ChIP MESCs Mouse; MAX ECC-1 hg19; POLR2AphosphoS5 H1-hESC hg19; MAX HCT116 hg19; SOX11 23321250 ChIP-ChIP Z138-A519-JVM2 Human; TCF3 18692474 ChIP-Seq MEFs Mouse; IRF8 22096565 ChIP-ChIP GC-B Human; ZKSCAN1 MEL cell line mm9; AR 21909140 ChIP-Seq LNCAP Human; SPI1 23547873 ChIP-Seq NB4 Human; PAX5 GM12892 hg19; STAT1 K562 hg19; FOXI1 human tf ARCHS4 coexpression; PBX3 human tf ARCHS4 coexpression; SOHLH1 human tf ARCHS4 coexpression; CDX2 human tf ARCHS4 coexpression; HEY1 human tf ARCHS4 coexpression; FEV human tf ARCHS4 coexpression; NR3C1 human tf ARCHS4 coexpression; ZBTB25 human tf ARCHS4 coexpression; ZBTB38 human tf ARCHS4 coexpression; MAF human tf ARCHS4 coexpression; ZBTB7A human tf ARCHS4 coexpression; EGR3 human tf ARCHS4 coexpression; GSX2 human tf ARCHS4 coexpression; HOXC9 human tf ARCHS4 coexpression; MAFF human tf ARCHS4 coexpression; ZSCAN2 human tf ARCHS4 coexpression; MIXL1 human tf ARCHS4 coexpression; NOTO human tf ARCHS4 coexpression; HMX1 human tf ARCHS4 coexpression; ZNF839 human tf ARCHS4 coexpression; CREB3L3 human tf ARCHS4 coexpression; PRDM6 human tf ARCHS4 coexpression; ZNF446 human tf ARCHS4 coexpression; EBF4 human tf ARCHS4 coexpression; coexpression; POU3F3 human tf ARCHS4 coexpression; GMEB1 human tf ARCHS4 coexpression; FOSL1 human tf ARCHS4 coexpression; ZNF274 human tf ARCHS4 coexpression; ZFHX4 human tf ARCHS4 coexpression; ADNP human tf ARCHS4 coexpression; ADNP2 human tf ARCHS4 coexpression; ZKSCAN1 human tf ARCHS4 coexpression; ZNF684 human tf ARCHS4 coexpression; NKX2-4 human tf ARCHS4 coexpression; ZNF484 human tf ARCHS4 coexpression; ETV5 human tf ARCHS4 coexpression; MNX1 human tf ARCHS4 coexpression; SHOX2 human tf ARCHS4 coexpression; ATF5 human tf ARCHS4 coexpression; ZEB2 human tf ARCHS4 coexpression; GSX1 human tf ARCHS4 coexpression; HOXD4 human tf ARCHS4 coexpression; ATF7 human tf ARCHS4 coexpression; NEUROD2 human tf ARCHS4 coexpression; ZNF30 human tf ARCHS4 coexpression; JAZF1 human tf ARCHS4 coexpression; EHF human tf ARCHS4 coexpression; POU4F2 human tf ARCHS4 coexpression; LHX2 human tf ARCHS4 coexpression; DBP human tf ARCHS4 coexpression; ZNF175 human tf ARCHS4 coexpression; VSX1 human tf ARCHS4 coexpression; SIX4 human tf ARCHS4 coexpression; ESR1 human tf ARCHS4 coexpression; TP73 human tf ARCHS4 coexpression; ALX4 human tf ARCHS4 coexpression; NR2E3 human tf ARCHS4 coexpression; E2F2 human tf ARCHS4 coexpression; TFAP2D human tf ARCHS4 coexpression; NR3C2 human tf ARCHS4 coexpression; ZNF410 human tf ARCHS4 coexpression; HOXB1 human tf ARCHS4 coexpression; MSX2 human tf ARCHS4 coexpression; SNAIL human tf ARCHS4 coexpression; HIF3A human tf ARCHS4 coexpression; RUNX2 human tf ARCHS4 coexpression; HOXD11 human tf ARCHS4 coexpression; SOX5 human tf ARCHS4 coexpression; FOXN1 human tf ARCHS4 coexpression; GCM1 human tf ARCHS4 coexpression; SIX3 human tf ARCHS4 coexpression; ESR2 human tf ARCHS4 coexpression; TCF12 human tf ARCHS4 coexpression; BARHL2 human tf ARCHS4 coexpression; MAEL human tf ARCHS4 coexpression; RFX5 human tf ARCHS4 coexpression; SOX13 human tf ARCHS4 coexpression; SATB2 human tf ARCHS4 coexpression; CREB1 human tf ARCHS4 coexpression; ATF6 human tf ARCHS4 coexpression; TLX2 human tf ARCHS4 coexpression; CDX1 human tf ARCHS4 coexpression; EBF3 human tf ARCHS4 coexpression; ZNF226 human tf ARCHS4 coexpression; ZNF304 human tf ARCHS4 coexpression; CASZ1 human tf ARCHS4 coexpression; DBX2 human tf ARCHS4 coexpression; ZNF696 human tf ARCHS4 coexpression; ARGFX human tf ARCHS4 coexpression; MYT1L human tf ARCHS4 coexpression; ZNF559 human tf ARCHS4 coexpression; ZNF177 human tf ARCHS4 coexpression; ZNF563 human tf ARCHS4 coexpression; ZNF292 human tf ARCHS4 coexpression; ZNF502 human tf ARCHS4 coexpression; ZNF688 human tf ARCHS4 coexpression; ZNF517 human tf ARCHS4 coexpression; ZNF121 human tf ARCHS4 coexpression; ZNF560 human tf ARCHS4 coexpression; ZNF84 human tf ARCHS4 coexpression; ZNF251 human tf ARCHS4 coexpression; coexpression; ZNF485 human tf ARCHS4 coexpression; ZNF627 human tf ARCHS4 coexpression; ZNF652 human tf ARCHS4 coexpression; DUXA human tf ARCHS4 coexpression; ZNF253 human tf ARCHS4 coexpression; AFF3 human tf ARCHS4 coexpression; SLC30A9 human tf ARCHS4 coexpression; ZNF74 human tf ARCHS4 coexpression; RC3H2 human tf ARCHS4 coexpression; ZBTB7C human tf ARCHS4 coexpression; ZNF320 human tf ARCHS4 coexpression; ZNF414 human tf ARCHS4 coexpression; TBR1 human tf ARCHS4 coexpression; JDP2 human tf ARCHS4 coexpression; ZNF473 human tf ARCHS4 coexpression; ZNF18 human tf ARCHS4 coexpression; ZNF208 human tf ARCHS4 coexpression; ZNF496 human tf ARCHS4 coexpression; ZNF704 human tf ARCHS4 coexpression; ZNF337 human tf ARCHS4 coexpression; ZNF526 human tf ARCHS4 coexpression; ZNF558 human tf ARCHS4 coexpression; INSM2 human tf ARCHS4 coexpression; ZNF318 human tf ARCHS4 coexpression; KLF17 human tf ARCHS4 coexpression; DMRTA1 human tf ARCHS4 coexpression; ZNF454 human tf ARCHS4 coexpression; ZNF713 human tf ARCHS4 coexpression; ZNF154 human tf ARCHS4 coexpression; ZFP3 human tf ARCHS4 coexpression; ZNF443 human tf ARCHS4 coexpression; ZNF572 human tf ARCHS4 coexpression; HKR1 human tf ARCHS4 coexpression; ZNF329 human tf ARCHS4 coexpression; TFDP3 human tf ARCHS4 coexpression; MBNL3 human tf ARCHS4 coexpression; ZNF511 human tf ARCHS4 coexpression; PLEK human tf ARCHS4 coexpression; UNK human tf ARCHS4 coexpression; NCOR1 human tf ARCHS4 coexpression; FAM171B human tf ARCHS4 coexpression; SORBS2 human tf ARCHS4 coexpression; DZIP1L human tf ARCHS4 coexpression; CPSF4 human tf ARCHS4 coexpression; SOHLH2 human tf ARCHS4 coexpression; PBRM1 human tf ARCHS4 coexpression; PKHD1 human tf ARCHS4 coexpression; SMARCC1 human tf ARCHS4 coexpression; CHD2 human tf ARCHS4 coexpression; CBX2 human tf ARCHS4 coexpression; TIGD5 human tf ARCHS4 coexpression; H1FX human tf ARCHS4 coexpression; TOX human tf ARCHS4 coexpression; KRTAP5-1 human tf ARCHS4 coexpression; MBD4 human tf ARCHS4 coexpression; NROB2 human tf ARCHS4 coexpression; TGIF2 human tf ARCHS4 coexpression; NCOA3 human tf ARCHS4 coexpression; ZNF835 human tf ARCHS4 coexpression; ZNF428 human tf ARCHS4 coexpression; ZNF776 human tf ARCHS4 coexpression; DNAJC21 human tf ARCHS4 coexpression; AFF1 human tf ARCHS4 coexpression; ZNF791 human tf ARCHS4 coexpression; ZBED2 human tf ARCHS4 coexpression; ZNF792 human tf ARCHS4 coexpression; ZNF716 human tf ARCHS4 coexpression; FOXI2 human tf ARCHS4 coexpression; HMX2 human tf ARCHS4 coexpression; DEPDC7 human tf ARCHS4 coexpression; ZFP92 human tf ARCHS4 coexpression; ZNF772 human tf ARCHS4 coexpression; PLXND1 human tf ARCHS4 coexpression; RAPGEF3 human tf ARCHS4 coexpression; PDS5B human tf ARCHS4 coexpression; DSP human tf ARCHS4 coexpression; ZMAT5 human tf ARCHS4 coexpression; SMARCA1 human tf ARCHS4 coexpression; PPP1R13L human tf ARCHS4 coexpression; RGS9 human tf ARCHS4 coexpression; ZNF676 human tf ARCHS4 coexpression; CTCF BJ hg19; EGR1 GM12878 hg19; STAT3 1855785 ChIP-Seq MESCs Mouse; GATA2 21666600 ChIP-Seq HMVEC Human; TCF12 A549 hg19; NR2C2 HeLa—S3 hg19; SP1 GM12878 hg19; NANOG 18347094 ChIP-ChIP MESCs Mouse; FOXO1 23066095 ChIP-Seq LIVER Mouse; SIN3A SK—N—SH hg19; ELK1 22589737 ChIP-Seq MCF10A Human; NR4A2 19515692 ChIP-ChIP MN9D Mouse; CTCF limb mm9; CTCF IMR-90 hg19; ETS1 22383799 ChIP-Seq GIME Mouse; USF2 HepG2 hg19; POLR2AphosphoS2 HeLa—S3 hg19; YY1 GM12878 hg19; SMC3 CH12.LX mm9; NRF1 H1-hESC hg19; ELK1 K562 hg19; NRF1 SK—N—SH hg19; MAZ CH12.LX mm9; POLR2A GM19099 hg19; MYC CH12.LX mm9; POLR2A CH12.LX mm9; NRF1 HeLa—S3 hg19; TCF7L2 MCF-7 hg19; HDAC1 K562 hg19; CTCF GM13976 hg19; TCF7L2 HCT116 hg19; TBP H1-hESC hg19; TCF4 22108803 ChIP-Seq LS180 Human; OCT4 20526341 ChIP-Seq ESCs Human; CHD1 26751641 Chip-Seq LNCaP Human; MYC 27129775 Chip-Seq CORNEA Mouse; BCL6 27268052 Chip-Seq Bcells Human; CHD2 HepG2 hg19; RFX5 GM12878 hg19; MAX GM12878 hg19; NFYB GM12878 hg19; CTCF HL-60 hg19; CTCF cortical plate mm9; MAX endothelial cell of umbilical vein hg19; RFX5 HeLa—S3 hg19; CTCF NB4 hg19; E2F4 CH12.LX mm9; ZNF384 CH12.LX mm9; NFE2 GM12878 hg19; TAL1 K562 hg19; CEBPB HeLa—S3 hg19; HMGN3 K562 hg19; HNFA 21074721 ChIP-Seq CACO-2 Human; CBP 21632823 ChIP-Seq H3396 Human; JUN 26020271 ChIP-Seq SMOOTH MUSCLE Human; ERG 20517297 ChIP-Seq VCAP Human; POLR2A endothelial cell of umbilical vein hg19; MYC H1-hESC hg19; REST PFSK-1 hg19; TET1 21451524 ChIP-Seq MESCs Mouse; TCF3 18347094 ChIP-ChIP MESCs Mouse; KAT2A HeLa—S3 hg19; USF1 SK—N—SH hg19; IRF1 K562 hg19; Nerf2 26677805 Chip-Seq MACROPHAGESS Mouse; RAD21 K562 hg19; CTCF kidney epithelial cell hg19; CTCF retinal pigment epithelial cell hg19; MYBL2 HepG2 hg19; HNF4A 19822575 ChIP-Seq HepG2 Human; CTCF fibroblast of gingiva hg19; EP300 HeLa—S3 hg19; ESR1 20079471 ChIP-ChIP T-47D Human; YY1 SK—N—SH hg19; BRD4 25478319 ChIP-Seq HGPS Human; PAX5 GM12891 hg19; ESRRA HepG2 hg19; SIN3A MCF-7 hg19; ATF3 K562 hg19; SOX2 18692474 ChIP-Seq MEFs Mouse; CEBPD 21427703 ChIP-Seq 3T3-L1 Mouse; USF1 GM12878 hg19; CDX2 20551321 ChIP-Seq CACO-2 Human; SP4 H1-hESC hg19; CTCF GM12872 hg19; FOSL2 HepG2 hg19; CHD1 H1-hESC hg19; JUN HeLa—S3 hg19; IRF8 21731497 ChIP-ChIP J774 Mouse; KLF5 25053715 ChIP-Seq YYC3 Human; MYC MEL cell line mm9; NEUROD2 26341353 ChIP-Seq CORTEX Mouse; PU.1 20513432 ChIP-Seq MACROPHAGES Mouse; P63 20808887 ChIP-Seq KERATINOCYTES Human; SOX3 22085726 ChIP-Seq NPCs Mouse; RUNX1 22897851 ChIP-Seq JUKARTE6-1 Human; CJUN 26792858 Chip-Seq BT549 Human; FLI1 26923725 Chip-Seq MACROPHAGESS Mouse; KDM4A H1-hESC hg19; CTCF lung mm9; POLR2A olfactory bulb mm9; eGFP-GATA2 K562 hg19; POLR2A HGPS cell hg19; SMC3 GM12878 hg19; TBP MEL cell line mm9; CTCF GM20000 hg19; EP300 heart mm9; EZH2 astrocyte hg19; ETS1 CH12.LX mm9; EZH2 K562 hg19; POLR2AphosphoS2 myocyte mm9; CTCF HGPS cell hg19; RELA GM12891 hg19; NRF1 K562 hg19; TBP GM12878 hg19; BHLHE40 K562 hg19; MAFF K562 hg19; MXII SK—N—SH hg19; MAFK IMR-90 hg19; EOMES 20176728 ChIP-ChIP TSCs Mouse; STAT2 K562 hg19; CTCF GM12869 hg19; CTCF GM12867 hg19; SUPT20H HeLa—S3 hg19; SETDB1 K562 hg19; KLF1 20508144 ChIP-Seq FETAL-LIVER-ERYTHROID Mouse; GABPA H1-hESC hg19; ZBTB33 SK—N—SH hg19; MYCN 21190229 ChIP-Seq SHEP-21N Human; USF1 K562 hg19; ATF3 23680149 ChIP-Seq GBM1-GSC Human; MAFK HepG2 hg19; CEBPB 20176806 ChIP-Seq THIOMACROPHAGE Mouse; MYC HeLa—S3 hg19; PBX3 SK—N—SH hg19; USF2 MEL cell line mm9; PRDM16 22522345 ChIP-ChIP PALATE MESENCHYMAL Mouse; KAT2A GM12878 hg19; ESR1 T47D hg19; REST H1-hESC hg19; TCFAP2C 20176728 ChIP-ChIP TROPHOBLAST STEM CELLS Mouse; CTCF MEL cell line mm9; CREB1 GM12878 hg19; POLR2A MCF-7 hg19; FLI1 20887958 ChIP-Seq HPC-7 Mouse; POLR2A GM12891 hg19; CTCF choroid plexus epithelial cell hg19; NR2C2 K562 hg19; SRF H1-hESC hg19; REST neural cell hg19; YY1 HCT116 hg19; CTCF GM12875 hg19; GATA1 G1E-ER4 mm9; USF1 HepG2 hg19; MAFK ES-E14 mm9; TAL1 21186366 ChIP-Seq BM-HSCs Mouse; LDB1 21186366 ChIP-Seq BM-HSCs Mouse; E2F1 20622854 ChIP-Seq HELA Human; FOXA1 25329375 ChIP-Seq VCAP Human; POLR2A embryonic fibroblast mm9; MAZ GM12878 hg19; RELA GM19099 hg19; SOX2 21211035 ChIP-Seq LN229 Human; SMAD2/3 21741376 ChIP-Seq ESCs Human; ERA 21632823 ChIP-Seq H3396 Human; GATA3 24758297 ChIP-Seq MCF-7 Human; CTCF 26484167 Chip-Seq Bcells Mouse; FOXA1 27270436 Chip-Seq PROSTATE Human; TBP HepG2 hg19; SOX3 22085726 ChIP-Seq MUSCLE Mouse; CBX8 K562 hg19; CDX2 21074721 ChIP-Seq CACO-2 Mouse; POLR2A erythroblast hg19; KAT2A CH12.LX mm9; SMARCB1 HeLa—S3 hg19; BHLHE40 CH12.LX mm9; CCNT2 K562 hg19; BRCA1 H1-hESC hg19; BHLHE40 MEL cell line mm9; EZH2 HeLa—S3 hg19; CTCF embryonic fibroblast mm9; RELA GM15510 hg19; CTCF ES-Bruce4 mm9; BRCA1 GM12878 hg19; RAD21 HepG2 hg19; NFE2 K562 hg19; HNF4A 19761587 ChIP-ChIP CACO-2 Human; CTCF 18555785 ChIP-Seq MESCs Mouse; ESR1 22446102 ChIP-Seq UTERUS Mouse; EWS-ERG 20517297 ChIP-Seq CADO-ESI Human; PML K562 hg19; STAT3 24763339 ChIP-Seq IMN-ESCs Mouse; REST A549 hg19; STAT3 MCF 10A hg19; IGFIR 20145208 ChIP-Seq DFB Human; CCND1 20090754 ChIP-ChIP RETINA Mouse; ZFP57 27257070 Chip-Seq ESCs Mouse; GATA1 MEL cell line mm9; NFATC1 GM12878 hg19; STAT3 23295773 ChIP-Seq U87 Human; PML GM12878 hg19; MYC MCF-7 hg19; AUTS2 25519132 ChIP-Seq 293T-REX Human; TEAD4 K562 hg19; CTCF fibroblast of skin of abdomen hg19; CTCF fibroblast of pedal digit skin hg19; ZBTB33 GM12878 hg19; STAT3 HeLa—S3 hg19; RUNX1 17652178 ChIP-ChIP JURKAT Human; MYC 20876797 ChIP-ChIP MEDULLOBLASTOMA Human; CTCF endothelial cell of umbilical vein hg19; ESR1 17901129 ChIP-ChIP LIVER Mouse; PPAR 26484153 Chip-Seq NCI-H1993 Human; IRF1 21803131 ChIP-Seq MONOCYTES Human; JUN 21703547 ChIP-Seq K562 Human; YY1 K562 hg19; CTCF Caco-2 hg19; TAF1 HepG2 hg19; SPII K562 hg19; SFPI1 20887958 ChIP-Seq HPC-7 Mouse; CTBP1 25329375 ChIP-Seq LNCAP Human; NOTCH1 17114293 ChIP-ChIP T-ALL Human; ER 23166858 ChIP-Seq MCF-7 Human; YY1 HepG2 hg19; CTCF GM12866 hg19; PAX5 GM12878 hg19; GLI1 17442700 ChIP-ChIP MESCs Mouse; EKLF 21900194 ChIP-Seq ERYTHROCYTE Mouse; SMARCD1 25818293 ChIP-Seq ESCs Mouse; BATF3 human tf ARCHS4 coexpression; ZNF331 human tf ARCHS4 coexpression; ZBTB24 human tf ARCHS4 coexpression; AEBP2 human tf ARCHS4 coexpression; DMRTC2 human tf ARCHS4 coexpression; RERE human tf ARCHS4 coexpression; DMRTA2 human tf ARCHS4 coexpression; PDX1 human tf ARCHS4 coexpression; DMRT1 human tf ARCHS4 coexpression; DPF2 human tf ARCHS4 coexpression; NPAS1 human tf ARCHS4 coexpression; ZNF430 human tf ARCHS4 coexpression; TOE1 human tf ARCHS4 coexpression; ZNF697 human tf ARCHS4 coexpression; GLIS3 human tf ARCHS4 coexpression; ZNF268 human tf ARCHS4 coexpression; PRDM8 human tf ARCHS4 coexpression; ZNF117 human tf ARCHS4 coexpression; ZNF599 human tf ARCHS4 coexpression; ZNF234 human tf ARCHS4 coexpression; ZNF235 human tf ARCHS4 coexpression; ZNF761 human tf ARCHS4 coexpression; ZNF75A human tf ARCHS4 coexpression; ZNF513 human tf ARCHS4 coexpression; FOXQ1 human tf ARCHS4 coexpression; UNCX human tf ARCHS4 human tf ARCHS4 coexpression; NF1I3 human tf ARCHS4 coexpression; ZNF229 coexpression; DMRTB1 human tf ARCHS4 coexpression; ZNF500 human tf ARCHS4 coexpression; OLIG2 human tf ARCHS4 coexpression; ZNF416 human tf ARCHS4 coexpression; VAX1 human tf ARCHS4 coexpression; BACH1 human tf ARCHS4 coexpression; ERG human tf ARCHS4 coexpression; NR2F6 human tf ARCHS4 coexpression; PKNOX1 human tf ARCHS4 coexpression; LMX1A human tf ARCHS4 coexpression; MEIS1 human tf ARCHS4 coexpression; ZNF2 human tf ARCHS4 coexpression; EOMES human tf ARCHS4 coexpression; ZNF3 human tf ARCHS4 coexpression; ZNF91 human tf ARCHS4 coexpression; ZNF180 human tf ARCHS4 coexpression; RBPJ human tf ARCHS4 coexpression; ZNF646 human tf ARCHS4 coexpression; ELF3 human tf ARCHS4 coexpression; ZNF213 human tf ARCHS4 coexpression; NR113 human tf ARCHS4 coexpression; NROB1 human tf ARCHS4 coexpression; ZNF671 human tf ARCHS4 coexpression; ZNF43 human tf ARCHS4 coexpression; HOXC4 human tf ARCHS4 coexpression; ZNF273 human tf ARCHS4 coexpression; TEAD4 human tf ARCHS4 coexpression; ZNF398 human tf ARCHS4 coexpression; SMAD4 human tf ARCHS4 coexpression; SP1 human tf ARCHS4 coexpression; CREB3L2 human tf ARCHS4 coexpression; GATA2 human tf ARCHS4 coexpression; LCORL human tf ARCHS4 coexpression; TGIF1 human tf ARCHS4 coexpression; SOX11 human tf ARCHS4 coexpression; NR1D2 human tf ARCHS4 coexpression; HOXB4 human tf ARCHS4 coexpression; ZNF610 human tf ARCHS4 coexpression; HELZ human tf ARCHS4 coexpression; ZNF507 human tf ARCHS4 coexpression; TRIM23 human tf ARCHS4 coexpression; GRM6 human tf ARCHS4 coexpression; EEA1 human tf ARCHS4 coexpression; PLEK2 human tf ARCHS4 coexpression; DEPDC5 human tf ARCHS4 coexpression; BAZ2A human tf ARCHS4 coexpression; MKRN2 human tf ARCHS4 coexpression; PMS1 human tf ARCHS4 coexpression; RAD51 human tf ARCHS4 coexpression; CUL3 human tf ARCHS4 coexpression; AKAP8L human tf ARCHS4 coexpression; YBX2 human tf ARCHS4 coexpression; RBM5 human tf ARCHS4 coexpression; ZNF316 human tf ARCHS4 coexpression; ZSCAN5B human tf ARCHS4 coexpression; ZNF774 human tf ARCHS4 coexpression; ZNF804B human tf ARCHS4 coexpression; ZNF594 human tf ARCHS4 coexpression; ZNF518B human tf ARCHS4 coexpression; ZNF843 human tf ARCHS4 coexpression; ZNF804A human tf ARCHS4 coexpression; ZNF768 human tf ARCHS4 coexpression; ZMAT4 human tf ARCHS4 coexpression; THAP8 human tf ARCHS4 coexpression; ZNF773 human tf ARCHS4 coexpression; PLXNA1 human tf ARCHS4 coexpression; ZC3H13 human tf ARCHS4 coexpression; SLC2A4RG human tf ARCHS4 coexpression; RC3H1 human tf ARCHS4 coexpression; HLA-DQB2 human tf ARCHS4 coexpression; RPA4 human tf ARCHS4 coexpression; FOXO6 human tf ARCHS4 coexpression; RHOXF2B human tf ARCHS4 coexpression; KRTAP5-9 human tf ARCHS4 coexpression; HOXD10 human tf ARCHS4 coexpression; SLC39A10 human tf ARCHS4 coexpression; PLXNB2 human tf ARCHS4 coexpression; ARID2 human tf ARCHS4 coexpression; CPSF4L human tf ARCHS4 coexpression; CHD9 human tf ARCHS4 coexpression; GTF2IRD2B human tf ARCHS4 coexpression; SCAPER human tf ARCHS4 coexpression; SSH3 human tf ARCHS4 coexpression; ZRSR2 human tf ARCHS4 coexpression; HIST1H1E human tf ARCHS4 coexpression; ZMAT1 human tf ARCHS4 coexpression; LARP6 human tf ARCHS4 coexpression; DHX57 human tf ARCHS4 coexpression; BPNT1 human tf ARCHS4 coexpression; CUL4B human tf ARCHS4 coexpression; SMARCA5 human tf ARCHS4 coexpression; POGZ human tf ARCHS4 coexpression; DUS3L human tf ARCHS4 coexpression; SMAD6 human tf ARCHS4 coexpression; RAPGEF5 human tf ARCHS4 coexpression; RNASE2 human tf ARCHS4 coexpression; ZNF345 human tf ARCHS4 coexpression; ZNF391 human tf ARCHS4 coexpression; ZNF821 human tf ARCHS4 coexpression; ZNF546 human tf ARCHS4 coexpression; ZNF17 human tf ARCHS4 coexpression; ZNF284 human tf ARCHS4 coexpression; ZNF224 human tf ARCHS4 coexpression; ZNF566 human tf ARCHS4 coexpression; ZNF623 human tf ARCHS4 coexpression; ZNF662 human tf ARCHS4 coexpression; ZNF101 human tf ARCHS4 coexpression; ZBTB20 human tf ARCHS4 coexpression; ZNF707 human tf ARCHS4 coexpression; ZSCAN4 human tf ARCHS4 coexpression; TSHZ1 human tf ARCHS4 coexpression; ZFP42 human tf ARCHS4 coexpression; TPRX1 human tf ARCHS4 coexpression; ZNF366 human tf ARCHS4 coexpression; ZNF575 human tf ARCHS4 coexpression; FOXR1 human tf ARCHS4 coexpression; ZNF266 human tf ARCHS4 coexpression; KLF14 human tf ARCHS4 coexpression; ZNF554 human tf ARCHS4 coexpression; ZNF556 human tf ARCHS4 coexpression; ZNF570 human tf ARCHS4 coexpression; PRDM10 human tf ARCHS4 coexpression; ZNF280A human tf ARCHS4 coexpression; ZNF354A human tf ARCHS4 coexpression; ZNF70 human tf ARCHS4 coexpression; ZSCAN23 human tf ARCHS4 coexpression; ZNF527 human tf ARCHS4 coexpression; ZKSCAN3 human tf ARCHS4 coexpression; CXXC1 human tf ARCHS4 coexpression; BMP2 human tf ARCHS4 coexpression; SIM1 human tf ARCHS4 coexpression; AHR human tf ARCHS4 coexpression; NRF1 human tf ARCHS4 coexpression; ZNF705D human tf ARCHS4 coexpression; ZNF134 human tf ARCHS4 coexpression; ZNF155 human tf ARCHS4 coexpression; ZNF616 human tf ARCHS4 coexpression; DPRX human tf ARCHS4 coexpression; ZBTB22 human tf ARCHS4 coexpression; ZFP2 human tf ARCHS4 coexpression; KIAA1549 human tf ARCHS4 coexpression; ZNF658 human tf ARCHS4 coexpression; ZNF433 human tf ARCHS4 coexpression; ZNF615 human tf ARCHS4 coexpression; DMBX1 human tf ARCHS4 coexpression; ZNF665 human tf ARCHS4 coexpression; ZNF695 human tf ARCHS4 coexpression; ZNF679 human tf ARCHS4 coexpression; ZNF100 human tf ARCHS4 coexpression; ZNF33B human tf ARCHS4 coexpression; ZKSCAN5 human tf ARCHS4 coexpression; ZNF569 human tf ARCHS4 coexpression; ZNF34 human tf ARCHS4 coexpression; ZNF699 human tf ARCHS4 coexpression; ZNF461 human tf ARCHS4 coexpression; SOX4 human tf ARCHS4 coexpression; HOXB13 human tf ARCHS4 coexpression; ZNF644 human tf ARCHS4 coexpression; HOXA7 human tf ARCHS4 coexpression; ARNTL2 human tf ARCHS4 coexpression; NR1H4 human tf ARCHS4 coexpression; BARX2 human tf ARCHS4 coexpression; ZIC2 human tf ARCHS4 coexpression; DMRT3 human tf ARCHS4 coexpression; NR2C1 human tf ARCHS4 coexpression; HOXB8 human tf ARCHS4 coexpression; PLAGL1 human tf ARCHS4 coexpression; NANOG human tf ARCHS4 coexpression; VDR human tf ARCHS4 coexpression; PRDM4 human tf ARCHS4 coexpression; HOXA1 human tf ARCHS4 coexpression; ZNF14 human tf ARCHS4 coexpression; CTCF human tf ARCHS4 coexpression; NFAT5 human tf ARCHS4 coexpression; TSC22D1 human tf ARCHS4 coexpression; CDC5L human tf ARCHS4 coexpression; ZNF10 human tf ARCHS4 coexpression; TFAP4 human tf ARCHS4 coexpression; PAX2 human tf ARCHS4 coexpression; TFE3 human tf ARCHS4 coexpression; DLX3 human tf ARCHS4 coexpression; TEAD4 22529382 ChIP-Seq TROPHECTODERM Mouse; TCF7L2 HepG2 hg19; CHD1 MEL cell line mm9; YY1 23942234 ChIP-Seq MYOBLASTS AND MYOTUBES Mouse; MAFK GM12878 hg19; CEBPB HepG2 hg19; SIX5 K562 hg19; POLR2A GM12892 hg19; CEBPD 23245923 ChIP-Seq MEFs Mouse; CEBPB HCT116 hg19; SP1 A549 hg19; POLR2A limb mm9; CTCF skin fibroblast hg19; KAT2A MEL cell line mm9; CHD1 GM12878 hg19; NELFE CH12.LX mm9; USF2 CH12.LX mm9; UBTF MEL cell line mm9; MAX CH12.LX mm9; NFYB K562 hg19; USF1 myocyte mm9; CHD1 CH12.LX mm9; ZNF143 HeLa—S3 hg19; CTCF GM10266 hg19; MAFK K562 hg19; POLR2A GM19193 hg19; CTCF astrocyte hg19; PHF8 K562 hg19; CTCF cardiac fibroblast hg19; CTCF brain mm9; FOXH1 21741376 ChIP-Seq ESCs Human; MAZ MEL cell line mm9; NRF1 GM12878 hg19; MYC 19829295 ChIP-Seq ESCs Human; GATA3 21878914 ChIP-Seq MCF-7 Human; BCL6 25482012 ChIP-Seq CML-JURL-MK1 Human; AR 21915096 ChIP-Seq LNCaP-IF5 Human; P53 21459846 ChIP-Seq SAOS-2 Human; BCAT 22108803 ChIP-Seq LS180 Human; ESRRA GM12878 hg19; STAT5A GM12878 hg19; ELF1 GM12878 hg19; ZBTB33 HCT116 hg19; POLR2AphosphoS5 MEL cell line mm9; POLR2A HepG2 hg19; POU5F1 18347094 ChIP-ChIP MESCs Mouse; POLR2AphosphoS2 K562 hg19; ZIC3 20872845 ChIP-ChIP MESCs Mouse; ASXL1 24218140 ChIP-Seq BMDM Mouse; SIN3A A549 hg19; BATF GM12878 hg19; TP53 23651856 ChIP-Seq MEFs Mouse; GATA1 megakaryocyte mm9; TAF1 K562 hg19; CRX 20693478 ChIP-Seq ADULT RETINA Mouse; POLR2AphosphoS5 endothelial cell of umbilical vein hg19; GATA3 SK—N—SH hg19; CNOT3 19339689 ChIP-ChIP MESCs Mouse; TAF1 PFSK-1 hg19; TCF3 GM12878 hg19; SIN3A 21632747 ChIP-Seq MESCs Mouse; OCT4 18692474 ChIP-Seq MEFs Mouse; TAF1 GM12891 hg19; NR2F2 HepG2 hg19; EWS-FLI1 20517297 ChIP-Seq SK—N-MC Human; SREBF1 GM12878 hg19; UBTF K562 hg19; STAT3 18555785 ChIP-Seq MESCs Mouse; MYC K562 hg19; BCLAF1 K562 hg19; CTCF C2C12 mm9; KDM5B K562 hg19; CTCF lung hg19; POLR2AphosphoS2 HepG2 hg19; POLR2AphosphoS2 GM12878 hg19; RELA GM10847 hg19; GABPA CH12.LX mm9; CHD7 K562 hg19; CTCF thymus mm9; CTCF GM19239 hg19; POLR2AphosphoS2 CH12.LX mm9; SMC3 HeLa—S3 hg19; CHD2 K562 hg19; RCOR1 HepG2 hg19; MXI1 GM12878 hg19; USF1 C2C12 mm9; EP300 ES-Bruce4 mm9; CTCF pancreas hg19; ELK4 HeLa—S3 hg19; CHD1 IMR-90 hg19; RELA GM18951 hg19; NRF1 HepG2 hg19; POLR2A lung mm9; CBX2 K562 hg19; RELA GM19193 hg19; RARB 24833708 ChIP-Seq LIVER Mouse; NANOG 18555785 Chip-Seq ESCs Mouse; OCT4 18555785 Chip-Seq ESCs Mouse; CEBPA 20513432 ChIP-Seq MACROPHAGES Mouse; NF1 21473784 ChIP-Seq ESCs Mouse; SMAD2/3 21741376 ChIP-Seq EPCs Human; ETS1 21867929 ChIP-Seq TH2 Mouse; RUNX1 22412390 ChIP-Seq EML Mouse; UBF1/2 26484160 Chip-Seq FIBROBLAST Human; CEBPB 26923725 Chip-Seq MACROPHAGESS Mouse; CEBPB 20176806 ChIP-Seq MACROPHAGES Mouse; CEBPB 21427703 ChIP-Seq 3T3-L1 Mouse; NFE2L2 20460467 ChIP-Seq MEFs Mouse; NRF2 20460467 ChIP-Seq MEFs Mouse; NROB1 18358816 ChIP-ChIP MESCs Mouse; REST K562 hg19; KDM5A 27292631 Chip-Seq BREAST Human; GATA3 MCF-7 hg19; ZNF217 MCF-7 hg19; ZBTB33 HepG2 hg19; EP300 21415370 ChIP-Seq HL-1 Mouse; CTCF WERI-Rb-1 hg19; CREB1 20920259 ChIP-Seq GC1-SPG Mouse; YY1 ECC-1 hg19; SPI1 20517297 ChIP-Seq HL60 Human; CTCF HeLa—S3 hg19; CEBPB K562 hg19; NR2C2 HepG2 hg19; CEBPA 23403033 ChIP-Seq LIVER Mouse; CTCF bronchial epithelial cell hg19; POLR2A GM12878 hg19; EGR1 MCF-7 hg19; NR2C2 GM12878 hg19; FOS MCF 10A hg19; FOXA1 27197147 Chip-Seq ENDOMETRIOID-ADENOCARCINOMA Human; TCFCP2L1 18555785 ChIP-Seq MESCs Mouse; POLR2AphosphoS5 GM12891 hg19; NANOG 18692474 ChIP-Seq MEFs Mouse; NR112 20693526 ChIP-Seq LIVER Mouse; SRF 21415370 ChIP-Seq HL-1 Mouse; CTCF GM12871 hg19; MYC 22102868 ChIP-Seq BL Human; ATF2 H1-hESC hg19; KLF4 19030024 ChIP-ChIP MESCs Mouse; ELK4 HEK293 hg19; E2F4 myocyte mm9; ZMIZ1 K562 hg19; POLR2A myocyte mm9; ELK1 HeLa—S3 hg19; CTCF CD14-positive monocyte hg19; PHF8 H1-hESC hg19; POLR2A GM10847 hg19; SIRT6 K562 hg19; CHD2 H1-hESC hg19; POLR2A testis mm9; CTCF fibroblast of dermis hg19; SIN3A CH12.LX mm9; TBLIXR1 GM12878 hg19; POLR2A IMR-90 hg19; KAT2B K562 hg19; MYC A549 hg19; MAFK H1-hESC hg19; HDAC6 H1-hESC hg19; GATA3 22897851 ChIP-Seq JUKARTE6-1 Human; PU1 27457419 Chip-Seq LIVER Mouse; GATA3 26560356 Chip-Seq TH1 Human; NCOR 22424771 ChIP-Seq 293T Human; PU.1 20176806 ChIP-Seq MACROPHAGES Mouse; PHF8 20622853 ChIP-Seq HELA Human; GATA2 21186366 ChIP-Seq BM-HSCs Mouse; DPY 21335234 ChIP-Seq ESCs Mouse; RBPJ 21746931 ChIP-Seq IB4 Human; SOX11 22085726 ChIP-Seq ESNs Mouse; PCGF4 22325352 ChIP-Seq 293T-Rex Human; EP300 K562 hg19; IRF4 GM12878 hg19; POLR2A K562 hg19; CHD2 MEL cell line mm9; GBX2 23144817 ChIP-Seq PC3 Human; SCL 21571218 ChIP-Seq MEGAKARYOCYTES Human; POLR2AphosphoS5 SK—N—SH hg19; CTCF fibroblast of lung hg19; RCOR2 21632747 ChIP-Seq MESCs Mouse; MAX HepG2 hg19; CTBP2 25329375 ChIP-Seq LNCAP Human; MEIS1 26923725 Chip-Seq HEMOGENIC-ENDOTHELIUM Mouse; CTCF GM12874 hg19; RUNX2 22187159 ChIP-Seq PCA Human; TAL1 20887958 ChIP-Seq HPC-7 Mouse; eGFP-NR4A1 K562 hg19; CTCF GM12878 hg19; ESR1 15608294 ChIP-ChIP MCF-7 Human; GABPA GM12878 hg19; NR2F2 MCF-7 hg19; MYC 18555785 ChIP-Seq MESCs Mouse; TCF3 18467660 ChIP-ChIP MESCs Mouse; MYCN 19997598 ChIP-ChIP NEUROBLASTOMA Human; CTCF fibroblast of upper leg skin hg19; BCL3 A549 hg19; CTCF GM12864 hg19; SRF myocyte mm9; TAF1 ECC-1 hg19; MAX myocyte mm9; POLR2AphosphoS5 G1E-ER4 mm9; TBX3 20139965 ChIP-Seq MESCs Mouse; GPATCH8 human tf ARCHS4 coexpression; TERF1 human tf ARCHS4 coexpression; NPAS3 human tf ARCHS4 coexpression; NFXL1 human tf ARCHS4 coexpression; BOLA1 human tf ARCHS4 coexpression; NOC4L human tf ARCHS4 coexpression; ZNF836 human tf ARCHS4 coexpression; CHRAC1 human tf ARCHS4 coexpression; DMC1 human tf ARCHS4 coexpression; RAPGEF4 human tf ARCHS4 coexpression; MRPL28 human tf ARCHS4 coexpression; SMARCEL human tf ARCHS4 coexpression; MBD3 human tf ARCHS4 coexpression; TRIT1 human tf ARCHS4 coexpression; CSDE1 human tf ARCHS4 coexpression; DVL2 human tf ARCHS4 coexpression; RBM6 human tf ARCHS4 coexpression; WDHD1 human tf ARCHS4 coexpression; ZNF808 human tf ARCHS4 coexpression; ZNF770 human tf ARCHS4 coexpression; ZNF790 human tf ARCHS4 coexpression; ZNF98 human tf ARCHS4 coexpression; ZNF763 human tf ARCHS4 coexpression; ZNF429 human tf ARCHS4 coexpression; DOT1L human tf ARCHS4 coexpression; ZRSR1 human tf ARCHS4 coexpression; LGR4 human tf ARCHS4 coexpression; FOXD4L5 human tf ARCHS4 coexpression; PRR3 human tf ARCHS4 coexpression; PPP1R10 human tf ARCHS4 coexpression; ZNF765 human tf ARCHS4 coexpression; REPIN1 human tf ARCHS4 coexpression; TRMT1 human tf ARCHS4 coexpression; ZFR2 human tf ARCHS4 coexpression; PLEKHA4 human tf ARCHS4 coexpression; ZC3H18 human tf ARCHS4 coexpression; U2AF1 human tf ARCHS4 coexpression; U2AF1L4 human tf ARCHS4 coexpression; ZDHHC19 human tf ARCHS4 coexpression; PLXNB1 human tf ARCHS4 coexpression; TIGD6 human tf ARCHS4 coexpression; FMNL2 human tf ARCHS4 coexpression; RAG1 human tf ARCHS4 coexpression; MECP2 human tf ARCHS4 coexpression; ZNF674 human tf ARCHS4 coexpression; FARSA human tf ARCHS4 coexpression; TIGD2 human tf ARCHS4 coexpression; YOD1 human tf ARCHS4 coexpression; EWSR1 human tf ARCHS4 coexpression; USP39 human tf ARCHS4 coexpression; GTF2H2 human tf ARCHS4 coexpression; MINK1 human tf ARCHS4 coexpression; CBLL1 human tf ARCHS4 coexpression; DVL1 human tf ARCHS4 coexpression; POLE4 human tf ARCHS4 coexpression; CASP8AP2 human tf ARCHS4 coexpression; GTF3A human tf ARCHS4 coexpression; BAZ2B human tf ARCHS4 coexpression; POGK human tf ARCHS4 coexpression; RNF113A human tf ARCHS4 coexpression; SLC26A10 human tf ARCHS4 coexpression; NUPL2 human tf ARCHS4 coexpression; APTX human tf ARCHS4 coexpression; RBM26 human tf ARCHS4 coexpression; HMG20A human tf ARCHS4 coexpression; NCOA2 human tf ARCHS4 coexpression; ZC3H10 human tf ARCHS4 coexpression; GTF2IRD2 human tf ARCHS4 coexpression; ZNF787 human tf ARCHS4 coexpression; HMGA1 human tf ARCHS4 coexpression; ZNF793 human tf ARCHS4 coexpression; ZC3H3 human tf ARCHS4 coexpression; ZNF622 human tf ARCHS4 coexpression; ZNF160 human tf ARCHS4 coexpression; FOXN2 human tf ARCHS4 coexpression; TBX10 human tf ARCHS4 coexpression; IRX6 human tf ARCHS4 coexpression; ZNF19 human tf ARCHS4 coexpression; GABPA human tf ARCHS4 coexpression; POU4F1 human tf ARCHS4 coexpression; TBX6 human tf ARCHS4 coexpression; NR6A1 human tf ARCHS4 coexpression; RBAK human tf ARCHS4 coexpression; NR112 human tf ARCHS4 coexpression; SP1B human tf ARCHS4 coexpression; TP53 human tf ARCHS4 coexpression; PRDM5 human tf ARCHS4 coexpression; HOXB2 human tf ARCHS4 coexpression; ZBTB4 human tf ARCHS4 coexpression; DEAF1 human tf ARCHS4 coexpression; ZNF708 human tf ARCHS4 coexpression; ZBTB45 human tf ARCHS4 coexpression; ZNF541 human tf ARCHS4 coexpression; DR1 human tf ARCHS4 coexpression; BBX human tf ARCHS4 coexpression; ZNF550 human tf ARCHS4 coexpression; ZNF90 human tf ARCHS4 coexpression; ZNF184 human tf ARCHS4 coexpression; POU2F3 human tf ARCHS4 coexpression; IKZF5 human tf ARCHS4 coexpression; PHF20 human tf ARCHS4 coexpression; ZNF28 human tf ARCHS4 coexpression; MAFG human tf ARCHS4 coexpression; PAX5 human tf ARCHS4 coexpression; FOXD3 human tf ARCHS4 coexpression; HSF1 human tf ARCHS4 coexpression; OLIG1 human tf ARCHS4 coexpression; ZNF280C human tf ARCHS4 coexpression; MYC human tf ARCHS4 coexpression; HEY2 human tf ARCHS4 coexpression; GSC human tf ARCHS4 coexpression; UBTF human tf ARCHS4 coexpression; HOXA2 human tf ARCHS4 coexpression; GMEB2 human tf ARCHS4 coexpression; TCFL5 human tf ARCHS4 coexpression; SOX10 human tf ARCHS4 coexpression; TCF3 human tf ARCHS4 coexpression; ZFY human tf ARCHS4 coexpression; MLX human tf ARCHS4 coexpression; POU1F1 human tf ARCHS4 coexpression; ZNF200 human tf ARCHS4 coexpression; ETV7 human tf ARCHS4 coexpression; ETV1 human tf ARCHS4 coexpression; DLX6 human tf ARCHS4 coexpression; SOX8 human tf ARCHS4 coexpression; ARX human tf ARCHS4 coexpression; THAP10 human tf ARCHS4 coexpression; MEF2B human tf ARCHS4 coexpression; BCL11A human tf ARCHS4 coexpression; SMAD5 human tf ARCHS4 coexpression; MYB human tf ARCHS4 coexpression; E2F5 human tf ARCHS4 coexpression; PPARG human tf ARCHS4 coexpression; RFX1 human tf ARCHS4 coexpression; ZNF236 human tf ARCHS4 coexpression; KLF16 human tf ARCHS4 coexpression; SOX15 human tf ARCHS4 coexpression; HOXD3 human tf ARCHS4 coexpression; VAX2 human tf ARCHS4 coexpression; FOXP2 human tf ARCHS4 coexpression; ZNF384 human tf ARCHS4 coexpression; NR1D1 human tf ARCHS4 coexpression; TCF15 human tf ARCHS4 coexpression; FOXA2 human tf ARCHS4 coexpression; MXD4 human tf ARCHS4 coexpression; HOXC12 human tf ARCHS4 coexpression; HOXB3 human tf ARCHS4 coexpression; ATF4 human tf ARCHS4 coexpression; ZNF706 human tf ARCHS4 coexpression; SIX6 human tf ARCHS4 coexpression; ESX1 human tf ARCHS4 coexpression; IKZF4 human tf ARCHS4 coexpression; ZNF347 human tf ARCHS4 coexpression; ZNF311 human tf ARCHS4 coexpression; ZNF585B human tf ARCHS4 coexpression; ZNF81 human tf ARCHS4 coexpression; ZNF460 human tf ARCHS4 coexpression; ZNF470 human tf ARCHS4 coexpression; ZNF544 human tf ARCHS4 coexpression; ZNF136 human tf ARCHS4 coexpression; ZNF565 human tf ARCHS4 coexpression; ZBTB44 human tf ARCHS4 coexpression; ZNF33A human tf ARCHS4 coexpression; ZNF567 human tf ARCHS4 coexpression; ZNF548 human tf ARCHS4 coexpression; HMX3 human tf ARCHS4 coexpression; ZNF490 human tf ARCHS4 coexpression; ZNF777 human tf ARCHS4 coexpression; TAL2 human tf ARCHS4 coexpression; ZNF334 human tf ARCHS4 coexpression; ZNF583 human tf ARCHS4 coexpression; NFRKB human tf ARCHS4 coexpression; RFXANK human tf ARCHS4 coexpression; NKRF human tf ARCHS4 coexpression; PAWR human tf ARCHS4 coexpression; PREB human tf ARCHS4 coexpression; ZNF407 human tf ARCHS4 coexpression; ZNF718 human tf ARCHS4 coexpression; ZNF442 human tf ARCHS4 coexpression; ZBTB9 human tf ARCHS4 coexpression; ONECUT3 human tf ARCHS4 coexpression; ZFP57 human tf ARCHS4 coexpression; ZNF277 human tf ARCHS4 coexpression; ZNF830 human tf ARCHS4 coexpression; ZNF512 human tf ARCHS4 coexpression; ZNF480 human tf ARCHS4 coexpression; ZXDB human tf ARCHS4 coexpression; ZNF625 human tf ARCHS4 coexpression; ZFP1 human tf ARCHS4 coexpression; ZNF71 human tf ARCHS4 coexpression; ZFP41 human tf ARCHS4 coexpression; ZSCAN1 human tf ARCHS4 coexpression; ZNF256 human tf ARCHS4 coexpression; ZNF385D human tf ARCHS4 coexpression; VENTX human tf ARCHS4 coexpression; HMBOX1 human tf ARCHS4 coexpression; ZNF92 human tf ARCHS4 coexpression; ZNF660 human tf ARCHS4 coexpression; ZNF514 human tf ARCHS4 coexpression; ZFP14 human tf ARCHS4 coexpression; ZNF365 human tf ARCHS4 coexpression; ZFP37 human tf ARCHS4 coexpression; ZFHX2 human tf ARCHS4 coexpression; ZSCAN5A human tf ARCHS4 coexpression; ZNF141 human tf ARCHS4 coexpression; TSHZ2 human tf ARCHS4 coexpression; MIER3 human tf ARCHS4 coexpression; ZNF333 human tf ARCHS4 coexpression; ZNF689 human tf ARCHS4 coexpression; DRGX human tf ARCHS4 coexpression; ZNF571 human tf ARCHS4 coexpression; ZNF771 human tf ARCHS4 coexpression; ZBTB41 human tf ARCHS4 coexpression; ZNF518A human tf ARCHS4 coexpression; ZNF620 human tf ARCHS4 coexpression; ZNF404 human tf ARCHS4 coexpression; MGA human tf ARCHS4 coexpression; ZNF577 human tf ARCHS4 coexpression; ZNF596 human tf ARCHS4 coexpression; RAX2 human tf ARCHS4 coexpression; CAMTA1 human tf ARCHS4 coexpression; ZIK1 human tf ARCHS4 coexpression; ZBTB5 human tf ARCHS4 coexpression; GBX2 human tf ARCHS4 coexpression; ZNF701 human tf ARCHS4 coexpression; ZNF606 human tf ARCHS4 coexpression; ZMAT3 human tf ARCHS4 coexpression; ZNF426 human tf ARCHS4 coexpression; TBX3 20139965 ChIP-Seq ESCs Mouse; TP53 22127205 ChIP-Seq IMR90 Human; HDAC6 K562 hg19; CTCF kidney mm9; REST myocyte mm9; USF2 H1-hESC hg19; RFX5 HepG2 hg19; JUND 26020271 ChIP-Seq SMOOTH MUSCLE Human; TAL1 22897851 ChIP-Seq JUKARTE6-1 Human; CDX2 22108803 ChIP-Seq LS180 Human; CTCF GM12891 hg19; CHD2 CH12.LX mm9; CTCF myotube hg19; POLR2AphosphoS2 A549 hg19; RELA GM18505 hg19; CTCF GM12892 hg19; NANOG 20526341 ChIP-Seq ESCs Human; EBNA1 20929547 Chip-Seq RAJ1-cells Human; CTCF 20526341 ChIP-Seq ESCs Human; KDM2B 26808549 Chip-Seq REH Human; TAF2 19829295 ChIP-Seq ESCs Human; OCT4 19829295 ChIP-Seq ESCs Human; SUZ12 18555785 Chip-Seq ESCs Mouse; NMYC 18555785 Chip-Seq ESCs Mouse; CTCF 18555785 Chip-Seq ESCs Mouse; CEBPA 26348894 ChIP-Seq LIVER Mouse; GATA6 21074721 ChIP-Seq CACO-2 Mouse; GATA6 21074721 ChIP-Seq CACO-2 Human; P53 22387025 ChIP-Seq ESCs Mouse; GATA3 21867929 ChIP-Seq CD8 Mouse; GABPA MCF-7 hg19; VDR 23849224 ChIP-Seq CD4+ Human; TRIM28 19339689 ChIP-ChIP MESCs Mouse; CTCF HepG2 hg19; TAF7 H1-hESC hg19; CHD1 19587682 ChIP-ChIP MESCs Mouse; TAF7 K562 hg19; SPI1 20176806 ChIP-Seq THIOMACROPHAGE Mouse; MEF2A 21415370 ChIP-Seq HL-1 Mouse; TAF1 SK—N—SH hg19; SOX2 18692474 ChIP-Seq MESCs Mouse; NANOG 18555785 ChIP-Seq MESCs Mouse; PRDM14 21183938 ChIP-Seq MESCs Mouse; PPARG 20176806 ChIP-Seq 3T3-L1 Mouse; CEBPB 26923725 Chip-Seq HEMANGIOBLAST Mouse; GABPA A549 hg19; SPI1 22096565 ChIP-ChIP GC-B Mouse; FOS HeLa—S3 hg19; TCF4 23295773 ChIP-Seq U87 Human; CTCF osteoblast hg19; MYC HepG2 hg19; CTCF testis mm9; JARID1B-DAIN 22020125 ChIP-Seq ESCs Mouse; PU.1 20513432 ChIP-Seq Bcells Mouse; P300 18555785 Chip-Seq ESCs Mouse; KLF4 18555785 Chip-Seq ESCs Mouse; E2F1 18555785 Chip-Seq ESCs Mouse; SOX2 18555785 Chip-Seq ESCs Mouse; RXRA 24833708 ChIP-Seq LIVER Mouse; WHSC1 K562 hg19; SAP30 K562 hg19; CHD2 GM12878 hg19; STAT3 GM12878 hg19; SIN3A MEL cell line mm9; CTCF T-cell acute lymphoblastic leukemia hg19; RCOR1 GM12878 hg19; MYC GM12878 hg19; NFIC HepG2 hg19; CREB1 K562 hg19; SALL4 18804426 ChIP-ChIP MESCs Mouse; SIX5 H1-hESC hg19; ETS1 A549 hg19; RXRA H1-hESC hg19; ATF2 GM12878 hg19; E2F1 18555785 ChIP-Seq MESCs Mouse; SIN3B 21632747 ChIP-Seq MESCs Mouse; RUNX1 21571218 ChIP-Seq MEGAKARYOCYTES Human; ZNF274 21170338 ChIP-Seq K562 Hela; CREB1 ECC-1 hg19; IRF8 22096565 ChIP-ChIP GC-B Mouse; TBX5 21415370 ChIP-Seq HL-1 Mouse; CEBPB ECC-1 hg19; CTCF brain microvascular endothelial cell hg19; CEBPB 26923725 Chip-Seq MESODERM Mouse; PCGF2 27294783 Chip-Seq NPCs Mouse; STAT6 20620947 ChIP-Seq CD4 POS T Human; DMRT1 23473982 ChIP-Seq TESTES Mouse; EZH2 27294783 Chip-Seq NPCs Mouse; HTT 18923047 ChIP-ChIP STHdh Human; SRF GM12878 hg19; SOX2 21211035 ChIP-Seq LN229 Gbm; YY1 GM12891 hg19; CTCF CH12.LX mm9; POLR2AphosphoS5 HepG2 hg19; EGR1 20690147 ChIP-Seq ERYTHROLEUKEMIA Human; KDM5A H1-hESC hg19; LUZP1 20508642 ChIP-Seq ESCs Mouse; CRX 20693478 ChIP-Seq RETINA Mouse; ETV1 20927104 ChIP-Seq GIST48 Human; OCT4 21477851 ChIP-Seq ESCs Mouse; SOX6 21985497 ChIP-Seq MYOTUBES Mouse; EBF1 22473956 ChIP-Seq LYMPHODE Mouse; SPIL 26923725 Chip-Seq MACROPHAGESS Mouse; SOX2 22085726 ChIP-Seq NPCs Mouse; ATF1 K562 hg19; CREBBP K562 hg19; CTCF B cell hg19; CTCF GM19240 hg19; POLR2A GM15510 hg19; MAZ HeLa—S3 hg19; STATI HeLa—S3 hg19; PRDM14 20953172 ChIP-Seq ESCs Human; E2F4 21247883 ChIP-Seq LYMPHOBLASTOID Human; YY1 22570637 ChIP-Seq MALME-3M Human; EST1 17652178 ChIP-ChIP JURKAT Human; GFIIB 20887958 ChIP-Seq HPC-7 Mouse; TCF3 18692474 ChIP-Seq MESCs Mouse; FOSL2 SK—N—SH hg19; JUND HCT116 hg19; HSF1 23293686 ChIP-Seq STHDH STRIATAL Mouse; SIN3A HepG2 hg19; SETDB1 19884255 ChIP-Seq MESCs Mouse; KLF4 25985364 ChIP-Seq ATHEROSCLEROSIS LESION Mouse; ETS2 20176728 ChIP-ChIP TROPHOBLAST STEM CELLS Mouse; POU5F1 18358816 ChIP-ChIP MESCs Mouse; FOXP2 21765815 ChIP-ChIP NEURO2A Mouse; NKX2-5 21415370 ChIP-Seq HL-1 Mouse; TAF7L 23326641 ChIP-Seq C3H10T1-2 Mouse; FLI1 21571218 ChIP-Seq MEGAKARYOCYTES Human; POLR2AphosphoS5 GM12892 hg19; ELK1 19687146 ChIP-ChIP HELA Human; TEAD4 H1-hESC hg19; GABPA HL-60 hg19; GABPA HeLa—S3 hg19; TAL1 megakaryocyte mm9; FOSL1 HCT116 hg19; IRF8 27001747 Chip-Seq BMDM Mouse; CBX2 22325352 ChIP-Seq 293T-Rex Human; CEBPB 22108803 ChIP-Seq LS180 Human; MXII MEL cell line mm9; HCFC1 ES-E14 mm9; CHD2 HeLa—S3 hg19; CTCF GM10248 hg19; SETDBI U20S hg19; CTCF spleen mm9; CDX2 21402776 ChIP-Seq INTESTINAL-VILLUS Mouse; CMYC 18555785 Chip-Seq ESCs Mouse; STAT3 18555785 Chip-Seq ESCs Mouse; ZFX 18555785 Chip-Seq ESCs Mouse; SMAD1 18555785 Chip-Seq ESCs Mouse; HOXB7 26014856 ChIP-Seq BT474 Human; ESRRB 18555785 Chip-Seq ESCs Mouse; SUPT20H GM12878 hg19; GATA1 26923725 Chip-Seq HPCs Mouse; BCL11A GM12878 hg19; ZNF140 human tf ARCHS4 coexpression; ZNF605 human tf ARCHS4 coexpression; ZNF431 human tf ARCHS4 coexpression; ZNF700 human tf ARCHS4 coexpression; ZNF705A human tf ARCHS4 coexpression; ZNF138 human tf ARCHS4 coexpression; ZNF420 human tf ARCHS4 coexpression; ZNF628 human tf ARCHS4 coexpression; ZNF823 human tf ARCHS4 coexpression; ZNF667 human tf ARCHS4 coexpression; ZNF69 human tf ARCHS4 coexpression; ZNF280B human tf ARCHS4 coexpression; ZBTB12 human tf ARCHS4 coexpression; HOMEZ human tf ARCHS4 coexpression; ZNF579 human tf ARCHS4 coexpression; BCLAF1 human tf ARCHS4 coexpression; TUB human tf ARCHS4 coexpression; TCF19 human tf ARCHS4 coexpression; THAP3 human tf ARCHS4 coexpression; ZNF346 human tf ARCHS4 coexpression; TOX2 human tf ARCHS4 coexpression; E2F8 human tf ARCHS4 coexpression; TERF2 human tf ARCHS4 coexpression; ZNF471 human tf ARCHS4 coexpression; ZMAT2 human tf ARCHS4 coexpression; ZNF107 human tf ARCHS4 coexpression; ZNF669 human tf ARCHS4 coexpression; ZNF449 human tf ARCHS4 coexpression; ZNF417 human tf ARCHS4 coexpression; EVX2 human tf ARCHS4 coexpression; ZNF408 human tf ARCHS4 coexpression; DRAP1 human tf ARCHS4 coexpression; ZNF611 human tf ARCHS4 coexpression; ZNF283 human tf ARCHS4 coexpression; ZNF491 human tf ARCHS4 coexpression; ZNF354B human tf ARCHS4 coexpression; ZNF223 human tf ARCHS4 coexpression; ZNF552 human tf ARCHS4 coexpression; LBX2 human tf ARCHS4 coexpression; FIZ1 human tf ARCHS4 coexpression; RFX7 human tf ARCHS4 coexpression; ZSCAN22 human tf ARCHS4 coexpression; ZNF530 human tf ARCHS4 coexpression; FOXD4L1 human tf ARCHS4 coexpression; ZNF445 human tf ARCHS4 coexpression; ZBTB37 human tf ARCHS4 coexpression; ZNF555 human tf ARCHS4 coexpression; ZNF501 human tf ARCHS4 coexpression; FOXE3 human tf ARCHS4 coexpression; ZNF383 human tf ARCHS4 coexpression; ZNF681 human tf ARCHS4 coexpression; GATAD1 human tf ARCHS4 coexpression; YEATS2 human tf ARCHS4 coexpression; FOXK1 human tf ARCHS4 coexpression; COPS4 human tf ARCHS4 coexpression; RNF113B human tf ARCHS4 coexpression; SLC4A10 human tf ARCHS4 coexpression; POLE3 human tf ARCHS4 coexpression; CHD1 human tf ARCHS4 coexpression; GPR155 human tf ARCHS4 coexpression; MSTIR human tf ARCHS4 coexpression; HMGB2 human tf ARCHS4 coexpression; TERF2IP human tf ARCHS4 coexpression; ZNF738 human tf ARCHS4 coexpression; ANAPC2 human tf ARCHS4 coexpression; RBM10 human tf ARCHS4 coexpression; RGS7 human tf ARCHS4 coexpression; OTOP3 human tf ARCHS4 coexpression; MTA1 human tf ARCHS4 coexpression; SF3A3 human tf ARCHS4 coexpression; HIST1HIB human tf ARCHS4 coexpression; KNTC1 human tf ARCHS4 coexpression; ZFP90 human tf ARCHS4 coexpression; HIST1HIC human tf ARCHS4 coexpression; HILS1 human tf ARCHS4 coexpression; H1F0 human tf ARCHS4 coexpression; GTF2E2 human tf ARCHS4 coexpression; XPA human tf ARCHS4 coexpression; RNF138 human tf ARCHS4 coexpression; HP1BP3 human tf ARCHS4 coexpression; CENPB human tf ARCHS4 coexpression; ZNF367 human tf ARCHS4 coexpression; E2F7 human tf ARCHS4 coexpression; ZNF598 human tf ARCHS4 coexpression; ZNF778 human tf ARCHS4 coexpression; THAP6 human tf ARCHS4 coexpression; ZNF717 human tf ARCHS4 coexpression; ZNF829 human tf ARCHS4 coexpression; ZNF788 human tf ARCHS4 coexpression; ZNF766 human tf ARCHS4 coexpression; ZNF799 human tf ARCHS4 coexpression; ZNF846 human tf ARCHS4 coexpression; ZNF680 human tf ARCHS4 coexpression; ZNF720 human tf ARCHS4 coexpression; HMG20B human tf ARCHS4 coexpression; ZNF638 human tf ARCHS4 coexpression; RGS11 human tf ARCHS4 coexpression; UBE2K human tf ARCHS4 coexpression; RBM27 human tf ARCHS4 coexpression; MET human tf ARCHS4 coexpression; EXOC2 human tf ARCHS4 coexpression; RPA2 human tf ARCHS4 coexpression; HIST1HID human tf ARCHS4 coexpression; DEK human tf ARCHS4 coexpression; TTF1 human tf ARCHS4 coexpression; ZNF780A human tf ARCHS4 coexpression; ZNF621 human tf ARCHS4 coexpression; ZNF581 human tf ARCHS4 coexpression; TLX1 human tf ARCHS4 coexpression; HOXB6 human tf ARCHS4 coexpression; DLX4 human tf ARCHS4 coexpression; PPARD human tf ARCHS4 coexpression; TFDP2 human tf ARCHS4 coexpression; ID2 human tf ARCHS4 coexpression; DLX2 human tf ARCHS4 coexpression; ATF2 human tf ARCHS4 coexpression; ASH1L human tf ARCHS4 coexpression; NR5A2 human tf ARCHS4 coexpression; SMAD9 human tf ARCHS4 coexpression; NFYB human tf ARCHS4 coexpression; ZNF205 human tf ARCHS4 coexpression; BARHL1 human tf ARCHS4 coexpression; PLAGL2 human tf ARCHS4 coexpression; PRDM7 human tf ARCHS4 coexpression; HOXD1 human tf ARCHS4 coexpression; HOXD13 human tf ARCHS4 coexpression; CDX4 human tf ARCHS4 coexpression; BARX1 human tf ARCHS4 coexpression; DMTF1 human tf ARCHS4 coexpression; SKIL human tf ARCHS4 coexpression; GATA6 human tf ARCHS4 coexpression; LHX3 human tf ARCHS4 coexpression; DLX1 human tf ARCHS4 coexpression; EZH2 human tf ARCHS4 coexpression; MAZ human tf ARCHS4 coexpression; HSF2 human tf ARCHS4 coexpression; NFE2L3 human tf ARCHS4 coexpression; HES2 human tf ARCHS4 coexpression; SREBF1 human tf ARCHS4 coexpression; RARB human tf ARCHS4 coexpression; CIC human tf ARCHS4 coexpression; REST human tf ARCHS4 coexpression; NFX1 human tf ARCHS4 coexpression; HIVEP1 human tf ARCHS4 coexpression; YY1 human tf ARCHS4 coexpression; GATA1 human tf ARCHS4 coexpression; KLF8 human tf ARCHS4 coexpression; KLF5 human tf ARCHS4 coexpression; HSF4 human tf ARCHS4 coexpression; CRX human tf ARCHS4 coexpression; ZNF7 human tf ARCHS4 coexpression; ALX3 human tf ARCHS4 coexpression; ISL2 human tf ARCHS4 coexpression; PRDM13 human tf ARCHS4 coexpression; ZNF142 human tf ARCHS4 coexpression; RLF human tf ARCHS4 coexpression; ZNF549 human tf ARCHS4 coexpression; ZNF639 human tf ARCHS4 coexpression; ZSCAN20 human tf ARCHS4 coexpression; ZNF341 human tf ARCHS4 coexpression; ZNF227 human tf ARCHS4 coexpression; ZNF20 human tf ARCHS4 coexpression; ZNF670 human tf ARCHS4 coexpression; ZNF280D human tf ARCHS4 coexpression; ZIM3 human tf ARCHS4 coexpression; ZNF300 human tf ARCHS4 coexpression; ZSCAN12 human tf ARCHS4 coexpression; ZNF233 human tf ARCHS4 coexpression; ZNF382 human tf ARCHS4 coexpression; ZNF326 human tf ARCHS4 coexpression; PKNOX2 human tf ARCHS4 coexpression; ZNF83 human tf ARCHS4 coexpression; ZNF296 human tf ARCHS4 coexpression; ZNF415 human tf ARCHS4 coexpression; ZNF439 human tf ARCHS4 coexpression; ZNF440 human tf ARCHS4 coexpression; ZNF85 human tf ARCHS4 coexpression; ZNF516 human tf ARCHS4 coexpression; ZNF8 human tf ARCHS4 coexpression; ZNF221 human tf ARCHS4 coexpression; ZNF230 human tf ARCHS4 coexpression; HESX1 human tf ARCHS4 coexpression; ZNF22 human tf ARCHS4 coexpression; E4F1 human tf ARCHS4 coexpression; E2F6 human tf ARCHS4 coexpression; ZFPM2 human tf ARCHS4 coexpression; KLF11 human tf ARCHS4 coexpression; ARNT2 human tf ARCHS4 coexpression; SMAD2 human tf ARCHS4 coexpression; ZNF25 human tf ARCHS4 coexpression; HSF5 human tf ARCHS4 coexpression; ZNF562 human tf ARCHS4 coexpression; ZNF260 human tf ARCHS4 coexpression; POU6F1 human tf ARCHS4 coexpression; ZNF197 human tf ARCHS4 coexpression; ZNF181 human tf ARCHS4 coexpression; ZNF44 human tf ARCHS4 coexpression; TFDP1 human tf ARCHS4 coexpression; MAFK human tf ARCHS4 coexpression; RORB human tf ARCHS4 coexpression; ZNF551 human tf ARCHS4 coexpression; ZNF254 human tf ARCHS4 coexpression; ZNF195 human tf ARCHS4 coexpression; ZNF263 human tf ARCHS4 coexpression; HES7 human tf ARCHS4 coexpression; EP300 GM12878 hg19; SUZ12 27294783 Chip-Seq NPCs Mouse; TRIM28 K562 hg19; NFIC GM12878 hg19; MYBL2 22936984 ChIP-ChIP MESCs Mouse; GABPA K562 hg19; EGR1 23403033 ChIP-Seq LIVER Mouse; PPARG 23326641 ChIP-Seq C3H10T1-2 Mouse; SETDB1 19884257 ChIP-Seq MESCs Mouse; USF1 HCT116 hg19; MYCN 18555785 ChIP-Seq MESCs Mouse; POLR2AphosphoS5 SK—N-MC hg19; CEBPB 23403033 ChIP-Seq LIVER Mouse; AR 20517297 ChIP-Seq VCAP Human; SIRT6 H1-hESC hg19; EBF1 22473956 ChIP-Seq BONE MARROW Mouse; CEBPB 20513432 ChIP-Seq MACROPHAGES Mouse; ESET 19884257 ChIP-Seq ESCs Mouse; RARA 24833708 ChIP-Seq LIVER Mouse; KLF4 19829295 ChIP-Seq ESCs Human; MYC 18940864 ChIP-ChIP HL60 Human; MYC 19030024 ChIP-ChIP MESCs Mouse; ASH2L 23239880 ChIP-Seq MESCs Mouse; ELF1 20517297 ChIP-Seq JURKAT Human; THAP11 20581084 ChIP-Seq MESCs Mouse; FLI1 27457419 Chip-Seq LIVER Mouse; PBX1 22567123 ChIP-ChIP OVCAR3 Human; NFE2 27457419 Chip-Seq LIVER Mouse; ELF1 HepG2 hg19; FOSL2 MCF-7 hg19; ATF3 GM12878 hg19; E2F1 21310950 ChIP-Seq MCF-7 Human; SREBP2 21459322 ChIP-Seq LIVER Mouse; POU3F2 20337985 ChIP-ChIP 501MEL Human; ZBTB33 K562 hg19; CTCF K562 hg19; HCFC1 CH12.LX mm9; SMAD3 21741376 ChIP-Seq ESCs Human; CTCF GM19238 hg19; GATA3 SH-SY5Y hg19; CTCF GM13977 hg19; POLR2AphosphoS2 MEL cell line mm9; STAT1 17558387 ChIP-Seq HELA Human; MAX HeLa—S3 hg19; TBP 23326641 ChIP-Seq C3H10T1-2 Mouse; REST 18959480 ChIP-ChIP MESCs Mouse; ZFP42 18358816 ChIP-ChIP MESCs Mouse; NANOG 18692474 ChIP-Seq MESCs Mouse; GABP 19822575 ChIP-Seq HepG2 Human; ESRRB 18555785 ChIP-Seq MESCs Mouse; ATF3 H1-hESC hg19; NANOG 18358816 ChIP-ChIP MESCs Mouse; AR 19668381 ChIP-Seq PC3 Human; TEAD4 26923725 Chip-Seq HEMANGIOBLAST Mouse; POUSF1 26923725 Chip-Seq MESODERM Mouse; ELF1 A549 hg19; TP53 22573176 ChIP-Seq HFKS Human; FOXA1 21572438 ChIP-Seq LNCaP Human; FOXO3 22982991 ChIP-Seq MACROPHAGES Mouse; CTCF skeletal muscle myoblast hg19; POLR2A GM18505 hg19; RAC3 21632823 ChIP-Seq H3396 Human; FOXH1 21741376 ChIP-Seq EPCs Human; RUNX1 27457419 Chip-Seq LIVER Mouse; ELF1 MCF-7 hg19; SIN3A K562 hg19; PCGF2 27294783 Chip-Seq ESCs Mouse; ATF3 HepG2 hg19; PAX6 23342162 ChIP-ChIP BETA-FORBRAIN-LENS Mouse; ZNF626 human tf ARCHS4 coexpression; ZNF250 human tf ARCHS4 coexpression; ZNF493 human tf ARCHS4 coexpression; ZNF124 human tf ARCHS4 coexpression; ZNF286A human tf ARCHS4 coexpression; ZNF600 human tf ARCHS4 coexpression; NFYA human tf ARCHS4 coexpression; FOXP4 human tf ARCHS4 coexpression; EMX1 human tf ARCHS4 coexpression; RHOXF2 human tf ARCHS4 coexpression; ZNF557 human tf ARCHS4 coexpression; MLLT1 human tf ARCHS4 coexpression; ZNF576 human tf ARCHS4 coexpression; ZSCAN18 human tf ARCHS4 coexpression; PRDM15 human tf ARCHS4 coexpression; CAMTA2 human tf ARCHS4 coexpression; RHOXF1 human tf ARCHS4 coexpression; ZNF264 human tf ARCHS4 coexpression; ZNF93 human tf ARCHS4 coexpression; ZBTB11 human tf ARCHS4 coexpression; WIZ human tf ARCHS4 coexpression; E2F4 human tf ARCHS4 coexpression; LCOR human tf ARCHS4 coexpression; EVX1 human tf ARCHS4 coexpression; NR2C2 human tf ARCHS4 coexpression; ZNF711 human tf ARCHS4 coexpression; ZNF225 human tf ARCHS4 coexpression; ZNF721 human tf ARCHS4 coexpression; ZNF678 human tf ARCHS4 coexpression; ZNF664 human tf ARCHS4 coexpression; ZNF543 human tf ARCHS4 coexpression; ZNF613 human tf ARCHS4 coexpression; ZNF519 human tf ARCHS4 coexpression; ZIC4 human tf ARCHS4 coexpression; CIZ1 human tf ARCHS4 coexpression; ZNF561 human tf ARCHS4 coexpression; ZBTB43 human tf ARCHS4 coexpression; ZNF597 human tf ARCHS4 coexpression; ZNF668 human tf ARCHS4 coexpression; HDX human tf ARCHS4 coexpression; PRDM9 human tf ARCHS4 coexpression; ZNF691 human tf ARCHS4 coexpression; ZNF486 human tf ARCHS4 coexpression; ZNF692 human tf ARCHS4 coexpression; ZHX3 human tf ARCHS4 coexpression; HOXC5 human tf ARCHS4 coexpression; ZNF16 human tf ARCHS4 coexpression; ESRRB human tf ARCHS4 coexpression; PROX2 human tf ARCHS4 coexpression; TFAP2E human tf ARCHS4 coexpression; CEBPZ human tf ARCHS4 coexpression; SRF human tf ARCHS4 coexpression; E2F3 human tf ARCHS4 coexpression; FOXM1 human tf ARCHS4 coexpression; HOXB7 human tf ARCHS4 coexpression; WHSC1 human tf ARCHS4 coexpression; ETV2 human tf ARCHS4 coexpression; ZNF174 human tf ARCHS4 coexpression; E2F1 human tf ARCHS4 coexpression; MYBL2 human tf ARCHS4 coexpression; CEBPE human tf ARCHS4 coexpression; ZNF37A human tf ARCHS4 coexpression; SOX30 human tf ARCHS4 coexpression; POU6F2 human tf ARCHS4 coexpression; LHX4 human tf ARCHS4 coexpression; NEUROG3 human tf ARCHS4 coexpression; ATF1 human tf ARCHS4 coexpression; ZNF282 human tf ARCHS4 coexpression; ZNF444 human tf ARCHS4 coexpression; ZNF146 human tf ARCHS4 coexpression; TEF human tf ARCHS4 coexpression; ZNF143 human tf ARCHS4 coexpression; SNAPC4 human tf ARCHS4 coexpression; ZHX1 human tf ARCHS4 coexpression; ZNF12 human tf ARCHS4 coexpression; T human tf ARCHS4 coexpression; REL human tf ARCHS4 coexpression; AIRE human tf ARCHS4 coexpression; SK1 human tf ARCHS4 coexpression; CEBPG human tf ARCHS4 coexpression; CREBZF human tf ARCHS4 coexpression; TFCP2 human tf ARCHS4 coexpression; GCM2 human tf ARCHS4 coexpression; CTCFL human tf ARCHS4 coexpression; ZFP62 human tf ARCHS4 coexpression; ZNF418 human tf ARCHS4 coexpression; ZNF529 human tf ARCHS4 coexpression; ZBTB3 human tf ARCHS4 coexpression; ZNF677 human tf ARCHS4 coexpression; ZNF257 human tf ARCHS4 coexpression; ZNF441 human tf ARCHS4 coexpression; ZNF585A human tf ARCHS4 coexpression; ZNF607 human tf ARCHS4 coexpression; ZSCAN16 human tf ARCHS4 coexpression; ZNF587 human tf ARCHS4 coexpression; ZNF675 human tf ARCHS4 coexpression; ZNF358 human tf ARCHS4 coexpression; ZNF578 human tf ARCHS4 coexpression; ZNF805 human tf ARCHS4 coexpression; INF2 human tf ARCHS4 coexpression; FOXJ3 human tf ARCHS4 coexpression; PLXNB3 human tf ARCHS4 coexpression; SF3A2 human tf ARCHS4 coexpression; ZC3H14 human tf ARCHS4 coexpression; NUFIP1 human tf ARCHS4 coexpression; DUSP12 human tf ARCHS4 coexpression; SRCAP human tf ARCHS4 coexpression; CUL1 human tf ARCHS4 coexpression; DEPDC1B human tf ARCHS4 coexpression; ZNF789 human tf ARCHS4 coexpression; ZNF506 human tf ARCHS4 coexpression; ZNF385C human tf ARCHS4 coexpression; ZNF487 human tf ARCHS4 coexpression; THAP4 human tf ARCHS4 coexpression; THAP2 human tf ARCHS4 coexpression; THAP9 human tf ARCHS4 coexpression; NKX6-3 human tf ARCHS4 coexpression; ZNF780B human tf ARCHS4 coexpression; AFF4 human tf ARCHS4 coexpression; PHB2 human tf ARCHS4 coexpression; PCGF6 human tf ARCHS4 coexpression; MIER2 human tf ARCHS4 coexpression; PSMD11 human tf ARCHS4 coexpression; FBXO41 human tf ARCHS4 coexpression; ATMIN human tf ARCHS4 coexpression; GATAD2A human tf ARCHS4 coexpression; ZNF528 human tf ARCHS4 coexpression; MTA2 human tf ARCHS4 coexpression; SSRP1 human tf ARCHS4 coexpression; TUT1 human tf ARCHS4 coexpression; COPS3 human tf ARCHS4 coexpression; LARP1 human tf ARCHS4 coexpression; HOPX human tf ARCHS4 coexpression; TIGD7 human tf ARCHS4 coexpression; ADAMTS17 human tf ARCHS4 coexpression; YEATS4 human tf ARCHS4 coexpression; CCDC71 human tf ARCHS4 coexpression; POLR2L human tf ARCHS4 coexpression; EIF3K human tf ARCHS4 coexpression; PRMT3 human tf ARCHS4 coexpression; CSDC2 human tf ARCHS4 coexpression; DVL3 human tf ARCHS4 coexpression; YAP1 20516196 ChIP-Seq MESCs Mouse; TCF3/E2A 22897851 ChIP-Seq JUKARTE6-1 Human; MYC 18358816 ChIP-ChIP MESCs Mouse; POLR2A GM18951 hg19; HCFC1 HeLa—S3 hg19; HCFC1 MEL cell line mm9; NR3C1 HepG2 hg19; PRDM5 23873026 ChIP-Seq MEFs Mouse; ZZZ3 HeLa—S3 hg19; RUNX3 GM12878 hg19; GABPA HepG2 hg19; KDM6A 18722178 ChIP-ChIP U937 AND SAOS2 Human; NELFA 20434984 ChIP-Seq ESCs Mouse; ELKI GM12878 hg19; KLF4 18555785 ChIP-Seq MESCs Mouse; BCLAF1 GM12878 hg19; GABP 17652178 ChIP-ChIP JURKAT Human; REST 21632747 ChIP-Seq MESCs Mouse; GABPA SK—N—SH hg19; POU5F1 18692474 ChIP-Seq MESCs Mouse; FLI1 megakaryocyte mm9; ETS1 20019798 ChIP-Seq JURKAT Human; NELFE MEL cell line mm9; KDM5B 21448134 ChIP-Seq MESCs Mouse; ELF1 SK—N—SH hg19; MYC 19915707 ChIP-ChIP AK7 Human; HOXB4 20404135 ChIP-ChIP EML Mouse; POLR2AphosphoS5 HL-60 hg19; SOX2 19829295 ChIP-Seq ESCs Human; NANOG 19829295 ChIP-Seq ESCs Human; GATA3 21867929 ChIP-Seq TH1 Mouse; POLR2AphosphoS5 G1E mm9; ZNF714 human tf ARCHS4 coexpression; GTF2F1 human tf ARCHS4 coexpression; ZC3H6 human tf ARCHS4 coexpression; FOXK2 human tf ARCHS4 coexpression; CLOCK human tf ARCHS4 coexpression; ELK1 human tf ARCHS4 coexpression; DACH2 human tf ARCHS4 coexpression; ZNF451 human tf ARCHS4 coexpression; TFAM human tf ARCHS4 coexpression; CREB3 human tf ARCHS4 coexpression; DLX5 human tf ARCHS4 coexpression; ERF human tf ARCHS4 coexpression; RCOR1 human tf ARCHS4 coexpression; CBFB human tf ARCHS4 coexpression; YBX1 human tf ARCHS4 coexpression; NFIX human tf ARCHS4 coexpression; ZNF232 human tf ARCHS4 coexpression; ZNF584 human tf ARCHS4 coexpression; ZIC1 human tf ARCHS4 coexpression; UBP1 human tf ARCHS4 coexpression; ZNF35 human tf ARCHS4 coexpression; SCRT1 human tf ARCHS4 coexpression; ZNF547 human tf ARCHS4 coexpression; ZNF45 human tf ARCHS4 coexpression; ZNF211 human tf ARCHS4 coexpression; ZNF629 human tf ARCHS4 coexpression; ZNF510 human tf ARCHS4 coexpression; ZFAT human tf ARCHS4 coexpression; ZNF275 human tf ARCHS4 coexpression; ZNF80 human tf ARCHS4 coexpression; DPF1 human tf ARCHS4 coexpression; SREBF2 human tf ARCHS4 coexpression; ZNF396 human tf ARCHS4 coexpression; ALX1 human tf ARCHS4 coexpression; ZBTB33 human tf ARCHS4 coexpression; SP3 human tf ARCHS4 coexpression; ZNF24 human tf ARCHS4 coexpression; HOXB9 human tf ARCHS4 coexpression; ZNF207 human tf ARCHS4 coexpression; ZNF654 human tf ARCHS4 coexpression; ZNF77 human tf ARCHS4 coexpression; ZNF683 human tf ARCHS4 coexpression; RIOK2 human tf ARCHS4 coexpression; ZC3H7B human tf ARCHS4 coexpression; TRIM3 human tf ARCHS4 coexpression; FARSB human tf ARCHS4 coexpression; ZNF222 human tf ARCHS4 coexpression; PRR12 human tf ARCHS4 coexpression; UBR4 human tf ARCHS4 coexpression; ZFR human tf ARCHS4 coexpression; SSB human tf ARCHS4 coexpression; BDP1 human tf ARCHS4 coexpression; SHPRH human tf ARCHS4 coexpression; MRRF human tf ARCHS4 coexpression; LARP4 human tf ARCHS4 coexpression; MGMT human tf ARCHS4 coexpression; SUZ12 human tf ARCHS4 coexpression; EP400 human tf ARCHS4 coexpression; SETDB1 human tf ARCHS4 coexpression; HMGB1 human tf ARCHS4 coexpression; BRD9 human tf ARCHS4 coexpression; ZNF845 human tf ARCHS4 coexpression; KCMF1 human tf ARCHS4 coexpression; ZNF609 human tf ARCHS4 coexpression; ZFP82 human tf ARCHS4 coexpression; ZNF479 human tf ARCHS4 coexpression; ZFP91 human tf ARCHS4 coexpression; ZNF79 human tf ARCHS4 coexpression; ZNF682 human tf ARCHS4 coexpression; DEPDC1 human tf ARCHS4 coexpression; ZNF624 human tf ARCHS4 coexpression; TCF20 human tf ARCHS4 coexpression; GABPB1 human tf ARCHS4 coexpression; ZC3H11A human tf ARCHS4 coexpression; THAP1 human tf ARCHS4 coexpression; MYSM1 human tf ARCHS4 coexpression; BOLA3 human tf ARCHS4 coexpression; NOC3L human tf ARCHS4 coexpression; ZIM2 human tf ARCHS4 coexpression; ZNF57 human tf ARCHS4 coexpression; ZNF512B human tf ARCHS4 coexpression; NCOR2 human tf ARCHS4 coexpression; IRF4 20064451 ChIP-Seq CD4+ T Mouse; CBP 20019798 ChIP-Seq JUKART Human; CREM 20920259 ChIP-Seq GC1-SPG Mouse; E2F4 17652178 ChIP-ChIP JURKAT Human; ELF1 HCT116 hg19; POU5F1 16518401 ChIP-PET MESCs Mouse; JARIDIA 20064375 ChIP-Seq MESCs Mouse; CREB1 H1-hESC hg19; MYC 19079543 ChIP-ChIP MESCs Mouse; CREB1 HepG2 hg19; KAP1 22055183 ChIP-Seq ESCs Mouse; CUX1 19635798 ChIP-ChIP MULTIPLE HUMAN CANCER TYPES Human; STAT4 19710469 ChIP-ChIP THI Mouse; ZFX 18555785 ChIP-Seq MESCs Mouse; NANOG 16518401 ChIP-PET MESCs Mouse; STAT3 20064451 ChIP-Seq CD4+ T Mouse; REST 19997604 ChIP-ChIP NEURONS Mouse; RCOR1 19997604 ChIP-ChIP NEURONS Mouse; TRIM28 17542650 ChIP-ChIP NTERA2 Human; CREB1 15753290 ChIP-ChIP HEK293T Human.

1. A composition for promoting NR4A2 (nurr1) activity, CREB1 activity, NGF-independent TRKA activation, terpenoid backbone biosynthesis, or any combination thereof, in a cell, tissue, organ, subject, patient and/or organism,
wherein said composition comprises an aminopyridine, its derivatives, its metabolites, its analogs, and/or its isomers,
and
wherein the composition takes the form of a beverage, condiment, a nutritional drink, functional food, transdermal patch, topical formulation, aerosol, spray, nasal spray, solution, liquid, stable liquid, gel, suspension, polyethylene glycol-coated preparation, syrup, soft gel, emulsion, lotion, tablet, pill, pellet, capsule, sublingual composition, powder, sustained-release formulation, suppository, emulsion, aerosol, spray, drop, buccal or sublingual form, a transdermal patch, cream, cosmetic cream, lotion, ointment, shampoo, gum, troche, gummy, condiment, nutritional drink, polyethylene glycol-coated preparation, suspension, syrup, soft gel, topical formulation, nanoemulsion, nanoparticle preparation, powder mixture, topical gel, sunscreen, lozenge, cream, aqueous formulation, injectable formulation, sustained release formulation, or any combination thereof.

2. The composition of claim 1 wherein said composition is pleiotropic; wherein said composition provides multiple benefits selected from regeneration, recovery, repair, weight loss, reduced appetite, increased satiety, reduced obesity, reduced polypharmacy, amelioration of a condition, disease or disorder, or any combination thereof.

3. The composition of claim 1 further comprising l-arginine, a second agent, compound, natural compound, drug, or any combination thereof.

4. The composition of claim 1 wherein the composition ameliorates type 2 diabetes, promotes insulin secretion, signaling and processing, promotes glucose transport, reduces galactose catabolism promotes glucagon signaling, promotes glucagon like peptide regulating insulin secretion, promotes IRS activation in insulin signaling, promotes glucagon type ligand receptors, promotes orexin, neuropeptide FF and QRFP binding to their respective receptors, promotes glucose transport, or any combination thereof.

5. The composition of claim 1 wherein the composition promotes Leptin and Adiponectin signaling, promotes fatty acid metabolism, glycosphingolipid biosynthesis, promotes cholesterol biosynthesis, promotes biosynthesis of unsaturated fats, promotes SREBP signaling, promotes SREBF activation, or any combination thereof.

6. The composition of claim 1 wherein the composition promotes recovery from cardiac infarction, promotes nuclear receptors (especially for steroids and hormones which directly bind DNA and act as transcription factors), promotes PDGF pathway (postinfarction repair), regulates EGFR1 signaling (myocardial repair), regulates HSF1 mediated heat shock response, promotes G protein signaling, downregulates EGFR1 signaling, promotes nuclear receptors, promotes cardiac conduction, promotes transmission across gap junctions, promotes skeletal muscle hypertrophy, promotes muscle hypertrophy, promotes miRs in muscle differentiation, promotes transcriptional activation of mitochondrial biogenesis, or any combination thereof.

7. The composition of claim 1 wherein the composition, improves recovery from injury, improves cognition, promotes RSK activation (cognition learning) improved learning, improved memory, neuroprotection, neuoregeneration, ameliorates amyotrophic lateral sclerosis, ameliorates Parkinson's like signs/symptoms, ameliorates a psychiatric or neurodegenerative disorder, ameliorates Alzheimer's like signs/symptoms, promotes transmission across chemical synapses, regulates neurite outgrowth, promotes Ephrin signaling, promotes neurotransmission, promotes NGF independent trkA activation, promotes neurotrophin signaling, promotes NGF signaling via trkA, promotes BDNF signaling, or any combination thereof.

8. The composition of claim 1 wherein the composition promotes neurotransmitter release, promotes GABA synthesis, release, uptake and degradation, promotes GABA A receptor activation, promotes glutamate neurotransmitter release, promotes activation of kainate receptors upon glutamate binding, promotes presynaptic function of kainate receptors, promotes ADP signaling through P2Y purinoreceptor, promotes norepinephrine neurotransmitter release, reduces nicotinic activity on dopaminergic neurons, reduces activation of postsynaptic nicotinic receptors, promotes serotonin neurotransmission, promotes Serotonin Receptor 4/6/7 and NR3C Signaling, promotes dopaminergic neurotransmitter release, promotes acetylcholine neurotransmitter release, promotes acetylcholine signaling, reduces choline catabolism promotes MAPK activity, reduces prion disease and/or promotes metal SLC transporters, or any combination thereof.

9. The composition of claim 1 wherein the composition promotes IFN stimulated Antiviral mechanisms, reduces TNFR1-induced NFkappaB signaling pathway, regulates the cd28 dependent vav1 pathway, or any combination thereof.

10. The composition of claim 1 wherein the composition promotes terpenoid backbone biosynthesis and sesquiterpene activity, promotes Linoleic acid (LA) metabolism, or any combination thereof.

11. The composition of claim 1 wherein the composition promotes IFN stimulated Antiviral mechanisms, reduces TNFR1-induced NFkappaB signaling pathway, promotes prostacyclin signaling, or any combination thereof.

12. The composition of claim 1 wherein the composition ameliorates arthritis, promotes glycosaminoglycan biosynthesis, promotes hyaluronan synthesis, promotes chondroitin sulfate biosynthesis, promotes glycosaminoglycan biosynthesis, regulates osteoclast function, or any combination thereof.

13. The composition of claim 1 wherein the composition promotes POU5F1 (OCT4), SOX2, KLF4 and/or NANOG activation, genes related to proliferation and pluripotency.

14. The composition of claim 1 wherein the composition ameliorates cancer, reduces chemotherapeutic resistance, reduces Imatinib Resistance, reduces oncogene expression, integrated cancer pathway, inhibits basal cell carcinoma, reduces EGR1, reduces EGR2, regulates EGFR expression, regulates activation of NIMA receptors, and/or promotes TP53 regulation of transcription of death receptors and ligands, promotes PUMA activation and translocation to the mitochondria, promotes fas pathway, reduces oncostatin M signaling, reduces WNT signaling, downregulates Beta-catenin phosphorylation cascade, reduces Glioblastoma signaling pathways, downregulates Matrix Metalloproteinases, downregulates MFAP5-mediated ovarian cancer cell motility and invasiveness, reduces DNA replication, reduces Notch1 signaling, reduces Endometrial cancer pathways, downregulates Gastric Cancer Network pathways, downregulates viral carcinogenesis, downregulates thyroid cancer pathways, or any combination thereof.

15. The composition of claim 1 wherein the composition downregulates the extrinsic clot formation pathway, reduces clot formation, reduces blood clotting cascade, regulates estrogen biosynthesis, or any combination thereof.

16. The composition of claim 1 wherein the composition promotes selenium and selenoprotein metabolism, reduces cysteine and homocysteine degradation, reduces Melanogenesis, or any combination thereof.

17. The composition of claim 1 further comprising, an approved drug, an FDA approved drug, bicuculline, baclofen, zolpidem, progabide, azd 355, tramiprosate, gaboxadol, adipiplon, arbaclofen, placarbil, lesogaberan, muscimol, or any combination thereof.

18. The composition of claim 1 wherein the composition further regulates the expression of NR4A2 targets and or CREB1 targets, or any combination thereof.

19. The composition of claim 1 wherein the composition comprises at least one diluent, at least one stabilizer, at least one surfactant, at least one buffering agent, or any combination thereof.

20. The composition of claim 19, wherein said buffering agent is selected from the group comprising sodium biphosphate, potassium biphosphate, sodium bicarbonate, potassium bicarbonate, carboxylic acids and their salts, or any combination thereof.

21. The composition of claim 1, wherein said composition takes the form of nanoparticles, nanovaults, nanofibers, nanotubes, liposomes, nanoemulsion, or any combination thereof.

22. A method for promoting NR4A2 (nurr1) activity, CREB1 activity, NGF-independent TRKA activation, terpenoid backbone biosynthesis, or any combination thereof, in a cell, tissue, organ, subject, patient and/or organism,
wherein said method comprises treatment of said cell, tissue, organ, organism, individual, and/or patient with a composition comprising aminopyridine, its derivatives, its metabolites, its analogs, and/or its isomers,
and
wherein the composition takes the form of a beverage, condiment, a nutritional drink, functional food, transdermal patch, topical formulation, aerosol, spray, nasal spray, solution, liquid, stable liquid, gel, suspension, polyethylene glycol-coated preparation, syrup, soft gel, emulsion, lotion, tablet, pill, pellet, capsule, sublingual composition, powder, sustained-release formulation, suppository, emulsion, aerosol, spray, drop, buccal or sublingual form, a transdermal patch, cream, cosmetic cream, lotion, ointment, shampoo, gum, troche, gummy, condiment, nutritional drink, polyethylene glycol-coated preparation, suspension, syrup, soft gel, topical formulation, nanoemulsion, nanoparticle preparation, powder mixture, topical gel, sunscreen, lozenge, cream, aqueous formulation, injectable formulation, sustained release formulation, or any combination thereof.

23. The method of claim 22 wherein said composition is pleiotropic; wherein said composition provides multiple benefits selected from regeneration, recovery, repair, weight loss, reduced appetite, increased satiety, reduced obesity, reduced polypharmacy, amelioration of a condition, disease or disorder, or any combination thereof.

24. The method of claim 22 wherein the method further comprises treatment with a second agent, compound, natural compound, drug, or any combination thereof.

25. The method of claim 22 wherein the method further comprises chemotherapy or radiation therapy, or any combination thereof.

26. The composition of claim 1 wherein the composition comprises one or more second agent, compound or drug selected from a drug class represented by one or more of the following exemplars: Abacavir, Abametapir, Abarelix, Abatacept, Abciximab, Abilify, Abreva, Abrocitinib, Acalabrutinib, Acamprosate, Acarbose, Accolate, Accretropin, Acebutolol, Acellular, Acetaminophen, Acetate, Acetazolamide, Acetic, Acetohexamide, Acetohydroxamic, Acetonide, Acetrizoate, Aciphex, Aclidinium, Acrisorcin, Acrivastine, Actemra, Actiq, Activella, Actonel, Actoplus, Actos, Acular, Acuvail, Acyclovir, Adagrasib, Adalimumab, Adalimumab-aaty, Adalimumab-adbm, Adalimumab-afzb, Adalimumab-aqvh, Adalimumab-bwwd, Adalimumab-fkjp, Adapalene, Adcirca, Adderall, Adefovir, Aducanumab-avwa, Advicor, Advil, Aerolizer, Aerosol, Af, Afatinib, Afinitor, Aflibercept, Against, Agalsidase, Agenerase, Aggregated, Aggrenox, Agrylin, Ak-con-a, Akten, Alafenamide, Alamast, Albendazole, Albenza, Albumin, Albuterol, Alcohol, Aldara, Aldesleukin, Aldurazyme, Alefacept, Alendronate, Alesse, Alfacon-1, Alfa-tycv, Alfuzosin, Alirocumab, Alglucosidase, Alimta, Alinia, Aliskiren, Aliskiren+amlodipine, Alitretinoin, Allegra, Allegra-d, Allopurinol, Almotriptan, Alogliptin, Alora, Alosetron, Aloxi, Alphagan, Alphanine, Alprostadil, Alrex, Alseroxylon, Altabax, Altocor, Altretamine, Aluminum, Alvesco, Alvimopan, Amaryl, Ambrisentan, Amerge, Amevive, Amide, Amifampridine, Amifostine, Amiloride, Amino, Aminocaproic, Aminoglutethimide, Aminohippurate, Aminosalicylate, Aminosalicylic, Amisulpride, Amitiza, Amlexanox, Amlodipine, Amlodipine/atorvastatin, Ammonia, Amoxicillin/clavulanate, Amoxil, Amphetamine, Amodiaquine, Amoxicillin, Ampicillin/ampicillin, Amprenavir, Ampyra, Amrix, Anacaulase-bcdb, Anagrelide, Anastrozole, Androderm, Androgel, Aneuvysion, Anexsia, Angiomax, Angiotensin, Anidulafungin, Anifrolumab-fnia, Anisindione, Ansuvimab-zykl, Antazoline, Antihemophilic, Antithrombin, Antizol, Anzemet, Apalutamide, Aphthasol, Apixaban, Aplenzin, Apokyn, Apomorphine, Apraclonidine, Apremilast, Aprepitant, Apthasol, Aptivus, Arava, Arbutamine, Ardeparin, Aredia, Arestin, Arformoterol, Argatroban, Arginine, Aricept, Arimidex, Aripiprazole, Arixtra, Armodafinil, Aromasin, Arranon, Arsenic, Artemether, Artemether/lumefantrine, Arthrotec, Arzerra, Asacol, Asciminib, Ascorbate, Asenapine, Asfotase, Aspart, Aspirin, Astelin, Astepro, Atacand, Atazanavir, Atenolol, Atogepant, Atoltivimab, Atomoxetine, Atorvastatin, Atovaquone, Atracurium, Atridox, Atropine, Atrovent, Atryn, Augmentin, Auranofin, Avacopan, Avanafil, Avandamet, Avandia, Avastin, Avatrombopag, Avelox, Avinza, Avita, Avobenzone, Avonex, Axert, Axetil, Axid, Axona, Azacitidine, Azasite, Azatadine, Azathioprine, Azelaic, Azelastine, Azithromycin, Azmacort, Azor, Azt, Aztreonam, Azulfidine, Bacitracin, Baclofen, Bactroban, Baloxavir, Balsalazide, Banzel, Baraclude, Barium, Baycol, Bayer, Becaplermin, Beclomethasone, Belantamab, Belatacept, Bempedoic, Benazepril, Bendamustine, Benefix, Benicar, Benralizumab, Bentiromide, Benzamycin, Benzhydrocodone, Benzoate, Benzoyl, Benzyl, Bepotastine, Bepreve, Bepridil, Beractant, Berinert, Berotralstat, Besifloxacin, Besilate, Besivance, Besylate, Betamethasone, Betapace, Betaxolol, Betaxon, Bethanechol, Bevacizumab, Bevacizumab-awwb, Bevacizumab-bvzr, Bexarotene, Bextra, Bexxar, Biaxin, Bicarbonate, Bicisate, Bictegravir, Bicuculline, Bidil, Bimatoprost, Binimetinib, Biotin, Biperiden, Bisacodyl, Bismuth, Bisoprolol, Bisulfate, Bitartrate, Bivalent, Bivalirudin, Bleomycin, Boniva, Bortezomib, Bosentan, Botox, Botulinum, Bravelle, Bretylium, Brexpiprazole, Brigatinib, Brimonidine, Brincidofovir, Bromfenac, Brinzolamide, Bromide, Bromocriptine, Bromodiphenhydramine, Brovana, Buccal, Budesonide, Buffered, Bupivacaine, Buprenorphine, Buprenorphine/naloxone, Bupropion, Busulfan, Busulflex, Butabarbital, Butalbital, Butenafine, Butoconazole, Butorphanol, Butoxide, Butyrate, Byetta, C-13, Cabazitaxel, Cabergoline, Cabotegravir, Cabozantinib, Caduet, Cafcit, Caffeine, Calaspargase, Calcipotriene, Calcitonin, Calcitonin-salmon, Calcium, Calfactant, Cambia, Campath, Campostar, Campral, Camptosar, Camsylate, Canagliflozin, Canakinumab, Canasa, Cancidas, Candesartan, Candicidin, Capecitabine, Capreomycin, Caproate, Capromab, Caprylidene, Capsaicin, Capsule, Capsules, Captopril, Carbaglu, Carbamazepine, Carbatrol, Carbenicillin, Carbidopa, Carbon, Carbonate, Carboplatin, Cardizemâ®, Carglumic, Cariprazine, Carisoprodol, Carmustine, Carvedilol, Casimersen, Caverject, Cayston, Cea-scan, Cedax, Cedazuridine, Cefaclor, Cefamandole, Cefazolin, Cefdinir, Cefditoren, Cefiderocol, Cefotaxime, Cefoxitin, Cefpiramide, Cefprozil, Ceftaroline, Ceftazidime, Ceftibuten, Ceftin, Ceftolozane, Ceftriaxone, Cefuroxime, Celexa, Cellcept, Cellulose, Cemiplimab-rwlc, Cenestin, Cenobamate, Cephapirin, Cerivastatin, Cerliponase, Cernevit, Certolizumab, Cervarix, Cetirizine, Cetrotide, Cetuximab, Cevimeline, Chantix, Chenodiol, Chlophedianol, Chlorambucil, Chloramphenicol, Chloraprep, Chlordiazepoxide, Chlorhexidine, Chloroquine, Chlorothiazide, Chlorotrianisene, Chlorpheniramine, Chlorprothixene, Chlorthalidone, Cholestyramine, Choriogonadotropin, Chorionic, Chromate, Chromic, Chymopapain, Cialis, Ciclesonide, Ciclopirox, Cidofovir, Cilexetil, Cilostazol, Cimetadine, Cimetidine, Cimzia, Cinacalcet, Cinoxacin, Cinryze, Ciprofloxacin, Cisatracurium, Cisplatin, Citrate, Citric, Cladribine, Clarinex, Clarithromycin, Claritin, Claritin-d, Clascoterone, Clavulanate, Clemastine, Cleocin, Clevidipine, Cleviprex, Climara, Clindamycin, Clioquinol, Clobazam, Clobetasol, Clofarabine, Clolar, Clomiphene, Clomipramine, Clonazepam, Clonidine, Clopidogrel, Clostridium, Clotrimazole, Clotrimazole/betamethasone, Clozapine, Co-57, Co-58, Coagulation, Coartem, Cobicistat, Cocaine, Codeine, Colazal, Colchicine, Colcrys, Cold, Colesevelam, Colfosceril, Collagenase, Colloid, Combination, Combivir, Complete, Complex, Comtan, Concent, Concerta, Condylox, Confide, Conivaptan, Contraceptive, Copaxone, Corlopam, Corticorelin, Corticotropin-zinc, Cortisone, Corvert, Cosopt, Cosyntropin, Covera-hs, Cq, Cr, Cr-51, Crestor, Crinone, Crixivan, 20 Crizanlizumab-tmca, Cromolyn, Cryptenamine, Curosurf, Cuvposa, Cyanocobalamin, Cyclobenzaprine, Cyclophosphamide, Cycloset, Cyclosporine, Cyclothiazide, Cycrimine, Cylert, Cymbalta, Cypionate, Cysteamine, Cytovene, Dabigatran, Dabrafenib, Daclatasvir, Daclizumab, Dacogen, Dactinomycin, Dalbavancin, Dalfampridine, Danazol, Dantrolene, Dapagliflozin, Dapiprazole, Dapsone, Daptacel, Daptomycin, Daratumumab, Darbepoetin, Darolutamide, Darunavir, Dasabuvir, Dasatinib, Daunorubicin, Ddavp, Decanoate, Decitabine, Defibrotide, Deflazacort, Degarelix, Degludec, Delavirdine, Demecarium, Denosumab, Dentipatch, Depakote, Depot, Depreotide, Derisomaltose, Dermagel, Dermagraft-tc, Deruxtecan-nxki, Deserpidine, Deslanoside, Desloratadine, Desmopressin, Desogestrel, Desonate, Desonide, Desoximetasone, Detrol, Deucravacitinib, Dexamethasone, Dexbrompheniramine, Dexchlorpheniramine, Dexfenfluramine, Dexmethylphenidate, Dextromethorphan, Dextrose, Diclofenac, Didanosine, Differin, Difluprednate, Dihydrate, Dihydrochloride, Diltiazem, Dimesylate, Dinitrate/hydralazine, Diovan, Dipivoxil, Dipropionate, Disodium, Disoproxil, Ditropan, Divalproex, Docetaxel, Docosanol, Donepezil, Doribax, Doripenem, Dornase, Dostinex, Double, Doxepin, Doxercalciferol, Doxil, Doxorubicin, Doxycycline, Dronedarone, Drospirenone/ethinyl, Drotrecogin, Droxia, Dulera, Duloxetine, Duoneb, Durezol, Dutasteride, Dutasteride+tamsulosin, Dynabac, Dynacirc, Ec, Ecallantide, Eculizumab, Edex, Edluar, Efferdose, Effexor, Efient, Egrifta, Elaprase, Electrolyte/dextrose, Elestrin, Eletriptan, Elidel, Eligard, Elitek, Ella, Ellence, Elmiron, Eloxatin, Eltrombopag, Embeda, Emend, Enalapril, Enbrel, Enfuvirtide, Enoxaparin, en-tabs, Entecavir, Entereg, Entocort, Epivir, Eplerenone, Epogen, Eprosartan, Eraxis, Erbitux, Eribulin, Erlotinib, Erythromycin, Escitalopram, Esclim, Esomeprazole, Esterase, Estradiol, Estradiol/norethindrone, Estratab, Estrogel, Estrogens, Estrogens/medroxyprogesterone, Estrostep, Eszopiclone, Etanercept, Etexilate, Ethinyl, Ethinylestradiol+levonorgestrel, Ethyol, Etodolac, Etravirine, Eulexin, Evamist, Everolimus, Evista, Evolocumab, Evoxac, Evra, Exalgo, Excedrin, Exelon, Exenatide, Extavia, Extina, Ezetimibe, Fabrazyme, Factor/coagulation, Famciclovir, Famvir, Fanapt, Faslodex, Febuxostat, Felodipine, Femara, Femhrt, Fempatch, Femstat, Fenofibrate, Fentanyl, Feraheme, Feridex, Ferrlecit, Fertinex, Ferumoxytol, Fesoterodine, Fexofenadine, Finacea, Finevin, Fingolimod, Flagyl, Flomax, Flonase, Flovent, Floxin, Flumist, Fluorouracil/leucovorin, Fluoxetine, Flutamide, Fluticasone, Fluvastatin, Fluvoxamine, Fluzone, Foam, Focalin, Follistima, Follitropin, Folotyn, Foradil, Formoterol, Forteo, Fortovase, Fosamax, Fosamil, Fosamprenavir, Fospropofol, Fosrenol, Fragmin, Frova, Frovatnptan, Fsh, Fulvestrant, Fumarate, Furoate, Furoate+formoterol, Fusilev, Fuzeon, Gabapentin, Galantamine, Galsulfase, Galzin, Ganciclovir, Gardasil, Gastrocrom, Gastromark, Gatifloxacin, Gefitinib, Gelnique, Gemcitabine, Gemtuzumab, Gemzar, Genotropin, Geodon, Geref, Gilenya, Glargine, Gleevec, Gliadel, Glimepiride, Glipizide, Glipizide/metformin, Globulin, Glucagon, Gluconate, Glyburide, Glycol-epoetin, Glycopyrrolate, Glyset, Golimumab, Gonadotropin, Gonal-f, Goserelin, Granisetron, Grepafloxacin, Guanfacine, Halaven, Handihaler, Havrix, Hectorol, Hepsera, Herceptin, Hfa, Hiberix, Histolyticum, Histrelin, Humalog, Humatrope, Humira, Hycamtin, Hyclate, Hydrochlorothiazide, Hydrochlorotiazide, Hydrocodone, Hydromorphone, Hylan, Ibandronate, Ibritumomab, Ibuprofen, Ibutilide, Idursulfase, Ilaris, Iloperidone, Imagent, Imatinib, Imiquimod, Imitrex, Immune, Implant, Inactivated, Incobotulinumtoxina, Increlex, Indinavir, Infanrix, Infasurf, Infergen, Infliximab, Inform, Innohep, Inspra, Insulin, Integrilin, Intelence, Interferon, Interstim, Intuniv, Invanz, Invega, Invirase, Iodixanol, Iontocaine, Ipratropium, Iressa, Iron, Irrigating, Isentress, Isopropyl, Isosorbide, Istodax, Itraconazole, Ivermectin, Ivyblock, Ixabepilone, Ixempra, Ixiaro, Jalyn, Januvia, Jevtana, Kadian, Kalbitor, Kaletra, Kapvay, Keppra, Ketek, Ketoconazole, Ketoprofen, Ketorolac, Kineret, Kit, Klaron, Kogenate, Krystexxa, Kunecatechins, Kuvan, Kytril, Lacosamide, Lamictal, Lamin, Lamisil, Lamivudine, Lamotrigine, Lanreotide, Lansopraxole, Lanthanum, Lantus, Lapatinib, Laronidase, Latuda, Lenalidomide, Lescol, Letairis, Letrozole, Leukine, Leuprolide, Levaquin, Levitra, Levocetirizine, Levofloxacin, Levoleucovorin, Levonorgestrel/20, Levonorgestrel-releasing, Levo-t, Levothyroxine, Levoxyl, Lexapro, Lexidronam, Lexiva, Lexxel, Lidocaine, Lidoderm, Liraglutide, Lisdexamfetamine, Lisinopril, Lispro, Lithium, Lithobid, Livalo, Lodine, Loratadine, Lotemax, Lotion, Lotrisone, Lotronex, Lovastatin, Lovenox, Lubiprostone, Lucentis, Lumigan, Lunesta, Lupron, Lurasidone, Lusedra, Lustra, Luvox, Luxiq, Lyrica, Lysteda, Macugen, Malarone, Maleate/diltiazem, Maraviroc, Marplan, Mavik, Maxalt, Mecasermin, Medoxomil, Medoxomil+amlodipine+hydrochlorothiazide, Meloxicam, Memantine, Menotropins, Mentax, Menveo, Meridia, Meropenem, Merrem, Mesalamine, Mesnex, Mesylate, Metadate, Metaglip, Metaprotereol, Metformin, Methylphenidate, Metoclopramide, Metoprolol, Metozolv, Metrolotion, Mevacor, Mg, Miacalcin, Micardis, Miconazole, Microspheres, Microzide, Midazolam, Midodrine, Miglitol, Migranal, Milnacipran, Minocycline, Minoxidil, Miraluma, Mirapex, Mircera, Mircette, Mirena, Mirtazapine, Mitoxantrone, Mobic, Mometasone, Monistat, Monurol, Monviala®, Morphine, Motrin, Mousse, Moxatag, Moxifloxacin, Mozobil, Multaq, Mupirocin, Muse, Mylotarg, Myobloc, Myozyme, Naglazyme, Naltrexone, Namenda, Naphazoline, Naprelan, Naproxen, Naproxen+esomeprazole, Nasacort, Nasal, Nasalcrom, Nascobal, Nasonex, Natalizumab, Natazia, Natrecor, Nedocromil, Nelarabine, Nelfinavir, Nesiritide, Neulasta, Neumega, Neupogen, Neupro, Neurontin, Nevirapine, Nexavar, Nexium, Niacin/lovastatin, Niaspan, Nicoderm, Nicorette, Nicotine, Nicotrol, Nilotinib, Nitazoxanide, Nitisinone, Nitrate, Nitroglycerin, Nitrostat, Nizatidine, Nolvadex, Norco, Norditropin, Norethindrone, Norgestimate/ethinyl, Noritate, Normiflo, Norvir, Novantrone, Novolog, Novothyrox, Noxafil, Nplate, Nuedexta, Nutropin, Nuvaring, Nuvigil, Ocuflox, Ocuhist, Odt, Ofatumumab, Ofloxacin, Oleptro, Olmesartan, Olopatadine, Omalizumab, Omeprazole, Omnicef, Onabotulinumtoxina, Ondansetron, Onglyza, Onsolis, Ophthalmic, Opthalmic, Oravig, Orencia, Orfadin, prefest, Osi, Osmocyte, Otic, Ovidrel, Oxaliplatin/5-, Oxcarbazepine, Oxybate, Oxybutynin, Oxycodone, Oxycontin, Oxytrol, Ozogamicin, Ozurdex, Paliperidone, Palonosetron, Pamidronate, Pamoate, Pancreaze, Pancrelipase, Panitumumab, Panretin, Pantoprazole, Paroxetine, Patanase, Patch, Paxil, Pazopanib, PCSK9 inhibitor, Pediarix, Pegaptanib, Pegasys, Peginterferon, Pegloticase, Pegol, Pegvisomant, Pemetrexed, Pennsaid, Pentavalent, Pentosan, Pentoxifylline, Pepcid, Perflexane, Periostat, Phoslo, Phosphate, Photofrin, Pindolol, Pioglitazone, Piperacillin, Pitavastatin, Plavix, Plenaxis, Plerixafor, Pneumococcal, Pokofilox, Polacrilex, Polifeprosan, Polyethylene, Polysulfate, Posaconazole, Posicor, Pradaxa, Pralatrexate, Praluent, Pramipexole, Pramlintide, Prandin, Prasugrel, Pravachol, Pravastatin, Precose, Pregabalin, Premarin, Premphase, Prempro, Prevacid, Preven, Prevnar, Prevpac, Prezista, Priftin, Prilosec, Prinivil, Proamatine, Procainamide, Procanbid, Prochloroperazine, Prochlorperazine, Progabide, Progesterone, Prograf, Proguanil, Proleukin, Prolia, Promacta, Prometrium, Propecia, Proscar, Protonix, Protopic, Provenge, Proventil, Prozac, Pseudoephedrine, Pulmozyme, Quadramet, Quadrivalent, Quetiapine, Quinidine, Quixin, Qutenza, Qvar, Rabeprazole, Raloxifene, Raltegravir, Ramelteon, Ranexa, Ranibizumab, Ranitidine, Ranolazine, Rapamune, Raplon, Rasburicase, Raxar, Rdna, Rebetol, Rebetrona,¢, Rebif, Reclast, Recombinant, Reconstitution, Reditabs, Reduction, Redux, Refludan, Regranex, Release, Relenza, Relpax, Remeron, Remicade, Reminyl, Remodulin, Renagel, Renagelrenagel, Renova, Renvela, Reopro, Repatha, Repro, Requip, Rescriptor, Rescula, Respigam, Respiratory, Restasis, Retapamulin, Retavase, Reteplase, Retin-a, Revlimid, Reyataz, Rhinocort, Ribavirin, Rid, Rifaximin, Right, Rilutek, Riluzole, Risperdal, Ritalin, Ritonavir, Rituxan, Rivastigmine, Rizaben, Rocephin, Rofecoxib, Romidepsin, Romiplostim, Ropinirole, Rosiglitazone, Rosuvastatin, Rotadisk, Rotarix, Rotateq, Rotavirus, Rotigotine, Rozerem, Rufinamide, Rx, Rythmol, Sabril, Saizen, Salagen, Salts, Samarium, Samsca, Sanctura, Sancuso, Saphris, Sapropterin, Saquinavir, Sargramostim, Savella, Saxagliptin, Sclerosol, Sd, Seasonale, Seasonique, Secreflo, Secretin, Selegiline, Self-examination, Selzentry, Sensipar, Seprafilm, Serevent, Sermorelin, Seroquelâ®, Serpacwa, Sertraline, Sevelamer, Silenor, Simponi, Simulect, Single-entity, Singulair, Sipuleucel-t, Sirolimus, Sitagliptin, Skelid, Sodium, Sodium/tazobactam, Solifenacin, Soliris, Soltab, Soluble, Solution, Somatropin, Somatropin-rdna, Somatuline, Somavert, Sonata, Sorafenib, Sparfloxacin, Spectracef, Spiriva, Sporanox, Spray, Sprix, Sprycel, Stavudine, Stavzor, Stelara, Sterile, Strattera, Strength, Stromectol, Subutex/suboxone, Succinate, Sucrose, Sulfacet, Sulfamylon, Sulfasalazine, Sulfate, Sumatriptan, Sunitinib, Supartz, Supprelin, Suspension, Sustained-release, Sustiva, Sutent, Symlin, Synagis, Syncitial, Synercid, Synthroid, Synvisc, Synvisc-one, T4, Tacrolimus, Tadalafil, Tamiflu, Tarceva, Tartrate, Tasigna, Tasmar, Tavist, Taxol, Taxotere, Tazorac, Teczem, Teflaro, Tegaserod, Tegretol, Tekamlo, Tekturna, Telavancin, Telbivudine, Telithromycin, Telmisartan, Temodar, Temsirolimus, Tenofovir, Tequin, Terbinafine, Teriparatide, Tesamorelin, Test, Testim, Testoderm, Testosterone, Tetanus, Tetrabenazine, Teveten, Thalomid, Therapy, Tiazac, Tigecycline, Tikosyn, Tilade, Tiludronate, Timentin, Tindamax, Tinidazole, Tinzaparin, Tiotropium, Tipranavir, Tiuxetan, Tizanidine, Tobi, Tocilizumab, Tolmetin, Tolterodine, Tolvaptan, Topamax, Topical, Topiramate, Topotecan, Toprol-xl, Torisel, Toviaz, Toxin, Toxoid, Toxoids, Tracleer, Tramadol, Trandolapril, Tranexamic, Tranilast, Transdermal, Transoral, Trastuzumab, Travatan, Travoprost, Trazadone, Trazodone, Treanda, Trelstar, Treprostinil, Tretinoin, Triamcinolone, Tribenzor, Tricor, Tri-cyclen, Trileptal, Trilipix, Tri-nasal, Trioxide, Tripedia, Triptorelin, Trisenox, Trivagizole, Trivora-21, Trivora-28, Trizivir, Tromethamine, Trospium, Trovan, Tts, Twinrix, Tygacil, Tykerb, Type, Types, Tysabri, Tyvaso, Tyzeka, Ulipristal, Uloric, Ultracet, Ultraject, Unoprostone, Urofollitropin, Uroxatral, Urso, Usp, Ustekinumab, Uvadex, Vaccine, Vaginal, Valacyclovir, Valcyte, Valerate, Valerate+dienogest, Valganciclovir, Valproic, Valsartan, Valstar, Valtrex, Vancenase, Vanceril, Vaprisol, Vardenafil, Varenicline, Vectibix, Velaglucerase, Velcade, Veltin, Venlafaxin, Venofer, Ventolin, Veramyst, Verapamil, Verdeso, Veregen, Versed, Verteporfin, Vesicare, Vfend, Viadur, Viagra, Vibativ, Victoza, Vidaza, Videx, Vigabatrin, Viii, Vimovo, Vimpat, Vioxx, Viracept, Viramune, Viread, Viroptic, Virus, Visicol, Visipaque, Vistide, Visudyne, Vitrasert, Vitravene, Vivelle, Vivelle-dot, Vivitrol, Von, Voriconazole, Votrient, Vpriv, Vyvanse, Wafer, Warfare, Warfarin, Weekly, Welchol, Western, Wilate, Willebrand, With, Women, Wound, Xeloda, Xenazine, Xenical/orlistat, Xeomin, Xgeva, Xiaflex, Xifaxan, Xigris, Xolair, Xopenex, Xr, Xyrem, Xyzal, Yasmin, Zaditor, Zagam, Zanaflex, Zantac, Zelnorm, Zemaira, Zemplar, Zenapax, Zenpep, Zerit, Zestril, Zevalin, Zidovudine, Zileuton, Zinc, Ziprasidone, Zipsor, Zirgan, Zithromax, Zocor, Zofran, Zoladex, Zoledronic, Zolmitriptan, Zoloft, Zolpidem, Zometa, Zomig, Zonegran, Zonisamide, Zortress, Zosyn, Zuplenz, Zyban, Zyclara, Zyflo, Zymaxid, Zyprexa, Zyrtec, or any combination thereof.

27. The composition of claim 1 wherein the composition comprises one or more second agent, drug, compound or extract derived from an oil or extract in the classes represented by the following exemplars: Agarwood; Agarwood; Almond, Aloe Vera; Bitter; Amber Oil, Avocado, Fossilized; Amber Oil, Fossilized; Ambrette Seed Fine; Ambrette Seed; Amyris; Angelica Root; Angelica Seed; arborvitae; Armoise (Mugwort); Balsam of Peru Oil; Balsam of Peru Resin; Basil, Sweet ct Linalool; Basil, Sweet ct Linalool; Basil, Sweet ct Methyl Chavicol; Beeswax Absolute; Bergamot; Bergamot k; Bergamot; Bergamot; Black Cumin; Black Currant; Caraway; Cardamom; Carnation Absolute; Carnation Extract; Carrot Seed; Cassie Absolute; Cedarwood, Atlas; Cedarwood, Himalayan; Cedarwood, Texas; Cedarwood, Virginia; Celery Seed; Chamomile, Blue; Chamomile, Roman; Champaca; Cilantro; Cinnamon; Cinnamon Bark; Cistus Traditional; Citronella; Citronella Wild; Clary Sage Absolute; Clary Sage, Bulgaria; Clary Sage, Russia; Clary Sage, USA; Clove Bud; Clove; Cocao Absolute; Coconut; Coffee Bean; Coffee Bean Oil; Cognac, Green; Coriander Seed; Coriander Seed; Cucumber Hydrosol; Cumin Seed; Cypress Leaf; Cypress, Blue; Davana; Eucalyptus, Blue Gum; Eucalyptus, Blue Mallee; Eucalyptus, Lemon; Eucalyptus, Narrow Leaf; Eucalyptus, Narrow Leaf; Fennel, Sweet; Fennel, Sweet; Fenugreek; Fir Needle; Fir, Balsam; Fir, Balsam Absolute; Fir, Balsam Absolute; Fir, Douglas; Fir, Silver; Frankincense; Frankincense, Somalia; Frankincense Frereana; Frankincense, Oman; Frankincense, Oman Rare; Frankincense, Somalia; Galbanum; Geranium Absolute; Geranium, Egypt; Geranium, Rose; Geranium, South Africa; Ginger; Ginger; Ginger; Ginger Lily; Ginger, Fresh; Goji; Grapefruit, Pink; Grapefruit, Ruby Red; Grapefruit, White; Hay Absolute; Helichrysum, Albania; Helichrysum, Croatia; Hemp; Hyssop Decumbens; Immortelle Absolute; Jasmine Absolute, Egypt; Jasmine Absolute, Egypt; Jasmine Absolute; Jasmine Absolute; Jasmine; Jasmine Concrete; Jasmine Extract; Jasmine Sambac Absolute; Jasmine Sambac Absolute; Juniper Berry; Juniper Berry; Juniper Leaf/Berry, Nepal; Juniper Leaf/Branch; Kava Kava; Kava Kava; Labdanum Absolute, Clear; Laurel Leaf; Lavandin, Grosso; Lavender High Elevation; Lavender Wild; Lavender Absolute; Lavender Hydrosol; Lavender, Bulgaria; Lavender, France; Lavender, Maillette; Lemon; Lemon Tea Tree; Lemongrass; Lemongrass Wild; Lime Distilled; Lime Expressed; Lime Essence Oil; Lime, Distilled; Liquidambar (Styrax; Lotus Absolute, Pink; Lotus Absolute, White; Availability; Mandarin, Green; Mandarin, Red; Mandarin, Yellow; Mango' Marjoram; Melaleuca; Melissa; Myrrh; Myrrh, Somalia; Myrrh, Somalia; Myrtle, Green; Nagarmotha (Cypriol; Neroli Extra; Neroli Extra; Neroli, Egypt; Neroli, Egypt; Neroli, France; Neroli, France; Neroli, Morocco; Niaouli; Oakmoss Absolute; Orange Wild; Orange Blossom Absolute; Orange Blossom Absolute Fine; Orange Blossom Extract; Orange Essence Oil; Orange, Bitter Green; Orange, Bitter Red; Orange, Blood; Orange, Wild; Orange, Sweet; Oregano, Turkey; Orris Butter (15 irones; Osmanthus Absolute; Palmarosa, Nepal Wild; Palmarosa, Sri Lanka; Palo Santo; Patchouli Double Distilled; Patchouli; Patchouli, Dark; Patchouli, Light; Patchouli, Sri Lanka; Pepper, Black; Peppercorn, Pink; Peppermint, Chocolate; Peppermint, France; Peppermint; Peppermint, USA; Petitgrain Absolute; Petitgrain Bigarade; Petitgrain sur Fleurs; Petitgrain, Mandarin; Petitgrain, Mandarin; Piper, Aduncum; Piper, malacophyllum; Pomegranate Seed; Ravensara Wild; Rhododendron Leaf; Rosalina; Rose Absolute, Bulgaria; Rose Absolute, Bulgaria; Rose Absolute, Egypt; Rose Absolute, Egypt; Rose Absolute, Morocco; Rose Absolute, Morocco; Rose de Mai Absolute; Rose de Mai Concrete; Rose de Mai Extract; Rose Hip Seed; Rose Otto, Bulgaria; Rose Otto, Turkey; Rose Otto, White; Rosemary Antioxidant; Rosemary ct Cineole; Rosemary ct Cineole; Rosemary ct Verbenone; Sage; Sandalwood Rare; Sandalwood Absolute, New Caledonia; Sandalwood, Australian Premium; Sandalwood, New Caledonia; Sandalwood, New Caledonia Extra; Sandalwood, Royal Hawaiian; Sea Buckthorn; Seaweed Absolute; Spearmint; Spearmint, USA; Spikenard; Spikenard, Green Wild; Spruce, Black; St. John's Wort; Tagetes; Tamanu (Foraha Oil; Tangerine Murcott; Tansy, Blue; Tea Tree; Thyme ct Linalool; Tobacco Absolute; Tonka Bean Absolute; Tonka Bean Absolute 20; Tuberose Absolute; Tuberose Extract; Turmeric; Turmeric; Vanilla Absolute; Vanilla Bourbon; Vanilla Bourbon; Vanilla Bourbon; Verbena; Vetiver Double Distilled; Vetiver, El Salvador; Vernonia, polyanthes; Vetiver, Haiti; Vetiver, Sri Lanka; Violet Leaf Absolute; Violet Leaf Absolute; Virola, surinamensis; Vitamin E Oil; White Sage; Wintergreen Wild; Yarrow, Blue; Ylang Ylang Absolute; Ylang Ylang Complete, Comoros; Ylang Ylang Extra; Ylang Ylang I; Ylang Ylang II; Ylang Ylang III; Ylang Ylang, Fine; and Yuzu, or any combination thereof.
